SEQUENCE LISTING (1009 sequences)

SEQ ID NO: 1 - protein, 546 aa, synthetic construct
  REGION 1..546: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 2 - protein, 26 aa, synthetic construct
  REGION 1..26: note = Recombinant collagen fragment
AAGARGNDGARGSDGQPGPPGPPGTA

SEQ ID NO: 3 - protein, 35 aa, synthetic construct
  REGION 1..35: note = Recombinant collagen fragment
AAGARGNDGARGSDGQPGPPGPPGTAGFPGSPGAK

SEQ ID NO: 4 - protein, 41 aa, synthetic construct
  REGION 1..41: note = Recombinant collagen fragment
AAGARGNDGARGSDGQPGPPGPPGTAGFPGSPGAKGEVGPA

SEQ ID NO: 5 - protein, 21 aa, synthetic construct
  REGION 1..21: note = Recombinant collagen fragment
AAGEPGRDGVPGGPGMRGMPG

SEQ ID NO: 6 - protein, 27 aa, synthetic construct
  REGION 1..27: note = Recombinant collagen fragment
AAGEPGRDGVPGGPGMRGMPGSPGGPG

SEQ ID NO: 7 - protein, 17 aa, synthetic construct
  REGION 1..17: note = Recombinant collagen fragment
AAGERGAPGFRGPAGPN

SEQ ID NO: 8 - protein, 13 aa, synthetic construct
  REGION 1..13: note = Recombinant collagen fragment
AGAQGPPGPPGIN

SEQ ID NO: 9 - protein, 34 aa, synthetic construct
  REGION 1..34: note = Recombinant collagen fragment
AGAQGPPGPPGINGSPGGKGEMGPAGIPGAPGLM

SEQ ID NO: 10 - protein, 25 aa, synthetic construct
  REGION 1..25: note = Recombinant collagen fragment
AGARGNDGARGSDGQPGPPGPPGTA

SEQ ID NO: 11 - protein, 40 aa, synthetic construct
  REGION 1..40: note = Recombinant collagen fragment
AGARGNDGARGSDGQPGPPGPPGTAGFPGSPGAKGEVGPA

SEQ ID NO: 12 - protein, 26 aa, synthetic construct
  REGION 1..26: note = Recombinant collagen fragment
AGEPGRDGVPGGPGMRGMPGSPGGPG

SEQ ID NO: 13 - protein, 16 aa, synthetic construct
  REGION 1..16: note = Recombinant collagen fragment
AGERGAPGFRGPAGPN

SEQ ID NO: 14 - protein, 10 aa, synthetic construct
  REGION 1..10: note = Recombinant collagen fragment
AGIPGAPGLM

SEQ ID NO: 15 - protein, 28 aa, synthetic construct
  REGION 1..28: note = Recombinant collagen fragment
AGIPGAPGLMGARGPPGPAGANGAPGLR

SEQ ID NO: 16 - protein, 12 aa, synthetic construct
  REGION 1..12: note = Recombinant collagen fragment
AGIPGFPGMKGH

SEQ ID NO: 17 - protein, 14 aa, synthetic construct
  REGION 1..14: note = Recombinant collagen fragment
AGIPGFPGMKGHRG

SEQ ID NO: 18 - protein, 15 aa, synthetic construct
  REGION 1..15: note = Recombinant collagen fragment
AGIPGFPGMKGHRGF

SEQ ID NO: 19 - protein, 19 aa, synthetic construct
  REGION 1..19: note = Recombinant collagen fragment
AGIPGFPGMKGHRGFDGRN

SEQ ID NO: 20 - protein, 31 aa, synthetic construct
  REGION 1..31: note = Recombinant collagen fragment
AGIPGFPGMKGHRGFDGRNGEKGETGAPGLK

SEQ ID NO: 21 - protein, 34 aa, synthetic construct
  REGION 1..34: note = Recombinant collagen fragment
AGIPGFPGMKGHRGFDGRNGEKGETGAPGLKGEN

SEQ ID NO: 22 - protein, 25 aa, synthetic construct
  REGION 1..25: note = Recombinant collagen fragment
AGKDGESGRPGRPGERGLPGPPGIK

SEQ ID NO: 23 - protein, 28 aa, synthetic construct
  REGION 1..28: note = Recombinant collagen fragment
AGKDGESGRPGRPGERGLPGPPGIKGPA

SEQ ID NO: 24 - protein, 13 aa, synthetic construct
  REGION 1..13: note = Recombinant collagen fragment
AGPPGPPGPPGTS

SEQ ID NO: 25 - protein, 15 aa, synthetic construct
  REGION 1..15: note = Recombinant collagen fragment
AGPPGPPGPPGTSGH

SEQ ID NO: 26 - protein, 16 aa, synthetic construct
  REGION 1..16: note = Recombinant collagen fragment
AGPSGPPGPPGAIGPS

SEQ ID NO: 27 - protein, 43 aa, synthetic construct
  REGION 1..43: note = Recombinant collagen fragment
AGPSGPPGPPGAIGPSGPAGKDGESGRPGRPGERGLPGPPGIK

SEQ ID NO: 28 - protein, 55 aa, synthetic construct
  REGION 1..55: note = Recombinant collagen fragment
AGSPGSNGAPGQRGEPGPQGHAGAQGPPGPPGINGSPGGKGEMGPAGIPGAPGLM

SEQ ID NO: 29 - protein, 19 aa, synthetic construct
  REGION 1..19: note = Recombinant collagen fragment
AGYPGPAGPPGPPGPPGTS

SEQ ID NO: 30 - protein, 55 aa, synthetic construct
  REGION 1..55: note = Recombinant collagen fragment
AGYPGPAGPPGPPGPPGTSGHPGSPGSPGYQGPPGEPGQAGPSGPPGPPGAIGPS

SEQ ID NO: 31 - protein, 32 aa, synthetic construct
  REGION 1..32: note = Recombinant collagen fragment
AIGPSGPAGKDGESGRPGRPGERGLPGPPGIK

SEQ ID NO: 32 - protein, 27 aa, synthetic construct
  REGION 1..27: note = Recombinant collagen fragment
AKGEVGPAGSPGSNGAPGQRGEPGPQG

SEQ ID NO: 33 - protein, 41 aa, synthetic construct
  REGION 1..41: note = Recombinant collagen fragment
AKGEVGPAGSPGSNGAPGQRGEPGPQGHAGAQGPPGPPGIN

SEQ ID NO: 34 - protein, 26 aa, synthetic construct
  REGION 1..26: note = Recombinant collagen fragment
ANGAPGLRGGAGEPGKNGAKGEPGPR

SEQ ID NO: 35 - protein, 23 aa, synthetic construct
  REGION 1..23: note = Recombinant collagen fragment
ANGLPGAAGERGAPGFRGPAGPN

SEQ ID NO: 36 - protein, 17 aa, synthetic construct
  REGION 1..17: note = Recombinant collagen fragment
APGERGRPGLPGAAGAR

SEQ ID NO: 37 - protein, 38 aa, synthetic construct
  REGION 1..38: note = Recombinant collagen fragment
APGERGRPGLPGAAGARGNDGARGSDGQPGPPGPPGTA

SEQ ID NO: 38 - protein, 14 aa, synthetic construct
  REGION 1..14: note = Recombinant collagen fragment
APGLKGENGLPGEN

SEQ ID NO: 39 - protein, 23 aa, synthetic construct
  REGION 1..23: note = Recombinant collagen fragment
APGLKGENGLPGENGAPGPMGPR

SEQ ID NO: 40 - protein, 14 aa, synthetic construct
  REGION 1..14: note = Recombinant collagen fragment
APGLMGARGPPGPA

SEQ ID NO: 41 - protein, 17 aa, synthetic construct
  REGION 1..17: note = Recombinant collagen fragment
APGLMGARGPPGPAGAN

SEQ ID NO: 42 - protein, 23 aa, synthetic construct
  REGION 1..23: note = Recombinant collagen fragment
APGLMGARGPPGPAGANGAPGLR

SEQ ID NO: 43 - protein, 26 aa, synthetic construct
  REGION 1..26: note = Recombinant collagen fragment
APGQRGEPGPQGHAGAQGPPGPPGIN

SEQ ID NO: 44 - protein, 11 aa, synthetic construct
  REGION 1..11: note = Recombinant collagen fragment
AQGPPGPPGIN

SEQ ID NO: 45 - protein, 15 aa, synthetic construct
  REGION 1..15: note = Recombinant collagen fragment
AQGPPGPPGINGSPG

SEQ ID NO: 46 - protein, 17 aa, synthetic construct
  REGION 1..17: note = Recombinant collagen fragment
AQGPPGPPGINGSPGGK

SEQ ID NO: 47 - protein, 23 aa, synthetic construct
  REGION 1..23: note = Recombinant collagen fragment
AQGPPGPPGINGSPGGKGEMGPA

SEQ ID NO: 48 - protein, 35 aa, synthetic construct
  REGION 1..35: note = Recombinant collagen fragment
AQGPPGPPGINGSPGGKGEMGPAGIPGAPGLMGAR

SEQ ID NO: 49 - protein, 23 aa, synthetic construct
  REGION 1..23: note = Recombinant collagen fragment
ARGNDGARGSDGQPGPPGPPGTA

SEQ ID NO: 50 - protein, 25 aa, synthetic construct
  REGION 1..25: note = Recombinant collagen fragment
ARGNDGARGSDGQPGPPGPPGTAGF

SEQ ID NO: 51 - protein, 38 aa, synthetic construct
  REGION 1..38: note = Recombinant collagen fragment
ARGNDGARGSDGQPGPPGPPGTAGFPGSPGAKGEVGPA

SEQ ID NO: 52 - protein, 44 aa, synthetic construct
  REGION 1..44: note = Recombinant collagen fragment
ARGNDGARGSDGQPGPPGPPGTAGFPGSPGAKGEVGPAGSPGSN

SEQ ID NO: 53 - protein, 17 aa, synthetic construct
  REGION 1..17: note = Recombinant collagen fragment
ARGPPGPAGANGAPGLR

SEQ ID NO: 54 - protein, 26 aa, synthetic construct
  REGION 1..26: note = Recombinant collagen fragment
ARGPPGPAGANGAPGLRGGAGEPGKN

SEQ ID NO: 55 - protein, 17 aa, synthetic construct
  REGION 1..17: note = Recombinant collagen fragment
ARGSDGQPGPPGPPGTA

SEQ ID NO: 56 - protein, 26 aa, synthetic construct
  REGION 1..26: note = Recombinant collagen fragment
ARGSDGQPGPPGPPGTAGFPGSPGAK

SEQ ID NO: 57 - protein, 32 aa, synthetic construct
  REGION 1..32: note = Recombinant collagen fragment
ARGSDGQPGPPGPPGTAGFPGSPGAKGEVGPA

SEQ ID NO: 58 - protein, 23 aa, synthetic construct
  REGION 1..23: note = Recombinant collagen fragment
DAGAPGAPGGKGDAGAPGERGPP

SEQ ID NO: 59 - protein, 23 aa, synthetic construct
  REGION 1..23: note = Recombinant collagen fragment
DGAPGKNGERGGPGGPGPQGPPG

SEQ ID NO: 60 - protein, 25 aa, synthetic construct
  REGION 1..25: note = Recombinant collagen fragment
DGAPGKNGERGGPGGPGPQGPPGKN

SEQ ID NO: 61 - protein, 27 aa, synthetic construct
  REGION 1..27: note = Recombinant collagen fragment
DGAPGKNGERGGPGGPGPQGPPGKNGE

SEQ ID NO: 62 - protein, 29 aa, synthetic construct
  REGION 1..29: note = Recombinant collagen fragment
DGAPGKNGERGGPGGPGPQGPPGKNGETG

SEQ ID NO: 63 - protein, 30 aa, synthetic construct
  REGION 1..30: note = Recombinant collagen fragment
DGAPGKNGERGGPGGPGPQGPPGKNGETGP

SEQ ID NO: 64 - protein, 31 aa, synthetic construct
  REGION 1..31: note = Recombinant collagen fragment
DGAPGKNGERGGPGGPGPQGPPGKNGETGPQ

SEQ ID NO: 65 - protein, 32 aa, synthetic construct
  REGION 1..32: note = Recombinant collagen fragment
DGAPGKNGERGGPGGPGPQGPPGKNGETGPQG

SEQ ID NO: 66 - protein, 35 aa, synthetic construct
  REGION 1..35: note = Recombinant collagen fragment
DGAPGKNGERGGPGGPGPQGPPGKNGETGPQGPPG

SEQ ID NO: 67 - protein, 36 aa, synthetic construct
  REGION 1..36: note = Recombinant collagen fragment
DGAPGKNGERGGPGGPGPQGPPGKNGETGPQGPPGP

SEQ ID NO: 68 - protein, 38 aa, synthetic construct
  REGION 1..38: note = Recombinant collagen fragment
DGAPGKNGERGGPGGPGPQGPPGKNGETGPQGPPGPTG

SEQ ID NO: 69 - protein, 40 aa, synthetic construct
  REGION 1..40: note = Recombinant collagen fragment
DGAPGKNGERGGPGGPGPQGPPGKNGETGPQGPPGPTGPG

SEQ ID NO: 70 - protein, 41 aa, synthetic construct
  REGION 1..41: note = Recombinant collagen fragment
DGAPGKNGERGGPGGPGPQGPPGKNGETGPQGPPGPTGPGG

SEQ ID NO: 71 - protein, 17 aa, synthetic construct
  REGION 1..17: note = Recombinant collagen fragment
DGARGSDGQPGPPGPPG

SEQ ID NO: 72 - protein, 19 aa, synthetic construct
  REGION 1..19: note = Recombinant collagen fragment
DGARGSDGQPGPPGPPGTA

SEQ ID NO: 73 - protein, 20 aa, synthetic construct
  REGION 1..20: note = Recombinant collagen fragment
DGARGSDGQPGPPGPPGTAG

SEQ ID NO: 74 - protein, 21 aa, synthetic construct
  REGION 1..21: note = Recombinant collagen fragment
DGARGSDGQPGPPGPPGTAGF

SEQ ID NO: 75 - protein, 23 aa, synthetic construct
  REGION 1..23: note = Recombinant collagen fragment
DGARGSDGQPGPPGPPGTAGFPG

SEQ ID NO: 76 - protein, 28 aa, synthetic construct
  REGION 1..28: note = Recombinant collagen fragment
DGARGSDGQPGPPGPPGTAGFPGSPGAK

SEQ ID NO: 77 - protein, 29 aa, synthetic construct
  REGION 1..29: note = Recombinant collagen fragment
DGARGSDGQPGPPGPPGTAGFPGSPGAKG

SEQ ID NO: 78 - protein, 34 aa, synthetic construct
  REGION 1..34: note = Recombinant collagen fragment
DGARGSDGQPGPPGPPGTAGFPGSPGAKGEVGPA

SEQ ID NO: 79 - protein, 35 aa, synthetic construct
  REGION 1..35: note = Recombinant collagen fragment
DGARGSDGQPGPPGPPGTAGFPGSPGAKGEVGPAG

SEQ ID NO: 80 - protein, 38 aa, synthetic construct
  REGION 1..38: note = Recombinant collagen fragment
DGARGSDGQPGPPGPPGTAGFPGSPGAKGEVGPAGSPG

SEQ ID NO: 81 - protein, 40 aa, synthetic construct
  REGION 1..40: note = Recombinant collagen fragment
DGARGSDGQPGPPGPPGTAGFPGSPGAKGEVGPAGSPGSN

SEQ ID NO: 82 - protein, 52 aa, synthetic construct
  REGION 1..52: note = Recombinant collagen fragment
DGARGSDGQPGPPGPPGTAGFPGSPGAKGEVGPAGSPGSNGAPGQRGEPGPQ

SEQ ID NO: 83 - protein, 22 aa, synthetic construct
  REGION 1..22: note = Recombinant collagen fragment
DGESGRPGRPGERGLPGPPGIK

SEQ ID NO: 84 - protein, 25 aa, synthetic construct
  REGION 1..25: note = Recombinant collagen fragment
DGESGRPGRPGERGLPGPPGIKGPA

SEQ ID NO: 85 - protein, 31 aa, synthetic construct
  REGION 1..31: note = Recombinant collagen fragment
DGKPGPPGSQGESGRPGPPGPSGPRGQPGVM

SEQ ID NO: 86 - protein, 13 aa, synthetic construct
  REGION 1..13: note = Recombinant collagen fragment
DGQPGPPGPPGTA

SEQ ID NO: 87 - protein, 28 aa, synthetic construct
  REGION 1..28: note = Recombinant collagen fragment
DGQPGPPGPPGTAGFPGSPGAKGEVGPA

SEQ ID NO: 88 - protein, 16 aa, synthetic construct
  REGION 1..16: note = Recombinant collagen fragment
DGRNGEKGETGAPGLK

SEQ ID NO: 89 - protein, 25 aa, synthetic construct
  REGION 1..25: note = Recombinant collagen fragment
DGRNGEKGETGAPGLKGENGLPGEN

SEQ ID NO: 90 - protein, 34 aa, synthetic construct
  REGION 1..34: note = Recombinant collagen fragment
DGRNGEKGETGAPGLKGENGLPGENGAPGPMGPR

SEQ ID NO: 91 - protein, 49 aa, synthetic construct
  REGION 1..49: note = Recombinant collagen fragment
DGRNGEKGETGAPGLKGENGLPGENGAPGPMGPRGAPGERGRPGLPGAA

SEQ ID NO: 92 - protein, 52 aa, synthetic construct
  REGION 1..52: note = Recombinant collagen fragment
DGRNGEKGETGAPGLKGENGLPGENGAPGPMGPRGAPGERGRPGLPGAAGAR

SEQ ID NO: 93 - protein, 29 aa, synthetic construct
  REGION 1..29: note = Recombinant collagen fragment
EKGETGAPGLKGENGLPGENGAPGPMGPR

SEQ ID NO: 94 - protein, 17 aa, synthetic construct
  REGION 1..17: note = Recombinant collagen fragment
EMGPAGIPGAPGLMGAR

SEQ ID NO: 95 - protein, 17 aa, synthetic construct
  REGION 1..17: note = Recombinant collagen fragment
ENGLPGENGAPGPMGPR

SEQ ID NO: 96 - protein, 35 aa, synthetic construct
  REGION 1..35: note = Recombinant collagen fragment
ENGLPGENGAPGPMGPRGAPGERGRPGLPGAAGAR

SEQ ID NO: 97 - protein, 20 aa, synthetic construct
  REGION 1..20: note = Recombinant collagen fragment
EPGQAGPSGPPGPPGAIGPS

SEQ ID NO: 98 - protein, 14 aa, synthetic construct
  REGION 1..14: note = Recombinant collagen fragment
ERGLPGPPGIKGPA

SEQ ID NO: 99 - protein, 20 aa, synthetic construct
  REGION 1..20: note = Recombinant collagen fragment
ESGRPGPPGPSGPRGQPGVM

SEQ ID NO: 100 - protein, 28 aa, synthetic construct
  REGION 1..28: note = Recombinant collagen fragment
ESGRPGPPGPSGPRGQPGVMGFPGPKGN

SEQ ID NO: 101 - protein, 20 aa, synthetic construct
  REGION 1..20: note = Recombinant collagen fragment
ESGRPGRPGERGLPGPPGIK

SEQ ID NO: 102 - protein, 17 aa, synthetic construct
  REGION 1..17: note = Recombinant collagen fragment
ETGAPGLKGENGLPGEN

SEQ ID NO: 103 - protein, 26 aa, synthetic construct
  REGION 1..26: note = Recombinant collagen fragment
ETGAPGLKGENGLPGENGAPGPMGPR

SEQ ID NO: 104 - protein, 16 aa, synthetic construct
  REGION 1..16: note = Recombinant collagen fragment
ETGPQGPPGPTGPGGD

SEQ ID NO: 105 - protein, 23 aa, synthetic construct
  REGION 1..23: note = Recombinant collagen fragment
EVGPAGSPGSNGAPGQRGEPGPQ

SEQ ID NO: 106 - protein, 25 aa, synthetic construct
  REGION 1..25: note = Recombinant collagen fragment
EVGPAGSPGSNGAPGQRGEPGPQGH

SEQ ID NO: 107 - protein, 27 aa, synthetic construct
  REGION 1..27: note = Recombinant collagen fragment
EVGPAGSPGSNGAPGQRGEPGPQGHAG

SEQ ID NO: 108 - protein, 29 aa, synthetic construct
  REGION 1..29: note = Recombinant collagen fragment
EVGPAGSPGSNGAPGQRGEPGPQGHAGAQ

SEQ ID NO: 109 - protein, 38 aa, synthetic construct
  REGION 1..38: note = Recombinant collagen fragment
EVGPAGSPGSNGAPGQRGEPGPQGHAGAQGPPGPPGIN

SEQ ID NO: 110 - protein, 44 aa, synthetic construct
  REGION 1..44: note = Recombinant collagen fragment
EVGPAGSPGSNGAPGQRGEPGPQGHAGAQGPPGPPGINGSPGGK

SEQ ID NO: 111 - protein, 20 aa, synthetic construct
  REGION 1..20: note = Recombinant collagen fragment
FDGRNGEKGETGAPGLKGEN

SEQ ID NO: 112 - protein, 26 aa, synthetic construct
  REGION 1..26: note = Recombinant collagen fragment
FDGRNGEKGETGAPGLKGENGLPGEN

SEQ ID NO: 113 - protein, 35 aa, synthetic construct
  REGION 1..35: note = Recombinant collagen fragment
FDGRNGEKGETGAPGLKGENGLPGENGAPGPMGPR

SEQ ID NO: 114 - protein, 14 aa, synthetic construct
  REGION 1..14: note = Recombinant collagen fragment
FPGSPGAKGEVGPA

SEQ ID NO: 115 - protein, 20 aa, synthetic construct
  REGION 1..20: note = Recombinant collagen fragment
FPGSPGAKGEVGPAGSPGSN

SEQ ID NO: 116 - protein, 24 aa, synthetic construct
  REGION 1..24: note = Recombinant collagen fragment
GAAGARGNDGARGSDGQPGPPGPP

SEQ ID NO: 117 - protein, 27 aa, synthetic construct
  REGION 1..27: note = Recombinant collagen fragment
GAAGARGNDGARGSDGQPGPPGPPGTA

SEQ ID NO: 118 - protein, 36 aa, synthetic construct
  REGION 1..36: note = Recombinant collagen fragment
GAAGARGNDGARGSDGQPGPPGPPGTAGFPGSPGAK

SEQ ID NO: 119 - protein, 42 aa, synthetic construct
  REGION 1..42: note = Recombinant collagen fragment
GAAGARGNDGARGSDGQPGPPGPPGTAGFPGSPGAKGEVGPA

SEQ ID NO: 120 - protein, 22 aa, synthetic construct
  REGION 1..22: note = Recombinant collagen fragment
GAAGEPGRDGVPGGPGMRGMPG

SEQ ID NO: 121 - protein, 28 aa, synthetic construct
  REGION 1..28: note = Recombinant collagen fragment
GAAGEPGRDGVPGGPGMRGMPGSPGGPG

SEQ ID NO: 122 - protein, 18 aa, synthetic construct
  REGION 1..18: note = Recombinant collagen fragment
GAAGERGAPGFRGPAGPN

SEQ ID NO: 123 - protein, 24 aa, synthetic construct
  REGION 1..24: note = Recombinant collagen fragment
GAIGPSGPAGKDGESGRPGRPGER

SEQ ID NO: 124 - protein, 30 aa, synthetic construct
  REGION 1..30: note = Recombinant collagen fragment
GAIGPSGPAGKDGESGRPGRPGERGLPGPP

SEQ ID NO: 125 - protein, 33 aa, synthetic construct
  REGION 1..33: note = Recombinant collagen fragment
GAIGPSGPAGKDGESGRPGRPGERGLPGPPGIK

SEQ ID NO: 126 - protein, 22 aa, synthetic construct
  REGION 1..22: note = Recombinant collagen fragment
GAKGEPGPRGERGEAGIPGVPG

SEQ ID NO: 127 - protein, 24 aa, synthetic construct
  REGION 1..24: note = Recombinant collagen fragment
GAKGEPGPRGERGEAGIPGVPGAK

SEQ ID NO: 128 - protein, 25 aa, synthetic construct
  REGION 1..25: note = Recombinant collagen fragment
GAKGEPGPRGERGEAGIPGVPGAKG

SEQ ID NO: 129 - protein, 29 aa, synthetic construct
  REGION 1..29: note = Recombinant collagen fragment
GAKGEPGPRGERGEAGIPGVPGAKGEDGK

SEQ ID NO: 130 - protein, 31 aa, synthetic construct
  REGION 1..31: note = Recombinant collagen fragment
GAKGEPGPRGERGEAGIPGVPGAKGEDGKDG

SEQ ID NO: 131 - protein, 32 aa, synthetic construct
  REGION 1..32: note = Recombinant collagen fragment
GAKGEPGPRGERGEAGIPGVPGAKGEDGKDGS

SEQ ID NO: 132 - protein, 39 aa, synthetic construct
  REGION 1..39: note = Recombinant collagen fragment
GAKGEPGPRGERGEAGIPGVPGAKGEDGKDGSPGEPGAN

SEQ ID NO: 133 - protein, 27 aa, synthetic construct
  REGION 1..27: note = Recombinant collagen fragment
GAKGEVGPAGSPGSNGAPGQRGEPGPQ

SEQ ID NO: 134 - protein, 28 aa, synthetic construct
  REGION 1..28: note = Recombinant collagen fragment
GAKGEVGPAGSPGSNGAPGQRGEPGPQG

SEQ ID NO: 135 - protein, 29 aa, synthetic construct
  REGION 1..29: note = Recombinant collagen fragment
GAKGEVGPAGSPGSNGAPGQRGEPGPQGH

SEQ ID NO: 136 - protein, 33 aa, synthetic construct
  REGION 1..33: note = Recombinant collagen fragment
GAKGEVGPAGSPGSNGAPGQRGEPGPQGHAGAQ

SEQ ID NO: 137 - protein, 42 aa, synthetic construct
  REGION 1..42: note = Recombinant collagen fragment
GAKGEVGPAGSPGSNGAPGQRGEPGPQGHAGAQGPPGPPGIN

SEQ ID NO: 138 - protein, 9 aa, synthetic construct
  REGION 1..9: note = Recombinant collagen fragment
GANGAPGLR

SEQ ID NO: 139 - protein, 18 aa, synthetic construct
  REGION 1..18: note = Recombinant collagen fragment
GANGAPGLRGGAGEPGKN

SEQ ID NO: 140 - protein, 21 aa, synthetic construct
  REGION 1..21: note = Recombinant collagen fragment
GANGAPGLRGGAGEPGKNGAK

SEQ ID NO: 141 - protein, 27 aa, synthetic construct
  REGION 1..27: note = Recombinant collagen fragment
GANGAPGLRGGAGEPGKNGAKGEPGPR

SEQ ID NO: 142 - protein, 28 aa, synthetic construct
  REGION 1..28: note = Recombinant collagen fragment
GANGAPGLRGGAGEPGKNGAKGEPGPRG

SEQ ID NO: 143 - protein, 30 aa, synthetic construct
  REGION 1..30: note = Recombinant collagen fragment
GANGAPGLRGGAGEPGKNGAKGEPGPRGER

SEQ ID NO: 144 - protein, 31 aa, synthetic construct
  REGION 1..31: note = Recombinant collagen fragment
GANGAPGLRGGAGEPGKNGAKGEPGPRGERG

SEQ ID NO: 145 - protein, 34 aa, synthetic construct
  REGION 1..34: note = Recombinant collagen fragment
GANGAPGLRGGAGEPGKNGAKGEPGPRGERGEAG

SEQ ID NO: 146 - protein, 15 aa, synthetic construct
  REGION 1..15: note = Recombinant collagen fragment
GAPGERGRPGLPGAA

SEQ ID NO: 147 - protein, 16 aa, synthetic construct
  REGION 1..16: note = Recombinant collagen fragment
GAPGERGRPGLPGAAG

SEQ ID NO: 148 - protein, 17 aa, synthetic construct
  REGION 1..17: note = Recombinant collagen fragment
GAPGERGRPGLPGAAGA

SEQ ID NO: 149 - protein, 18 aa, synthetic construct
  REGION 1..18: note = Recombinant collagen fragment
GAPGERGRPGLPGAAGAR

SEQ ID NO: 150 - protein, 20 aa, synthetic construct
  REGION 1..20: note = Recombinant collagen fragment
GAPGERGRPGLPGAAGARGN

SEQ ID NO: 151 - protein, 21 aa, synthetic construct
  REGION 1..21: note = Recombinant collagen fragment
GAPGERGRPGLPGAAGARGND

SEQ ID NO: 152 - protein, 22 aa, synthetic construct
  REGION 1..22: note = Recombinant collagen fragment
GAPGERGRPGLPGAAGARGNDG

SEQ ID NO: 153 - protein, 23 aa, synthetic construct
  REGION 1..23: note = Recombinant collagen fragment
GAPGERGRPGLPGAAGARGNDGA

SEQ ID NO: 154 - protein, 24 aa, synthetic construct
  REGION 1..24: note = Recombinant collagen fragment
GAPGERGRPGLPGAAGARGNDGAR

SEQ ID NO: 155 - protein, 25 aa, synthetic construct
  REGION 1..25: note = Recombinant collagen fragment
GAPGERGRPGLPGAAGARGNDGARG

SEQ ID NO: 156 - protein, 26 aa, synthetic construct
  REGION 1..26: note = Recombinant collagen fragment
GAPGERGRPGLPGAAGARGNDGARGS

SEQ ID NO: 157 - protein, 27 aa, synthetic construct
  REGION 1..27: note = Recombinant collagen fragment
GAPGERGRPGLPGAAGARGNDGARGSD

SEQ ID NO: 158 - protein, 28 aa, synthetic construct
  REGION 1..28: note = Recombinant collagen fragment
GAPGERGRPGLPGAAGARGNDGARGSDG

SEQ ID NO: 159 - protein, 29 aa, synthetic construct
  REGION 1..29: note = Recombinant collagen fragment
GAPGERGRPGLPGAAGARGNDGARGSDGQ

SEQ ID NO: 160 - protein, 31 aa, synthetic construct
  REGION 1..31: note = Recombinant collagen fragment
GAPGERGRPGLPGAAGARGNDGARGSDGQPG

SEQ ID NO: 161 - protein, 32 aa, synthetic construct
  REGION 1..32: note = Recombinant collagen fragment
GAPGERGRPGLPGAAGARGNDGARGSDGQPGP

SEQ ID NO: 162 - protein, 34 aa, synthetic construct
  REGION 1..34: note = Recombinant collagen fragment
GAPGERGRPGLPGAAGARGNDGARGSDGQPGPPG

SEQ ID NO: 163 - protein, 35 aa, synthetic construct
  REGION 1..35: note = Recombinant collagen fragment
GAPGERGRPGLPGAAGARGNDGARGSDGQPGPPGP

SEQ ID NO: 164 - protein, 37 aa, synthetic construct
  REGION 1..37: note = Recombinant collagen fragment
GAPGERGRPGLPGAAGARGNDGARGSDGQPGPPGPPG

SEQ ID NO: 165 - protein, 39 aa, synthetic construct
  REGION 1..39: note = Recombinant collagen fragment
GAPGERGRPGLPGAAGARGNDGARGSDGQPGPPGPPGTA

SEQ ID NO: 166 - protein, 48 aa, synthetic construct
  REGION 1..48: note = Recombinant collagen fragment
GAPGERGRPGLPGAAGARGNDGARGSDGQPGPPGPPGTAGFPGSPGAK

SEQ ID NO: 167 - protein, 52 aa, synthetic construct
  REGION 1..52: note = Recombinant collagen fragment
GAPGERGRPGLPGAAGARGNDGARGSDGQPGPPGPPGTAGFPGSPGAKGEVG

SEQ ID NO: 168 - protein, 54 aa, synthetic construct
  REGION 1..54: note = Recombinant collagen fragment
GAPGERGRPGLPGAAGARGNDGARGSDGQPGPPGPPGTAGFPGSPGAKGEVGPA

SEQ ID NO: 169 - protein, 55 aa, synthetic construct
  REGION 1..55: note = Recombinant collagen fragment
GAPGERGRPGLPGAAGARGNDGARGSDGQPGPPGPPGTAGFPGSPGAKGEVGPAG

SEQ ID NO: 170 - protein, 24 aa, synthetic construct
  REGION 1..24: note = Recombinant collagen fragment
GAPGLKGENGLPGENGAPGPMGPR

SEQ ID NO: 171 - protein, 15 aa, synthetic construct
  REGION 1..15: note = Recombinant collagen fragment
GAPGLMGARGPPGPA

SEQ ID NO: 172 - protein, 18 aa, synthetic construct
  REGION 1..18: note = Recombinant collagen fragment
GAPGLMGARGPPGPAGAN

SEQ ID NO: 173 - protein, 21 aa, synthetic construct
  REGION 1..21: note = Recombinant collagen fragment
GAPGLMGARGPPGPAGANGAP

SEQ ID NO: 174 - protein, 24 aa, synthetic construct
  REGION 1..24: note = Recombinant collagen fragment
GAPGLMGARGPPGPAGANGAPGLR

SEQ ID NO: 175 - protein, 33 aa, synthetic construct
  REGION 1..33: note = Recombinant collagen fragment
GAPGLMGARGPPGPAGANGAPGLRGGAGEPGKN

SEQ ID NO: 176 - protein, 15 aa, synthetic construct
  REGION 1..15: note = Recombinant collagen fragment
GAPGLRGGAGEPGKN

SEQ ID NO: 177 - protein, 30 aa, synthetic construct
  REGION 1..30: note = Recombinant collagen fragment
GAPGLRGGAGEPGKNGAKGEPGPRGERGEA

SEQ ID NO: 178 - protein, 22 aa, synthetic construct
  REGION 1..22: note = Recombinant collagen fragment
GAPGPAGPRGAAGEPGRDGVPG

SEQ ID NO: 179 - protein, 31 aa, synthetic construct
  REGION 1..31: note = Recombinant collagen fragment
GAPGPAGPRGAAGEPGRDGVPGGPGMRGMPG

SEQ ID NO: 180 - protein, 21 aa, synthetic construct
  REGION 1..21: note = Recombinant collagen fragment
GAPGPMGPRGAPGERGRPGLP

SEQ ID NO: 181 - protein, 22 aa, synthetic construct
  REGION 1..22: note = Recombinant collagen fragment
GAPGPMGPRGAPGERGRPGLPG

SEQ ID NO: 182 - protein, 24 aa, synthetic construct
  REGION 1..24: note = Recombinant collagen fragment
GAPGPMGPRGAPGERGRPGLPGAA

SEQ ID NO: 183 - protein, 25 aa, synthetic construct
  REGION 1..25: note = Recombinant collagen fragment
GAPGPMGPRGAPGERGRPGLPGAAG

SEQ ID NO: 184 - protein, 26 aa, synthetic construct
  REGION 1..26: note = Recombinant collagen fragment
GAPGPMGPRGAPGERGRPGLPGAAGA

SEQ ID NO: 185 - protein, 27 aa, synthetic construct
  REGION 1..27: note = Recombinant collagen fragment
GAPGPMGPRGAPGERGRPGLPGAAGAR

SEQ ID NO: 186 - protein, 29 aa, synthetic construct
  REGION 1..29: note = Recombinant collagen fragment
GAPGPMGPRGAPGERGRPGLPGAAGARGN

SEQ ID NO: 187 - protein, 33 aa, synthetic construct
  REGION 1..33: note = Recombinant collagen fragment
GAPGPMGPRGAPGERGRPGLPGAAGARGNDGAR

SEQ ID NO: 188 - protein, 34 aa, synthetic construct
  REGION 1..34: note = Recombinant collagen fragment
GAPGPMGPRGAPGERGRPGLPGAAGARGNDGARG

SEQ ID NO: 189 - protein, 48 aa, synthetic construct
  REGION 1..48: note = Recombinant collagen fragment
GAPGPMGPRGAPGERGRPGLPGAAGARGNDGARGSDGQPGPPGPPGTA

SEQ ID NO: 190 - protein, 24 aa, synthetic construct
  REGION 1..24: note = Recombinant collagen fragment
GAPGQRGEPGPQGHAGAQGPPGPP

SEQ ID NO: 191 - protein, 25 aa, synthetic construct
  REGION 1..25: note = Recombinant collagen fragment
GAPGQRGEPGPQGHAGAQGPPGPPG

SEQ ID NO: 192 - protein, 27 aa, synthetic construct
  REGION 1..27: note = Recombinant collagen fragment
GAPGQRGEPGPQGHAGAQGPPGPPGIN

SEQ ID NO: 193 - protein, 28 aa, synthetic construct
  REGION 1..28: note = Recombinant collagen fragment
GAPGQRGEPGPQGHAGAQGPPGPPGING

SEQ ID NO: 194 - protein, 29 aa, synthetic construct
  REGION 1..29: note = Recombinant collagen fragment
GAPGQRGEPGPQGHAGAQGPPGPPGINGS

SEQ ID NO: 195 - protein, 31 aa, synthetic construct
  REGION 1..31: note = Recombinant collagen fragment
GAPGQRGEPGPQGHAGAQGPPGPPGINGSPG

SEQ ID NO: 196 - protein, 32 aa, synthetic construct
  REGION 1..32: note = Recombinant collagen fragment
GAPGQRGEPGPQGHAGAQGPPGPPGINGSPGG

SEQ ID NO: 197 - protein, 33 aa, synthetic construct
  REGION 1..33: note = Recombinant collagen fragment
GAPGQRGEPGPQGHAGAQGPPGPPGINGSPGGK

SEQ ID NO: 198 - protein, 36 aa, synthetic construct
  REGION 1..36: note = Recombinant collagen fragment
GAPGQRGEPGPQGHAGAQGPPGPPGINGSPGGKGEM

SEQ ID NO: 199 - protein, 37 aa, synthetic construct
  REGION 1..37: note = Recombinant collagen fragment
GAPGQRGEPGPQGHAGAQGPPGPPGINGSPGGKGEMG

SEQ ID NO: 200 - protein, 39 aa, synthetic construct
  REGION 1..39: note = Recombinant collagen fragment
GAPGQRGEPGPQGHAGAQGPPGPPGINGSPGGKGEMGPA

SEQ ID NO: 201 - protein, 48 aa, synthetic construct
  REGION 1..48: note = Recombinant collagen fragment
GAPGQRGEPGPQGHAGAQGPPGPPGINGSPGGKGEMGPAGIPGAPGLM

SEQ ID NO: 202 - protein, 49 aa, synthetic construct
  REGION 1..49: note = Recombinant collagen fragment
GAPGQRGEPGPQGHAGAQGPPGPPGINGSPGGKGEMGPAGIPGAPGLMG

SEQ ID NO: 203 - protein, 50 aa, synthetic construct
  REGION 1..50: note = Recombinant collagen fragment
GAPGQRGEPGPQGHAGAQGPPGPPGINGSPGGKGEMGPAGIPGAPGLMGA

SEQ ID NO: 204 - protein, 51 aa, synthetic construct
  REGION 1..51: note = Recombinant collagen fragment
GAPGQRGEPGPQGHAGAQGPPGPPGINGSPGGKGEMGPAGIPGAPGLMGAR

SEQ ID NO: 205 - protein, 12 aa, synthetic construct
  REGION 1..12: note = Recombinant collagen fragment
GAQGPPGPPGIN

SEQ ID NO: 206 - protein, 21 aa, synthetic construct
  REGION 1..21: note = Recombinant collagen fragment
GARGNDGARGSDGQPGPPGPP

SEQ ID NO: 207 - protein, 24 aa, synthetic construct
  REGION 1..24: note = Recombinant collagen fragment
GARGNDGARGSDGQPGPPGPPGTA

SEQ ID NO: 208 - protein, 28 aa, synthetic construct
  REGION 1..28: note = Recombinant collagen fragment
GARGNDGARGSDGQPGPPGPPGTAGFPG

SEQ ID NO: 209 - protein, 32 aa, synthetic construct
  REGION 1..32: note = Recombinant collagen fragment
GARGNDGARGSDGQPGPPGPPGTAGFPGSPGA

SEQ ID NO: 210 - protein, 33 aa, synthetic construct
  REGION 1..33: note = Recombinant collagen fragment
GARGNDGARGSDGQPGPPGPPGTAGFPGSPGAK

SEQ ID NO: 211 - protein, 39 aa, synthetic construct
  REGION 1..39: note = Recombinant collagen fragment
GARGNDGARGSDGQPGPPGPPGTAGFPGSPGAKGEVGPA

SEQ ID NO: 212 - protein, 40 aa, synthetic construct
  REGION 1..40: note = Recombinant collagen fragment
GARGNDGARGSDGQPGPPGPPGTAGFPGSPGAKGEVGPAG

SEQ ID NO: 213 - protein, 43 aa, synthetic construct
  REGION 1..43: note = Recombinant collagen fragment
GARGNDGARGSDGQPGPPGPPGTAGFPGSPGAKGEVGPAGSPG

SEQ ID NO: 214 - protein, 45 aa, synthetic construct
  REGION 1..45: note = Recombinant collagen fragment
GARGNDGARGSDGQPGPPGPPGTAGFPGSPGAKGEVGPAGSPGSN

SEQ ID NO: 215 - protein, 12 aa, synthetic construct
  REGION 1..12: note = Recombinant collagen fragment
GARGPPGPAGAN

SEQ ID NO: 216 - protein, 17 aa, synthetic construct
  REGION 1..17: note = Recombinant collagen fragment
GARGPPGPAGANGAPGL

SEQ ID NO: 217 - protein, 18 aa, synthetic construct
  REGION 1..18: note = Recombinant collagen fragment
GARGPPGPAGANGAPGLR

SEQ ID NO: 218 - protein, 19 aa, synthetic construct
  REGION 1..19: note = Recombinant collagen fragment
GARGPPGPAGANGAPGLRG

SEQ ID NO: 219 - protein, 20 aa, synthetic construct
  REGION 1..20: note = Recombinant collagen fragment
GARGPPGPAGANGAPGLRGG

SEQ ID NO: 220 - protein, 21 aa, synthetic construct
  REGION 1..21: note = Recombinant collagen fragment
GARGPPGPAGANGAPGLRGGA

SEQ ID NO: 221 - protein, 22 aa, synthetic construct
  REGION 1..22: note = Recombinant collagen fragment
GARGPPGPAGANGAPGLRGGAG

SEQ ID NO: 222 - protein, 23 aa, synthetic construct
  REGION 1..23: note = Recombinant collagen fragment
GARGPPGPAGANGAPGLRGGAGE

SEQ ID NO: 223 - protein, 25 aa, synthetic construct
  REGION 1..25: note = Recombinant collagen fragment
GARGPPGPAGANGAPGLRGGAGEPG

SEQ ID NO: 224 - protein, 27 aa, synthetic construct
  REGION 1..27: note = Recombinant collagen fragment
GARGPPGPAGANGAPGLRGGAGEPGKN

SEQ ID NO: 225 - protein, 28 aa, synthetic construct
  REGION 1..28: note = Recombinant collagen fragment
GARGPPGPAGANGAPGLRGGAGEPGKNG

SEQ ID NO: 226 - protein, 29 aa, synthetic construct
  REGION 1..29: note = Recombinant collagen fragment
GARGPPGPAGANGAPGLRGGAGEPGKNGA

SEQ ID NO: 227 - protein, 30 aa, synthetic construct
  REGION 1..30: note = Recombinant collagen fragment
GARGPPGPAGANGAPGLRGGAGEPGKNGAK

SEQ ID NO: 228 - protein, 32 aa, synthetic construct
  REGION 1..32: note = Recombinant collagen fragment
GARGPPGPAGANGAPGLRGGAGEPGKNGAKGE

SEQ ID NO: 229 - protein, 34 aa, synthetic construct
  REGION 1..34: note = Recombinant collagen fragment
GARGPPGPAGANGAPGLRGGAGEPGKNGAKGEPG

SEQ ID NO: 230 - protein, 36 aa, synthetic construct
  REGION 1..36: note = Recombinant collagen fragment
GARGPPGPAGANGAPGLRGGAGEPGKNGAKGEPGPR

SEQ ID NO: 231 - protein, 39 aa, synthetic construct
  REGION 1..39: note = Recombinant collagen fragment
GARGPPGPAGANGAPGLRGGAGEPGKNGAKGEPGPRGER

SEQ ID NO: 232 - protein, 18 aa, synthetic construct
  REGION 1..18: note = Recombinant collagen fragment
GARGSDGQPGPPGPPGTA

SEQ ID NO: 233 - protein, 27 aa, synthetic construct
  REGION 1..27: note = Recombinant collagen fragment
GARGSDGQPGPPGPPGTAGFPGSPGAK

SEQ ID NO: 234 - protein, 33 aa, synthetic construct
  REGION 1..33: note = Recombinant collagen fragment
GARGSDGQPGPPGPPGTAGFPGSPGAKGEVGPA

SEQ ID NO: 235 - protein, 27 aa, synthetic construct
  REGION 1..27: note = Recombinant collagen fragment
GDKGDTGPPGPQGLQGLPGTGGPPGEN

SEQ ID NO: 236 - protein, 12 aa, synthetic construct
  REGION 1..12: note = Recombinant collagen fragment
GEKGETGAPGLK

SEQ ID NO: 237 - protein, 21 aa, synthetic construct
  REGION 1..21: note = Recombinant collagen fragment
GEKGETGAPGLKGENGLPGEN

SEQ ID NO: 238 - protein, 27 aa, synthetic construct
  REGION 1..27: note = Recombinant collagen fragment
GEKGETGAPGLKGENGLPGENGAPGPM

SEQ ID NO: 239 - protein, 30 aa, synthetic construct
  REGION 1..30: note = Recombinant collagen fragment
GEKGETGAPGLKGENGLPGENGAPGPMGPR

SEQ ID NO: 240 - protein, 45 aa, synthetic construct
  REGION 1..45: note = Recombinant collagen fragment
GEKGETGAPGLKGENGLPGENGAPGPMGPRGAPGERGRPGLPGAA

SEQ ID NO: 241 - protein, 48 aa, synthetic construct
  REGION 1..48: note = Recombinant collagen fragment
GEKGETGAPGLKGENGLPGENGAPGPMGPRGAPGERGRPGLPGAAGAR

SEQ ID NO: 242 - protein, 15 aa, synthetic construct
  REGION 1..15: note = Recombinant collagen fragment
GEMGPAGIPGAPGLM

SEQ ID NO: 243 - protein, 18 aa, synthetic construct
  REGION 1..18: note = Recombinant collagen fragment
GEMGPAGIPGAPGLMGAR

SEQ ID NO: 244 - protein, 24 aa, synthetic construct
  REGION 1..24: note = Recombinant collagen fragment
GEMGPAGIPGAPGLMGARGPPGPA

SEQ ID NO: 245 - protein, 27 aa, synthetic construct
  REGION 1..27: note = Recombinant collagen fragment
GEMGPAGIPGAPGLMGARGPPGPAGAN

SEQ ID NO: 246 - protein, 33 aa, synthetic construct
  REGION 1..33: note = Recombinant collagen fragment
GEMGPAGIPGAPGLMGARGPPGPAGANGAPGLR

SEQ ID NO: 247 - protein, 42 aa, synthetic construct
  REGION 1..42: note = Recombinant collagen fragment
GEMGPAGIPGAPGLMGARGPPGPAGANGAPGLRGGAGEPGKN

SEQ ID NO: 248 - protein, 15 aa, synthetic construct
  REGION 1..15: note = Recombinant collagen fragment
GENGLPGENGAPGPM

SEQ ID NO: 249 - protein, 18 aa, synthetic construct
  REGION 1..18: note = Recombinant collagen fragment
GENGLPGENGAPGPMGPR

SEQ ID NO: 250 - protein, 19 aa, synthetic construct
  REGION 1..19: note = Recombinant collagen fragment
GENGLPGENGAPGPMGPRG

SEQ ID NO: 251 - protein, 22 aa, synthetic construct
  REGION 1..22: note = Recombinant collagen fragment
GENGLPGENGAPGPMGPRGAPG

SEQ ID NO: 252 - protein, 24 aa, synthetic construct
  REGION 1..24: note = Recombinant collagen fragment
GENGLPGENGAPGPMGPRGAPGER

SEQ ID NO: 253 - protein, 25 aa, synthetic construct
  REGION 1..25: note = Recombinant collagen fragment
GENGLPGENGAPGPMGPRGAPGERG

SEQ ID NO: 254 - protein, 26 aa, synthetic construct
  REGION 1..26: note = Recombinant collagen fragment
GENGLPGENGAPGPMGPRGAPGERGR

SEQ ID NO: 255 - protein, 33 aa, synthetic construct
  REGION 1..33: note = Recombinant collagen fragment
GENGLPGENGAPGPMGPRGAPGERGRPGLPGAA

SEQ ID NO: 256 - protein, 35 aa, synthetic construct
  REGION 1..35: note = Recombinant collagen fragment
GENGLPGENGAPGPMGPRGAPGERGRPGLPGAAGA

SEQ ID NO: 257 - protein, 36 aa, synthetic construct
  REGION 1..36: note = Recombinant collagen fragment
GENGLPGENGAPGPMGPRGAPGERGRPGLPGAAGAR

SEQ ID NO: 258 - protein, 38 aa, synthetic construct
  REGION 1..38: note = Recombinant collagen fragment
GENGLPGENGAPGPMGPRGAPGERGRPGLPGAAGARGN

SEQ ID NO: 259 - protein, 27 aa, synthetic construct
  REGION 1..27: note = Recombinant collagen fragment
GEPGANGLPGAAGERGAPGFRGPAGPN

SEQ ID NO: 260 - protein, 25 aa, synthetic construct
  REGION 1..25: note = Recombinant collagen fragment
GEPGPKGDAGAPGAPGGKGDAGAPG

SEQ ID NO: 261 - protein, 26 aa, synthetic construct
  REGION 1..26: note = Recombinant collagen fragment
GEPGPKGDAGAPGAPGGKGDAGAPGE

SEQ ID NO: 262 - protein, 21 aa, synthetic construct
  REGION 1..21: note = Recombinant collagen fragment
GEPGPQGHAGAQGPPGPPGIN

SEQ ID NO: 263 - protein, 33 aa, synthetic construct
  REGION 1..33: note = Recombinant collagen fragment
GEPGPQGHAGAQGPPGPPGINGSPGGKGEMGPA

SEQ ID NO: 264 - protein, 42 aa, synthetic construct
  REGION 1..42: note = Recombinant collagen fragment
GEPGPQGHAGAQGPPGPPGINGSPGGKGEMGPAGIPGAPGLM

SEQ ID NO: 265 - protein, 28 aa, synthetic construct
  REGION 1..28: note = Recombinant collagen fragment
GEPGPRGERGEAGIPGVPGAKGEDGKDG

SEQ ID NO: 266 - protein, 21 aa, synthetic construct
  REGION 1..21: note = Recombinant collagen fragment
GEPGQAGPSGPPGPPGAIGPS

SEQ ID NO: 267 - protein, 27 aa, synthetic construct
  REGION 1..27: note = Recombinant collagen fragment
GEPGQAGPSGPPGPPGAIGPSGPAGKD

SEQ ID NO: 268 - protein, 25 aa, synthetic construct
  REGION 1..25: note = Recombinant collagen fragment
GEPGRDGVPGGPGMRGMPGSPGGPG

SEQ ID NO: 269 - protein, 16 aa, synthetic construct
  REGION 1..16: note = Recombinant collagen fragment
GERGEAGIPGVPGAKG

SEQ ID NO: 270 - protein, 22 aa, synthetic construct
  REGION 1..22: note = Recombinant collagen fragment
GERGEAGIPGVPGAKGEDGKDG

SEQ ID NO: 271 - protein, 30 aa, synthetic construct
  REGION 1..30: note = Recombinant collagen fragment
GERGEAGIPGVPGAKGEDGKDGSPGEPGAN

SEQ ID NO: 272 - protein, 17 aa, synthetic construct
  REGION 1..17: note = Recombinant collagen fragment
GERGGPGGPGPQGPPGK

SEQ ID NO: 273 - protein, 18 aa, synthetic construct
  REGION 1..18: note = Recombinant collagen fragment
GERGGPGGPGPQGPPGKN

SEQ ID NO: 274 - protein, 19 aa, synthetic construct
  REGION 1..19: note = Recombinant collagen fragment
GERGGPGGPGPQGPPGKNG

SEQ ID NO: 275 - protein, 23 aa, synthetic construct
  REGION 1..23: note = Recombinant collagen fragment
GERGGPGGPGPQGPPGKNGETGP

SEQ ID NO: 276 - protein, 24 aa, synthetic construct
  REGION 1..24: note = Recombinant collagen fragment
GERGGPGGPGPQGPPGKNGETGPQ

SEQ ID NO: 277 - protein, 25 aa, synthetic construct
  REGION 1..25: note = Recombinant collagen fragment
GERGGPGGPGPQGPPGKNGETGPQG

SEQ ID NO: 278 - protein, 27 aa, synthetic construct
  REGION 1..27: note = Recombinant collagen fragment
GERGGPGGPGPQGPPGKNGETGPQGPP

SEQ ID NO: 279 - protein, 28 aa, synthetic construct
  REGION 1..28: note = Recombinant collagen fragment
GERGGPGGPGPQGPPGKNGETGPQGPPG

SEQ ID NO: 280 - protein, 29 aa, synthetic construct
  REGION 1..29: note = Recombinant collagen fragment
GERGGPGGPGPQGPPGKNGETGPQGPPGP

SEQ ID NO: 281 - protein, 30 aa, synthetic construct
  REGION 1..30: note = Recombinant collagen fragment
GERGGPGGPGPQGPPGKNGETGPQGPPGPT

SEQ ID NO: 282 - protein, 31 aa, synthetic construct
  REGION 1..31: note = Recombinant collagen fragment
GERGGPGGPGPQGPPGKNGETGPQGPPGPTG

SEQ ID NO: 283 - protein, 33 aa, synthetic construct
  REGION 1..33: note = Recombinant collagen fragment
GERGGPGGPGPQGPPGKNGETGPQGPPGPTGPG

SEQ ID NO: 284 - protein, 34 aa, synthetic construct
  REGION 1..34: note = Recombinant collagen fragment
GERGGPGGPGPQGPPGKNGETGPQGPPGPTGPGG

SEQ ID NO: 285 - protein, 35 aa, synthetic construct
  REGION 1..35: note = Recombinant collagen fragment
GERGGPGGPGPQGPPGKNGETGPQGPPGPTGPGGD

SEQ ID NO: 286 - protein, 36 aa, synthetic construct
  REGION 1..36: note = Recombinant collagen fragment
GERGGPGGPGPQGPPGKNGETGPQGPPGPTGPGGDK

SEQ ID NO: 287 - protein, 37 aa, synthetic construct
  REGION 1..37: note = Recombinant collagen fragment
GERGGPGGPGPQGPPGKNGETGPQGPPGPTGPGGDKG

SEQ ID NO: 288 - protein, 38 aa, synthetic construct
  REGION 1..38: note = Recombinant collagen fragment
GERGGPGGPGPQGPPGKNGETGPQGPPGPTGPGGDKGD

SEQ ID NO: 289 - protein, 40 aa, synthetic construct
  REGION 1..40: note = Recombinant collagen fragment
GERGGPGGPGPQGPPGKNGETGPQGPPGPTGPGGDKGDTG

SEQ ID NO: 290 - protein, 43 aa, synthetic construct
  REGION 1..43: note = Recombinant collagen fragment
GERGGPGGPGPQGPPGKNGETGPQGPPGPTGPGGDKGDTGPPG

SEQ ID NO: 291 - protein, 45 aa, synthetic construct
  REGION 1..45: note = Recombinant collagen fragment
GERGGPGGPGPQGPPGKNGETGPQGPPGPTGPGGDKGDTGPPGPQ

SEQ ID NO: 292 - protein, 46 aa, synthetic construct
  REGION 1..46: note = Recombinant collagen fragment
GERGGPGGPGPQGPPGKNGETGPQGPPGPTGPGGDKGDTGPPGPQG

SEQ ID NO: 293 - protein, 48 aa, synthetic construct
  REGION 1..48: note = Recombinant collagen fragment
GERGGPGGPGPQGPPGKNGETGPQGPPGPTGPGGDKGDTGPPGPQGLQ

SEQ ID NO: 294 - protein, 49 aa, synthetic construct
  REGION 1..49: note = Recombinant collagen fragment
GERGGPGGPGPQGPPGKNGETGPQGPPGPTGPGGDKGDTGPPGPQGLQG

SEQ ID NO: 295 - protein, 12 aa, synthetic construct
  REGION 1..12: note = Recombinant collagen fragment
GERGLPGPPGIK

SEQ ID NO: 296 - protein, 15 aa, synthetic construct
  REGION 1..15: note = Recombinant collagen fragment
GERGLPGPPGIKGPA

SEQ ID NO: 297 - protein, 24 aa, synthetic construct
  REGION 1..24: note = Recombinant collagen fragment
GERGLPGPPGIKGPAGIPGFPGMK

SEQ ID NO: 298 - protein, 21 aa, synthetic construct
  REGION 1..21: note = Recombinant collagen fragment
GESGRPGPPGPSGPRGQPGVM

SEQ ID NO: 299 - protein, 29 aa, synthetic construct
  REGION 1..29: note = Recombinant collagen fragment
GESGRPGPPGPSGPRGQPGVMGFPGPKGN

SEQ ID NO: 300 - protein, 21 aa, synthetic construct
  REGION 1..21: note = Recombinant collagen fragment
GESGRPGRPGERGLPGPPGIK

SEQ ID NO: 301 - protein, 22 aa, synthetic construct
  REGION 1..22: note = Recombinant collagen fragment
GESGRPGRPGERGLPGPPGIKG

SEQ ID NO: 302 - protein, 24 aa, synthetic construct
  REGION 1..24: note = Recombinant collagen fragment
GESGRPGRPGERGLPGPPGIKGPA

SEQ ID NO: 303 - protein, 33 aa, synthetic construct
  REGION 1..33: note = Recombinant collagen fragment
GESGRPGRPGERGLPGPPGIKGPAGIPGFPGMK

SEQ ID NO: 304 - protein, 18 aa, synthetic construct
  REGION 1..18: note = Recombinant collagen fragment
GETGAPGLKGENGLPGEN

SEQ ID NO: 305 - protein, 27 aa, synthetic construct
  REGION 1..27: note = Recombinant collagen fragment
GETGAPGLKGENGLPGENGAPGPMGPR

SEQ ID NO: 306 - protein, 45 aa, synthetic construct
  REGION 1..45: note = Recombinant collagen fragment
GETGAPGLKGENGLPGENGAPGPMGPRGAPGERGRPGLPGAAGAR

SEQ ID NO: 307 - protein, 47 aa, synthetic construct
  REGION 1..47: note = Recombinant collagen fragment
GETGAPGLKGENGLPGENGAPGPMGPRGAPGERGRPGLPGAAGARGN

SEQ ID NO: 308 - protein, 15 aa, synthetic construct
  REGION 1..15: note = Recombinant collagen fragment
GETGPQGPPGPTGPG

SEQ ID NO: 309 - protein, 17 aa, synthetic construct
  REGION 1..17: note = Recombinant collagen fragment
GETGPQGPPGPTGPGGD

SEQ ID NO: 310 - protein, 22 aa, synthetic construct
  REGION 1..22: note = Recombinant collagen fragment
GETGPQGPPGPTGPGGDKGDTG

SEQ ID NO: 311 - protein, 25 aa, synthetic construct
  REGION 1..25: note = Recombinant collagen fragment
GETGPQGPPGPTGPGGDKGDTGPPG

SEQ ID NO: 312 - protein, 27 aa, synthetic construct
  REGION 1..27: note = Recombinant collagen fragment
GETGPQGPPGPTGPGGDKGDTGPPGPQ

SEQ ID NO: 313 - protein, 28 aa, synthetic construct
  REGION 1..28: note = Recombinant collagen fragment
GETGPQGPPGPTGPGGDKGDTGPPGPQG

SEQ ID NO: 314 - protein, 16 aa, synthetic construct
  REGION 1..16: note = Recombinant collagen fragment
GEVGPAGSPGSNGAPG

SEQ ID NO: 315 - protein, 17 aa, synthetic construct
  REGION 1..17: note = Recombinant collagen fragment
GEVGPAGSPGSNGAPGQ

SEQ ID NO: 316 - protein, 18 aa, synthetic construct
  REGION 1..18: note = Recombinant collagen fragment
GEVGPAGSPGSNGAPGQR

SEQ ID NO: 317 - protein, 20 aa, synthetic construct
  REGION 1..20: note = Recombinant collagen fragment
GEVGPAGSPGSNGAPGQRGE

SEQ ID NO: 318 - protein, 21 aa, synthetic construct
  REGION 1..21: note = Recombinant collagen fragment
GEVGPAGSPGSNGAPGQRGEP

SEQ ID NO: 319 - protein, 22 aa, synthetic construct
  REGION 1..22: note = Recombinant collagen fragment
GEVGPAGSPGSNGAPGQRGEPG

SEQ ID NO: 320 - protein, 23 aa, synthetic construct
  REGION 1..23: note = Recombinant collagen fragment
GEVGPAGSPGSNGAPGQRGEPGP

SEQ ID NO: 321 - protein, 24 aa, synthetic construct
  REGION 1..24: note = Recombinant collagen fragment
GEVGPAGSPGSNGAPGQRGEPGPQ

SEQ ID NO: 322 - protein, 25 aa, synthetic construct
  REGION 1..25: note = Recombinant collagen fragment
GEVGPAGSPGSNGAPGQRGEPGPQG

SEQ ID NO: 323 - protein, 26 aa, synthetic construct
  REGION 1..26: note = Recombinant collagen fragment
GEVGPAGSPGSNGAPGQRGEPGPQGH

SEQ ID NO: 324 - protein, 27 aa, synthetic construct
  REGION 1..27: note = Recombinant collagen fragment
GEVGPAGSPGSNGAPGQRGEPGPQGHA

SEQ ID NO: 325 - protein, 28 aa, synthetic construct
  REGION 1..28: note = Recombinant collagen fragment
GEVGPAGSPGSNGAPGQRGEPGPQGHAG

SEQ ID NO: 326 - protein, 29 aa, synthetic construct
  REGION 1..29: note = Recombinant collagen fragment
GEVGPAGSPGSNGAPGQRGEPGPQGHAGA

SEQ ID NO: 327 - protein, 30 aa, synthetic construct
  REGION 1..30: note = Recombinant collagen fragment
GEVGPAGSPGSNGAPGQRGEPGPQGHAGAQ

SEQ ID NO: 328 - protein, 31 aa, synthetic construct
  REGION 1..31: note = Recombinant collagen fragment
GEVGPAGSPGSNGAPGQRGEPGPQGHAGAQG

SEQ ID NO: 329 - protein, 32 aa, synthetic construct
  REGION 1..32: note = Recombinant collagen fragment
GEVGPAGSPGSNGAPGQRGEPGPQGHAGAQGP

SEQ ID NO: 330 - protein, 33 aa, synthetic construct
  REGION 1..33: note = Recombinant collagen fragment
GEVGPAGSPGSNGAPGQRGEPGPQGHAGAQGPP

SEQ ID NO: 331 - protein, 34 aa, synthetic construct
  REGION 1..34: note = Recombinant collagen fragment
GEVGPAGSPGSNGAPGQRGEPGPQGHAGAQGPPG

SEQ ID NO: 332 - protein, 35 aa, synthetic construct
  REGION 1..35: note = Recombinant collagen fragment
GEVGPAGSPGSNGAPGQRGEPGPQGHAGAQGPPGP

SEQ ID NO: 333 - protein, 36 aa, synthetic construct
  REGION 1..36: note = Recombinant collagen fragment
GEVGPAGSPGSNGAPGQRGEPGPQGHAGAQGPPGPP

SEQ ID NO: 334 - protein, 37 aa, synthetic construct
  REGION 1..37: note = Recombinant collagen fragment
GEVGPAGSPGSNGAPGQRGEPGPQGHAGAQGPPGPPG

SEQ ID NO: 335 - protein, 39 aa, synthetic construct
  REGION 1..39: note = Recombinant collagen fragment
GEVGPAGSPGSNGAPGQRGEPGPQGHAGAQGPPGPPGIN

SEQ ID NO: 336 - protein, 40 aa, synthetic construct
  REGION 1..40: note = Recombinant collagen fragment
GEVGPAGSPGSNGAPGQRGEPGPQGHAGAQGPPGPPGING

SEQ ID NO: 337 - protein, 41 aa, synthetic construct
  REGION 1..41: note = Recombinant collagen fragment
GEVGPAGSPGSNGAPGQRGEPGPQGHAGAQGPPGPPGINGS

SEQ ID NO: 338 - protein, 43 aa, synthetic construct
  REGION 1..43: note = Recombinant collagen fragment
GEVGPAGSPGSNGAPGQRGEPGPQGHAGAQGPPGPPGINGSPG

SEQ ID NO: 339 - protein, 44 aa, synthetic construct
  REGION 1..44: note = Recombinant collagen fragment
GEVGPAGSPGSNGAPGQRGEPGPQGHAGAQGPPGPPGINGSPGG

SEQ ID NO: 340 - protein, 45 aa, synthetic construct
  REGION 1..45: note = Recombinant collagen fragment
GEVGPAGSPGSNGAPGQRGEPGPQGHAGAQGPPGPPGINGSPGGK

SEQ ID NO: 341 - protein, 46 aa, synthetic construct
  REGION 1..46: note = Recombinant collagen fragment
GEVGPAGSPGSNGAPGQRGEPGPQGHAGAQGPPGPPGINGSPGGKG

SEQ ID NO: 342 - protein, 48 aa, synthetic construct
  REGION 1..48: note = Recombinant collagen fragment
GEVGPAGSPGSNGAPGQRGEPGPQGHAGAQGPPGPPGINGSPGGKGEM

SEQ ID NO: 343 - protein, 49 aa, synthetic construct
  REGION 1..49: note = Recombinant collagen fragment
GEVGPAGSPGSNGAPGQRGEPGPQGHAGAQGPPGPPGINGSPGGKGEMG

SEQ ID NO: 344 - protein, 51 aa, synthetic construct
  REGION 1..51: note = Recombinant collagen fragment
GEVGPAGSPGSNGAPGQRGEPGPQGHAGAQGPPGPPGINGSPGGKGEMGPA

SEQ ID NO: 345 - protein, 52 aa, synthetic construct
  REGION 1..52: note = Recombinant collagen fragment
GEVGPAGSPGSNGAPGQRGEPGPQGHAGAQGPPGPPGINGSPGGKGEMGPAG

SEQ ID NO: 346 - protein, 18 aa, synthetic construct
  REGION 1..18: note = Recombinant collagen fragment
GFDGRNGEKGETGAPGLK

SEQ ID NO: 347 - protein, 19 aa, synthetic construct
  REGION 1..19: note = Recombinant collagen fragment
GFDGRNGEKGETGAPGLKG

SEQ ID NO: 348 - protein, 21 aa, synthetic construct
  REGION 1..21: note = Recombinant collagen fragment
GFDGRNGEKGETGAPGLKGEN

SEQ ID NO: 349 - protein, 22 aa, synthetic construct
  REGION 1..22: note = Recombinant collagen fragment
GFDGRNGEKGETGAPGLKGENG

SEQ ID NO: 350 - protein, 27 aa, synthetic construct
  REGION 1..27: note = Recombinant collagen fragment
GFDGRNGEKGETGAPGLKGENGLPGEN

SEQ ID NO: 351 - protein, 36 aa, synthetic construct
  REGION 1..36: note = Recombinant collagen fragment
GFDGRNGEKGETGAPGLKGENGLPGENGAPGPMGPR

SEQ ID NO: 352 - protein, 51 aa, synthetic construct
  REGION 1..51: note = Recombinant collagen fragment
GFDGRNGEKGETGAPGLKGENGLPGENGAPGPMGPRGAPGERGRPGLPGAA

SEQ ID NO: 353 - protein, 27 aa, synthetic construct
  REGION 1..27: note = Recombinant collagen fragment
GFPGMKGHRGFDGRNGEKGETGAPGLK

SEQ ID NO: 354 - protein, 30 aa, synthetic construct
  REGION 1..30: note = Recombinant collagen fragment
GFPGMKGHRGFDGRNGEKGETGAPGLKGEN

SEQ ID NO: 355 - protein, 15 aa, synthetic construct
  REGION 1..15: note = Recombinant collagen fragment
GFPGPKGNDGAPGKN

SEQ ID NO: 356 - protein, 18 aa, synthetic construct
  REGION 1..18: note = Recombinant collagen fragment
GFPGPKGNDGAPGKNGER

SEQ ID NO: 357 - protein, 19 aa, synthetic construct
  REGION 1..19: note = Recombinant collagen fragment
GFPGPKGNDGAPGKNGERG

SEQ ID NO: 358 - protein, 20 aa, synthetic construct
  REGION 1..20: note = Recombinant collagen fragment
GFPGPKGNDGAPGKNGERGG

SEQ ID NO: 359 - protein, 21 aa, synthetic construct
  REGION 1..21: note = Recombinant collagen fragment
GFPGPKGNDGAPGKNGERGGP

SEQ ID NO: 360 - protein, 22 aa, synthetic construct
  REGION 1..22: note = Recombinant collagen fragment
GFPGPKGNDGAPGKNGERGGPG

SEQ ID NO: 361 - protein, 23 aa, synthetic construct
  REGION 1..23: note = Recombinant collagen fragment
GFPGPKGNDGAPGKNGERGGPGG

SEQ ID NO: 362 - protein, 24 aa, synthetic construct
  REGION 1..24: note = Recombinant collagen fragment
GFPGPKGNDGAPGKNGERGGPGGP

SEQ ID NO: 363 - protein, 25 aa, synthetic construct
  REGION 1..25: note = Recombinant collagen fragment
GFPGPKGNDGAPGKNGERGGPGGPG

SEQ ID NO: 364 - protein, 26 aa, synthetic construct
  REGION 1..26: note = Recombinant collagen fragment
GFPGPKGNDGAPGKNGERGGPGGPGP

SEQ ID NO: 365 - protein, 27 aa, synthetic construct
  REGION 1..27: note = Recombinant collagen fragment
GFPGPKGNDGAPGKNGERGGPGGPGPQ

SEQ ID NO: 366 - protein, 28 aa, synthetic construct
  REGION 1..28: note = Recombinant collagen fragment
GFPGPKGNDGAPGKNGERGGPGGPGPQG

SEQ ID NO: 367 - protein, 29 aa, synthetic construct
  REGION 1..29: note = Recombinant collagen fragment
GFPGPKGNDGAPGKNGERGGPGGPGPQGP

SEQ ID NO: 368 - protein, 30 aa, synthetic construct
  REGION 1..30: note = Recombinant collagen fragment
GFPGPKGNDGAPGKNGERGGPGGPGPQGPP

SEQ ID NO: 369 - protein, 31 aa, synthetic construct
  REGION 1..31: note = Recombinant collagen fragment
GFPGPKGNDGAPGKNGERGGPGGPGPQGPPG

SEQ ID NO: 370 - protein, 32 aa, synthetic construct
  REGION 1..32: note = Recombinant collagen fragment
GFPGPKGNDGAPGKNGERGGPGGPGPQGPPGK

SEQ ID NO: 371 - protein, 33 aa, synthetic construct
  REGION 1..33: note = Recombinant collagen fragment
GFPGPKGNDGAPGKNGERGGPGGPGPQGPPGKN

SEQ ID NO: 372 - protein, 34 aa, synthetic construct
  REGION 1..34: note = Recombinant collagen fragment
GFPGPKGNDGAPGKNGERGGPGGPGPQGPPGKNG

SEQ ID NO: 373 - protein, 35 aa, synthetic construct
  REGION 1..35: note = Recombinant collagen fragment
GFPGPKGNDGAPGKNGERGGPGGPGPQGPPGKNGE

SEQ ID NO: 374 - protein, 36 aa, synthetic construct
  REGION 1..36: note = Recombinant collagen fragment
GFPGPKGNDGAPGKNGERGGPGGPGPQGPPGKNGET

SEQ ID NO: 375 - protein, 37 aa, synthetic construct
  REGION 1..37: note = Recombinant collagen fragment
GFPGPKGNDGAPGKNGERGGPGGPGPQGPPGKNGETG

SEQ ID NO: 376 - protein, 38 aa, synthetic construct
  REGION 1..38: note = Recombinant collagen fragment
GFPGPKGNDGAPGKNGERGGPGGPGPQGPPGKNGETGP

SEQ ID NO: 377 - protein, 39 aa, synthetic construct
  REGION 1..39: note = Recombinant collagen fragment
GFPGPKGNDGAPGKNGERGGPGGPGPQGPPGKNGETGPQ

SEQ ID NO: 378 - protein, 40 aa, synthetic construct
  REGION 1..40: note = Recombinant collagen fragment
GFPGPKGNDGAPGKNGERGGPGGPGPQGPPGKNGETGPQG

SEQ ID NO: 379 - protein, 43 aa, synthetic construct
  REGION 1..43: note = Recombinant collagen fragment
GFPGPKGNDGAPGKNGERGGPGGPGPQGPPGKNGETGPQGPPG

SEQ ID NO: 380 - protein, 46 aa, synthetic construct
  REGION 1..46: note = Recombinant collagen fragment
GFPGPKGNDGAPGKNGERGGPGGPGPQGPPGKNGETGPQGPPGPTG

SEQ ID NO: 381 - protein, 50 aa, synthetic construct
  REGION 1..50: note = Recombinant collagen fragment
GFPGPKGNDGAPGKNGERGGPGGPGPQGPPGKNGETGPQGPPGPTGPGGD

SEQ ID NO: 382 - protein, 51 aa, synthetic construct
  REGION 1..51: note = Recombinant collagen fragment
GFPGPKGNDGAPGKNGERGGPGGPGPQGPPGKNGETGPQGPPGPTGPGGDK

SEQ ID NO: 383 - protein, 13 aa, synthetic construct
  REGION 1..13: note = Recombinant collagen fragment
GFPGSPGAKGEVG

SEQ ID NO: 384 - protein, 14 aa, synthetic construct
  REGION 1..14: note = Recombinant collagen fragment
GFPGSPGAKGEVGP

SEQ ID NO: 385 - protein, 15 aa, synthetic construct
  REGION 1..15: note = Recombinant collagen fragment
GFPGSPGAKGEVGPA

SEQ ID NO: 386 - protein, 16 aa, synthetic construct
  REGION 1..16: note = Recombinant collagen fragment
GFPGSPGAKGEVGPAG

SEQ ID NO: 387 - protein, 17 aa, synthetic construct
  REGION 1..17: note = Recombinant collagen fragment
GFPGSPGAKGEVGPAGS

SEQ ID NO: 388 - protein, 19 aa, synthetic construct
  REGION 1..19: note = Recombinant collagen fragment
GFPGSPGAKGEVGPAGSPG

SEQ ID NO: 389 - protein, 21 aa, synthetic construct
  REGION 1..21: note = Recombinant collagen fragment
GFPGSPGAKGEVGPAGSPGSN

SEQ ID NO: 390 - protein, 22 aa, synthetic construct
  REGION 1..22: note = Recombinant collagen fragment
GFPGSPGAKGEVGPAGSPGSNG

SEQ ID NO: 391 - protein, 23 aa, synthetic construct
  REGION 1..23: note = Recombinant collagen fragment
GFPGSPGAKGEVGPAGSPGSNGA

SEQ ID NO: 392 - protein, 25 aa, synthetic construct
  REGION 1..25: note = Recombinant collagen fragment
GFPGSPGAKGEVGPAGSPGSNGAPG

SEQ ID NO: 393 - protein, 26 aa, synthetic construct
  REGION 1..26: note = Recombinant collagen fragment
GFPGSPGAKGEVGPAGSPGSNGAPGQ

SEQ ID NO: 394 - protein, 27 aa, synthetic construct
  REGION 1..27: note = Recombinant collagen fragment
GFPGSPGAKGEVGPAGSPGSNGAPGQR

SEQ ID NO: 395 - protein, 28 aa, synthetic construct
  REGION 1..28: note = Recombinant collagen fragment
GFPGSPGAKGEVGPAGSPGSNGAPGQRG

SEQ ID NO: 396 - protein, 29 aa, synthetic construct
  REGION 1..29: note = Recombinant collagen fragment
GFPGSPGAKGEVGPAGSPGSNGAPGQRGE

SEQ ID NO: 397 - protein, 30 aa, synthetic construct
  REGION 1..30: note = Recombinant collagen fragment
GFPGSPGAKGEVGPAGSPGSNGAPGQRGEP

SEQ ID NO: 398 - protein, 31 aa, synthetic construct
  REGION 1..31: note = Recombinant collagen fragment
GFPGSPGAKGEVGPAGSPGSNGAPGQRGEPG

SEQ ID NO: 399 - protein, 33 aa, synthetic construct
  REGION 1..33: note = Recombinant collagen fragment
GFPGSPGAKGEVGPAGSPGSNGAPGQRGEPGPQ

SEQ ID NO: 400 - protein, 34 aa, synthetic construct
  REGION 1..34: note = Recombinant collagen fragment
GFPGSPGAKGEVGPAGSPGSNGAPGQRGEPGPQG

SEQ ID NO: 401 - protein, 35 aa, synthetic construct
  REGION 1..35: note = Recombinant collagen fragment
GFPGSPGAKGEVGPAGSPGSNGAPGQRGEPGPQGH

SEQ ID NO: 402 - protein, 36 aa, synthetic construct
  REGION 1..36: note = Recombinant collagen fragment
GFPGSPGAKGEVGPAGSPGSNGAPGQRGEPGPQGHA

SEQ ID NO: 403 - protein, 37 aa, synthetic construct
  REGION 1..37: note = Recombinant collagen fragment
GFPGSPGAKGEVGPAGSPGSNGAPGQRGEPGPQGHAG

SEQ ID NO: 404 - protein, 38 aa, synthetic construct
  REGION 1..38: note = Recombinant collagen fragment
GFPGSPGAKGEVGPAGSPGSNGAPGQRGEPGPQGHAGA

SEQ ID NO: 405 - protein, 39 aa, synthetic construct
  REGION 1..39: note = Recombinant collagen fragment
GFPGSPGAKGEVGPAGSPGSNGAPGQRGEPGPQGHAGAQ

SEQ ID NO: 406 - protein, 40 aa, synthetic construct
  REGION 1..40: note = Recombinant collagen fragment
GFPGSPGAKGEVGPAGSPGSNGAPGQRGEPGPQGHAGAQG

SEQ ID NO: 407 - protein, 42 aa, synthetic construct
  REGION 1..42: note = Recombinant collagen fragment
GFPGSPGAKGEVGPAGSPGSNGAPGQRGEPGPQGHAGAQGPP

SEQ ID NO: 408 - protein, 43 aa, synthetic construct
  REGION 1..43: note = Recombinant collagen fragment
GFPGSPGAKGEVGPAGSPGSNGAPGQRGEPGPQGHAGAQGPPG

SEQ ID NO: 409 - protein, 46 aa, synthetic construct
  REGION 1..46: note = Recombinant collagen fragment
GFPGSPGAKGEVGPAGSPGSNGAPGQRGEPGPQGHAGAQGPPGPPG

SEQ ID NO: 410 - protein, 48 aa, synthetic construct
  REGION 1..48: note = Recombinant collagen fragment
GFPGSPGAKGEVGPAGSPGSNGAPGQRGEPGPQGHAGAQGPPGPPGIN

SEQ ID NO: 411 - protein, 49 aa, synthetic construct
  REGION 1..49: note = Recombinant collagen fragment
GFPGSPGAKGEVGPAGSPGSNGAPGQRGEPGPQGHAGAQGPPGPPGING

SEQ ID NO: 412 - protein, 52 aa, synthetic construct
  REGION 1..52: note = Recombinant collagen fragment
GFPGSPGAKGEVGPAGSPGSNGAPGQRGEPGPQGHAGAQGPPGPPGINGSPG

SEQ ID NO: 413 - protein, 54 aa, synthetic construct
  REGION 1..54: note = Recombinant collagen fragment
GFPGSPGAKGEVGPAGSPGSNGAPGQRGEPGPQGHAGAQGPPGPPGINGSPGGK

SEQ ID NO: 414 - protein, 27 aa, synthetic construct
  REGION 1..27: note = Recombinant collagen fragment
GFRGPAGPNGIPGEKGPAGERGAPGPA

SEQ ID NO: 415 - protein, 18 aa, synthetic construct
  REGION 1..18: note = Recombinant collagen fragment
GGAGEPGKNGAKGEPGPR

SEQ ID NO: 416 - protein, 27 aa, synthetic construct
  REGION 1..27: note = Recombinant collagen fragment
GGAGEPGKNGAKGEPGPRGERGEAGIP

SEQ ID NO: 417 - protein, 28 aa, synthetic construct
  REGION 1..28: note = Recombinant collagen fragment
GGAGEPGKNGAKGEPGPRGERGEAGIPG

SEQ ID NO: 418 - protein, 40 aa, synthetic construct
  REGION 1..40: note = Recombinant collagen fragment
GGAGEPGKNGAKGEPGPRGERGEAGIPGVPGAKGEDGKDG

SEQ ID NO: 419 - protein, 48 aa, synthetic construct
  REGION 1..48: note = Recombinant collagen fragment
GGAGEPGKNGAKGEPGPRGERGEAGIPGVPGAKGEDGKDGSPGEPGAN

SEQ ID NO: 420 - protein, 18 aa, synthetic construct
  REGION 1..18: note = Recombinant collagen fragment
GGKGEMGPAGIPGAPGLM

SEQ ID NO: 421 - protein, 20 aa, synthetic construct
  REGION 1..20: note = Recombinant collagen fragment
GGKGEMGPAGIPGAPGLMGA

SEQ ID NO: 422 - protein, 21 aa, synthetic construct
  REGION 1..21: note = Recombinant collagen fragment
GGKGEMGPAGIPGAPGLMGAR

SEQ ID NO: 423 - protein, 32 aa, synthetic construct
  REGION 1..32: note = Recombinant collagen fragment
GGPGGPGPQGPPGKNGETGPQGPPGPTGPGGD

SEQ ID NO: 424 - protein, 48 aa, synthetic construct
  REGION 1..48: note = Recombinant collagen fragment
GGPGMRGMPGSPGGPGSDGKPGPPGSQGESGRPGPPGPSGPRGQPGVM

SEQ ID NO: 425 - protein, 25 aa, synthetic construct
  REGION 1..25: note = Recombinant collagen fragment
GGPGSDGKPGPPGSQGESGRPGPPG

SEQ ID NO: 426 - protein, 27 aa, synthetic construct
  REGION 1..27: note = Recombinant collagen fragment
GGPGSDGKPGPPGSQGESGRPGPPGPS

SEQ ID NO: 427 - protein, 30 aa, synthetic construct
  REGION 1..30: note = Recombinant collagen fragment
GGPGSDGKPGPPGSQGESGRPGPPGPSGPR

SEQ ID NO: 428 - protein, 32 aa, synthetic construct
  REGION 1..32: note = Recombinant collagen fragment
GGPGSDGKPGPPGSQGESGRPGPPGPSGPRGQ

SEQ ID NO: 429 - protein, 36 aa, synthetic construct
  REGION 1..36: note = Recombinant collagen fragment
GGPGSDGKPGPPGSQGESGRPGPPGPSGPRGQPGVM

SEQ ID NO: 430 - protein, 15 aa, synthetic construct
  REGION 1..15: note = Recombinant collagen fragment
GHAGAQGPPGPPGIN

SEQ ID NO: 431 - protein, 16 aa, synthetic construct
  REGION 1..16: note = Recombinant collagen fragment
GHAGAQGPPGPPGING

SEQ ID NO: 432 - protein, 19 aa, synthetic construct
  REGION 1..19: note = Recombinant collagen fragment
GHAGAQGPPGPPGINGSPG

SEQ ID NO: 433 - protein, 21 aa, synthetic construct
  REGION 1..21: note = Recombinant collagen fragment
GHAGAQGPPGPPGINGSPGGK

SEQ ID NO: 434 - protein, 25 aa, synthetic construct
  REGION 1..25: note = Recombinant collagen fragment
GHAGAQGPPGPPGINGSPGGKGEMG

SEQ ID NO: 435 - protein, 27 aa, synthetic construct
  REGION 1..27: note = Recombinant collagen fragment
GHAGAQGPPGPPGINGSPGGKGEMGPA

SEQ ID NO: 436 - protein, 36 aa, synthetic construct
  REGION 1..36: note = Recombinant collagen fragment
GHAGAQGPPGPPGINGSPGGKGEMGPAGIPGAPGLM

SEQ ID NO: 437 - protein, 37 aa, synthetic construct
  REGION 1..37: note = Recombinant collagen fragment
GHAGAQGPPGPPGINGSPGGKGEMGPAGIPGAPGLMG

SEQ ID NO: 438 - protein, 38 aa, synthetic construct
  REGION 1..38: note = Recombinant collagen fragment
GHAGAQGPPGPPGINGSPGGKGEMGPAGIPGAPGLMGA

SEQ ID NO: 439 - protein, 39 aa, synthetic construct
  REGION 1..39: note = Recombinant collagen fragment
GHAGAQGPPGPPGINGSPGGKGEMGPAGIPGAPGLMGAR

SEQ ID NO: 440 - protein, 45 aa, synthetic construct
  REGION 1..45: note = Recombinant collagen fragment
GHAGAQGPPGPPGINGSPGGKGEMGPAGIPGAPGLMGARGPPGPA

SEQ ID NO: 441 - protein, 48 aa, synthetic construct
  REGION 1..48: note = Recombinant collagen fragment
GHAGAQGPPGPPGINGSPGGKGEMGPAGIPGAPGLMGARGPPGPAGAN

SEQ ID NO: 442 - protein, 54 aa, synthetic construct
  REGION 1..54: note = Recombinant collagen fragment
GHAGAQGPPGPPGINGSPGGKGEMGPAGIPGAPGLMGARGPPGPAGANGAPGLR

SEQ ID NO: 443 - protein, 19 aa, synthetic construct
  REGION 1..19: note = Recombinant collagen fragment
GHPGSPGSPGYQGPPGEPG

SEQ ID NO: 444 - protein, 21 aa, synthetic construct
  REGION 1..21: note = Recombinant collagen fragment
GHPGSPGSPGYQGPPGEPGQA

SEQ ID NO: 445 - protein, 22 aa, synthetic construct
  REGION 1..22: note = Recombinant collagen fragment
GHPGSPGSPGYQGPPGEPGQAG

SEQ ID NO: 446 - protein, 24 aa, synthetic construct
  REGION 1..24: note = Recombinant collagen fragment
GHPGSPGSPGYQGPPGEPGQAGPS

SEQ ID NO: 447 - protein, 36 aa, synthetic construct
  REGION 1..36: note = Recombinant collagen fragment
GHPGSPGSPGYQGPPGEPGQAGPSGPPGPPGAIGPS

SEQ ID NO: 448 - protein, 39 aa, synthetic construct
  REGION 1..39: note = Recombinant collagen fragment
GHPGSPGSPGYQGPPGEPGQAGPSGPPGPPGAIGPSGPA

SEQ ID NO: 449 - protein, 42 aa, synthetic construct
  REGION 1..42: note = Recombinant collagen fragment
GHPGSPGSPGYQGPPGEPGQAGPSGPPGPPGAIGPSGPAGKD

SEQ ID NO: 450 - protein, 45 aa, synthetic construct
  REGION 1..45: note = Recombinant collagen fragment
GHPGSPGSPGYQGPPGEPGQAGPSGPPGPPGAIGPSGPAGKDGES

SEQ ID NO: 451 - protein, 54 aa, synthetic construct
  REGION 1..54: note = Recombinant collagen fragment
GHPGSPGSPGYQGPPGEPGQAGPSGPPGPPGAIGPSGPAGKDGESGRPGRPGER

SEQ ID NO: 452 - protein, 21 aa, synthetic construct
  REGION 1..21: note = Recombinant collagen fragment
GHRGFDGRNGEKGETGAPGLK

SEQ ID NO: 453 - protein, 24 aa, synthetic construct
  REGION 1..24: note = Recombinant collagen fragment
GHRGFDGRNGEKGETGAPGLKGEN

SEQ ID NO: 454 - protein, 25 aa, synthetic construct
  REGION 1..25: note = Recombinant collagen fragment
GHRGFDGRNGEKGETGAPGLKGENG

SEQ ID NO: 455 - protein, 30 aa, synthetic construct
  REGION 1..30: note = Recombinant collagen fragment
GHRGFDGRNGEKGETGAPGLKGENGLPGEN

SEQ ID NO: 456 - protein, 36 aa, synthetic construct
  REGION 1..36: note = Recombinant collagen fragment
GHRGFDGRNGEKGETGAPGLKGENGLPGENGAPGPM

SEQ ID NO: 457 - protein, 39 aa, synthetic construct
  REGION 1..39: note = Recombinant collagen fragment
GHRGFDGRNGEKGETGAPGLKGENGLPGENGAPGPMGPR

SEQ ID NO: 458 - protein, 45 aa, synthetic construct
  REGION 1..45: note = Recombinant collagen fragment
GHRGFDGRNGEKGETGAPGLKGENGLPGENGAPGPMGPRGAPGER

SEQ ID NO: 459 - protein, 9 aa, synthetic construct
  REGION 1..9: note = Recombinant collagen fragment
GIKGPAGIP

SEQ ID NO: 460 - protein, 13 aa, synthetic construct
  REGION 1..13: note = Recombinant collagen fragment
GIKGPAGIPGFPG

SEQ ID NO: 461 - protein, 16 aa, synthetic construct
  REGION 1..16: note = Recombinant collagen fragment
GIKGPAGIPGFPGMKG

SEQ ID NO: 462 - protein, 9 aa, synthetic construct
  REGION 1..9: note = Recombinant collagen fragment
GIPGAPGLM

SEQ ID NO: 463 - protein, 10 aa, synthetic construct
  REGION 1..10: note = Recombinant collagen fragment
GIPGAPGLMG

SEQ ID NO: 464 - protein, 11 aa, synthetic construct
  REGION 1..11: note = Recombinant collagen fragment
GIPGAPGLMGA

SEQ ID NO: 465 - protein, 12 aa, synthetic construct
  REGION 1..12: note = Recombinant collagen fragment
GIPGAPGLMGAR

SEQ ID NO: 466 - protein, 16 aa, synthetic construct
  REGION 1..16: note = Recombinant collagen fragment
GIPGAPGLMGARGPPG

SEQ ID NO: 467 - protein, 18 aa, synthetic construct
  REGION 1..18: note = Recombinant collagen fragment
GIPGAPGLMGARGPPGPA

SEQ ID NO: 468 - protein, 19 aa, synthetic construct
  REGION 1..19: note = Recombinant collagen fragment
GIPGAPGLMGARGPPGPAG

SEQ ID NO: 469 - protein, 20 aa, synthetic construct
  REGION 1..20: note = Recombinant collagen fragment
GIPGAPGLMGARGPPGPAGA

SEQ ID NO: 470 - protein, 21 aa, synthetic construct
  REGION 1..21: note = Recombinant collagen fragment
GIPGAPGLMGARGPPGPAGAN

SEQ ID NO: 471 - protein, 27 aa, synthetic construct
  REGION 1..27: note = Recombinant collagen fragment
GIPGAPGLMGARGPPGPAGANGAPGLR

SEQ ID NO: 472 - protein, 28 aa, synthetic construct
  REGION 1..28: note = Recombinant collagen fragment
GIPGAPGLMGARGPPGPAGANGAPGLRG

SEQ ID NO: 473 - protein, 36 aa, synthetic construct
  REGION 1..36: note = Recombinant collagen fragment
GIPGAPGLMGARGPPGPAGANGAPGLRGGAGEPGKN

SEQ ID NO: 474 - protein, 18 aa, synthetic construct
  REGION 1..18: note = Recombinant collagen fragment
GIPGEKGPAGERGAPGPA

SEQ ID NO: 475 - protein, 19 aa, synthetic construct
  REGION 1..19: note = Recombinant collagen fragment
GIPGEKGPAGERGAPGPAG

SEQ ID NO: 476 - protein, 21 aa, synthetic construct
  REGION 1..21: note = Recombinant collagen fragment
GIPGEKGPAGERGAPGPAGPR

SEQ ID NO: 477 - protein, 22 aa, synthetic construct
  REGION 1..22: note = Recombinant collagen fragment
GIPGEKGPAGERGAPGPAGPRG

SEQ ID NO: 478 - protein, 23 aa, synthetic construct
  REGION 1..23: note = Recombinant collagen fragment
GIPGEKGPAGERGAPGPAGPRGA

SEQ ID NO: 479 - protein, 24 aa, synthetic construct
  REGION 1..24: note = Recombinant collagen fragment
GIPGEKGPAGERGAPGPAGPRGAA

SEQ ID NO: 480 - protein, 25 aa, synthetic construct
  REGION 1..25: note = Recombinant collagen fragment
GIPGEKGPAGERGAPGPAGPRGAAG

SEQ ID NO: 481 - protein, 28 aa, synthetic construct
  REGION 1..28: note = Recombinant collagen fragment
GIPGEKGPAGERGAPGPAGPRGAAGEPG

SEQ ID NO: 482 - protein, 29 aa, synthetic construct
  REGION 1..29: note = Recombinant collagen fragment
GIPGEKGPAGERGAPGPAGPRGAAGEPGR

SEQ ID NO: 483 - protein, 30 aa, synthetic construct
  REGION 1..30: note = Recombinant collagen fragment
GIPGEKGPAGERGAPGPAGPRGAAGEPGRD

SEQ ID NO: 484 - protein, 34 aa, synthetic construct
  REGION 1..34: note = Recombinant collagen fragment
GIPGEKGPAGERGAPGPAGPRGAAGEPGRDGVPG

SEQ ID NO: 485 - protein, 35 aa, synthetic construct
  REGION 1..35: note = Recombinant collagen fragment
GIPGEKGPAGERGAPGPAGPRGAAGEPGRDGVPGG

SEQ ID NO: 486 - protein, 37 aa, synthetic construct
  REGION 1..37: note = Recombinant collagen fragment
GIPGEKGPAGERGAPGPAGPRGAAGEPGRDGVPGGPG

SEQ ID NO: 487 - protein, 39 aa, synthetic construct
  REGION 1..39: note = Recombinant collagen fragment
GIPGEKGPAGERGAPGPAGPRGAAGEPGRDGVPGGPGMR

SEQ ID NO: 488 - protein, 11 aa, synthetic construct
  REGION 1..11: note = Recombinant collagen fragment
GIPGFPGMKGH

SEQ ID NO: 489 - protein, 12 aa, synthetic construct
  REGION 1..12: note = Recombinant collagen fragment
GIPGFPGMKGHR

SEQ ID NO: 490 - protein, 14 aa, synthetic construct
  REGION 1..14: note = Recombinant collagen fragment
GIPGFPGMKGHRGF

SEQ ID NO: 491 - protein, 15 aa, synthetic construct
  REGION 1..15: note = Recombinant collagen fragment
GIPGFPGMKGHRGFD

SEQ ID NO: 492 - protein, 17 aa, synthetic construct
  REGION 1..17: note = Recombinant collagen fragment
GIPGFPGMKGHRGFDGR

SEQ ID NO: 493 - protein, 18 aa, synthetic construct
  REGION 1..18: note = Recombinant collagen fragment
GIPGFPGMKGHRGFDGRN

SEQ ID NO: 494 - protein, 21 aa, synthetic construct
  REGION 1..21: note = Recombinant collagen fragment
GIPGFPGMKGHRGFDGRNGEK

SEQ ID NO: 495 - protein, 22 aa, synthetic construct
  REGION 1..22: note = Recombinant collagen fragment
GIPGFPGMKGHRGFDGRNGEKG

SEQ ID NO: 496 - protein, 30 aa, synthetic construct
  REGION 1..30: note = Recombinant collagen fragment
GIPGFPGMKGHRGFDGRNGEKGETGAPGLK

SEQ ID NO: 497 - protein, 33 aa, synthetic construct
  REGION 1..33: note = Recombinant collagen fragment
GIPGFPGMKGHRGFDGRNGEKGETGAPGLKGEN

SEQ ID NO: 498 - protein, 39 aa, synthetic construct
  REGION 1..39: note = Recombinant collagen fragment
GIPGFPGMKGHRGFDGRNGEKGETGAPGLKGENGLPGEN

SEQ ID NO: 499 - protein, 48 aa, synthetic construct
  REGION 1..48: note = Recombinant collagen fragment
GIPGFPGMKGHRGFDGRNGEKGETGAPGLKGENGLPGENGAPGPMGPR

SEQ ID NO: 500 - protein, 24 aa, synthetic construct
  REGION 1..24: note = Recombinant collagen fragment
GKDGESGRPGRPGERGLPGPPGIK

SEQ ID NO: 501 - protein, 27 aa, synthetic construct
  REGION 1..27: note = Recombinant collagen fragment
GKDGESGRPGRPGERGLPGPPGIKGPA

SEQ ID NO: 502 - protein, 36 aa, synthetic construct
  REGION 1..36: note = Recombinant collagen fragment
GKDGESGRPGRPGERGLPGPPGIKGPAGIPGFPGMK

SEQ ID NO: 503 - protein, 17 aa, synthetic construct
  REGION 1..17: note = Recombinant collagen fragment
GKDGSPGEPGANGLPGA

SEQ ID NO: 504 - protein, 25 aa, synthetic construct
  REGION 1..25: note = Recombinant collagen fragment
GKDGSPGEPGANGLPGAAGERGAPG

SEQ ID NO: 505 - protein, 33 aa, synthetic construct
  REGION 1..33: note = Recombinant collagen fragment
GKDGSPGEPGANGLPGAAGERGAPGFRGPAGPN

SEQ ID NO: 506 - protein, 17 aa, synthetic construct
  REGION 1..17: note = Recombinant collagen fragment
GKGEMGPAGIPGAPGLM

SEQ ID NO: 507 - protein, 18 aa, synthetic construct
  REGION 1..18: note = Recombinant collagen fragment
GKGEMGPAGIPGAPGLMG

SEQ ID NO: 508 - protein, 19 aa, synthetic construct
  REGION 1..19: note = Recombinant collagen fragment
GKGEMGPAGIPGAPGLMGA

SEQ ID NO: 509 - protein, 20 aa, synthetic construct
  REGION 1..20: note = Recombinant collagen fragment
GKGEMGPAGIPGAPGLMGAR

SEQ ID NO: 510 - protein, 26 aa, synthetic construct
  REGION 1..26: note = Recombinant collagen fragment
GKGEMGPAGIPGAPGLMGARGPPGPA

SEQ ID NO: 511 - protein, 29 aa, synthetic construct
  REGION 1..29: note = Recombinant collagen fragment
GKGEMGPAGIPGAPGLMGARGPPGPAGAN

SEQ ID NO: 512 - protein, 35 aa, synthetic construct
  REGION 1..35: note = Recombinant collagen fragment
GKGEMGPAGIPGAPGLMGARGPPGPAGANGAPGLR

SEQ ID NO: 513 - protein, 28 aa, synthetic construct
  REGION 1..28: note = Recombinant collagen fragment
GKPGEPGPKGDAGAPGAPGGKGDAGAPG

SEQ ID NO: 514 - protein, 29 aa, synthetic construct
  REGION 1..29: note = Recombinant collagen fragment
GKPGEPGPKGDAGAPGAPGGKGDAGAPGE

SEQ ID NO: 515 - protein, 30 aa, synthetic construct
  REGION 1..30: note = Recombinant collagen fragment
GKPGEPGPKGDAGAPGAPGGKGDAGAPGER

SEQ ID NO: 516 - protein, 31 aa, synthetic construct
  REGION 1..31: note = Recombinant collagen fragment
GKPGEPGPKGDAGAPGAPGGKGDAGAPGERG

SEQ ID NO: 517 - protein, 33 aa, synthetic construct
  REGION 1..33: note = Recombinant collagen fragment
GKPGEPGPKGDAGAPGAPGGKGDAGAPGERGPP

SEQ ID NO: 518 - protein, 24 aa, synthetic construct
  REGION 1..24: note = Recombinant collagen fragment
GKPGPPGSQGESGRPGPPGPSGPR

SEQ ID NO: 519 - protein, 26 aa, synthetic construct
  REGION 1..26: note = Recombinant collagen fragment
GKPGPPGSQGESGRPGPPGPSGPRGQ

SEQ ID NO: 520 - protein, 30 aa, synthetic construct
  REGION 1..30: note = Recombinant collagen fragment
GKPGPPGSQGESGRPGPPGPSGPRGQPGVM

SEQ ID NO: 521 - protein, 21 aa, synthetic construct
  REGION 1..21: note = Recombinant collagen fragment
GLAGYPGPAGPPGPPGPPGTS

SEQ ID NO: 522 - protein, 57 aa, synthetic construct
  REGION 1..57: note = Recombinant collagen fragment
GLAGYPGPAGPPGPPGPPGTSGHPGSPGSPGYQGPPGEPGQAGPSGPPGPPGAIGPS

SEQ ID NO: 523 - protein, 21 aa, synthetic construct
  REGION 1..21: note = Recombinant collagen fragment
GLKGENGLPGENGAPGPMGPR

SEQ ID NO: 524 - protein, 21 aa, synthetic construct
  REGION 1..21: note = Recombinant collagen fragment
GLMGARGPPGPAGANGAPGLR

SEQ ID NO: 525 - protein, 30 aa, synthetic construct
  REGION 1..30: note = Recombinant collagen fragment
GLPGAAGARGNDGARGSDGQPGPPGPPGTA

SEQ ID NO: 526 - protein, 45 aa, synthetic construct
  REGION 1..45: note = Recombinant collagen fragment
GLPGAAGARGNDGARGSDGQPGPPGPPGTAGFPGSPGAKGEVGPA

SEQ ID NO: 527 - protein, 14 aa, synthetic construct
  REGION 1..14: note = Recombinant collagen fragment
GLPGAAGERGAPGF

SEQ ID NO: 528 - protein, 15 aa, synthetic construct
  REGION 1..15: note = Recombinant collagen fragment
GLPGAAGERGAPGFR

SEQ ID NO: 529 - protein, 16 aa, synthetic construct
  REGION 1..16: note = Recombinant collagen fragment
GLPGAAGERGAPGFRG

SEQ ID NO: 530 - protein, 19 aa, synthetic construct
  REGION 1..19: note = Recombinant collagen fragment
GLPGAAGERGAPGFRGPAG

SEQ ID NO: 531 - protein, 21 aa, synthetic construct
  REGION 1..21: note = Recombinant collagen fragment
GLPGAAGERGAPGFRGPAGPN

SEQ ID NO: 532 - protein, 28 aa, synthetic construct
  REGION 1..28: note = Recombinant collagen fragment
GLPGAAGERGAPGFRGPAGPNGIPGEKG

SEQ ID NO: 533 - protein, 33 aa, synthetic construct
  REGION 1..33: note = Recombinant collagen fragment
GLPGAAGERGAPGFRGPAGPNGIPGEKGPAGER

SEQ ID NO: 534 - protein, 37 aa, synthetic construct
  REGION 1..37: note = Recombinant collagen fragment
GLPGAAGERGAPGFRGPAGPNGIPGEKGPAGERGAPG

SEQ ID NO: 535 - protein, 42 aa, synthetic construct
  REGION 1..42: note = Recombinant collagen fragment
GLPGAAGERGAPGFRGPAGPNGIPGEKGPAGERGAPGPAGPR

SEQ ID NO: 536 - protein, 12 aa, synthetic construct
  REGION 1..12: note = Recombinant collagen fragment
GLPGENGAPGPM

SEQ ID NO: 537 - protein, 13 aa, synthetic construct
  REGION 1..13: note = Recombinant collagen fragment
GLPGENGAPGPMG

SEQ ID NO: 538 - protein, 15 aa, synthetic construct
  REGION 1..15: note = Recombinant collagen fragment
GLPGENGAPGPMGPR

SEQ ID NO: 539 - protein, 16 aa, synthetic construct
  REGION 1..16: note = Recombinant collagen fragment
GLPGENGAPGPMGPRG

SEQ ID NO: 540 - protein, 19 aa, synthetic construct
  REGION 1..19: note = Recombinant collagen fragment
GLPGENGAPGPMGPRGAPG

SEQ ID NO: 541 - protein, 23 aa, synthetic construct
  REGION 1..23: note = Recombinant collagen fragment
GLPGENGAPGPMGPRGAPGERGR

SEQ ID NO: 542 - protein, 25 aa, synthetic construct
  REGION 1..25: note = Recombinant collagen fragment
GLPGENGAPGPMGPRGAPGERGRPG

SEQ ID NO: 543 - protein, 33 aa, synthetic construct
  REGION 1..33: note = Recombinant collagen fragment
GLPGENGAPGPMGPRGAPGERGRPGLPGAAGAR

SEQ ID NO: 544 - protein, 35 aa, synthetic construct
  REGION 1..35: note = Recombinant collagen fragment
GLPGENGAPGPMGPRGAPGERGRPGLPGAAGARGN

SEQ ID NO: 545 - protein, 40 aa, synthetic construct
  REGION 1..40: note = Recombinant collagen fragment
GLPGENGAPGPMGPRGAPGERGRPGLPGAAGARGNDGARG

SEQ ID NO: 546 - protein, 44 aa, synthetic construct
  REGION 1..44: note = Recombinant collagen fragment
GLPGENGAPGPMGPRGAPGERGRPGLPGAAGARGNDGARGSDGQ

SEQ ID NO: 547 - protein, 54 aa, synthetic construct
  REGION 1..54: note = Recombinant collagen fragment
GLPGENGAPGPMGPRGAPGERGRPGLPGAAGARGNDGARGSDGQPGPPGPPGTA

SEQ ID NO: 548 - protein, 9 aa, synthetic construct
  REGION 1..9: note = Recombinant collagen fragment
GLPGPPGIK

SEQ ID NO: 549 - protein, 10 aa, synthetic construct
  REGION 1..10: note = Recombinant collagen fragment
GLPGPPGIKG

SEQ ID NO: 550 - protein, 11 aa, synthetic construct
  REGION 1..11: note = Recombinant collagen fragment
GLPGPPGIKGP

SEQ ID NO: 551 - protein, 12 aa, synthetic construct
  REGION 1..12: note = Recombinant collagen fragment
GLPGPPGIKGPA

SEQ ID NO: 552 - protein, 13 aa, synthetic construct
  REGION 1..13: note = Recombinant collagen fragment
GLPGPPGIKGPAG

SEQ ID NO: 553 - protein, 15 aa, synthetic construct
  REGION 1..15: note = Recombinant collagen fragment
GLPGPPGIKGPAGIP

SEQ ID NO: 554 - protein, 16 aa, synthetic construct
  REGION 1..16: note = Recombinant collagen fragment
GLPGPPGIKGPAGIPG

SEQ ID NO: 555 - protein, 17 aa, synthetic construct
  REGION 1..17: note = Recombinant collagen fragment
GLPGPPGIKGPAGIPGF

SEQ ID NO: 556 - protein, 20 aa, synthetic construct
  REGION 1..20: note = Recombinant collagen fragment
GLPGPPGIKGPAGIPGFPGM

SEQ ID NO: 557 - protein, 21 aa, synthetic construct
  REGION 1..21: note = Recombinant collagen fragment
GLPGPPGIKGPAGIPGFPGMK

SEQ ID NO: 558 - protein, 22 aa, synthetic construct
  REGION 1..22: note = Recombinant collagen fragment
GLPGPPGIKGPAGIPGFPGMKG

SEQ ID NO: 559 - protein, 23 aa, synthetic construct
  REGION 1..23: note = Recombinant collagen fragment
GLPGPPGIKGPAGIPGFPGMKGH

SEQ ID NO: 560 - protein, 24 aa, synthetic construct
  REGION 1..24: note = Recombinant collagen fragment
GLPGPPGIKGPAGIPGFPGMKGHR

SEQ ID NO: 561 - protein, 26 aa, synthetic construct
  REGION 1..26: note = Recombinant collagen fragment
GLPGPPGIKGPAGIPGFPGMKGHRGF

SEQ ID NO: 562 - protein, 12 aa, synthetic construct
  REGION 1..12: note = Recombinant collagen fragment
GLPGTGGPPGEN

SEQ ID NO: 563 - protein, 30 aa, synthetic construct
  REGION 1..30: note = Recombinant collagen fragment
GLRGGAGEPGKNGAKGEPGPRGERGEAGIP

SEQ ID NO: 564 - protein, 19 aa, synthetic construct
  REGION 1..19: note = Recombinant collagen fragment
GMPGSPGGPGSDGKPGPPG

SEQ ID NO: 565 - protein, 21 aa, synthetic construct
  REGION 1..21: note = Recombinant collagen fragment
GMPGSPGGPGSDGKPGPPGSQ

SEQ ID NO: 566 - protein, 22 aa, synthetic construct
  REGION 1..22: note = Recombinant collagen fragment
GMPGSPGGPGSDGKPGPPGSQG

SEQ ID NO: 567 - protein, 36 aa, synthetic construct
  REGION 1..36: note = Recombinant collagen fragment
GMPGSPGGPGSDGKPGPPGSQGESGRPGPPGPSGPR

SEQ ID NO: 568 - protein, 42 aa, synthetic construct
  REGION 1..42: note = Recombinant collagen fragment
GMPGSPGGPGSDGKPGPPGSQGESGRPGPPGPSGPRGQPGVM

SEQ ID NO: 569 - protein, 45 aa, synthetic construct
  REGION 1..45: note = Recombinant collagen fragment
GMRGMPGSPGGPGSDGKPGPPGSQGESGRPGPPGPSGPRGQPGVM

SEQ ID NO: 570 - protein, 27 aa, synthetic construct
  REGION 1..27: note = Recombinant collagen fragment
GNDGAPGKNGERGGPGGPGPQGPPGKN

SEQ ID NO: 571 - protein, 34 aa, synthetic construct
  REGION 1..34: note = Recombinant collagen fragment
GNDGAPGKNGERGGPGGPGPQGPPGKNGETGPQG

SEQ ID NO: 572 - protein, 38 aa, synthetic construct
  REGION 1..38: note = Recombinant collagen fragment
GNDGAPGKNGERGGPGGPGPQGPPGKNGETGPQGPPGP

SEQ ID NO: 573 - protein, 19 aa, synthetic construct
  REGION 1..19: note = Recombinant collagen fragment
GNDGARGSDGQPGPPGPPG

SEQ ID NO: 574 - protein, 21 aa, synthetic construct
  REGION 1..21: note = Recombinant collagen fragment
GNDGARGSDGQPGPPGPPGTA

SEQ ID NO: 575 - protein, 22 aa, synthetic construct
  REGION 1..22: note = Recombinant collagen fragment
GNDGARGSDGQPGPPGPPGTAG

SEQ ID NO: 576 - protein, 23 aa, synthetic construct
  REGION 1..23: note = Recombinant collagen fragment
GNDGARGSDGQPGPPGPPGTAGF

SEQ ID NO: 577 - protein, 25 aa, synthetic construct
  REGION 1..25: note = Recombinant collagen fragment
GNDGARGSDGQPGPPGPPGTAGFPG

SEQ ID NO: 578 - protein, 26 aa, synthetic construct
  REGION 1..26: note = Recombinant collagen fragment
GNDGARGSDGQPGPPGPPGTAGFPGS

SEQ ID NO: 579 - protein, 28 aa, synthetic construct
  REGION 1..28: note = Recombinant collagen fragment
GNDGARGSDGQPGPPGPPGTAGFPGSPG

SEQ ID NO: 580 - protein, 30 aa, synthetic construct
  REGION 1..30: note = Recombinant collagen fragment
GNDGARGSDGQPGPPGPPGTAGFPGSPGAK

SEQ ID NO: 581 - protein, 31 aa, synthetic construct
  REGION 1..31: note = Recombinant collagen fragment
GNDGARGSDGQPGPPGPPGTAGFPGSPGAKG

SEQ ID NO: 582 - protein, 32 aa, synthetic construct
  REGION 1..32: note = Recombinant collagen fragment
GNDGARGSDGQPGPPGPPGTAGFPGSPGAKGE

SEQ ID NO: 583 - protein, 34 aa, synthetic construct
  REGION 1..34: note = Recombinant collagen fragment
GNDGARGSDGQPGPPGPPGTAGFPGSPGAKGEVG

SEQ ID NO: 584 - protein, 35 aa, synthetic construct
  REGION 1..35: note = Recombinant collagen fragment
GNDGARGSDGQPGPPGPPGTAGFPGSPGAKGEVGP

SEQ ID NO: 585 - protein, 36 aa, synthetic construct
  REGION 1..36: note = Recombinant collagen fragment
GNDGARGSDGQPGPPGPPGTAGFPGSPGAKGEVGPA

SEQ ID NO: 586 - protein, 37 aa, synthetic construct
  REGION 1..37: note = Recombinant collagen fragment
GNDGARGSDGQPGPPGPPGTAGFPGSPGAKGEVGPAG

SEQ ID NO: 587 - protein, 38 aa, synthetic construct
  REGION 1..38: note = Recombinant collagen fragment
GNDGARGSDGQPGPPGPPGTAGFPGSPGAKGEVGPAGS

SEQ ID NO: 588 - protein, 40 aa, synthetic construct
  REGION 1..40: note = Recombinant collagen fragment
GNDGARGSDGQPGPPGPPGTAGFPGSPGAKGEVGPAGSPG

SEQ ID NO: 589 - protein, 42 aa, synthetic construct
  REGION 1..42: note = Recombinant collagen fragment
GNDGARGSDGQPGPPGPPGTAGFPGSPGAKGEVGPAGSPGSN

SEQ ID NO: 590 - protein, 43 aa, synthetic construct
  REGION 1..43: note = Recombinant collagen fragment
GNDGARGSDGQPGPPGPPGTAGFPGSPGAKGEVGPAGSPGSNG

SEQ ID NO: 591 - protein, 47 aa, synthetic construct
  REGION 1..47: note = Recombinant collagen fragment
GNDGARGSDGQPGPPGPPGTAGFPGSPGAKGEVGPAGSPGSNGAPGQ

SEQ ID NO: 592 - protein, 48 aa, synthetic construct
  REGION 1..48: note = Recombinant collagen fragment
GNDGARGSDGQPGPPGPPGTAGFPGSPGAKGEVGPAGSPGSNGAPGQR

SEQ ID NO: 593 - protein, 50 aa, synthetic construct
  REGION 1..50: note = Recombinant collagen fragment
GNDGARGSDGQPGPPGPPGTAGFPGSPGAKGEVGPAGSPGSNGAPGQRGE

SEQ ID NO: 594 - protein, 54 aa, synthetic construct
  REGION 1..54: note = Recombinant collagen fragment
GNDGARGSDGQPGPPGPPGTAGFPGSPGAKGEVGPAGSPGSNGAPGQRGEPGPQ

SEQ ID NO: 595 - protein, 55 aa, synthetic construct
  REGION 1..55: note = Recombinant collagen fragment
GNDGARGSDGQPGPPGPPGTAGFPGSPGAKGEVGPAGSPGSNGAPGQRGEPGPQG

SEQ ID NO: 596 - protein, 19 aa, synthetic construct
  REGION 1..19: note = Recombinant collagen fragment
GPAGANGAPGLRGGAGEPG

SEQ ID NO: 597 - protein, 12 aa, synthetic construct
  REGION 1..12: note = Recombinant collagen fragment
GPAGIPGAPGLM

SEQ ID NO: 598 - protein, 13 aa, synthetic construct
  REGION 1..13: note = Recombinant collagen fragment
GPAGIPGAPGLMG

SEQ ID NO: 599 - protein, 14 aa, synthetic construct
  REGION 1..14: note = Recombinant collagen fragment
GPAGIPGAPGLMGA

SEQ ID NO: 600 - protein, 15 aa, synthetic construct
  REGION 1..15: note = Recombinant collagen fragment
GPAGIPGAPGLMGAR

SEQ ID NO: 601 - protein, 30 aa, synthetic construct
  REGION 1..30: note = Recombinant collagen fragment
GPAGIPGAPGLMGARGPPGPAGANGAPGLR

SEQ ID NO: 602 - protein, 9 aa, synthetic construct
  REGION 1..9: note = Recombinant collagen fragment
GPAGIPGFP

SEQ ID NO: 603 - protein, 10 aa, synthetic construct
  REGION 1..10: note = Recombinant collagen fragment
GPAGIPGFPG

SEQ ID NO: 604 - protein, 11 aa, synthetic construct
  REGION 1..11: note = Recombinant collagen fragment
GPAGIPGFPGM

SEQ ID NO: 605 - protein, 12 aa, synthetic construct
  REGION 1..12: note = Recombinant collagen fragment
GPAGIPGFPGMK

SEQ ID NO: 606 - protein, 13 aa, synthetic construct
  REGION 1..13: note = Recombinant collagen fragment
GPAGIPGFPGMKG

SEQ ID NO: 607 - protein, 14 aa, synthetic construct
  REGION 1..14: note = Recombinant collagen fragment
GPAGIPGFPGMKGH

SEQ ID NO: 608 - protein, 15 aa, synthetic construct
  REGION 1..15: note = Recombinant collagen fragment
GPAGIPGFPGMKGHR

SEQ ID NO: 609 - protein, 16 aa, synthetic construct
  REGION 1..16: note = Recombinant collagen fragment
GPAGIPGFPGMKGHRG

SEQ ID NO: 610 - protein, 17 aa, synthetic construct
  REGION 1..17: note = Recombinant collagen fragment
GPAGIPGFPGMKGHRGF

SEQ ID NO: 611 - protein, 18 aa, synthetic construct
  REGION 1..18: note = Recombinant collagen fragment
GPAGIPGFPGMKGHRGFD

SEQ ID NO: 612 - protein, 19 aa, synthetic construct
  REGION 1..19: note = Recombinant collagen fragment
GPAGIPGFPGMKGHRGFDG

SEQ ID NO: 613 - protein, 20 aa, synthetic construct
  REGION 1..20: note = Recombinant collagen fragment
GPAGIPGFPGMKGHRGFDGR

SEQ ID NO: 614 - protein, 21 aa, synthetic construct
  REGION 1..21: note = Recombinant collagen fragment
GPAGIPGFPGMKGHRGFDGRN

SEQ ID NO: 615 - protein, 22 aa, synthetic construct
  REGION 1..22: note = Recombinant collagen fragment
GPAGIPGFPGMKGHRGFDGRNG

SEQ ID NO: 616 - protein, 23 aa, synthetic construct
  REGION 1..23: note = Recombinant collagen fragment
GPAGIPGFPGMKGHRGFDGRNGE

SEQ ID NO: 617 - protein, 24 aa, synthetic construct
  REGION 1..24: note = Recombinant collagen fragment
GPAGIPGFPGMKGHRGFDGRNGEK

SEQ ID NO: 618 - protein, 25 aa, synthetic construct
  REGION 1..25: note = Recombinant collagen fragment
GPAGIPGFPGMKGHRGFDGRNGEKG

SEQ ID NO: 619 - protein, 26 aa, synthetic construct
  REGION 1..26: note = Recombinant collagen fragment
GPAGIPGFPGMKGHRGFDGRNGEKGE

SEQ ID NO: 620 - protein, 27 aa, synthetic construct
  REGION 1..27: note = Recombinant collagen fragment
GPAGIPGFPGMKGHRGFDGRNGEKGET

SEQ ID NO: 621 - protein, 28 aa, synthetic construct
  REGION 1..28: note = Recombinant collagen fragment
GPAGIPGFPGMKGHRGFDGRNGEKGETG

SEQ ID NO: 622 - protein, 29 aa, synthetic construct
  REGION 1..29: note = Recombinant collagen fragment
GPAGIPGFPGMKGHRGFDGRNGEKGETGA

SEQ ID NO: 623 - protein, 30 aa, synthetic construct
  REGION 1..30: note = Recombinant collagen fragment
GPAGIPGFPGMKGHRGFDGRNGEKGETGAP

SEQ ID NO: 624 - protein, 31 aa, synthetic construct
  REGION 1..31: note = Recombinant collagen fragment
GPAGIPGFPGMKGHRGFDGRNGEKGETGAPG

SEQ ID NO: 625 - protein, 32 aa, synthetic construct
  REGION 1..32: note = Recombinant collagen fragment
GPAGIPGFPGMKGHRGFDGRNGEKGETGAPGL

SEQ ID NO: 626 - protein, 33 aa, synthetic construct
  REGION 1..33: note = Recombinant collagen fragment
GPAGIPGFPGMKGHRGFDGRNGEKGETGAPGLK

SEQ ID NO: 627 - protein, 34 aa, synthetic construct
  REGION 1..34: note = Recombinant collagen fragment
GPAGIPGFPGMKGHRGFDGRNGEKGETGAPGLKG

SEQ ID NO: 628 - protein, 36 aa, synthetic construct
  REGION 1..36: note = Recombinant collagen fragment
GPAGIPGFPGMKGHRGFDGRNGEKGETGAPGLKGEN

SEQ ID NO: 629 - protein, 40 aa, synthetic construct
  REGION 1..40: note = Recombinant collagen fragment
GPAGIPGFPGMKGHRGFDGRNGEKGETGAPGLKGENGLPG

SEQ ID NO: 630 - protein, 42 aa, synthetic construct
  REGION 1..42: note = Recombinant collagen fragment
GPAGIPGFPGMKGHRGFDGRNGEKGETGAPGLKGENGLPGEN

SEQ ID NO: 631 - protein, 48 aa, synthetic construct
  REGION 1..48: note = Recombinant collagen fragment
GPAGIPGFPGMKGHRGFDGRNGEKGETGAPGLKGENGLPGENGAPGPM

SEQ ID NO: 632 - protein, 51 aa, synthetic construct
  REGION 1..51: note = Recombinant collagen fragment
GPAGIPGFPGMKGHRGFDGRNGEKGETGAPGLKGENGLPGENGAPGPMGPR

SEQ ID NO: 633 - protein, 24 aa, synthetic construct
  REGION 1..24: note = Recombinant collagen fragment
GPAGKDGESGRPGRPGERGLPGPP

SEQ ID NO: 634 - protein, 25 aa, synthetic construct
  REGION 1..25: note = Recombinant collagen fragment
GPAGKDGESGRPGRPGERGLPGPPG

SEQ ID NO: 635 - protein, 27 aa, synthetic construct
  REGION 1..27: note = Recombinant collagen fragment
GPAGKDGESGRPGRPGERGLPGPPGIK

SEQ ID NO: 636 - protein, 30 aa, synthetic construct
  REGION 1..30: note = Recombinant collagen fragment
GPAGKDGESGRPGRPGERGLPGPPGIKGPA

SEQ ID NO: 637 - protein, 39 aa, synthetic construct
  REGION 1..39: note = Recombinant collagen fragment
GPAGKDGESGRPGRPGERGLPGPPGIKGPAGIPGFPGMK

SEQ ID NO: 638 - protein, 27 aa, synthetic construct
  REGION 1..27: note = Recombinant collagen fragment
GPAGPNGIPGEKGPAGERGAPGPAGPR

SEQ ID NO: 639 - protein, 12 aa, synthetic construct
  REGION 1..12: note = Recombinant collagen fragment
GPAGPPGPPGPP

SEQ ID NO: 640 - protein, 24 aa, synthetic construct
  REGION 1..24: note = Recombinant collagen fragment
GPGMRGMPGSPGGPGSDGKPGPPG

SEQ ID NO: 641 - protein, 47 aa, synthetic construct
  REGION 1..47: note = Recombinant collagen fragment
GPGMRGMPGSPGGPGSDGKPGPPGSQGESGRPGPPGPSGPRGQPGVM

SEQ ID NO: 642 - protein, 24 aa, synthetic construct
  REGION 1..24: note = Recombinant collagen fragment
GPGSDGKPGPPGSQGESGRPGPPG

SEQ ID NO: 643 - protein, 27 aa, synthetic construct
  REGION 1..27: note = Recombinant collagen fragment
GPGSDGKPGPPGSQGESGRPGPPGPSG

SEQ ID NO: 644 - protein, 29 aa, synthetic construct
  REGION 1..29: note = Recombinant collagen fragment
GPGSDGKPGPPGSQGESGRPGPPGPSGPR

SEQ ID NO: 645 - protein, 35 aa, synthetic construct
  REGION 1..35: note = Recombinant collagen fragment
GPGSDGKPGPPGSQGESGRPGPPGPSGPRGQPGVM

SEQ ID NO: 646 - protein, 43 aa, synthetic construct
  REGION 1..43: note = Recombinant collagen fragment
GPGSDGKPGPPGSQGESGRPGPPGPSGPRGQPGVMGFPGPKGN

SEQ ID NO: 647 - protein, 21 aa, synthetic construct
  REGION 1..21: note = Recombinant collagen fragment
GPMGPRGAPGERGRPGLPGAA

SEQ ID NO: 648 - protein, 24 aa, synthetic construct
  REGION 1..24: note = Recombinant collagen fragment
GPMGPRGAPGERGRPGLPGAAGAR

SEQ ID NO: 649 - protein, 21 aa, synthetic construct
  REGION 1..21: note = Recombinant collagen fragment
GPNGIPGEKGPAGERGAPGPA

SEQ ID NO: 650 - protein, 27 aa, synthetic construct
  REGION 1..27: note = Recombinant collagen fragment
GPPGAIGPSGPAGKDGESGRPGRPGER

SEQ ID NO: 651 - protein, 36 aa, synthetic construct
  REGION 1..36: note = Recombinant collagen fragment
GPPGAIGPSGPAGKDGESGRPGRPGERGLPGPPGIK

SEQ ID NO: 652 - protein, 19 aa, synthetic construct
  REGION 1..19: note = Recombinant collagen fragment
GPPGEPGQAGPSGPPGPPG

SEQ ID NO: 653 - protein, 24 aa, synthetic construct
  REGION 1..24: note = Recombinant collagen fragment
GPPGEPGQAGPSGPPGPPGAIGPS

SEQ ID NO: 654 - protein, 30 aa, synthetic construct
  REGION 1..30: note = Recombinant collagen fragment
GPPGEPGQAGPSGPPGPPGAIGPSGPAGKD

SEQ ID NO: 655 - protein, 51 aa, synthetic construct
  REGION 1..51: note = Recombinant collagen fragment
GPPGEPGQAGPSGPPGPPGAIGPSGPAGKDGESGRPGRPGERGLPGPPGIK

SEQ ID NO: 656 - protein, 18 aa, synthetic construct
  REGION 1..18: note = Recombinant collagen fragment
GPPGIKGPAGIPGFPGMK

SEQ ID NO: 657 - protein, 27 aa, synthetic construct
  REGION 1..27: note = Recombinant collagen fragment
GPPGINGSPGGKGEMGPAGIPGAPGLM

SEQ ID NO: 658 - protein, 15 aa, synthetic construct
  REGION 1..15: note = Recombinant collagen fragment
GPPGPAGANGAPGLR

SEQ ID NO: 659 - protein, 16 aa, synthetic construct
  REGION 1..16: note = Recombinant collagen fragment
GPPGPAGANGAPGLRG

SEQ ID NO: 660 - protein, 17 aa, synthetic construct
  REGION 1..17: note = Recombinant collagen fragment
GPPGPAGANGAPGLRGG

SEQ ID NO: 661 - protein, 18 aa, synthetic construct
  REGION 1..18: note = Recombinant collagen fragment
GPPGPAGANGAPGLRGGA

SEQ ID NO: 662 - protein, 19 aa, synthetic construct
  REGION 1..19: note = Recombinant collagen fragment
GPPGPAGANGAPGLRGGAG

SEQ ID NO: 663 - protein, 20 aa, synthetic construct
  REGION 1..20: note = Recombinant collagen fragment
GPPGPAGANGAPGLRGGAGE

SEQ ID NO: 664 - protein, 21 aa, synthetic construct
  REGION 1..21: note = Recombinant collagen fragment
GPPGPAGANGAPGLRGGAGEP

SEQ ID NO: 665 - protein, 22 aa, synthetic construct
  REGION 1..22: note = Recombinant collagen fragment
GPPGPAGANGAPGLRGGAGEPG

SEQ ID NO: 666 - protein, 23 aa, synthetic construct
  REGION 1..23: note = Recombinant collagen fragment
GPPGPAGANGAPGLRGGAGEPGK

SEQ ID NO: 667 - protein, 24 aa, synthetic construct
  REGION 1..24: note = Recombinant collagen fragment
GPPGPAGANGAPGLRGGAGEPGKN

SEQ ID NO: 668 - protein, 25 aa, synthetic construct
  REGION 1..25: note = Recombinant collagen fragment
GPPGPAGANGAPGLRGGAGEPGKNG

SEQ ID NO: 669 - protein, 26 aa, synthetic construct
  REGION 1..26: note = Recombinant collagen fragment
GPPGPAGANGAPGLRGGAGEPGKNGA

SEQ ID NO: 670 - protein, 27 aa, synthetic construct
  REGION 1..27: note = Recombinant collagen fragment
GPPGPAGANGAPGLRGGAGEPGKNGAK

SEQ ID NO: 671 - protein, 28 aa, synthetic construct
  REGION 1..28: note = Recombinant collagen fragment
GPPGPAGANGAPGLRGGAGEPGKNGAKG

SEQ ID NO: 672 - protein, 29 aa, synthetic construct
  REGION 1..29: note = Recombinant collagen fragment
GPPGPAGANGAPGLRGGAGEPGKNGAKGE

SEQ ID NO: 673 - protein, 31 aa, synthetic construct
  REGION 1..31: note = Recombinant collagen fragment
GPPGPAGANGAPGLRGGAGEPGKNGAKGEPG

SEQ ID NO: 674 - protein, 33 aa, synthetic construct
  REGION 1..33: note = Recombinant collagen fragment
GPPGPAGANGAPGLRGGAGEPGKNGAKGEPGPR

SEQ ID NO: 675 - protein, 34 aa, synthetic construct
  REGION 1..34: note = Recombinant collagen fragment
GPPGPAGANGAPGLRGGAGEPGKNGAKGEPGPRG

SEQ ID NO: 676 - protein, 35 aa, synthetic construct
  REGION 1..35: note = Recombinant collagen fragment
GPPGPAGANGAPGLRGGAGEPGKNGAKGEPGPRGE

SEQ ID NO: 677 - protein, 36 aa, synthetic construct
  REGION 1..36: note = Recombinant collagen fragment
GPPGPAGANGAPGLRGGAGEPGKNGAKGEPGPRGER

SEQ ID NO: 678 - protein, 37 aa, synthetic construct
  REGION 1..37: note = Recombinant collagen fragment
GPPGPAGANGAPGLRGGAGEPGKNGAKGEPGPRGERG

SEQ ID NO: 679 - protein, 38 aa, synthetic construct
  REGION 1..38: note = Recombinant collagen fragment
GPPGPAGANGAPGLRGGAGEPGKNGAKGEPGPRGERGE

SEQ ID NO: 680 - protein, 39 aa, synthetic construct
  REGION 1..39: note = Recombinant collagen fragment
GPPGPAGANGAPGLRGGAGEPGKNGAKGEPGPRGERGEA

SEQ ID NO: 681 - protein, 12 aa, synthetic construct
  REGION 1..12: note = Recombinant collagen fragment
GPPGPPGAIGPS

SEQ ID NO: 682 - protein, 15 aa, synthetic construct
  REGION 1..15: note = Recombinant collagen fragment
GPPGPPGAIGPSGPA

SEQ ID NO: 683 - protein, 30 aa, synthetic construct
  REGION 1..30: note = Recombinant collagen fragment
GPPGPPGAIGPSGPAGKDGESGRPGRPGER

SEQ ID NO: 684 - protein, 39 aa, synthetic construct
  REGION 1..39: note = Recombinant collagen fragment
GPPGPPGAIGPSGPAGKDGESGRPGRPGERGLPGPPGIK

SEQ ID NO: 685 - protein, 21 aa, synthetic construct
  REGION 1..21: note = Recombinant collagen fragment
GPPGPPGINGSPGGKGEMGPA

SEQ ID NO: 686 - protein, 30 aa, synthetic construct
  REGION 1..30: note = Recombinant collagen fragment
GPPGPPGINGSPGGKGEMGPAGIPGAPGLM

SEQ ID NO: 687 - protein, 32 aa, synthetic construct
  REGION 1..32: note = Recombinant collagen fragment
GPPGPPGINGSPGGKGEMGPAGIPGAPGLMGA

SEQ ID NO: 688 - protein, 33 aa, synthetic construct
  REGION 1..33: note = Recombinant collagen fragment
GPPGPPGINGSPGGKGEMGPAGIPGAPGLMGAR

SEQ ID NO: 689 - protein, 48 aa, synthetic construct
  REGION 1..48: note = Recombinant collagen fragment
GPPGPPGINGSPGGKGEMGPAGIPGAPGLMGARGPPGPAGANGAPGLR

SEQ ID NO: 690 - protein, 12 aa, synthetic construct
  REGION 1..12: note = Recombinant collagen fragment
GPPGPPGPPGTS

SEQ ID NO: 691 - protein, 16 aa, synthetic construct
  REGION 1..16: note = Recombinant collagen fragment
GPPGPPGPPGTSGHPG

SEQ ID NO: 692 - protein, 48 aa, synthetic construct
  REGION 1..48: note = Recombinant collagen fragment
GPPGPPGPPGTSGHPGSPGSPGYQGPPGEPGQAGPSGPPGPPGAIGPS

SEQ ID NO: 693 - protein, 18 aa, synthetic construct
  REGION 1..18: note = Recombinant collagen fragment
GPPGPPGTAGFPGSPGAK

SEQ ID NO: 694 - protein, 24 aa, synthetic construct
  REGION 1..24: note = Recombinant collagen fragment
GPPGPPGTAGFPGSPGAKGEVGPA

SEQ ID NO: 695 - protein, 15 aa, synthetic construct
  REGION 1..15: note = Recombinant collagen fragment
GPPGPSGPRGQPGVM

SEQ ID NO: 696 - protein, 30 aa, synthetic construct
  REGION 1..30: note = Recombinant collagen fragment
GPPGPSGPRGQPGVMGFPGPKGNDGAPGKN

SEQ ID NO: 697 - protein, 27 aa, synthetic construct
  REGION 1..27: note = Recombinant collagen fragment
GPPGSQGESGRPGPPGPSGPRGQPGVM

SEQ ID NO: 698 - protein, 21 aa, synthetic construct
  REGION 1..21: note = Recombinant collagen fragment
GPPGTAGFPGSPGAKGEVGPA

SEQ ID NO: 699 - protein, 18 aa, synthetic construct
  REGION 1..18: note = Recombinant collagen fragment
GPQGHAGAQGPPGPPGIN

SEQ ID NO: 700 - protein, 25 aa, synthetic construct
  REGION 1..25: note = Recombinant collagen fragment
GPRGAAGEPGRDGVPGGPGMRGMPG

SEQ ID NO: 701 - protein, 18 aa, synthetic construct
  REGION 1..18: note = Recombinant collagen fragment
GPRGAPGERGRPGLPGAA

SEQ ID NO: 702 - protein, 20 aa, synthetic construct
  REGION 1..20: note = Recombinant collagen fragment
GPRGAPGERGRPGLPGAAGA

SEQ ID NO: 703 - protein, 21 aa, synthetic construct
  REGION 1..21: note = Recombinant collagen fragment
GPRGAPGERGRPGLPGAAGAR

SEQ ID NO: 704 - protein, 23 aa, synthetic construct
  REGION 1..23: note = Recombinant collagen fragment
GPRGAPGERGRPGLPGAAGARGN

SEQ ID NO: 705 - protein, 28 aa, synthetic construct
  REGION 1..28: note = Recombinant collagen fragment
GPRGAPGERGRPGLPGAAGARGNDGARG

SEQ ID NO: 706 - protein, 42 aa, synthetic construct
  REGION 1..42: note = Recombinant collagen fragment
GPRGAPGERGRPGLPGAAGARGNDGARGSDGQPGPPGPPGTA

SEQ ID NO: 707 - protein, 17 aa, synthetic construct
  REGION 1..17: note = Recombinant collagen fragment
GPRGQPGVMGFPGPKGN

SEQ ID NO: 708 - protein, 24 aa, synthetic construct
  REGION 1..24: note = Recombinant collagen fragment
GPRGQPGVMGFPGPKGNDGAPGKN

SEQ ID NO: 709 - protein, 30 aa, synthetic construct
  REGION 1..30: note = Recombinant collagen fragment
GPSGPAGKDGESGRPGRPGERGLPGPPGIK

SEQ ID NO: 710 - protein, 33 aa, synthetic construct
  REGION 1..33: note = Recombinant collagen fragment
GPSGPAGKDGESGRPGRPGERGLPGPPGIKGPA

SEQ ID NO: 711 - protein, 15 aa, synthetic construct
  REGION 1..15: note = Recombinant collagen fragment
GPSGPPGPPGAIGPS

SEQ ID NO: 712 - protein, 27 aa, synthetic construct
  REGION 1..27: note = Recombinant collagen fragment
GPSGPRGQPGVMGFPGPKGNDGAPGKN

SEQ ID NO: 713 - protein, 18 aa, synthetic construct
  REGION 1..18: note = Recombinant collagen fragment
GQAGPSGPPGPPGAIGPS

SEQ ID NO: 714 - protein, 12 aa, synthetic construct
  REGION 1..12: note = Recombinant collagen fragment
GQPGPPGPPGTA

SEQ ID NO: 715 - protein, 21 aa, synthetic construct
  REGION 1..21: note = Recombinant collagen fragment
GQPGPPGPPGTAGFPGSPGAK

SEQ ID NO: 716 - protein, 27 aa, synthetic construct
  REGION 1..27: note = Recombinant collagen fragment
GQPGPPGPPGTAGFPGSPGAKGEVGPA

SEQ ID NO: 717 - protein, 12 aa, synthetic construct
  REGION 1..12: note = Recombinant collagen fragment
GQPGVMGFPGPK

SEQ ID NO: 718 - protein, 14 aa, synthetic construct
  REGION 1..14: note = Recombinant collagen fragment
GQPGVMGFPGPKGN

SEQ ID NO: 719 - protein, 21 aa, synthetic construct
  REGION 1..21: note = Recombinant collagen fragment
GQPGVMGFPGPKGNDGAPGKN

SEQ ID NO: 720 - protein, 24 aa, synthetic construct
  REGION 1..24: note = Recombinant collagen fragment
GQRGEPGPQGHAGAQGPPGPPGIN

SEQ ID NO: 721 - protein, 22 aa, synthetic construct
  REGION 1..22: note = Recombinant collagen fragment
GRDGVPGGPGMRGMPGSPGGPG

SEQ ID NO: 722 - protein, 54 aa, synthetic construct
  REGION 1..54: note = Recombinant collagen fragment
GRDGVPGGPGMRGMPGSPGGPGSDGKPGPPGSQGESGRPGPPGPSGPRGQPGVM

SEQ ID NO: 723 - protein, 24 aa, synthetic construct
  REGION 1..24: note = Recombinant collagen fragment
GRNGEKGETGAPGLKGENGLPGEN

SEQ ID NO: 724 - protein, 33 aa, synthetic construct
  REGION 1..33: note = Recombinant collagen fragment
GRNGEKGETGAPGLKGENGLPGENGAPGPMGPR

SEQ ID NO: 725 - protein, 15 aa, synthetic construct
  REGION 1..15: note = Recombinant collagen fragment
GRPGERGLPGPPGIK

SEQ ID NO: 726 - protein, 18 aa, synthetic construct
  REGION 1..18: note = Recombinant collagen fragment
GRPGERGLPGPPGIKGPA

SEQ ID NO: 727 - protein, 12 aa, synthetic construct
  REGION 1..12: note = Recombinant collagen fragment
GRPGLPGAAGAR

SEQ ID NO: 728 - protein, 33 aa, synthetic construct
  REGION 1..33: note = Recombinant collagen fragment
GRPGLPGAAGARGNDGARGSDGQPGPPGPPGTA

SEQ ID NO: 729 - protein, 42 aa, synthetic construct
  REGION 1..42: note = Recombinant collagen fragment
GRPGLPGAAGARGNDGARGSDGQPGPPGPPGTAGFPGSPGAK

SEQ ID NO: 730 - protein, 48 aa, synthetic construct
  REGION 1..48: note = Recombinant collagen fragment
GRPGLPGAAGARGNDGARGSDGQPGPPGPPGTAGFPGSPGAKGEVGPA

SEQ ID NO: 731 - protein, 18 aa, synthetic construct
  REGION 1..18: note = Recombinant collagen fragment
GRPGPPGPSGPRGQPGVM

SEQ ID NO: 732 - protein, 18 aa, synthetic construct
  REGION 1..18: note = Recombinant collagen fragment
GRPGRPGERGLPGPPGIK

SEQ ID NO: 733 - protein, 21 aa, synthetic construct
  REGION 1..21: note = Recombinant collagen fragment
GRPGRPGERGLPGPPGIKGPA

SEQ ID NO: 734 - protein, 33 aa, synthetic construct
  REGION 1..33: note = Recombinant collagen fragment
GSDGKPGPPGSQGESGRPGPPGPSGPRGQPGVM

SEQ ID NO: 735 - protein, 15 aa, synthetic construct
  REGION 1..15: note = Recombinant collagen fragment
GSDGQPGPPGPPGTA

SEQ ID NO: 736 - protein, 24 aa, synthetic construct
  REGION 1..24: note = Recombinant collagen fragment
GSDGQPGPPGPPGTAGFPGSPGAK

SEQ ID NO: 737 - protein, 30 aa, synthetic construct
  REGION 1..30: note = Recombinant collagen fragment
GSDGQPGPPGPPGTAGFPGSPGAKGEVGPA

SEQ ID NO: 738 - protein, 30 aa, synthetic construct
  REGION 1..30: note = Recombinant collagen fragment
GSNGAPGQRGEPGPQGHAGAQGPPGPPGIN

SEQ ID NO: 739 - protein, 30 aa, synthetic construct
  REGION 1..30: note = Recombinant collagen fragment
GSPGEPGANGLPGAAGERGAPGFRGPAGPN

SEQ ID NO: 740 - protein, 19 aa, synthetic construct
  REGION 1..19: note = Recombinant collagen fragment
GSPGGKGEMGPAGIPGAPG

SEQ ID NO: 741 - protein, 21 aa, synthetic construct
  REGION 1..21: note = Recombinant collagen fragment
GSPGGKGEMGPAGIPGAPGLM

SEQ ID NO: 742 - protein, 22 aa, synthetic construct
  REGION 1..22: note = Recombinant collagen fragment
GSPGGKGEMGPAGIPGAPGLMG

SEQ ID NO: 743 - protein, 23 aa, synthetic construct
  REGION 1..23: note = Recombinant collagen fragment
GSPGGKGEMGPAGIPGAPGLMGA

SEQ ID NO: 744 - protein, 24 aa, synthetic construct
  REGION 1..24: note = Recombinant collagen fragment
GSPGGKGEMGPAGIPGAPGLMGAR

SEQ ID NO: 745 - protein, 25 aa, synthetic construct
  REGION 1..25: note = Recombinant collagen fragment
GSPGGKGEMGPAGIPGAPGLMGARG

SEQ ID NO: 746 - protein, 28 aa, synthetic construct
  REGION 1..28: note = Recombinant collagen fragment
GSPGGKGEMGPAGIPGAPGLMGARGPPG

SEQ ID NO: 747 - protein, 30 aa, synthetic construct
  REGION 1..30: note = Recombinant collagen fragment
GSPGGKGEMGPAGIPGAPGLMGARGPPGPA

SEQ ID NO: 748 - protein, 31 aa, synthetic construct
  REGION 1..31: note = Recombinant collagen fragment
GSPGGKGEMGPAGIPGAPGLMGARGPPGPAG

SEQ ID NO: 749 - protein, 33 aa, synthetic construct
  REGION 1..33: note = Recombinant collagen fragment
GSPGGKGEMGPAGIPGAPGLMGARGPPGPAGAN

SEQ ID NO: 750 - protein, 39 aa, synthetic construct
  REGION 1..39: note = Recombinant collagen fragment
GSPGGKGEMGPAGIPGAPGLMGARGPPGPAGANGAPGLR

SEQ ID NO: 751 - protein, 48 aa, synthetic construct
  REGION 1..48: note = Recombinant collagen fragment
GSPGGKGEMGPAGIPGAPGLMGARGPPGPAGANGAPGLRGGAGEPGKN

SEQ ID NO: 752 - protein, 39 aa, synthetic construct
  REGION 1..39: note = Recombinant collagen fragment
GSPGGPGSDGKPGPPGSQGESGRPGPPGPSGPRGQPGVM

SEQ ID NO: 753 - protein, 18 aa, synthetic construct
  REGION 1..18: note = Recombinant collagen fragment
GSPGSNGAPGQRGEPGPQ

SEQ ID NO: 754 - protein, 24 aa, synthetic construct
  REGION 1..24: note = Recombinant collagen fragment
GSPGSNGAPGQRGEPGPQGHAGAQ

SEQ ID NO: 755 - protein, 26 aa, synthetic construct
  REGION 1..26: note = Recombinant collagen fragment
GSPGSNGAPGQRGEPGPQGHAGAQGP

SEQ ID NO: 756 - protein, 27 aa, synthetic construct
  REGION 1..27: note = Recombinant collagen fragment
GSPGSNGAPGQRGEPGPQGHAGAQGPP

SEQ ID NO: 757 - protein, 28 aa, synthetic construct
  REGION 1..28: note = Recombinant collagen fragment
GSPGSNGAPGQRGEPGPQGHAGAQGPPG

SEQ ID NO: 758 - protein, 29 aa, synthetic construct
  REGION 1..29: note = Recombinant collagen fragment
GSPGSNGAPGQRGEPGPQGHAGAQGPPGP

SEQ ID NO: 759 - protein, 30 aa, synthetic construct
  REGION 1..30: note = Recombinant collagen fragment
GSPGSNGAPGQRGEPGPQGHAGAQGPPGPP

SEQ ID NO: 760 - protein, 31 aa, synthetic construct
  REGION 1..31: note = Recombinant collagen fragment
GSPGSNGAPGQRGEPGPQGHAGAQGPPGPPG

SEQ ID NO: 761 - protein, 33 aa, synthetic construct
  REGION 1..33: note = Recombinant collagen fragment
GSPGSNGAPGQRGEPGPQGHAGAQGPPGPPGIN

SEQ ID NO: 762 - protein, 34 aa, synthetic construct
  REGION 1..34: note = Recombinant collagen fragment
GSPGSNGAPGQRGEPGPQGHAGAQGPPGPPGING

SEQ ID NO: 763 - protein, 35 aa, synthetic construct
  REGION 1..35: note = Recombinant collagen fragment
GSPGSNGAPGQRGEPGPQGHAGAQGPPGPPGINGS

SEQ ID NO: 764 - protein, 37 aa, synthetic construct
  REGION 1..37: note = Recombinant collagen fragment
GSPGSNGAPGQRGEPGPQGHAGAQGPPGPPGINGSPG

SEQ ID NO: 765 - protein, 38 aa, synthetic construct
  REGION 1..38: note = Recombinant collagen fragment
GSPGSNGAPGQRGEPGPQGHAGAQGPPGPPGINGSPGG

SEQ ID NO: 766 - protein, 39 aa, synthetic construct
  REGION 1..39: note = Recombinant collagen fragment
GSPGSNGAPGQRGEPGPQGHAGAQGPPGPPGINGSPGGK

SEQ ID NO: 767 - protein, 40 aa, synthetic construct
  REGION 1..40: note = Recombinant collagen fragment
GSPGSNGAPGQRGEPGPQGHAGAQGPPGPPGINGSPGGKG

SEQ ID NO: 768 - protein, 42 aa, synthetic construct
  REGION 1..42: note = Recombinant collagen fragment
GSPGSNGAPGQRGEPGPQGHAGAQGPPGPPGINGSPGGKGEM

SEQ ID NO: 769 - protein, 43 aa, synthetic construct
  REGION 1..43: note = Recombinant collagen fragment
GSPGSNGAPGQRGEPGPQGHAGAQGPPGPPGINGSPGGKGEMG

SEQ ID NO: 770 - protein, 45 aa, synthetic construct
  REGION 1..45: note = Recombinant collagen fragment
GSPGSNGAPGQRGEPGPQGHAGAQGPPGPPGINGSPGGKGEMGPA

SEQ ID NO: 771 - protein, 46 aa, synthetic construct
  REGION 1..46: note = Recombinant collagen fragment
GSPGSNGAPGQRGEPGPQGHAGAQGPPGPPGINGSPGGKGEMGPAG

SEQ ID NO: 772 - protein, 54 aa, synthetic construct
  REGION 1..54: note = Recombinant collagen fragment
GSPGSNGAPGQRGEPGPQGHAGAQGPPGPPGINGSPGGKGEMGPAGIPGAPGLM

SEQ ID NO: 773 - protein, 55 aa, synthetic construct
  REGION 1..55: note = Recombinant collagen fragment
GSPGSNGAPGQRGEPGPQGHAGAQGPPGPPGINGSPGGKGEMGPAGIPGAPGLMG

SEQ ID NO: 774 - protein, 56 aa, synthetic construct
  REGION 1..56: note = Recombinant collagen fragment
GSPGSNGAPGQRGEPGPQGHAGAQGPPGPPGINGSPGGKGEMGPAGIPGAPGLMGA

SEQ ID NO: 775 - protein, 24 aa, synthetic construct
  REGION 1..24: note = Recombinant collagen fragment
GSQGESGRPGPPGPSGPRGQPGVM

SEQ ID NO: 776 - protein, 18 aa, synthetic construct
  REGION 1..18: note = Recombinant collagen fragment
GTAGFPGSPGAKGEVGPA

SEQ ID NO: 777 - protein, 39 aa, synthetic construct
  REGION 1..39: note = Recombinant collagen fragment
GTSGHPGSPGSPGYQGPPGEPGQAGPSGPPGPPGAIGPS

SEQ ID NO: 778 - protein, 30 aa, synthetic construct
  REGION 1..30: note = Recombinant collagen fragment
GVPGGPGMRGMPGSPGGPGSDGKPGPPGSQ

SEQ ID NO: 779 - protein, 51 aa, synthetic construct
  REGION 1..51: note = Recombinant collagen fragment
GVPGGPGMRGMPGSPGGPGSDGKPGPPGSQGESGRPGPPGPSGPRGQPGVM

SEQ ID NO: 780 - protein, 17 aa, synthetic construct
  REGION 1..17: note = Recombinant collagen fragment
GYPGPAGPPGPPGPPGT

SEQ ID NO: 781 - protein, 18 aa, synthetic construct
  REGION 1..18: note = Recombinant collagen fragment
GYPGPAGPPGPPGPPGTS

SEQ ID NO: 782 - protein, 20 aa, synthetic construct
  REGION 1..20: note = Recombinant collagen fragment
GYPGPAGPPGPPGPPGTSGH

SEQ ID NO: 783 - protein, 22 aa, synthetic construct
  REGION 1..22: note = Recombinant collagen fragment
GYPGPAGPPGPPGPPGTSGHPG

SEQ ID NO: 784 - protein, 54 aa, synthetic construct
  REGION 1..54: note = Recombinant collagen fragment
GYPGPAGPPGPPGPPGTSGHPGSPGSPGYQGPPGEPGQAGPSGPPGPPGAIGPS

SEQ ID NO: 785 - protein, 57 aa, synthetic construct
  REGION 1..57: note = Recombinant collagen fragment
GYPGPAGPPGPPGPPGTSGHPGSPGSPGYQGPPGEPGQAGPSGPPGPPGAIGPSGPA

SEQ ID NO: 786 - protein, 14 aa, synthetic construct
  REGION 1..14: note = Recombinant collagen fragment
HAGAQGPPGPPGIN

SEQ ID NO: 787 - protein, 18 aa, synthetic construct
  REGION 1..18: note = Recombinant collagen fragment
HAGAQGPPGPPGINGSPG

SEQ ID NO: 788 - protein, 24 aa, synthetic construct
  REGION 1..24: note = Recombinant collagen fragment
HAGAQGPPGPPGINGSPGGKGEMG

SEQ ID NO: 789 - protein, 26 aa, synthetic construct
  REGION 1..26: note = Recombinant collagen fragment
HAGAQGPPGPPGINGSPGGKGEMGPA

SEQ ID NO: 790 - protein, 38 aa, synthetic construct
  REGION 1..38: note = Recombinant collagen fragment
HAGAQGPPGPPGINGSPGGKGEMGPAGIPGAPGLMGAR

SEQ ID NO: 791 - protein, 35 aa, synthetic construct
  REGION 1..35: note = Recombinant collagen fragment
HPGSPGSPGYQGPPGEPGQAGPSGPPGPPGAIGPS

SEQ ID NO: 792 - protein, 23 aa, synthetic construct
  REGION 1..23: note = Recombinant collagen fragment
HRGFDGRNGEKGETGAPGLKGEN

SEQ ID NO: 793 - protein, 38 aa, synthetic construct
  REGION 1..38: note = Recombinant collagen fragment
HRGFDGRNGEKGETGAPGLKGENGLPGENGAPGPMGPR

SEQ ID NO: 794 - protein, 31 aa, synthetic construct
  REGION 1..31: note = Recombinant collagen fragment
IGPSGPAGKDGESGRPGRPGERGLPGPPGIK

SEQ ID NO: 795 - protein, 12 aa, synthetic construct
  REGION 1..12: note = Recombinant collagen fragment
IKGPAGIPGFPG

SEQ ID NO: 796 - protein, 14 aa, synthetic construct
  REGION 1..14: note = Recombinant collagen fragment
IKGPAGIPGFPGMK

SEQ ID NO: 797 - protein, 26 aa, synthetic construct
  REGION 1..26: note = Recombinant collagen fragment
IPGAPGLMGARGPPGPAGANGAPGLR

SEQ ID NO: 798 - protein, 17 aa, synthetic construct
  REGION 1..17: note = Recombinant collagen fragment
IPGFPGMKGHRGFDGRN

SEQ ID NO: 799 - protein, 23 aa, synthetic construct
  REGION 1..23: note = Recombinant collagen fragment
KDGESGRPGRPGERGLPGPPGIK

SEQ ID NO: 800 - protein, 26 aa, synthetic construct
  REGION 1..26: note = Recombinant collagen fragment
KDGESGRPGRPGERGLPGPPGIKGPA

SEQ ID NO: 801 - protein, 32 aa, synthetic construct
  REGION 1..32: note = Recombinant collagen fragment
KDGSPGEPGANGLPGAAGERGAPGFRGPAGPN

SEQ ID NO: 802 - protein, 19 aa, synthetic construct
  REGION 1..19: note = Recombinant collagen fragment
KGENGLPGENGAPGPMGPR

SEQ ID NO: 803 - protein, 13 aa, synthetic construct
  REGION 1..13: note = Recombinant collagen fragment
KGPAGIPGFPGMK

SEQ ID NO: 804 - protein, 23 aa, synthetic construct
  REGION 1..23: note = Recombinant collagen fragment
KPGPPGSQGESGRPGPPGPSGPR

SEQ ID NO: 805 - protein, 29 aa, synthetic construct
  REGION 1..29: note = Recombinant collagen fragment
KPGPPGSQGESGRPGPPGPSGPRGQPGVM

SEQ ID NO: 806 - protein, 20 aa, synthetic construct
  REGION 1..20: note = Recombinant collagen fragment
LKGENGLPGENGAPGPMGPR

SEQ ID NO: 807 - protein, 20 aa, synthetic construct
  REGION 1..20: note = Recombinant collagen fragment
LMGARGPPGPAGANGAPGLR

SEQ ID NO: 808 - protein, 29 aa, synthetic construct
  REGION 1..29: note = Recombinant collagen fragment
LPGAAGARGNDGARGSDGQPGPPGPPGTA

SEQ ID NO: 809 - protein, 20 aa, synthetic construct
  REGION 1..20: note = Recombinant collagen fragment
LPGAAGERGAPGFRGPAGPN

SEQ ID NO: 810 - protein, 8 aa, synthetic construct
  REGION 1..8: note = Recombinant collagen fragment
LPGPPGIK

SEQ ID NO: 811 - protein, 11 aa, synthetic construct
  REGION 1..11: note = Recombinant collagen fragment
LPGPPGIKGPA

SEQ ID NO: 812 - protein, 20 aa, synthetic construct
  REGION 1..20: note = Recombinant collagen fragment
LPGPPGIKGPAGIPGFPGMK

SEQ ID NO: 813 - protein, 19 aa, synthetic construct
  REGION 1..19: note = Recombinant collagen fragment
MGARGPPGPAGANGAPGLR

SEQ ID NO: 814 - protein, 28 aa, synthetic construct
  REGION 1..28: note = Recombinant collagen fragment
MGARGPPGPAGANGAPGLRGGAGEPGKN

SEQ ID NO: 815 - protein, 31 aa, synthetic construct
  REGION 1..31: note = Recombinant collagen fragment
MGPAGIPGAPGLMGARGPPGPAGANGAPGLR

SEQ ID NO: 816 - protein, 22 aa, synthetic construct
  REGION 1..22: note = Recombinant collagen fragment
MGPRGAPGERGRPGLPGAAGAR

SEQ ID NO: 817 - protein, 41 aa, synthetic construct
  REGION 1..41: note = Recombinant collagen fragment
MPGSPGGPGSDGKPGPPGSQGESGRPGPPGPSGPRGQPGVM

SEQ ID NO: 818 - protein, 44 aa, synthetic construct
  REGION 1..44: note = Recombinant collagen fragment
MRGMPGSPGGPGSDGKPGPPGSQGESGRPGPPGPSGPRGQPGVM

SEQ ID NO: 819 - protein, 20 aa, synthetic construct
  REGION 1..20: note = Recombinant collagen fragment
NDGARGSDGQPGPPGPPGTA

SEQ ID NO: 820 - protein, 35 aa, synthetic construct
  REGION 1..35: note = Recombinant collagen fragment
NDGARGSDGQPGPPGPPGTAGFPGSPGAKGEVGPA

SEQ ID NO: 821 - protein, 15 aa, synthetic construct
  REGION 1..15: note = Recombinant collagen fragment
NGAPGPMGPRGAPGE

SEQ ID NO: 822 - protein, 28 aa, synthetic construct
  REGION 1..28: note = Recombinant collagen fragment
NGAPGPMGPRGAPGERGRPGLPGAAGAR

SEQ ID NO: 823 - protein, 31 aa, synthetic construct
  REGION 1..31: note = Recombinant collagen fragment
NGEKGETGAPGLKGENGLPGENGAPGPMGPR

SEQ ID NO: 824 - protein, 18 aa, synthetic construct
  REGION 1..18: note = Recombinant collagen fragment
NGETGPQGPPGPTGPGGD

SEQ ID NO: 825 - protein, 16 aa, synthetic construct
  REGION 1..16: note = Recombinant collagen fragment
NGLPGENGAPGPMGPR

SEQ ID NO: 826 - protein, 22 aa, synthetic construct
  REGION 1..22: note = Recombinant collagen fragment
NGSPGGKGEMGPAGIPGAPGLM

SEQ ID NO: 827 - protein, 40 aa, synthetic construct
  REGION 1..40: note = Recombinant collagen fragment
NGSPGGKGEMGPAGIPGAPGLMGARGPPGPAGANGAPGLR

SEQ ID NO: 828 - protein, 11 aa, synthetic construct
  REGION 1..11: note = Recombinant collagen fragment
PAGIPGAPGLM

SEQ ID NO: 829 - protein, 12 aa, synthetic construct
  REGION 1..12: note = Recombinant collagen fragment
PAGIPGAPGLMG

SEQ ID NO: 830 - protein, 11 aa, synthetic construct
  REGION 1..11: note = Recombinant collagen fragment
PAGIPGFPGMK

SEQ ID NO: 831 - protein, 12 aa, synthetic construct
  REGION 1..12: note = Recombinant collagen fragment
PAGIPGFPGMKG

SEQ ID NO: 832 - protein, 14 aa, synthetic construct
  REGION 1..14: note = Recombinant collagen fragment
PAGIPGFPGMKGHR

SEQ ID NO: 833 - protein, 15 aa, synthetic construct
  REGION 1..15: note = Recombinant collagen fragment
PAGIPGFPGMKGHRG

SEQ ID NO: 834 - protein, 16 aa, synthetic construct
  REGION 1..16: note = Recombinant collagen fragment
PAGIPGFPGMKGHRGF

SEQ ID NO: 835 - protein, 18 aa, synthetic construct
  REGION 1..18: note = Recombinant collagen fragment
PAGIPGFPGMKGHRGFDG

SEQ ID NO: 836 - protein, 20 aa, synthetic construct
  REGION 1..20: note = Recombinant collagen fragment
PAGIPGFPGMKGHRGFDGRN

SEQ ID NO: 837 - protein, 26 aa, synthetic construct
  REGION 1..26: note = Recombinant collagen fragment
PAGKDGESGRPGRPGERGLPGPPGIK

SEQ ID NO: 838 - protein, 19 aa, synthetic construct
  REGION 1..19: note = Recombinant collagen fragment
PGAAGERGAPGFRGPAGPN

SEQ ID NO: 839 - protein, 16 aa, synthetic construct
  REGION 1..16: note = Recombinant collagen fragment
PGANGLPGAAGERGAP

SEQ ID NO: 840 - protein, 25 aa, synthetic construct
  REGION 1..25: note = Recombinant collagen fragment
PGAPGLMGARGPPGPAGANGAPGLR

SEQ ID NO: 841 - protein, 27 aa, synthetic construct
  REGION 1..27: note = Recombinant collagen fragment
PGEPGPKGDAGAPGAPGGKGDAGAPGE

SEQ ID NO: 842 - protein, 13 aa, synthetic construct
  REGION 1..13: note = Recombinant collagen fragment
PGERGLPGPPGIK

SEQ ID NO: 843 - protein, 16 aa, synthetic construct
  REGION 1..16: note = Recombinant collagen fragment
PGERGRPGLPGAAGAR

SEQ ID NO: 844 - protein, 9 aa, synthetic construct
  REGION 1..9: note = Recombinant collagen fragment
PGFPGMKGH

SEQ ID NO: 845 - protein, 10 aa, synthetic construct
  REGION 1..10: note = Recombinant collagen fragment
PGFPGMKGHR

SEQ ID NO: 846 - protein, 12 aa, synthetic construct
  REGION 1..12: note = Recombinant collagen fragment
PGFPGMKGHRGF

SEQ ID NO: 847 - protein, 15 aa, synthetic construct
  REGION 1..15: note = Recombinant collagen fragment
PGFPGMKGHRGFDGR

SEQ ID NO: 848 - protein, 21 aa, synthetic construct
  REGION 1..21: note = Recombinant collagen fragment
PGLMGARGPPGPAGANGAPGL

SEQ ID NO: 849 - protein, 41 aa, synthetic construct
  REGION 1..41: note = Recombinant collagen fragment
PGLPGAAGARGNDGARGSDGQPGPPGPPGTAGFPGSPGAKG

SEQ ID NO: 850 - protein, 22 aa, synthetic construct
  REGION 1..22: note = Recombinant collagen fragment
PGPAGANGAPGLRGGAGEPGKN

SEQ ID NO: 851 - protein, 14 aa, synthetic construct
  REGION 1..14: note = Recombinant collagen fragment
PGPAGPPGPPGPPG

SEQ ID NO: 852 - protein, 16 aa, synthetic construct
  REGION 1..16: note = Recombinant collagen fragment
PGPAGPPGPPGPPGTS

SEQ ID NO: 853 - protein, 17 aa, synthetic construct
  REGION 1..17: note = Recombinant collagen fragment
PGPAGPPGPPGPPGTSG

SEQ ID NO: 854 - protein, 18 aa, synthetic construct
  REGION 1..18: note = Recombinant collagen fragment
PGPAGPPGPPGPPGTSGH

SEQ ID NO: 855 - protein, 42 aa, synthetic construct
  REGION 1..42: note = Recombinant collagen fragment
PGPAGPPGPPGPPGTSGHPGSPGSPGYQGPPGEPGQAGPSGP

SEQ ID NO: 856 - protein, 57 aa, synthetic construct
  REGION 1..57: note = Recombinant collagen fragment
PGPAGPPGPPGPPGTSGHPGSPGSPGYQGPPGEPGQAGPSGPPGPPGAIGPSGPAGK

SEQ ID NO: 857 - protein, 13 aa, synthetic construct
  REGION 1..13: note = Recombinant collagen fragment
PGPKGNDGAPGKN

SEQ ID NO: 858 - protein, 31 aa, synthetic construct
  REGION 1..31: note = Recombinant collagen fragment
PGPKGNDGAPGKNGERGGPGGPGPQGPPGKN

SEQ ID NO: 859 - protein, 25 aa, synthetic construct
  REGION 1..25: note = Recombinant collagen fragment
PGPMGPRGAPGERGRPGLPGAAGAR

SEQ ID NO: 860 - protein, 10 aa, synthetic construct
  REGION 1..10: note = Recombinant collagen fragment
PGPPGAIGPS

SEQ ID NO: 861 - protein, 19 aa, synthetic construct
  REGION 1..19: note = Recombinant collagen fragment
PGPPGIKGPAGIPGFPGMK

SEQ ID NO: 862 - protein, 19 aa, synthetic construct
  REGION 1..19: note = Recombinant collagen fragment
PGPPGPPGTAGFPGSPGAK

SEQ ID NO: 863 - protein, 16 aa, synthetic construct
  REGION 1..16: note = Recombinant collagen fragment
PGPPGPSGPRGQPGVM

SEQ ID NO: 864 - protein, 16 aa, synthetic construct
  REGION 1..16: note = Recombinant collagen fragment
PGRPGERGLPGPPGIK

SEQ ID NO: 865 - protein, 34 aa, synthetic construct
  REGION 1..34: note = Recombinant collagen fragment
PGSDGKPGPPGSQGESGRPGPPGPSGPRGQPGVM

SEQ ID NO: 866 - protein, 33 aa, synthetic construct
  REGION 1..33: note = Recombinant collagen fragment
PGSNGAPGQRGEPGPQGHAGAQGPPGPPGINGS

SEQ ID NO: 867 - protein, 13 aa, synthetic construct
  REGION 1..13: note = Recombinant collagen fragment
PGSPGAKGEVGPA

SEQ ID NO: 868 - protein, 26 aa, synthetic construct
  REGION 1..26: note = Recombinant collagen fragment
PPGAIGPSGPAGKDGESGRPGRPGER

SEQ ID NO: 869 - protein, 35 aa, synthetic construct
  REGION 1..35: note = Recombinant collagen fragment
PPGAIGPSGPAGKDGESGRPGRPGERGLPGPPGIK

SEQ ID NO: 870 - protein, 23 aa, synthetic construct
  REGION 1..23: note = Recombinant collagen fragment
PPGEPGQAGPSGPPGPPGAIGPS

SEQ ID NO: 871 - protein, 29 aa, synthetic construct
  REGION 1..29: note = Recombinant collagen fragment
PPGEPGQAGPSGPPGPPGAIGPSGPAGKD

SEQ ID NO: 872 - protein, 17 aa, synthetic construct
  REGION 1..17: note = Recombinant collagen fragment
PPGIKGPAGIPGFPGMK

SEQ ID NO: 873 - protein, 26 aa, synthetic construct
  REGION 1..26: note = Recombinant collagen fragment
PPGINGSPGGKGEMGPAGIPGAPGLM

SEQ ID NO: 874 - protein, 14 aa, synthetic construct
  REGION 1..14: note = Recombinant collagen fragment
PPGPAGANGAPGLR

SEQ ID NO: 875 - protein, 11 aa, synthetic construct
  REGION 1..11: note = Recombinant collagen fragment
PPGPPGAIGPS

SEQ ID NO: 876 - protein, 38 aa, synthetic construct
  REGION 1..38: note = Recombinant collagen fragment
PPGPPGAIGPSGPAGKDGESGRPGRPGERGLPGPPGIK

SEQ ID NO: 877 - protein, 29 aa, synthetic construct
  REGION 1..29: note = Recombinant collagen fragment
PPGPPGINGSPGGKGEMGPAGIPGAPGLM

SEQ ID NO: 878 - protein, 23 aa, synthetic construct
  REGION 1..23: note = Recombinant collagen fragment
PPGPPGTAGFPGSPGAKGEVGPA

SEQ ID NO: 879 - protein, 26 aa, synthetic construct
  REGION 1..26: note = Recombinant collagen fragment
PPGSQGESGRPGPPGPSGPRGQPGVM

SEQ ID NO: 880 - protein, 20 aa, synthetic construct
  REGION 1..20: note = Recombinant collagen fragment
PPGTAGFPGSPGAKGEVGPA

SEQ ID NO: 881 - protein, 17 aa, synthetic construct
  REGION 1..17: note = Recombinant collagen fragment
PQGHAGAQGPPGPPGIN

SEQ ID NO: 882 - protein, 29 aa, synthetic construct
  REGION 1..29: note = Recombinant collagen fragment
PSGPAGKDGESGRPGRPGERGLPGPPGIK

SEQ ID NO: 883 - protein, 14 aa, synthetic construct
  REGION 1..14: note = Recombinant collagen fragment
PSGPPGPPGAIGPS

SEQ ID NO: 884 - protein, 17 aa, synthetic construct
  REGION 1..17: note = Recombinant collagen fragment
QAGPSGPPGPPGAIGPS

SEQ ID NO: 885 - protein, 31 aa, synthetic construct
  REGION 1..31: note = Recombinant collagen fragment
QGPPGEPGQAGPSGPPGPPGAIGPSGPAGKD

SEQ ID NO: 886 - protein, 23 aa, synthetic construct
  REGION 1..23: note = Recombinant collagen fragment
QRGEPGPQGHAGAQGPPGPPGIN

SEQ ID NO: 887 - protein, 22 aa, synthetic construct
  REGION 1..22: note = Recombinant collagen fragment
RGFDGRNGEKGETGAPGLKGEN

SEQ ID NO: 888 - protein, 10 aa, synthetic construct
  REGION 1..10: note = Recombinant collagen fragment
RGLPGPPGIK

SEQ ID NO: 889 - protein, 13 aa, synthetic construct
  REGION 1..13: note = Recombinant collagen fragment
RGLPGPPGIKGPA

SEQ ID NO: 890 - protein, 22 aa, synthetic construct
  REGION 1..22: note = Recombinant collagen fragment
RGLPGPPGIKGPAGIPGFPGMK

SEQ ID NO: 891 - protein, 43 aa, synthetic construct
  REGION 1..43: note = Recombinant collagen fragment
RGMPGSPGGPGSDGKPGPPGSQGESGRPGPPGPSGPRGQPGVM

SEQ ID NO: 892 - protein, 22 aa, synthetic construct
  REGION 1..22: note = Recombinant collagen fragment
RGNDGARGSDGQPGPPGPPGTA

SEQ ID NO: 893 - protein, 31 aa, synthetic construct
  REGION 1..31: note = Recombinant collagen fragment
RGNDGARGSDGQPGPPGPPGTAGFPGSPGAK

SEQ ID NO: 894 - protein, 37 aa, synthetic construct
  REGION 1..37: note = Recombinant collagen fragment
RGNDGARGSDGQPGPPGPPGTAGFPGSPGAKGEVGPA

SEQ ID NO: 895 - protein, 15 aa, synthetic construct
  REGION 1..15: note = Recombinant collagen fragment
RGPPGPAGANGAPGL

SEQ ID NO: 896 - protein, 16 aa, synthetic construct
  REGION 1..16: note = Recombinant collagen fragment
RGPPGPAGANGAPGLR

SEQ ID NO: 897 - protein, 17 aa, synthetic construct
  REGION 1..17: note = Recombinant collagen fragment
RGPPGPAGANGAPGLRG

SEQ ID NO: 898 - protein, 25 aa, synthetic construct
  REGION 1..25: note = Recombinant collagen fragment
RGPPGPAGANGAPGLRGGAGEPGKN

SEQ ID NO: 899 - protein, 28 aa, synthetic construct
  REGION 1..28: note = Recombinant collagen fragment
RGPPGPAGANGAPGLRGGAGEPGKNGAK

SEQ ID NO: 900 - protein, 25 aa, synthetic construct
  REGION 1..25: note = Recombinant collagen fragment
RGSDGQPGPPGPPGTAGFPGSPGAK

SEQ ID NO: 901 - protein, 31 aa, synthetic construct
  REGION 1..31: note = Recombinant collagen fragment
RGSDGQPGPPGPPGTAGFPGSPGAKGEVGPA

SEQ ID NO: 902 - protein, 14 aa, synthetic construct
  REGION 1..14: note = Recombinant collagen fragment
RPGERGLPGPPGIK

SEQ ID NO: 903 - protein, 17 aa, synthetic construct
  REGION 1..17: note = Recombinant collagen fragment
RPGERGLPGPPGIKGPA

SEQ ID NO: 904 - protein, 28 aa, synthetic construct
  REGION 1..28: note = Recombinant collagen fragment
RPGLPGAAGARGNDGARGSDGQPGPPGP

SEQ ID NO: 905 - protein, 32 aa, synthetic construct
  REGION 1..32: note = Recombinant collagen fragment
RPGLPGAAGARGNDGARGSDGQPGPPGPPGTA

SEQ ID NO: 906 - protein, 17 aa, synthetic construct
  REGION 1..17: note = Recombinant collagen fragment
RPGPPGPSGPRGQPGVM

SEQ ID NO: 907 - protein, 17 aa, synthetic construct
  REGION 1..17: note = Recombinant collagen fragment
RPGRPGERGLPGPPGIK

SEQ ID NO: 908 - protein, 20 aa, synthetic construct
  REGION 1..20: note = Recombinant collagen fragment
RPGRPGERGLPGPPGIKGPA

SEQ ID NO: 909 - protein, 23 aa, synthetic construct
  REGION 1..23: note = Recombinant collagen fragment
SDGKPGPPGSQGESGRPGPPGPS

SEQ ID NO: 910 - protein, 24 aa, synthetic construct
  REGION 1..24: note = Recombinant collagen fragment
SDGKPGPPGSQGESGRPGPPGPSG

SEQ ID NO: 911 - protein, 26 aa, synthetic construct
  REGION 1..26: note = Recombinant collagen fragment
SDGKPGPPGSQGESGRPGPPGPSGPR

SEQ ID NO: 912 - protein, 27 aa, synthetic construct
  REGION 1..27: note = Recombinant collagen fragment
SDGKPGPPGSQGESGRPGPPGPSGPRG

SEQ ID NO: 913 - protein, 30 aa, synthetic construct
  REGION 1..30: note = Recombinant collagen fragment
SDGKPGPPGSQGESGRPGPPGPSGPRGQPG

SEQ ID NO: 914 - protein, 32 aa, synthetic construct
  REGION 1..32: note = Recombinant collagen fragment
SDGKPGPPGSQGESGRPGPPGPSGPRGQPGVM

SEQ ID NO: 915 - protein, 40 aa, synthetic construct
  REGION 1..40: note = Recombinant collagen fragment
SDGKPGPPGSQGESGRPGPPGPSGPRGQPGVMGFPGPKGN

SEQ ID NO: 916 - protein, 14 aa, synthetic construct
  REGION 1..14: note = Recombinant collagen fragment
SDGQPGPPGPPGTA

SEQ ID NO: 917 - protein, 23 aa, synthetic construct
  REGION 1..23: note = Recombinant collagen fragment
SDGQPGPPGPPGTAGFPGSPGAK

SEQ ID NO: 918 - protein, 29 aa, synthetic construct
  REGION 1..29: note = Recombinant collagen fragment
SDGQPGPPGPPGTAGFPGSPGAKGEVGPA

SEQ ID NO: 919 - protein, 28 aa, synthetic construct
  REGION 1..28: note = Recombinant collagen fragment
SGPAGKDGESGRPGRPGERGLPGPPGIK

SEQ ID NO: 920 - protein, 13 aa, synthetic construct
  REGION 1..13: note = Recombinant collagen fragment
SGPPGPPGAIGPS

SEQ ID NO: 921 - protein, 19 aa, synthetic construct
  REGION 1..19: note = Recombinant collagen fragment
SGPPGPPGAIGPSGPAGKD

SEQ ID NO: 922 - protein, 31 aa, synthetic construct
  REGION 1..31: note = Recombinant collagen fragment
SGPPGPPGAIGPSGPAGKDGESGRPGRPGER

SEQ ID NO: 923 - protein, 40 aa, synthetic construct
  REGION 1..40: note = Recombinant collagen fragment
SGPPGPPGAIGPSGPAGKDGESGRPGRPGERGLPGPPGIK

SEQ ID NO: 924 - protein, 43 aa, synthetic construct
  REGION 1..43: note = Recombinant collagen fragment
SGPPGPPGAIGPSGPAGKDGESGRPGRPGERGLPGPPGIKGPA

SEQ ID NO: 925 - protein, 18 aa, synthetic construct
  REGION 1..18: note = Recombinant collagen fragment
SGPRGQPGVMGFPGPKGN

SEQ ID NO: 926 - protein, 19 aa, synthetic construct
  REGION 1..19: note = Recombinant collagen fragment
SGRPGPPGPSGPRGQPGVM

SEQ ID NO: 927 - protein, 27 aa, synthetic construct
  REGION 1..27: note = Recombinant collagen fragment
SGRPGPPGPSGPRGQPGVMGFPGPKGN

SEQ ID NO: 928 - protein, 16 aa, synthetic construct
  REGION 1..16: note = Recombinant collagen fragment
SGRPGRPGERGLPGPP

SEQ ID NO: 929 - protein, 17 aa, synthetic construct
  REGION 1..17: note = Recombinant collagen fragment
SGRPGRPGERGLPGPPG

SEQ ID NO: 930 - protein, 19 aa, synthetic construct
  REGION 1..19: note = Recombinant collagen fragment
SGRPGRPGERGLPGPPGIK

SEQ ID NO: 931 - protein, 22 aa, synthetic construct
  REGION 1..22: note = Recombinant collagen fragment
SGRPGRPGERGLPGPPGIKGPA

SEQ ID NO: 932 - protein, 29 aa, synthetic construct
  REGION 1..29: note = Recombinant collagen fragment
SNGAPGQRGEPGPQGHAGAQGPPGPPGIN

SEQ ID NO: 933 - protein, 35 aa, synthetic construct
  REGION 1..35: note = Recombinant collagen fragment
SNGAPGQRGEPGPQGHAGAQGPPGPPGINGSPGGK

SEQ ID NO: 934 - protein, 41 aa, synthetic construct
  REGION 1..41: note = Recombinant collagen fragment
SNGAPGQRGEPGPQGHAGAQGPPGPPGINGSPGGKGEMGPA

SEQ ID NO: 935 - protein, 21 aa, synthetic construct
  REGION 1..21: note = Recombinant collagen fragment
SPGAKGEVGPAGSPGSNGAPG

SEQ ID NO: 936 - protein, 22 aa, synthetic construct
  REGION 1..22: note = Recombinant collagen fragment
SPGAKGEVGPAGSPGSNGAPGQ

SEQ ID NO: 937 - protein, 25 aa, synthetic construct
  REGION 1..25: note = Recombinant collagen fragment
SPGAKGEVGPAGSPGSNGAPGQRGE

SEQ ID NO: 938 - protein, 27 aa, synthetic construct
  REGION 1..27: note = Recombinant collagen fragment
SPGAKGEVGPAGSPGSNGAPGQRGEPG

SEQ ID NO: 939 - protein, 29 aa, synthetic construct
  REGION 1..29: note = Recombinant collagen fragment
SPGAKGEVGPAGSPGSNGAPGQRGEPGPQ

SEQ ID NO: 940 - protein, 30 aa, synthetic construct
  REGION 1..30: note = Recombinant collagen fragment
SPGAKGEVGPAGSPGSNGAPGQRGEPGPQG

SEQ ID NO: 941 - protein, 31 aa, synthetic construct
  REGION 1..31: note = Recombinant collagen fragment
SPGAKGEVGPAGSPGSNGAPGQRGEPGPQGH

SEQ ID NO: 942 - protein, 33 aa, synthetic construct
  REGION 1..33: note = Recombinant collagen fragment
SPGAKGEVGPAGSPGSNGAPGQRGEPGPQGHAG

SEQ ID NO: 943 - protein, 44 aa, synthetic construct
  REGION 1..44: note = Recombinant collagen fragment
SPGAKGEVGPAGSPGSNGAPGQRGEPGPQGHAGAQGPPGPPGIN

SEQ ID NO: 944 - protein, 29 aa, synthetic construct
  REGION 1..29: note = Recombinant collagen fragment
SPGEPGANGLPGAAGERGAPGFRGPAGPN

SEQ ID NO: 945 - protein, 20 aa, synthetic construct
  REGION 1..20: note = Recombinant collagen fragment
SPGGKGEMGPAGIPGAPGLM

SEQ ID NO: 946 - protein, 22 aa, synthetic construct
  REGION 1..22: note = Recombinant collagen fragment
SPGGKGEMGPAGIPGAPGLMGA

SEQ ID NO: 947 - protein, 23 aa, synthetic construct
  REGION 1..23: note = Recombinant collagen fragment
SPGGKGEMGPAGIPGAPGLMGAR

SEQ ID NO: 948 - protein, 32 aa, synthetic construct
  REGION 1..32: note = Recombinant collagen fragment
SPGGKGEMGPAGIPGAPGLMGARGPPGPAGAN

SEQ ID NO: 949 - protein, 27 aa, synthetic construct
  REGION 1..27: note = Recombinant collagen fragment
SPGGPGSDGKPGPPGSQGESGRPGPPG

SEQ ID NO: 950 - protein, 28 aa, synthetic construct
  REGION 1..28: note = Recombinant collagen fragment
SPGGPGSDGKPGPPGSQGESGRPGPPGP

SEQ ID NO: 951 - protein, 29 aa, synthetic construct
  REGION 1..29: note = Recombinant collagen fragment
SPGGPGSDGKPGPPGSQGESGRPGPPGPS

SEQ ID NO: 952 - protein, 32 aa, synthetic construct
  REGION 1..32: note = Recombinant collagen fragment
SPGGPGSDGKPGPPGSQGESGRPGPPGPSGPR

SEQ ID NO: 953 - protein, 33 aa, synthetic construct
  REGION 1..33: note = Recombinant collagen fragment
SPGGPGSDGKPGPPGSQGESGRPGPPGPSGPRG

SEQ ID NO: 954 - protein, 38 aa, synthetic construct
  REGION 1..38: note = Recombinant collagen fragment
SPGGPGSDGKPGPPGSQGESGRPGPPGPSGPRGQPGVM

SEQ ID NO: 955 - protein, 46 aa, synthetic construct
  REGION 1..46: note = Recombinant collagen fragment
SPGGPGSDGKPGPPGSQGESGRPGPPGPSGPRGQPGVMGFPGPKGN

SEQ ID NO: 956 - protein, 27 aa, synthetic construct
  REGION 1..27: note = Recombinant collagen fragment
SPGSNGAPGQRGEPGPQGHAGAQGPPG

SEQ ID NO: 957 - protein, 32 aa, synthetic construct
  REGION 1..32: note = Recombinant collagen fragment
SPGSNGAPGQRGEPGPQGHAGAQGPPGPPGIN

SEQ ID NO: 958 - protein, 36 aa, synthetic construct
  REGION 1..36: note = Recombinant collagen fragment
SPGSNGAPGQRGEPGPQGHAGAQGPPGPPGINGSPG

SEQ ID NO: 959 - protein, 38 aa, synthetic construct
  REGION 1..38: note = Recombinant collagen fragment
SPGSNGAPGQRGEPGPQGHAGAQGPPGPPGINGSPGGK

SEQ ID NO: 960 - protein, 42 aa, synthetic construct
  REGION 1..42: note = Recombinant collagen fragment
SPGSNGAPGQRGEPGPQGHAGAQGPPGPPGINGSPGGKGEMG

SEQ ID NO: 961 - protein, 44 aa, synthetic construct
  REGION 1..44: note = Recombinant collagen fragment
SPGSNGAPGQRGEPGPQGHAGAQGPPGPPGINGSPGGKGEMGPA

SEQ ID NO: 962 - protein, 32 aa, synthetic construct
  REGION 1..32: note = Recombinant collagen fragment
SPGSPGYQGPPGEPGQAGPSGPPGPPGAIGPS

SEQ ID NO: 963 - protein, 29 aa, synthetic construct
  REGION 1..29: note = Recombinant collagen fragment
SPGYQGPPGEPGQAGPSGPPGPPGAIGPS

SEQ ID NO: 964 - protein, 23 aa, synthetic construct
  REGION 1..23: note = Recombinant collagen fragment
SQGESGRPGPPGPSGPRGQPGVM

SEQ ID NO: 965 - protein, 31 aa, synthetic construct
  REGION 1..31: note = Recombinant collagen fragment
SQGESGRPGPPGPSGPRGQPGVMGFPGPKGN

SEQ ID NO: 966 - protein, 17 aa, synthetic construct
  REGION 1..17: note = Recombinant collagen fragment
TAGFPGSPGAKGEVGPA

SEQ ID NO: 967 - protein, 18 aa, synthetic construct
  REGION 1..18: note = Recombinant collagen fragment
TAGFPGSPGAKGEVGPAG

SEQ ID NO: 968 - protein, 21 aa, synthetic construct
  REGION 1..21: note = Recombinant collagen fragment
TAGFPGSPGAKGEVGPAGSPG

SEQ ID NO: 969 - protein, 23 aa, synthetic construct
  REGION 1..23: note = Recombinant collagen fragment
TAGFPGSPGAKGEVGPAGSPGSN

SEQ ID NO: 970 - protein, 25 aa, synthetic construct
  REGION 1..25: note = Recombinant collagen fragment
TGAPGLKGENGLPGENGAPGPMGPR

SEQ ID NO: 971 - protein, 15 aa, synthetic construct
  REGION 1..15: note = Recombinant collagen fragment
TGPQGPPGPTGPGGD

SEQ ID NO: 972 - protein, 23 aa, synthetic construct
  REGION 1..23: note = Recombinant collagen fragment
VGPAGSPGSNGAPGQRGEPGPQG

SEQ ID NO: 973 - protein, 1062 aa, synthetic construct
  REGION 1..1062: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRERGSEGSPGHPGQPGPPGPPGAPGPCCGGVGAAAIAGIGGEKAGGFAPYYG

SEQ ID NO: 974 - protein, 546 aa, synthetic construct; identical to SEQ ID NO: 1
  REGION 1..546: note = Recombinant collagen fragment

SEQ ID NO: 975 - protein, 546 aa, synthetic construct
  REGION 1..546: note = Recombinant collagen fragment
DVKSGVAVGGLAGYPGPAGAPGPPGPPGASGHPGSPGAPGYQGPPGAPGQAGPSGAPGPPGAIGASGPAGKDGASGRPGRPGERGLPGPPGAKGPAGIPGAPGMKGHRGFDGRNGEKGATGAPGLKGANGLPGENGAPGPMGPRGAPGERGRPGAPGAAGARGADGARGSDGAPGPPGPPGAAGFPGSPGAKGEVGPAGAPGSNGAPGARGEPGPQGAAGAQGPPGAPGINGSPGAKGEMGPAGAPGAPGLMGARGPPGPAGANGAPGLRGAAGEPGKNGAKGEPGPRGARGEAGIPGAPGAKGEDGADGSPGEPGANGLPGAAGARGAPGFRGAAGPNGIPGAKGPAGERGAPGPAGPRGAAGEPGRDGAPGGPGMRGAPGSPGGPGADGKPGPPGAQGESGRPGAPGPSGPRGAPGVMGFPGAKGNDGAPGANGERGGPGAPGPQGPPGANGETGPQGAPGPTGPGGAKGDTGPPGAQGLQGLPGAGGPPGENGAPGEPGPKGAAGAPGAPGAKGDAGAPGERGPPAIAGIGGEKAGGFAPYYG

SEQ ID NO: 976 - protein, 546 aa, synthetic construct
  REGION 1..546: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGEPGLMGPRGLPGSPGGPGPAGKEGPVGLPGIDGRPGPIGPAGARGEPGVMGFPGPKGNDGDPGKNGDKGHAGLAGARGAPGPDGETGAQGPPGPQGVQGGKGEQGPAGPPGFQGLPGTGGPAGEVGKPGEQGLHGEFGLPGPAGPRGERGAPGERGPPAIAGIGGEKAGGFAPYYG

SEQ ID NO: 977 - protein, 546 aa, synthetic construct
  REGION 1..546: note = Recombinant collagen fragment
DVKSGVAVGGLAGYPGPAGPPGPPGPPGTSGHPGSPGSPGYQGPPGEPGQAGPSGPPGPPGAIGPSGPAGKRGESGRPGRPGERGLPGPPGIKGPAGIPGFPGMKGHRGFRGRNGEKGETGAPGLKGENGLPGENGAPGPMGPRGAPGERGRPGLPGAAGARGNRGARGSDGQPGPPGPPGTAGFPGSPGAKGRVGPAGSPGSNGAPGQRGEPGPQGHAGAQGPPGPPGINGSPGGKGEMGPAGIPGAPGLMGARGPPGPAGANGAPGLRGGAGRPGKNGAKGEPGPRGERGRAGIPGVPGAKGERGKDGSPGEPGANGLPGAAGERGAPGFRGPAGPNGIPGEKGPAGERGAPGPAGPRGAAGEPGRRGVPGGPGMRGMPGSPGGPGSRGKPGPPGSQGESGRPGPPGPSGPRGQPGVMGFPGPKGNDGAPGKNGERGGPGGPGPQGPPGKNGETGPQGPPGPTGPGGRKGDTGPPGPQGLQGLPGTGGPPGENGKPGRPGPKGDAGAPGAPGGKGRAGAPGERGPPAIAGIGGEKAGGFAPYYG

SEQ ID NO: 978 - protein, 546 aa, synthetic construct
  REGION 1..546: note = Recombinant collagen fragment
DVKSGVAVGGLAGYPGPAGPPGPPGPPGASGHPGSPGSPGYQGPPGEPGQAGPAGPPGPPGAIGPSGPAGKDGESGRPGRPGERGLPGPPGIKGPAGIPGFPGMKGHRGFDGRNGEKGEAGAPGLKGENGLPGENGAPGPMGPRGAPGERGRPGLPGAAGARGNDGARGSDGQPGPPGPPGAAGFPGAPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGAQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 979 - protein, 546 aa, synthetic construct
  REGION 1..546: note = Recombinant collagen fragment
DVKSGVAVGGLAGYPGPAGPPGPPGPPGNSGHPGSPGSPGYQGPPGEPGQAGPNGPPGPPGAIGPSGPAGKDGESGRPGRPGERGLPGPPGIKGPAGIPGFPGMKGHRGFDGRNGEKGENGAPGLKGENGLPGENGAPGPMGPRGAPGERGRPGLPGAAGARGNDGARGSDGQPGPPGPPGNAGFPGNPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGNQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 980 - protein, 546 aa, synthetic construct; identical to SEQ ID NO: 975
  REGION 1..546: note = Recombinant collagen fragment

SEQ ID NO: 981 - protein, 549 aa, synthetic construct
  REGION 1..549: note = Recombinant collagen fragment
DSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 982 - protein, 318 aa, synthetic construct
  REGION 1..318: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPAIAGIGGEKAGGFAPYYG

SEQ ID NO: 983 - protein, 318 aa, synthetic construct; identical to SEQ ID NO: 982
  REGION 1..318: note = Recombinant collagen fragment

SEQ ID NO: 984 - protein, 438 aa, synthetic construct
  REGION 1..438: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPAIAGIGGEKAGGFAPYYG

SEQ ID NO: 985 - protein, 546 aa, synthetic construct
  REGION 1..438: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGHRGFPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 986 - protein, 556 aa, synthetic construct
  REGION 1..556: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGRPGRPGAAGLPGPPGIKGPAGIPGFPGMKGHRGFDGRNGEKGETGAPGLKGENGLPGAAGAPGPMGPRGAPGAAGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGPLVLRQLLVLVEKRPAVSLHIMVNQEDVRMPFA

SEQ ID NO: 987 - protein, 546 aa, synthetic construct
  REGION 1..546: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGKPGRPGERGLPGPPGIKGPAGIPGFPGMKGHRGFDGKNGEKGETGAPGLKGENGLPGENGAPGPMGPKGAPGERGKPGLPGAAGAKGNDGAKGSDGQPGPPGPPGTAGFPGSPGAKGEVGPAGSPGSNGAPGQKGEPGPQGHAGAQGPPGPPGINGSPGGKGEMGPAGIPGAPGLMGAKGPPGPAGANGAPGLKGGAGEPGKNGAKGEPGPKGEKGEAGIPGVPGAKGEDGKDGSPGEPGANGLPGAAGEKGAPGFKGPAGPNGIPGEKGPAGEKGAPGPAGPKGAAGEPGKDGVPGGPGMKGMPGSPGGPGSDGKPGPPGSQGESGKPGPPGPSGPKGQPGVMGFPGPKGNDGAPGKNGEKGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 988 - protein, 546 aa, synthetic construct
  REGION 1..546: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGAVGAKGEAGPQGPRGSEGPQGVRGEPGPPGPAGAAGPAGPPGADGQPGAKGAMGAPGIAGAPGFPGARGPPGPQGAGGPPGPKGGAGEPGAPGAKGDTGAKGERGPVGVQGPPGPAGEEGKRGARGEPGPTGLPGPPGERGGPGFRGFPGADGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 989 - protein, 546 aa, synthetic construct
  REGION 1..546: note = Recombinant collagen fragment
DVKSGVAVGGIAGYPGPAGPPGPPGPPGTSGHPGSPGSPGYQGPPGEPGQAGPSGPPGPPGALGPSGPAGKDGESGRPGRPGERGVPGPPGVKGPAGLPGFPGMKGHRGFDGRNGEKGETGAPGLKGENGLPGENGAPGPMGPRGAPGERGRPGLPGAAGARGNDGARGSDGQPGPPGPPGTAGFPGSPGAKGELGPAGSPGSNGAPGQRGEPGPQGHAGAQGPPGPPGLNGSPGGKGEMGPAGVPGAPGIMGARGPPGPAGANGAPGIRGGAGEPGKNGAKGEPGPRGERGEAGLPGLPGAKGEDGKDGSPGEPGANGVPGAAGERGAPGFRGPAGPNGLPGEKGPAGERGAPGPAGPRGAAGEPGRDGIPGGPGMRGMPGSPGGPGSDGKPGPPGSQGESGRPGPPGPSGPRGQPGLMGFPGPKGNDGAPGKNGERGGPGGPGPQGPPGKNGETGPQGPPGPTGPGGDKGDTGPPGPQGVQGLPGTGGPPGENGKPGEPGPKGDAGAPGAPGGKGDAGAPGERGPPAIAGIGGEKAGGFAPYYG

SEQ ID NO: 990 - protein, 546 aa, synthetic construct
  REGION 1..546: note = Recombinant collagen fragment
DVKSGVAVGGLAGYPGPAGPPGPPGPPGAAGHPGAPGAPGYQGPPGEPGQAGPAGPPGPPGAIGPAGPAGKDGEAGRPGRPGERGLPGPPGIKGPAGIPGFPGMKGHRGFDGRDGEKGEAGAPGLKGEDGLPGENGAPGPMGPRGAPGERGRPGLPGAAGARGDDGARGADGQPGPPGPPGAAGFPGAPGAKGEVGPAGAPGADGAPGQRGEPGPQGHAGAQGPPGPPGIDGAPGGKGEMGPAGIPGAPGLMGARGPPGPAGADGAPGLRGGAGEPGKDGAKGEPGPRGERGEAGIPGVPGAKGEDGKDGAPGEPGADGLPGAAGERGAPGFRGPAGPDGIPGEKGPAGERGAPGPAGPRGAAGEPGRDGVPGGPGMRGMPGAPGGPGADGKPGPPGAQGEAGRPGPPGPAGPRGQPGVMGFPGPKGDDGAPGKDGERGGPGGPGPQGPPGKDGEAGPQGPPGPAGPGGDKGDAGPPGPQGLQGLPGAGGPPGEDGKPGEPGPKGDAGAPGAPGGKGDAGAPGERGPPAIAGIGGEKAGGFAPYYG

SEQ ID NO: 991 - protein, 546 aa, synthetic construct
  REGION 1..546: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIEPGRPEGEKGGGRGGAPGAPGRGAGNGEGARPPPLMGGAGGNKAGPLAAPIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 992 - protein, 546 aa, synthetic construct; identical to SEQ ID NO: 991
  REGION 1..546: note = Recombinant collagen fragment

SEQ ID NO: 993 - protein, 546 aa, synthetic construct
  REGION 1..546: note = Recombinant collagen fragment
DVKSGVAVGGLAGYPGPAGPPGPPGPPGTSGHPGSPGSPGYQGPPGEPGQAGPSGPPGPPGAIGPSGPAGKRGESGRPGRPGERGLPGPPGIKGPAGIPGFPGMKGHRGFRGRNGEKGRTGAPGLKGENGLPGENGAPGPMGPRGAPGERGRPGLPGAAGARGNRGARGSRGQPGPPGPPGTAGFPGSPGAKGRVGPAGSPGSNGAPGQRGRPGPQGHAGAQGPPGPPGINGSPGGKGRMGPAGIPGAPGLMGARGPPGPAGANGAPGLRGGAGRPGKNGAKGRPGPRGERGRAGIPGVPGAKGRRGKRGSPGEPGANGLPGAAGERGAPGFRGPAGPNGIPGEKGPAGERGAPGPAGPRGAAGRPGRRGVPGGPGMRGMPGSPGGPGSRGKPGPPGSQGESGRPGPPGPSGPRGQPGVMGFPGPKGNRGAPGKNGRRGGPGGPGPQGPPGKNGETGPQGPPGPTGPGGRKGRTGPPGPQGLQGLPGTGGPPGENGKPGRPGPKGDAGAPGAPGGKGRAGAPGERGPPAIAGIGGEKAGGFAPYYG

SEQ ID NO: 994 - protein, 556 aa, synthetic construct
  REGION 1..556: note = Recombinant collagen fragment
DVKSGVAVGGMGGYPGPAGPPGPPGPPGVSGHPGAPGAPGYQGPPGEPGQAGPAGPPGPPGAMGPAGPAGKDGESGRPGRPGERGFPGPPGIKGPAGMPGFPGMKGHRGFDGRNGEKGDTGAPGLKGENGLPGENGAPGPMGPRGAPGERGRPGLPGAAGARGNDGARGSDGQPGPPGPPGTAGFPGSPGAKGEVGPAGAPGPNGAPGQRGEPGLQGQAGAPGPPGPPGINGSPGGKGEMGPAGIPGAPGLIGARGPPGPPGANGVPGQRGAAGEPGKNGAKGDPGARGERGEAGIPGIAGPKGEDGKDGSPGEPGANGLPGAPGERGPPGFRGAPGANGIPGEKGPPGERGGPGPAGPRGVAGEPGRDGLPGGPGLRGIPGSPGGPGSDGKPGPPGSQGESGRPGPPAHLVQGVSLVSWVSPVQRVMTEHRVKMVNVVAQVVQDCPVQQEKTVRQVLKDLQDLPVLQEIREMQDRQDLLDCKVCLVQEALPEKMVSLESQDQKAMSEPQVFPEVRETPVLRVSVVLRQLLVLVEKRPAVSLHIMVNQEDVRMPFA

SEQ ID NO: 995 - protein, 546 aa, synthetic construct
  REGION 1..546: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGVAGPKGPAGERGAPGPAGPKGAAGEAGRPGEAGLPGAKGMPGSPGGPGPDGKPGPPGPAGQDGRPGPPGPPGARGQAGVMGFPGPKGAAGEPGKAGERGVPGPPGAVGPAGKDGEAGAQGPPGPAGPAGERGEQGPAGPQGFQGLPGPAGPPGEAGKPGEQGVPGDLGAPGAPGARGERGAPGERGPPAIAGIGGEKAGGFAPYYG

SEQ ID NO: 996 - protein, 531 aa, synthetic construct
  REGION 1..531: note = Recombinant collagen fragment
DSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPP

SEQ ID NO: 997 - protein, 556 aa, synthetic construct
  REGION 1..556: note = Recombinant collagen fragment
DVKSGVAVGKTTKSGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPKTTKSAIAGIGGEKAGGFAPYYG

SEQ ID NO: 998 - protein, 556 aa, synthetic construct; identical to SEQ ID NO: 997
  REGION 1..556: note = Recombinant collagen fragment

SEQ ID NO: 999 - protein, 556 aa, synthetic construct
  REGION 1..556: note = Recombinant collagen fragment
DVKSGVAVGKTTKSGLAGYPGPAGPPGPPGPPGTSGHPGSPGSPGYQGPPGEPGQAGPSGPPGPPGAIGPSGPAGKDGESGRPGRPGAAGLPGPPGIKGPAGIPGFPGMKGHRGFDGRNGEKGETGAPGLKGENGLPGAAGAPGPMGPRGAPGAAGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGAAGPPKTTKSAIAGIGGEKAGGFAPYYG

SEQ ID NO: 1000 - protein, 546 aa, synthetic construct
  REGION 1..546: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEEGKRGARGEAGSAGPPGPPGLRGAPGFRGLPGADGRAGVMGPPGERGAPGPAGVRGAAGDAGRPGEPGLMGPRGLPGSPGGPGPAGKEGPVGLPGIDGRPGPIGPAGARGEPGVMGFPGPKGNDGDPGKNGDKGHAGLAGARGAPGPDGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 1001 - protein, 546 aa, synthetic construct
  REGION 1..546: note = Recombinant collagen fragment
DVKSGVAVGGMGGYPGPAGPPGPPGPPGVSGHPGAPGAPGYQGPPGEPGQAGPAGPPGPPGAMGPAGPAGKDGESGRPGRPGERGFPGPPGIKGPAGMPGFPGMKGHRGFDGRNGEKGDTGAPGLKGENGLPGENGAPGPMGPRGAPGERGRPGLPGAAGARGNDGARGSDGQPGPPGPPGTAGFPGSPGAKGEVGPAGAPGPNGAPGQRGEPGLQGQAGAPGPPGPPGINGSPGGKGEMGPAGIPGAPGLIGARGPPGPPGANGVPGQRGAAGEPGKNGAKGDPGARGERGEAGIPGIAGPKGEDGKDGSPGEPGANGLPGAPGERGPPGFRGAPGANGIPGEKGPPGERGGPGPAGPRGVAGEPGRDGLPGGPGLRGIPGSPGGPGSDGKPGPPGSQGESGRPGPPGSPGPRGQPGVMGFPGPKGNDGAPGKNGERGGPGGPGLPGPAGKNGETGPQGPPGPTGPAGDKGDAGPPGPPGLQGLPGTGGPPGENGKPGEPGPKGDVGAPGIPGGKGDAGAPGERGPPAIAGIGGEKAGGFAPYYG

SEQ ID NO: 1002 - protein, 546 aa, synthetic construct
  REGION 1..546: note = Recombinant collagen fragment
DVKSGVAVGGLAGYPGPAGPPGPPGPPGTSGHPGSPGSPGYQGPPGEPGQAGPAGPPGPIGNVGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 1003 - protein, 1466 aa, synthetic construct
  REGION 1..1466: note = human Col3A1
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVHLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 1004 - protein, 1463 aa, synthetic construct
  REGION 1..1463: note = bovine Col1A1
MFSFVDLRLLLLLAATALLTHGQEEGQEEGQEEDIPPVTCVQNGLRYHDRDVWKPVPCQICVCDNGNVLCDDVICDELKDCPNAKVPTDECCPVCPEGQESPTDQETTGVEGPKGDTGPRGPRGPAGPPGRDGIPGQPGLPGPPGPPGPPGPPGLGGNFAPQLSYGYDEKSTGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEGGPQGPRGSEGPQGVRGEPGPPGPAGAAGPAGNPGADGQPGAKGANGAPGIAGAPGFPGARGPSGPQGPSGPPGPKGNSGEPGAPGSKGDTGAKGEPGPTGIQGPPGPAGEEGKRGARGEPGPAGLPGPPGERGGPGSRGFPGADGVAGPKGPAGERGAPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGAPGKDGVRGLTGPIGPPGPAGAPGDKGEAGPSGPAGPTGARGAPGDRGEPGPPGPAGFAGPPGADGQPGAKGEPGDAGAKGDAGPPGPAGPAGPPGPIGNVGAPGPKGARGSAGPPGATGFPGAAGRVGPPGPSGNAGPPGPPGPAGKEGSKGPRGETGPAGRPGEVGPPGPPGPAGEKGAPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPIGPVGARGPAGPQGPRGDKGETGEQGDRGIKGHRGFSGLQGPPGPPGSPGEQGPSGASGPAGPRGPPGSAGSPGKDGLNGLPGPIGPPGPRGRTGDAGPAGPPGPPGPPGPPGPPSGGYDLSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKEKRHVWYGESMTGGFQFEYGGQGSDPADVAIQLTFLRLMSTEASQNITYHCKNSVAYMDQQTGNLKKALLLQGSNEIEIRAEGNSRFTYSVTYDGCTSHTGAWGKTVIEYKTTKTSRLPIIDVAPLDVGAPDQEFGFDVGPACFL

SEQ ID NO: 1005 - protein, 1364 aa, synthetic construct
  REGION 1..1364: note = bovine Col1A2
MLSFVDTRTLLLLAVTSCLATCQSLQEATARKGPSGDRGPRGERGPPGPPGRDGDDGIPGPPGPPGPPGPPGLGGNFAAQFDAKGGGPGPMGLMGPRGPPGASGAPGPQGFQGPPGEPGEPGQTGPAGARGPPGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGELGPVGNPGPAGPAGPRGEVGLPGLSGPVGPPGNPGANGLPGAKGAAGLPGVAGAPGLPGPRGIPGPVGAAGATGARGLVGEPGPAGSKGESGNKGEPGAVGQPGPPGPSGEEGKRGSTGEIGPAGPPGPPGLRGNPGSRGLPGADGRAGVMGPAGSRGATGPAGVRGPNGDSGRPGEPGLMGPRGFPGSPGNIGPAGKEGPVGLPGIDGRPGPIGPAGARGEPGNIGFPGPKGPSGDPGKAGEKGHAGLAGARGAPGPDGNNGAQGPPGLQGVQGGKGEQGPAGPPGFQGLPGPAGTAGEAGKPGERGIPGEFGLPGPAGARGERGPPGESGAAGPTGPIGSRGPSGPPGPDGNKGEPGVVGAPGTAGPSGPSGLPGERGAAGIPGGKGEKGETGLRGDIGSPGRDGARGAPGAIGAPGPAGANGDRGEAGPAGPAGPAGPRGSPGERGEVGPAGPNGFAGPAGAAGQPGAKGERGTKGPKGENGPVGPTGPVGAAGPSGPNGPPGPAGSRGDGGPPGATGFPGAAGRTGPPGPSGISGPPGPPGPAGKEGLRGPRGDQGPVGRSGETGASGPPGFVGEKGPSGEPGTAGPPGTPGPQGLLGAPGFLGLPGSRGERGLPGVAGSVGEPGPLGIAGPPGARGPPGNVGNPGVNGAPGEAGRDGNPGNDGPPGRDGQPGHKGERGYPGNAGPVGAAGAPGPQGPVGPVGKHGNRGEPGPAGAVGPAGAVGPRGPSGPQGIRGDKGEPGDKGPRGLPGLKGHNGLQGLPGLAGHHGDQGAPGAVGPAGPRGPAGPSGPAGKDGRIGQPGAVGPAGIRGSQGSQGPAGPPGPPGPPGPPGPSGGGYEFGFDGDFYRADQPRSPTSLRPKDYEVDATLKSLNNQIETLLTPEGSRKNPARTCRDLRLSHPEWSSGYYWIDPNQGCTMDAIKVYCDFSTGETCIRAQPEDIPVKNWYRNSKAKKHVWVGETINGGTQFEYNVEGVTTKEMATQLAFMRLLANHASQNITYHCKNSIAYMDEETGNLKKAVILQGSNDVELVAEGNSRFTYTVLVDGCSKKTNEWQKTIIEYKTNKPSRLPILDIAPLDIGGADQEIRLNIGPVCFK

SEQ ID NO: 1006 - protein, 1466 aa, synthetic construct
  REGION 1..1466: note = bovine Col3A1
MMSFVQKGTWLLFALLHPTVILAQQEAVDGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGPPGSPGSPGSPGPPGICESCPTGGQNYSPQYEAYDVKSGVAGGGIAGYPGPAGPPGPPGPPGTSGHPGAPGAPGYQGPPGEPGQAGPAGPPGPPGAIGPSGPAGKDGESGRPGRPGERGFPGPPGMKGPAGMPGFPGMKGHRGFDGRNGEKGETGAPGLKGENGVPGENGAPGPMGPRGAPGERGRPGLPGAAGARGNDGARGSDGQPGPPGPPGTAGFPGSPGAKGEVGPAGSPGSSGAPGQRGEPGPQGHAGAPGPPGPPGSNGSPGGKGEMGPAGIPGAPGLIGARGPPGPPGTNGVPGQRGAAGEPGKNGAKGDPGPRGERGEAGSPGIAGPKGEDGKDGSPGEPGANGLPGAAGERGVPGFRGPAGANGLPGEKGPPGDRGGPGPAGPRGVAGEPGRDGLPGGPGLRGIPGSPGGPGSDGKPGPPGSQGETGRPGPPGSPGPRGQPGVMGFPGPKGNDGAPGKNGERGGPGGPGPQGPAGKNGETGPQGPPGPTGPSGDKGDTGPPGPQGLQGLPGTSGPPGENGKPGEPGPKGEAGAPGIPGGKGDSGAPGERGPPGAGGPPGPRGGAGPPGPEGGKGAAGPPGPPGSAGTPGLQGMPGERGGPGGPGPKGDKGEPGSSGVDGAPGKDGPRGPTGPIGPPGPAGQPGDKGESGAPGVPGIAGPRGGPGERGEQGPPGPAGFPGAPGQNGEPGAKGERGAPGEKGEGGPPGAAGPAGGSGPAGPPGPQGVKGERGSPGGPGAAGFPGGRGPPGPPGSNGNPGPPGSSGAPGKDGPPGPPGSNGAPGSPGISGPKGDSGPPGERGAPGPQGPPGAPGPLGIAGLTGARGLAGPPGMPGARGSPGPQGIKGENGKPGPSGQNGERGPPGPQGLPGLAGTAGEPGRDGNPGSDGLPGRDGAPGAKGDRGENGSPGAPGAPGHPGPPGPVGPAGKSGDRGETGPAGPSGAPGPAGSRGPPGPQGPRGDKGETGERGAMGIKGHRGFPGNPGAPGSPGPAGHQGAVGSPGPAGPRGPVGPSGPPGKDGASGHPGPIGPPGPRGNRGERGSEGSPGHPGQPGPPGPPGAPGPCCGAGGVAAIAGVGAEKAGGFAPYYGDEPIDFKINTDEIMTSLKSVNGQIESLISPDGSRKNPARNCRDLKFCHPELQSGEYWVDPNQGCKLDAIKVYCNMETGETCISASPLTIPQKNWWTDSGAEKKHVWFGESMEGGFQFSYGNPELPEDVLDVQLAFLRLLSSRASQNITYHCKNSIAYMDHASGNVKKALKLMGSNEGEFKAEGNSKFTYTVLEDGCTKHTGEWGKTVFQYQTRKAVRLPIVDIAPYDIGGPDQEFGADIGPVCFL

SEQ ID NO: 1007 - protein, 546 aa, synthetic construct
  REGION 1..546: note = Recombinant Collagen Fragment
DVKSGVAVGGLAGYPGPAGPPGPPGPPGTSGHKGSPGSPGYQGPPGEPGQAGPAGPPGPIGNVGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPRPPGPPGTAGFPGSPGAKGEVGPAGSPGSNGAPGQRGEPGPQGHKGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 1008 - protein, 547 aa, synthetic construct
  REGION 1..547: note = Recombinant Collagen Fragment; note = 1..547
DSYDVKSGVAVGGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYG

SEQ ID NO: 1009 - protein, 1466 aa, synthetic construct; identical to SEQ ID NO: 1006
  source 1..1466: note = bovine collagen type III alpha 1

(19) United States
(12) Patent Application Publication
(10) Pub. No.: US 2026/0027187 A1
(43) Pub. Date: Jan. 29, 2026

(54) METHODS OF INHIBITING CANCER GROWTH

(71) Applicant: HTL BIOTECHNOLOGY INNOVATION, INC., Nutley, NJ (US)

(72) Inventors: Lixin DAI, Nutley, NJ (US); Dale L. HANDLIN, Jr., Nutley, NJ (US); Stanley LUE, Nutley, NJ (US); David WILLIAMSON, Nutley, NJ (US); Stuart NEWMAN, Nutley, NJ (US); Monique DE LEEUW, Nutley, NJ (US)

(21) Appl. No.: 18/868,280

(22) PCT Filed: May 31, 2023
(86) PCT No.: PCT/US2023/067727

§ 371 Date: Nov. 22, 2024

Related U.S. Application Data
(60) Provisional application No. 63/347,699, filed on Jun. 1, 2022.

(51) Int. Cl. A61K 38/39; A61K 47/34; A61P 35/00

(52) U.S. Cl. CPC A61K 38/39; A61K 47/34; A61P 35/00

(57) ABSTRACT

The present disclosure provides methods of treating cancer in a subject in need thereof, the method comprising contacting a cellular surface on the subject with an effective amount of a recombinant collagen fragment, including a fragment having a sequence identity of at least about 85% to the amino acid sequence set forth in SEQ ID NO: 1. Therapeutic biomaterials for use in the methods described herein are also provided.

### REFERENCE TO SEQUENCE LISTING SUBMITTED ELECTRONICALLY

The content of the electronically submitted sequence listing XML (Name: 4431_088PC01_SequenceListing_ST26.xml; Size: 1,312,028 bytes; and Date of Creation: May 30, 2023) filed with the application is incorporated herein by reference in its entirety.

### BACKGROUND

Collagen is one of the most important proteins in the human body, and is present in connective tissue such as cartilage, bones, tendons, ligaments, and skin. It is the major protein in the extra-cellular matrix of human cells. “Recombinant collagen” refers to the family of at least 28 distinct naturally occurring collagen types prepared using recombinant techniques.

Despite recent advances in cancer therapies, cancer is still a leading cause of death worldwide, accounting for nearly 10 million deaths in 2020. One defining feature of cancer is the rapid creation of abnormal cells that can invade and seed in distant organs, far from a primary tumor or neoplasm. These metastatic cells can remain dormant for many years before forming clinically detectable metastatic tumors. Widespread metastases are the primary cause of death from cancer. While cancer treatment modalities such as immunotherapy are a popular area of clinical research, there remains a need in the field of oncology for creative approaches and novel therapeutics for treating primary tumors and preventing metastatic seed cells from becoming incurable metastatic tumors.

### BRIEF SUMMARY

The present disclosure provides novel and inventive methods and compositions for inhibiting cancer growth in a subject in need thereof. In some embodiments, this disclosure provides a method of treating a cancer in a subject in need thereof, the method comprising contacting a cellular surface on the subject with an effective amount of a recombinant collagen fragment having a sequence identity of at least about 85% to the amino acid sequence set forth in SEQ ID NO: 1. In some embodiments, the recombinant collagen fragment is unhydroxylated. In some embodiments, the recombinant collagen fragment is hydroxylated.

In some embodiments, the collagen fragment has the amino acid sequence set forth in SEQ ID NO: 1. In some embodiments, the collagen fragment having the amino acid sequence set forth in SEQ ID NO: 1 is unhydroxylated. In some embodiments, the collagen fragment having the amino acid sequence set forth in SEQ ID NO: 1 is hydroxylated.

In some embodiments, this disclosure provides a method of treating a cancer in a subject in need thereof, the method comprising contacting a cellular surface on the subject with an effective amount of a recombinant collagen fragment having the amino acid sequence set forth in any one of SEQ ID NOs: 2-972.

In some embodiments, contacting the cellular surface with the fragment induces cellular dormancy.

In some embodiments, the cancer is characterized by the presence of a solid tumor. In some embodiments, the cancer is a cancer of the skin, colon, oral mucosa, rectum, esophagus, thyroid, liver, pancreas, kidney, bladder, lung, brain, breast, ovary, testicle, prostate, bones, or a combination of any of the foregoing.

In some embodiments, the subject has not undergone resection of a tumor. In some embodiments, the subject has undergone resection of a primary tumor. In some embodiments, the cellular surface of the subject comprises residual cancer cells following the resection of the primary tumor.

In some embodiments, contacting the cellular surface with the fragment maintains cellular dormancy.

In some embodiments, this disclosure provides a method of inhibiting proliferation of a cancer cell, the method comprising contacting the cancer cell with a recombinant collagen fragment having a sequence identity of at least about 85% to the amino acid sequence set forth in SEQ ID NO: 1. In some embodiments, the recombinant collagen fragment is unhydroxylated. In some embodiments, the recombinant collagen fragment is hydroxylated.

In some embodiments of the method of inhibiting proliferation of a cancer cell disclosed herein, the collagen fragment has the amino acid sequence set forth in SEQ ID NO: 1. In some embodiments, the recombinant collagen fragment is unhydroxylated. In some embodiments, the recombinant collagen fragment is hydroxylated.

In some embodiments, this disclosure provides a method of inhibiting proliferation of a cancer cell, the method comprising contacting the cancer cell with a recombinant collagen fragment having the amino acid sequence set forth in any one of SEQ ID NOs: 2-972.

In some embodiments of the methods disclosed herein, the collagen fragment is formulated in a pharmaceutically acceptable composition. In some embodiments, the fragment is formulated in a therapeutic biomaterial.

In some embodiments the polydispersity of the collagen fragments (the weight average molecular weight of the collagen fragment divided by the number average molecular weight of the collagen fragment) provided herein can be about 1. In some embodiments the polydispersity of the collagen fragments can range from about 1 to about 2. In some embodiments the polydispersity can range from about 1 to about 3 or from about 1 to about 5.

In some embodiments, the therapeutic biomaterial can be a protein polyurethane alloy. In some embodiments, the protein is dissolved in the polyurethane. In some embodiments, the protein migrates out of the alloy to contact the cellular surface when the alloy is exposed to water. In some embodiments, the protein migrates out of the alloy to contact the cellular surface when the alloy is exposed to a buffer solution. In some embodiments, the alloy can comprise about 10 wt % to about 50 wt % of the fragment and about 50 wt % to about 90 wt % of the polyurethane. In some embodiments, the alloy can comprise about 20 wt % to about 35 wt % of the protein and about 65 wt % to about 80 wt % of the polyurethane. In some embodiments, the alloy can be free of, or can be substantially free of, particles of the recombinant collagen fragment having an average diameter of greater than 1 micron.

In some embodiments, this disclosure provides a therapeutic biomaterial comprising a recombinant collagen fragment having a sequence identity of at least about 85% to the amino acid sequence set forth in SEQ ID NO: 1 and a pharmaceutically acceptable scaffold. In some embodiments, the recombinant collagen fragment within the pharmaceutically acceptable scaffold can be unhydroxylated. In some embodiments, the recombinant collagen fragment within the pharmaceutically acceptable scaffold can be hydroxylated.

In some embodiments, the recombinant collagen fragment within the pharmaceutically acceptable scaffold has the amino acid sequence set forth in SEQ ID NO: 1. In some embodiments, the recombinant collagen fragment within the pharmaceutically acceptable scaffold can be unhydroxylated. In some embodiments, the recombinant collagen fragment within the pharmaceutically acceptable scaffold can be hydroxylated

In some embodiments, this disclosure provides a therapeutic biomaterial comprising a recombinant collagen fragment having a sequence according to any one of SEQ ID NOs: 2-972, and a pharmaceutically acceptable scaffold.

In some embodiments, this disclosure provides a therapeutic biomaterial comprising a recombinant collagen fragment having a sequence identity of at least about 85% to the amino acid sequence set forth in SEQ ID NO: 1 and a pharmaceutically acceptable scaffold, for use in the treatment of a cancer in a subject in need thereof. In some embodiments, the cancer can be characterized by the presence of a solid tumor. In some embodiments, the cancer can be a cancer of the skin, colon, oral mucosa, rectum, esophagus, thyroid, liver, pancreas, kidney, bladder, lung, brain, breast, ovary, testicle, prostate, bones, or a combination of any of the foregoing.

In some embodiments, the pharmaceutically acceptable scaffold can be a protein polyurethane alloy wherein the protein is dissolved in the polyurethane. Protein polyurethane alloys are described, for example in U.S. 2021-0355326, the entirety of which is incorporated herein by reference. In some embodiments, the protein migrates out of the alloy to contact the cellular surface when the alloy is exposed to water or a buffer solution. In some embodiments, the protein migrates out of the alloy when the alloy is exposed to a cellular surface.

In some embodiments, the alloy comprises about 10 wt % to about 50 wt % of the fragment and about 50 wt % to about 90 wt % of the polyurethane. In some embodiments, the alloy comprises about 20 wt % to about 35 wt % of the protein and about 65 wt % to about 80 wt % of the polyurethane. In some embodiments, the alloy is free of, or substantially free of, particles of the recombinant collagen fragment having an average diameter of greater than 1 micron.

In some embodiments, the pharmaceutically acceptable scaffold comprises a protein hyaluronic acid alloy. In some embodiments the hyaluronic acid and the protein are crosslinked to themselves, to each other, or both. In some embodiments the protein is dissolved in a crosslinked hyaluronic matrix. In some embodiments the protein migrates out of the alloy when the alloy is exposed to water, a buffer solution, and/or a cellular surface.

In some embodiments, the pharmaceutically acceptable scaffold comprises a protein dissolved in a gelatin matrix. In some embodiments, the gelatin is crosslinked. In some embodiments, the protein migrates out of the alloy to contact the cellular surface when the alloy is exposed to water or a buffer solution.

In some embodiments, the biomaterial is adhered directly onto the cancer cells.

In some embodiments, this disclosure provides a use of the biomaterial described herein for treating a wound resulting from resection of a primary tumor.

In some embodiments, this disclosure provides a use of the biomaterial described herein, for maintaining cellular dormancy.

In some embodiments, this disclosure provides a therapeutic biomaterial comprising a recombinant collagen fragment having a sequence identity of at least about 85% to the amino acid sequence set forth in SEQ ID NO: 1 and a pharmaceutically acceptable scaffold. In some embodiments, the recombinant collagen fragment is unhydroxylated. In some embodiments, the recombinant collagen fragment is hydroxylated.

In some embodiments, the collagen fragment has the amino acid sequence set forth in SEQ ID NO: 1. In some embodiments, the recombinant collagen fragment is unhydroxylated. In some embodiments, the recombinant collagen fragment is hydroxylated

In some embodiments, this disclosure provides a therapeutic biomaterial comprising a recombinant collagen fragment having a sequence comprising the amino acid sequence set forth in any one of SEQ ID NOs: 2-972, or the amino acid sequence set forth in any one of SEQ ID NOs: 974-1002, and a pharmaceutically acceptable scaffold.

In some embodiments, this disclosure provides a therapeutic biomaterial comprising a recombinant collagen fragment having a sequence identity of at least about 85% to the amino acid sequence set forth in SEQ ID NO: 1 and a pharmaceutically acceptable scaffold, for use in the treatment of a cancer in a subject in need thereof. In some embodiments, the cancer is characterized by the presence of a solid tumor. In some embodiments the cancer is a cancer of the skin, colon, oral mucosa, rectum, esophagus, thyroid, liver, pancreas, kidney, bladder, lung, brain, breast, ovary, testicle, prostate, bones, or a combination of any of the foregoing.

In some embodiments, this disclosure provides a biomaterial comprising a pharmaceutically acceptable scaffold. In some embodiments, the pharmaceutically acceptable scaffold comprises a mixture of hyaluronic acid and the recombinant collagen fragment. In some embodiments, the recombinant collagen fragment is mixed with the hyaluronic acid. In some embodiments, the hyaluronic acid scaffold is crosslinked. In some embodiments, the hyaluronic acid is a biphasic crosslinked hyaluronic acid comprising crosslinked HA and uncrosslinked HA.

In some embodiments, this disclosure provides a biomaterial comprising a pharmaceutically acceptable scaffold, wherein the pharmaceutically acceptable scaffold comprises a mixture of polyvinylpyrrolidone and the recombinant collagen fragment.

In some embodiments, the pharmaceutically acceptable scaffold comprises a mixture of polyacrylamide and the recombinant collagen fragment

In some embodiments, the pharmaceutically acceptable scaffold comprises a mixture of poly(ethylene oxide) and the recombinant collagen fragment.

In some embodiments, the pharmaceutically acceptable scaffold comprises a mixture of poly(2-oxazoline)s and the recombinant collagen fragment

In some embodiments, the pharmaceutically acceptable scaffold comprises a mixture of polyethylenimine and the recombinant collagen fragment.

In some embodiments, the pharmaceutically acceptable scaffold comprises a mixture of carboxymethylcellulose and the recombinant collagen fragment.

In some embodiments, the pharmaceutically acceptable scaffold comprises a mixture of chondroitin sulfate and the recombinant collagen fragment.

In some embodiments, the pharmaceutically acceptable scaffold comprises a mixture of an acetylated hyaluronate and the recombinant collagen fragment.

In some embodiments, the pharmaceutically acceptable scaffold comprises a mixture of zinc hyaluronate and the recombinant collagen fragment.

In some embodiments, the biomaterial is adhered directly onto the cancer cells.

In some embodiments, this disclosure provides a use of the disclosed biomaterials for treating a wound resulting from resection of a primary tumor.

In some embodiments, this disclosure provides a use for maintaining cellular dormancy.

In some embodiments, this disclosure provides a method of treating a cancer in a subject in need thereof, the method comprising contacting a cellular surface on the subject with a therapeutic biomaterial comprising an effective amount of a recombinant collagen fragment having a sequence identity of at least about 85% to the amino acid sequence set forth in SEQ ID NO: 1. In some embodiments, the recombinant collagen is unhydroxylated. In some embodiments, the recombinant collagen is hydroxylated.

In some embodiments, this disclosure provides a method of inhibiting proliferation of a cancer cell, the method comprising contacting the cancer cell with a therapeutic biomaterial comprising a recombinant collagen fragment having the amino acid sequence set forth in SEQ ID NO: 973. In some embodiments, the recombinant collagen is unhydroxylated. In some embodiments, the recombinant collagen is hydroxylated.

In some embodiments, this disclosure provides a method of inducing cellular dormancy in a subject in need thereof, the method comprising contacting a cellular surface in the subject with a therapeutic biomaterial comprising a recombinant collagen having the amino acid sequence set forth in SEQ ID NO: 973. In some embodiments, the recombinant collagen is unhydroxylated. In some embodiments, the recombinant collagen is hydroxylated.

In some embodiments of the methods of treating cancer provided herein, the cancer is characterized by the presence of a solid tumor. In some embodiments, the cancer is a cancer of the skin, colon, oral mucosa, rectum, esophagus, thyroid, liver, pancreas, kidney, bladder, lung, brain, breast, ovary, testicle, prostate, bones, or a combination of any of the foregoing.

In some embodiments of the methods provided herein, the therapeutic biomaterial is a protein polyurethane alloy.

In some embodiments of the methods provided herein, the therapeutic biomaterial comprises a polyurethane, hyaluronic acid, or gelatin.

In some embodiments of the methods provided herein, the therapeutic biomaterial comprises polyvinylpyrrolidone.

In some embodiments of the methods provided herein, the therapeutic biomaterial comprises polyacrylamide

In some embodiments of the methods provided herein, the therapeutic biomaterial comprises poly(ethylene oxide).

In some embodiments of the methods provided herein, the therapeutic biomaterial comprises poly(2-oxazoline)s.

In some embodiments of the methods provided herein, the therapeutic biomaterial comprises polyethylenimine.

In some embodiments of the methods provided herein, the therapeutic biomaterial comprises carboxymethylcellulose.

In some embodiments of the methods provided herein, the therapeutic biomaterial comprises chondroitin sulfate.

In some embodiments of the methods provided herein, the therapeutic biomaterial comprises acetylated hyaluronate.

The method of any one of claims 60-66, wherein the therapeutic biomaterial comprises acetylated hyaluronate.

In some embodiments of the methods provided herein, the therapeutic biomaterial comprises zinc hyaluronate.

In some embodiments of the methods provided herein, the therapeutic biomaterial is adhered directly onto the cancer cells.

In some embodiments of the methods provided herein, polydispersity of the collagen fragments is 1 to 2, 1 to 3, or 1 to 5. In some embodiments, the polydispersity of the collagen fragments is 1 to 2, 1 to 3, or 1 to 5.

### BRIEF DESCRIPTION OF THE FIGURES

FIG. 1A is a graph showing mean tumor growth by group over time in days.

FIG. 1B is a graph showing median tumor growth by group over time in days.

FIGS. 2A-2J are graphs showing the effect of test collagens (different types and concentrations as described herein) compared to untreated controls over time in days.

FIG. 2A is a graph showing mean estimated tumor volume for 1.5 mg/mL bovine collagen compared to untreated control up to day 23.

FIG. 2B is a graph showing mean estimated tumor volume for 1.5 mg/mL bovine collagen and 10 mg/mL compared to untreated control up to day 23.

FIG. 2C is a graph showing mean estimated tumor volume for SEQ ID NO 1 at 200 mg/mL compared to untreated control up to day 23.

FIG. 2D is a graph showing mean estimated tumor volume for SEQ ID NO 1 at 10 mg/mL compared to untreated control up to day 23.

FIG. 2E is a graph showing mean estimated tumor volume for SEQ ID NO 1 at 1.5 mg/mL compared to untreated control up to day 23.

FIG. 2F is a graph showing mean estimated tumor volume for SEQ ID NO 1 at all three concentrations compared to untreated control up to day 23.

FIG. 2G is a graph showing mean estimated tumor volume for 1.5 mg/mL bovine collagen and 10 mg/mL hydroxylated SEQ ID NO 1 compared to untreated control up to day 23.

FIG. 2H is a graph showing mean estimated tumor volume for 10 mg/mL SEQ ID NO 1 and 10 mg/mL hydroxylated SEQ ID NO 1 compared to untreated control up to day 23.

FIG. 2I is a graph showing mean estimated tumor volume for 1.5 mg/mL bovine collagen and 1.5 mg/mL bovine collagen with 0.1% ethanol compared to untreated control up to day 23.

FIG. 2J is a graph showing mean estimated tumor volume for 1.5 mg/mL bovine collagen and 1.5 mg/mL bovine collagen with 0.1% ethanol up to day 23.

FIG. 3 is a graph showing progression-free survival by test group.

FIGS. 4A-4D are a series of graphs showing individual tumor growth curves by group.

FIGS. 5A-5E are a series of photographs showing tumors after necropsy.

FIGS. 6A-6J are a series of representative photomicrographs of subcutaneous tumors from untreated and treated BALB/c mice.

FIG. 7 shows the workflow of ECM extraction from tumor samples.

FIG. 8 shows enrichment of collagen type III in tumor samples.

FIGS. 9A-9F are in vitro proliferation assay results for bovine collagen.

FIG. 9A shows results in MCF-7 LUC cells.

FIG. 9B shows results in HCT-116 cells.

FIG. 9C shows results in HepG2 cells.

FIG. 9D shows results in 4T1 cells.

FIG. 9E shows cell image results in 4T1 cells.

FIG. 9F shows 4T1 cell proliferation assay reading at time zero.

FIGS. 10A-10E are in vitro proliferation assay results for SEQ ID NO 1.

FIG. 10A shows results in MCF-7 LUC cells.

FIG. 10B shows results in HCT-116 cells.

FIG. 10C shows results in HepG2 cells.

FIG. 10D shows results in 4T1 cells.

FIG. 10E shows cell image results in 4T1 cells.

FIG. 11A is a standard curve used to convert UV absorbance to concentration of protein by showing the relationship between absorbance (Y-axis) as a function of protein concentration (X-axis).

FIG. 11B shows concentration of protein released from a PBS solution of protein compared to a formulation of HA and protein in PBS (Y-axis) over time in days (X-axis). The measurements in FIGS. 11A-B were taken outside the 300 kDa molecular weight cutoff (MWCO) dialysis membrane.

FIG. 12A is a standard curve used to convert absorbance to concentration of protein by showing the relationship between absorbance (Y-axis) as a function of protein concentration (X-axis).

FIG. 12B shows concentration of protein released (Y-axis) from the PBS compared to the protein released from the HA Formulation over time in days (X-axis). The measurements for FIGS. 12A-12B taken from outside the 300 kDa MWCO dialysis membrane as in FIGS. 11A-11B.

FIG. 13A is a standard curve used to convert absorbance to concentration of protein by showing the relationship between absorbance (Y-axis) as a function of protein concentration (X-axis).

FIG. 13B shows protein concentrations plotted against timepoints to give a trend of the amount of protein remaining inside the PBS Solution and HA Formulation after dialysis. In this experiment, data was taken from inside the starting solutions as opposed to FIGS. 12-13 which were taken from outside the 300 kDa MWCO dialysis membrane.

FIG. 14A is a standard curve used to convert absorbance to concentration of protein by showing the relationship between absorbance (Y-axis) as a function of protein concentration (X-axis).

FIG. 14B shows concentration of protein released (Y-axis) from the PBS solution compared to the protein released from the HA Formulation over time in days (X-axis).

### DETAILED DESCRIPTION

### Definitions

The indefinite articles “a” and “an” to describe an element or component means that one or at least one of these elements or components is present. Although these articles are conventionally employed to signify that the modified noun is a singular noun, as used herein the articles “a” and “an” also include the plural, unless otherwise stated in specific instances. Similarly, the definite article “the,” as used herein, also signifies that the modified noun can be singular or plural, again unless otherwise stated in specific instances.

As used herein, the term “about” used with numerical values means within 10% of the stated value, unless expressly noted otherwise. For example, “about 5% by weight” means from 4.5% by weight to 5.5% by weight. The foregoing notwithstanding, “about” should not be understood to modify values to greater than or equal to 100%. For example, a composition comprising “about 95” weight percent of a given element could have from 85.5 to 99.999 weight percent of the component in the composition.

The term “collagen” refers to any one of the known collagen types, whether natural, synthetic, semi-synthetic, or recombinant. The term collagen includes collagen, collagen fragments, collagen-like proteins, triple helical collagen, alpha chains, monomers, gelatin, trimers and combinations thereof. It includes all of the collagens, modified collagens and collagen-like proteins described herein. The term also encompasses procollagens and collagen-like proteins or collagenous proteins comprising the motif (Gly-X-Y) n where n is an integer. It encompasses molecules of collagen and collagen-like proteins, trimers of collagen molecules, fibrils of collagen, and fibers of collagen fibrils. It also refers to chemically, enzymatically or recombinantly-modified collagens or collagen-like molecules that can be fibrillated as well as fragments of collagen, collagen-like molecules and collagenous molecules capable of assembling into a nanofiber. Recombinant collagen molecules whether native or engineered will generally comprise a repeated -(Gly-X-Y)n- sequence.

As used herein, collagen is a generic term for a family of at least 28 distinct collagen types. Various distinct collagen types have been identified in a range of species, including bovine, ovine, porcine, chicken, marine, plant, and human collagens. Animal skin is typically Type I collagen. The term “collagen” encompasses unprocessed (e.g., procollagens) as well as post-translationally modified and proteolyzed collagens having a triple helical structure. Type I collagen is the major fibrillar collagen of bone and skin comprising approximately 80-90% of an organism's total collagen. Type I collagen is the major structural macromolecule present in the extracellular matrix of multicellular organisms and comprises approximately 20% of total protein mass. Type I collagen is a heterotrimeric molecule comprising two α1(I) chains and one α2(I) chain, encoded by the COL1A1 and COL1A2 genes, respectively. In vivo assembly of Type I collagen fibrils, fibers, and fiber bundles takes place during development and provides mechanical support to the tissue while allowing for cellular motility and nutrient transport. Other collagen types are less abundant than type I collagen and exhibit different distribution patterns. Type III collagen is a major fibrillar collagen found in skin and vascular tissues. Type III collagen is a homotrimeric collagen comprising three identical α1(III) chains encoded by the COL3A1 gene.

The term “modified,” as applied to the collagen fragments disclosed herein, refers to collagen fragments comprising an amino acid sequence that is at least 70%, 80%, 90%, 95%, or 99% identical or similar to the amino acid sequence of a biologically active molecule. In some embodiments, the modified collagen fragment comprises an amino acid sequence that is at least 75%, 80%, 85%, 86%, 87%, 88%, 89%, 90%, 91%, 92%, 93%, 94%, 95%, 96%, 97%, 98%, or 99% identical to the amino acid sequence of a native or previously engineered sequence. The modified sequence can comprise additions, deletions, substitutions, or a combination thereof to the amino acid sequence of a native or previously engineered molecule. For example, a modified collagen fragment can incorporate or delete 1, 2, 3, 4, 5, 6, 7, 8, 9, 10 or more amino acid residues compared to a native collagen sequence. Such selections can be made to modify the looseness or tightness of a recombinant collagen. The degree of hydroxylation of collagen correlates with the looseness or tightness of the collagen triple helix. A modified collagen fragment can also include chemical modifications to a polypeptide, such as crosslinks between cysteine residues, or hydroxylated or glycosylated residues, or hydrolysis to a lower molecular weight by any mechanism.

The term “pharmaceutically acceptable” as applied to carriers, excipients, or stabilizers that may be used in the compositions described herein, refer to carriers, excipients, or stabilizers that are nontoxic to recipients at the dosages and concentrations employed.

As used herein, the term “protein” refers to any of the collagen fragments described herein including: 1) “50 kDa rCol”; 2) a recombinant collagen fragment having a sequence identity of at least about 85% to the amino acid sequence set forth in SEQ ID NO: 1; 3) a recombinant collagen fragment having at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity, or similarity to SEQ ID NO: 1; and 4) combinations of any of the foregoing.

As used herein, the term “hyaluronic acid” and “HA” refers to acidic polysaccharides with different molecular weights made up of residues of D-glucuronic and N-acetyl-D-glucosamine acids, and their corresponding salts, including but not limited sodium hyaluronate. As used herein, compositions, scaffolds, therapeutic biomaterials, or any contemplated embodiment comprising HA should be understood to comprise a combination of hyaluronic acid and sodium hyaluronate unless otherwise specified.

As used the terms “sodium carboxymethylcellulose,” herein, “carboxymethylcellulose” and “CMC” can be used interchangeably, and refer to a polymer with different ranges of molecular weights as a derivative of cellulose with carboxymethyl groups bound to some of the monomers that make up the cellulose backbone. Compositions described herein as comprising carboxymethylcellulose should be understood to comprise a combination of carboxymethylcellulose and sodium carboxymethylcellulose unless otherwise specified.

As used herein, the term “miscible” means that one substance is soluble with the other such that a molecularly homogeneous mixture is formed when the two components are mixed together in water or in a buffer system. In solution, collagen that is fully miscible with hyaluronic acid refers to a solution that is a uniformly mixed or blended combination that forms a transparent, single-phase under suitable process conditions wherein there are no visible collagen particles present in the solution. Solid films prepared from fully miscible solutions can appear visually transparent, hazy, and/or can show phase separation. Transparency can be readily determined by a person of ordinary skill in the art and can be measured, for example, using a haze meter.

In the context of a protein polyurethane alloy, a protein that is miscible with the polyurethane means that the protein can be miscible with only one of a plurality of phases of the polyurethane, or the plurality of polyurethanes, with which it is blended.

### A. Methods of Treating Cancer

In some embodiments, this disclosure provides a method of treating a cancer in a subject in need thereof, the method comprising contacting a cellular surface of the subject with an effective amount of a recombinant collagen fragment.

As used herein, the term “recombinant collagen” refers to the family of at least 28 distinct naturally occurring collagen types including, but not limited to collagen types I, II, III, IV, V, VI, VII, VIII, IX, X, XI, XII, XIII, XIV, XV, XVI, XVII, XVIII, XIX, and XX, prepared using recombinant techniques. The term collagen includes collagen, collagen fragments, collagen-like proteins, triple helical collagen, alpha chains, monomers, gelatin, trimers and combinations thereof. Recombinant expression of collagen and collagen-like proteins is known in the art (see, e.g., Bell, EP 1232182B1, Bovine collagen and method for producing recombinant gelatin; Olsen, et al., U.S. Pat. No. 6,428,978 and VanHeerde, et al., U.S. Pat. No. 8,188,230, incorporated by reference herein in their entireties). Collagens are characterized by a repeating triplet of amino acids, -(Gly-X-Y)n-, so that approximately one-third of the amino acid residues in collagen are glycine. X is often proline and Y is often hydroxyproline. The structure of collagen may consist of three intertwined peptide chains of differing lengths.

In some embodiments, the recombinant collagen described herein is a recombinant collagen fragment. A recombinant collagen fragment can be a fragment of the full amino acid sequence of a native collagen molecule capable of forming tropocollagen (trimeric collagen) or the fragment can be a fragment of a modified collagen molecule or truncated collagen molecule having an amino acid sequence at least 70, 80, 90, 95, 96, 97, 98, or 99% identical or similar to a native collagen amino acid sequence (or to a fibril forming region thereof or to a segment substantially comprising [Gly-X-Y]n).

Exemplary collagen sequences from which fragments can be derived include amino acid sequences of Col1A1, Col1A2, and Col3A1, such as those described by Accession Nos. P02461.4 (SEQ ID 1003; NO: human Col3A1) (www.ncbi.nlm.nih.gov/protein/124056490), P02452 (SEQ ID NO: 982; human Col1A1), P08123 (SEQ ID NO: 983; human Col1A2) NP_001029211.1 (SEQ ID NO: 1004; bovine Col1A1) (www.ncbi.nlm.nih.gov/protein/77404252), NP_776945.1 (SEQ ID NO: 1005; bovine Col1A2) (www.ncbi.nlm.nih.gov/protein/27806257) and NP_001070299.1 (SEQ ID NO: 1006; bovine Col3A1) (www.ncbi.nlm.nih.gov/protein/116003881), which are incorporated herein by reference.

In some embodiments, the collagen fragment has a sequence identity of at least about 85% to the amino acid sequence set forth in SEQ ID NO: 1. In some embodiments, the collagen fragment has the amino acid sequence set forth in SEQ ID NO: 1. In some embodiments, the collagen fragment has the amino acid sequence set forth in any one of SEQ ID NOs: 2-972.

In some embodiments, the collagen fragment can be a collagen fragment sequence variant having the amino acid sequence according to any one of SEQ ID NO:s 975-1002.

In some embodiments, this disclosure provides a method of treating a cancer in a subject in need thereof, comprising contacting a cellular surface on the subject with an effective amount of a recombinant collagen fragment having a sequence identity of at least about 85% to the amino acid sequence set forth in SEQ ID NO: 1. SEQ ID NO: 1 has a molecular weight of about 50 kDa. In some embodiments, the recombinant collagen fragment can have at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity, or similarity to SEQ ID NO: 1.

The amino acid sequence of SEQ ID NO: 1 is:

DVKSGVAVGGLAGYPGPAGPPGPPGPPGTSGHPGSPGSPGYQGPPGEPGQ
AGPSGPPGPPGAIGPSGPAGKDGESGRPGRPGERGLPGPPGIKGPAGIPG
FPGMKGHRGFDGRNGEKGETGAPGLKGENGLPGENGAPGPMGPRGAPGER
GRPGLPGAAGARGNDGARGSDGQPGPPGPPGTAGFPGSPGAKGEVGPAGS
PGSNGAPGQRGEPGPQGHAGAQGPPGPPGINGSPGGKGEMGPAGIPGAPG
LMGARGPPGPAGANGAPGLRGGAGEPGKNGAKGEPGPRGERGEAGIPGVP
GAKGEDGKDGSPGEPGANGLPGAAGERGAPGFRGPAGPNGIPGEKGPAGE
RGAPGPAGPRGAAGEPGRDGVPGGPGMRGMPGSPGGPGSDGKPGPPGSQG
ESGRPGPPGPSGPRGQPGVMGFPGPKGNDGAPGKNGERGGPGGPGPQGPP
GKNGETGPQGPPGPTGPGGDKGDTGPPGPQGLQGLPGTGGPPGENGKPGE
PGPKGDAGAPGAPGGKGDAGAPGERGPPAIAGIGGEKAGGFAPYYG

In some embodiments, the lysine, proline, or lysine and proline residues present in the recombinant collagen fragment are not hydroxylated. In other embodiments, the recombinant collagen fragment described herein can be hydroxylated. In some embodiments, at least 1%, 2%, 3%, 4%, 5%, 10%, 15%, 20%, 30%, 40%, 50%, 60%, 70%, 80%, 90%, 95% or 100% (or any intermediate value or subrange) of the lysine, proline, or lysine and proline residues in the recombinant collagen fragment can be hydroxylated. Hydroxylating collagen can build rheology and improve thermal stability of a collagen molecule or fragment. Hydroxylated collagen and hydroxylated collagen fragments are also resistant to high concentration pepsin digestion, for example at a pepsin: total protein ratio of 1:25 to 1:1. In some embodiments the recombinant collagen fragment having the amino acid sequence set forth in SEQ ID NO: 1 is unhydroxylated. In some embodiments, the recombinant collagen fragment having the amino acid sequence set forth in SEQ ID NO: 1 is hydroxylated.

In some embodiments, this disclosure provides a method of treating a cancer in a subject in need thereof, the method comprising contacting a cellular surface on the subject with an effective amount of a recombinant collagen fragment having the amino acid sequence set forth in any one of SEQ ID NOs: 2-972. In some embodiments, the recombinant collagen can comprise a hydrolysis product of a collagen fragment, wherein the hydrolysis product can have a sequence that is a portion of SEQ ID NO: 1. In some embodiments, the hydrolysis product can have a sequence according to one of SEQ ID NOs: 2-972.

In some embodiments, the methods of treating a cancer in a subject in need thereof provided herein, do not comprise contacting a cellular surface on the subject with an effective amount of a recombinant collagen having the amino acid sequence set forth in SEQ ID NO: 973 in the absence of a pharmaceutically acceptable scaffold.

In some embodiments, the recombinant collagen fragment described herein can have an amino acid sequence as set forth below in Table 1.

TABLE 1

| SEQ ID | |
|---|---|
| NO. | SEQUENCE |
| 1 | DVKSGVAVGGLAGYPGPAGPPGPPGPPGTSGHPGSPGSPGYQGPPGEPGQ |
| | AGPSGPPGPPGAIGPSGPAGKDGESGRPGRPGERGLPGPPGIKGPAGIPGFP |
| | GMKGHRGFDGRNGEKGETGAPGLKGENGLPGENGAPGPMGPRGAPGER |
| | GRPGLPGAAGARGNDGARGSDGQPGPPGPPGTAGFPGSPGAKGEVGPAG |
| | SPGSNGAPGQRGEPGPQGHAGAQGPPGPPGINGSPGGKGEMGPAGIPGAP |
| | GLMGARGPPGPAGANGAPGLRGGAGEPGKNGAKGEPGPRGERGEAGIPG |
| | VPGAKGEDGKDGSPGEPGANGLPGAAGERGAPGFRGPAGPNGIPGEKGP |
| | AGERGAPGPAGPRGAAGEPGRDGVPGGPGMRGMPGSPGGPGSDGKPGPP |
| | GSQGESGRPGPPGPSGPRGQPGVMGFPGPKGNDGAPGKNGERGGPGGPG |
| | PQGPPGKNGETGPQGPPGPTGPGGDKGDTGPPGPQGLQGLPGTGGPPGEN |
| | GKPGEPGPKGDAGAPGAPGGKGDAGAPGERGPPAIAGIGGEKAGGFAPY |
| | YG |
| 2 | AAGARGNDGARGSDGQPGPPGPPGTA |
| 3 | AAGARGNDGARGSDGQPGPPGPPGTAGFPGSPGAK |
| 4 | AAGARGNDGARGSDGQPGPPGPPGTAGFPGSPGAKGEVGPA |
| 5 | AAGEPGRDGVPGGPGMRGMPG |
| 6 | AAGEPGRDGVPGGPGMRGMPGSPGGPG |
| 7 | AAGERGAPGFRGPAGPN |
| 8 | AGAQGPPGPPGIN |
| 9 | AGAQGPPGPPGINGSPGGKGEMGPAGIPGAPGLM |
| 10 | AGARGNDGARGSDGQPGPPGPPGTA |
| 11 | AGARGNDGARGSDGQPGPPGPPGTAGFPGSPGAKGEVGPA |
| 12 | AGEPGRDGVPGGPGMRGMPGSPGGPG |
| 13 | AGERGAPGFRGPAGPN |
| 14 | AGIPGAPGLM |
| 15 | AGIPGAPGLMGARGPPGPAGANGAPGLR |
| 16 | AGIPGFPGMKGH |
| 17 | AGIPGFPGMKGHRG |
| 18 | AGIPGFPGMKGHRGF |
| 19 | AGIPGFPGMKGHRGFDGRN |
| 20 | AGIPGFPGMKGHRGFDGRNGEKGETGAPGLK |
| 21 | AGIPGFPGMKGHRGFDGRNGEKGETGAPGLKGEN |
| 22 | AGKDGESGRPGRPGERGLPGPPGIK |
| 23 | AGKDGESGRPGRPGERGLPGPPGIKGPA |
| 24 | AGPPGPPGPPGTS |
| 25 | AGPPGPPGPPGTSGH |
| 26 | AGPSGPPGPPGAIGPS |
| 27 | AGPSGPPGPPGAIGPSGPAGKDGESGRPGRPGERGLPGPPGIK |
| 28 | AGSPGSNGAPGQRGEPGPQGHAGAQGPPGPPGINGSPGGKGEMGPAGIPG |
| | APGLM |
| 29 | AGYPGPAGPPGPPGPPGTS |
| 30 | AGYPGPAGPPGPPGPPGTSGHPGSPGSPGYQGPPGEPGQAGPSGPPGPPGAI |
| | GPS |
| 31 | AIGPSGPAGKDGESGRPGRPGERGLPGPPGIK |
| 32 | AKGEVGPAGSPGSNGAPGQRGEPGPQG |
| 33 | AKGEVGPAGSPGSNGAPGQRGEPGPQGHAGAQGPPGPPGIN |
| 34 | ANGAPGLRGGAGEPGKNGAKGEPGPR |
| 35 | ANGLPGAAGERGAPGFRGPAGPN |
| 36 | APGERGRPGLPGAAGAR |
| 37 | APGERGRPGLPGAAGARGNDGARGSDGQPGPPGPPGTA |
| 38 | APGLKGENGLPGEN |
| 39 | APGLKGENGLPGENGAPGPMGPR |
| 40 | APGLMGARGPPGPA |
| 41 | APGLMGARGPPGPAGAN |
| 42 | APGLMGARGPPGPAGANGAPGLR |
| 43 | APGQRGEPGPQGHAGAQGPPGPPGIN |
| 44 | AQGPPGPPGIN |
| 45 | AQGPPGPPGINGSPG |
| 46 | AQGPPGPPGINGSPGGK |
| 47 | AQGPPGPPGINGSPGGKGEMGPA |
| 48 | AQGPPGPPGINGSPGGKGEMGPAGIPGAPGLMGAR |
| 49 | ARGNDGARGSDGQPGPPGPPGTA |
| 50 | ARGNDGARGSDGQPGPPGPPGTAGF |
| 51 | ARGNDGARGSDGQPGPPGPPGTAGFPGSPGAKGEVGPA |
| 52 | ARGNDGARGSDGQPGPPGPPGTAGFPGSPGAKGEVGPAGSPGSN |
| 53 | ARGPPGPAGANGAPGLR |
| 54 | ARGPPGPAGANGAPGLRGGAGEPGKN |
| 55 | ARGSDGQPGPPGPPGTA |
| 56 | ARGSDGQPGPPGPPGTAGFPGSPGAK |
| 57 | ARGSDGQPGPPGPPGTAGFPGSPGAKGEVGPA |
| 58 | DAGAPGAPGGKGDAGAPGERGPP |
| 59 | DGAPGKNGERGGPGGPGPQGPPG |
| 60 | DGAPGKNGERGGPGGPGPQGPPGKN |
| 61 | DGAPGKNGERGGPGGPGPQGPPGKNGE |
| 62 | DGAPGKNGERGGPGGPGPQGPPGKNGETG |
| 63 | DGAPGKNGERGGPGGPGPQGPPGKNGETGP |
| 64 | DGAPGKNGERGGPGGPGPQGPPGKNGETGPQ |
| 65 | DGAPGKNGERGGPGGPGPQGPPGKNGETGPQG |
| 66 | DGAPGKNGERGGPGGPGPQGPPGKNGETGPQGPPG |
| 67 | DGAPGKNGERGGPGGPGPQGPPGKNGETGPQGPPGP |
| 68 | DGAPGKNGERGGPGGPGPQGPPGKNGETGPQGPPGPTG |
| 69 | DGAPGKNGERGGPGGPGPQGPPGKNGETGPQGPPGPTGPG |
| 70 | DGAPGKNGERGGPGGPGPQGPPGKNGETGPQGPPGPTGPGG |
| 71 | DGARGSDGQPGPPGPPG |
| 72 | DGARGSDGQPGPPGPPGTA |
| 73 | DGARGSDGQPGPPGPPGTAG |
| 74 | DGARGSDGQPGPPGPPGTAGF |
| 75 | DGARGSDGQPGPPGPPGTAGFPG |
| 76 | DGARGSDGQPGPPGPPGTAGFPGSPGAK |
| 77 | DGARGSDGQPGPPGPPGTAGFPGSPGAKG |
| 78 | DGARGSDGQPGPPGPPGTAGFPGSPGAKGEVGPA |
| 79 | DGARGSDGQPGPPGPPGTAGFPGSPGAKGEVGPAG |
| 80 | DGARGSDGQPGPPGPPGTAGFPGSPGAKGEVGPAGSPG |
| 81 | DGARGSDGQPGPPGPPGTAGFPGSPGAKGEVGPAGSPGSN |
| 82 | DGARGSDGQPGPPGPPGTAGFPGSPGAKGEVGPAGSPGSNGAPGQRGEPG |
| | PQ |
| 83 | DGESGRPGRPGERGLPGPPGIK |
| 84 | DGESGRPGRPGERGLPGPPGIKGPA |
| 85 | DGKPGPPGSQGESGRPGPPGPSGPRGQPGVM |
| 86 | DGQPGPPGPPGTA |
| 87 | DGQPGPPGPPGTAGFPGSPGAKGEVGPA |
| 88 | DGRNGEKGETGAPGLK |
| 89 | DGRNGEKGETGAPGLKGENGLPGEN |
| 90 | DGRNGEKGETGAPGLKGENGLPGENGAPGPMGPR |
| 91 | DGRNGEKGETGAPGLKGENGLPGENGAPGPMGPRGAPGERGRPGLPGAA |
| 92 | DGRNGEKGETGAPGLKGENGLPGENGAPGPMGPRGAPGERGRPGLPGAA |
| | GAR |
| 93 | EKGETGAPGLKGENGLPGENGAPGPMGPR |
| 94 | EMGPAGIPGAPGLMGAR |
| 95 | ENGLPGENGAPGPMGPR |
| 96 | ENGLPGENGAPGPMGPRGAPGERGRPGLPGAAGAR |
| 97 | EPGQAGPSGPPGPPGAIGPS |
| 98 | ERGLPGPPGIKGPA |
| 99 | ESGRPGPPGPSGPRGQPGVM |
| 100 | ESGRPGPPGPSGPRGQPGVMGFPGPKGN |
| 101 | ESGRPGRPGERGLPGPPGIK |
| 102 | ETGAPGLKGENGLPGEN |
| 103 | ETGAPGLKGENGLPGENGAPGPMGPR |
| 104 | ETGPQGPPGPTGPGGD |
| 105 | EVGPAGSPGSNGAPGQRGEPGPQ |
| 106 | EVGPAGSPGSNGAPGQRGEPGPQGH |
| 107 | EVGPAGSPGSNGAPGQRGEPGPQGHAG |
| 108 | EVGPAGSPGSNGAPGQRGEPGPQGHAGAQ |
| 109 | EVGPAGSPGSNGAPGQRGEPGPQGHAGAQGPPGPPGIN |
| 110 | EVGPAGSPGSNGAPGQRGEPGPQGHAGAQGPPGPPGINGSPGGK |
| 111 | FDGRNGEKGETGAPGLKGEN |
| 112 | FDGRNGEKGETGAPGLKGENGLPGEN |
| 113 | FDGRNGEKGETGAPGLKGENGLPGENGAPGPMGPR |
| 114 | FPGSPGAKGEVGPA |
| 115 | FPGSPGAKGEVGPAGSPGSN |
| 116 | GAAGARGNDGARGSDGQPGPPGPP |
| 117 | GAAGARGNDGARGSDGQPGPPGPPGTA |
| 118 | GAAGARGNDGARGSDGQPGPPGPPGTAGFPGSPGAK |
| 119 | GAAGARGNDGARGSDGQPGPPGPPGTAGFPGSPGAKGEVGPA |
| 120 | GAAGEPGRDGVPGGPGMRGMPG |
| 121 | GAAGEPGRDGVPGGPGMRGMPGSPGGPG |
| 122 | GAAGERGAPGFRGPAGPN |
| 123 | GAIGPSGPAGKDGESGRPGRPGER |
| 124 | GAIGPSGPAGKDGESGRPGRPGERGLPGPP |
| 125 | GAIGPSGPAGKDGESGRPGRPGERGLPGPPGIK |
| 126 | GAKGEPGPRGERGEAGIPGVPG |
| 127 | GAKGEPGPRGERGEAGIPGVPGAK |
| 128 | GAKGEPGPRGERGEAGIPGVPGAKG |
| 129 | GAKGEPGPRGERGEAGIPGVPGAKGEDGK |
| 130 | GAKGEPGPRGERGEAGIPGVPGAKGEDGKDG |
| 131 | GAKGEPGPRGERGEAGIPGVPGAKGEDGKDGS |
| 132 | GAKGEPGPRGERGEAGIPGVPGAKGEDGKDGSPGEPGAN |
| 133 | GAKGEVGPAGSPGSNGAPGQRGEPGPQ |
| 134 | GAKGEVGPAGSPGSNGAPGQRGEPGPQG |
| 135 | GAKGEVGPAGSPGSNGAPGQRGEPGPQGH |
| 136 | GAKGEVGPAGSPGSNGAPGQRGEPGPQGHAGAQ |
| 137 | GAKGEVGPAGSPGSNGAPGQRGEPGPQGHAGAQGPPGPPGIN |
| 138 | GANGAPGLR |
| 139 | GANGAPGLRGGAGEPGKN |
| 140 | GANGAPGLRGGAGEPGKNGAK |
| 141 | GANGAPGLRGGAGEPGKNGAKGEPGPR |
| 142 | GANGAPGLRGGAGEPGKNGAKGEPGPRG |
| 143 | GANGAPGLRGGAGEPGKNGAKGEPGPRGER |
| 144 | GANGAPGLRGGAGEPGKNGAKGEPGPRGERG |
| 145 | GANGAPGLRGGAGEPGKNGAKGEPGPRGERGEAG |
| 146 | GAPGERGRPGLPGAA |
| 147 | GAPGERGRPGLPGAAG |
| 148 | GAPGERGRPGLPGAAGA |
| 149 | GAPGERGRPGLPGAAGAR |
| 150 | GAPGERGRPGLPGAAGARGN |
| 151 | GAPGERGRPGLPGAAGARGND |
| 152 | GAPGERGRPGLPGAAGARGNDG |
| 153 | GAPGERGRPGLPGAAGARGNDGA |
| 154 | GAPGERGRPGLPGAAGARGNDGAR |
| 155 | GAPGERGRPGLPGAAGARGNDGARG |
| 156 | GAPGERGRPGLPGAAGARGNDGARGS |
| 157 | GAPGERGRPGLPGAAGARGNDGARGSD |
| 158 | GAPGERGRPGLPGAAGARGNDGARGSDG |
| 159 | GAPGERGRPGLPGAAGARGNDGARGSDGQ |
| 160 | GAPGERGRPGLPGAAGARGNDGARGSDGQPG |
| 161 | GAPGERGRPGLPGAAGARGNDGARGSDGQPGP |
| 162 | GAPGERGRPGLPGAAGARGNDGARGSDGQPGPPG |
| 163 | GAPGERGRPGLPGAAGARGNDGARGSDGQPGPPGP |
| 164 | GAPGERGRPGLPGAAGARGNDGARGSDGQPGPPGPPG |
| 165 | GAPGERGRPGLPGAAGARGNDGARGSDGQPGPPGPPGTA |
| 166 | GAPGERGRPGLPGAAGARGNDGARGSDGQPGPPGPPGTAGFPGSPGAK |
| 167 | GAPGERGRPGLPGAAGARGNDGARGSDGQPGPPGPPGTAGFPGSPGAKG |
| | EVG |
| 168 | GAPGERGRPGLPGAAGARGNDGARGSDGQPGPPGPPGTAGFPGSPGAKG |
| | EVGPA |
| 169 | GAPGERGRPGLPGAAGARGNDGARGSDGQPGPPGPPGTAGFPGSPGAKG |
| | EVGPAG |
| 170 | GAPGLKGENGLPGENGAPGPMGPR |
| 171 | GAPGLMGARGPPGPA |
| 172 | GAPGLMGARGPPGPAGAN |
| 173 | GAPGLMGARGPPGPAGANGAP |
| 174 | GAPGLMGARGPPGPAGANGAPGLR |
| 175 | GAPGLMGARGPPGPAGANGAPGLRGGAGEPGKN |
| 176 | GAPGLRGGAGEPGKN |
| 177 | GAPGLRGGAGEPGKNGAKGEPGPRGERGEA |
| 178 | GAPGPAGPRGAAGEPGRDGVPG |
| 179 | GAPGPAGPRGAAGEPGRDGVPGGPGMRGMPG |
| 180 | GAPGPMGPRGAPGERGRPGLP |
| 181 | GAPGPMGPRGAPGERGRPGLPG |
| 182 | GAPGPMGPRGAPGERGRPGLPGAA |
| 183 | GAPGPMGPRGAPGERGRPGLPGAAG |
| 184 | GAPGPMGPRGAPGERGRPGLPGAAGA |
| 185 | GAPGPMGPRGAPGERGRPGLPGAAGAR |
| 186 | GAPGPMGPRGAPGERGRPGLPGAAGARGN |
| 187 | GAPGPMGPRGAPGERGRPGLPGAAGARGNDGAR |
| 188 | GAPGPMGPRGAPGERGRPGLPGAAGARGNDGARG |
| 189 | GAPGPMGPRGAPGERGRPGLPGAAGARGNDGARGSDGQPGPPGPPGTA |
| 190 | GAPGQRGEPGPQGHAGAQGPPGPP |
| 191 | GAPGQRGEPGPQGHAGAQGPPGPPG |
| 192 | GAPGQRGEPGPQGHAGAQGPPGPPGIN |
| 193 | GAPGQRGEPGPQGHAGAQGPPGPPGING |
| 194 | GAPGQRGEPGPQGHAGAQGPPGPPGINGS |
| 195 | GAPGQRGEPGPQGHAGAQGPPGPPGINGSPG |
| 196 | GAPGQRGEPGPQGHAGAQGPPGPPGINGSPGG |
| 197 | GAPGQRGEPGPQGHAGAQGPPGPPGINGSPGGK |
| 198 | GAPGQRGEPGPQGHAGAQGPPGPPGINGSPGGKGEM |
| 199 | GAPGQRGEPGPQGHAGAQGPPGPPGINGSPGGKGEMG |
| 200 | GAPGQRGEPGPQGHAGAQGPPGPPGINGSPGGKGEMGPA |
| 201 | GAPGQRGEPGPQGHAGAQGPPGPPGINGSPGGKGEMGPAGIPGAPGLM |
| 202 | GAPGQRGEPGPQGHAGAQGPPGPPGINGSPGGKGEMGPAGIPGAPGLMG |
| 203 | GAPGQRGEPGPQGHAGAQGPPGPPGINGSPGGKGEMGPAGIPGAPGLMG |
| | A |
| 204 | GAPGQRGEPGPQGHAGAQGPPGPPGINGSPGGKGEMGPAGIPGAPGLMG |
| | AR |
| 205 | GAQGPPGPPGIN |
| 206 | GARGNDGARGSDGQPGPPGPP |
| 207 | GARGNDGARGSDGQPGPPGPPGTA |
| 208 | GARGNDGARGSDGQPGPPGPPGTAGFPG |
| 209 | GARGNDGARGSDGQPGPPGPPGTAGFPGSPGA |
| 210 | GARGNDGARGSDGQPGPPGPPGTAGFPGSPGAK |
| 211 | GARGNDGARGSDGQPGPPGPPGTAGFPGSPGAKGEVGPA |
| 212 | GARGNDGARGSDGQPGPPGPPGTAGFPGSPGAKGEVGPAG |
| 213 | GARGNDGARGSDGQPGPPGPPGTAGFPGSPGAKGEVGPAGSPG |
| 214 | GARGNDGARGSDGQPGPPGPPGTAGFPGSPGAKGEVGPAGSPGSN |
| 215 | GARGPPGPAGAN |
| 216 | GARGPPGPAGANGAPGL |
| 217 | GARGPPGPAGANGAPGLR |
| 218 | GARGPPGPAGANGAPGLRG |
| 219 | GARGPPGPAGANGAPGLRGG |
| 220 | GARGPPGPAGANGAPGLRGGA |
| 221 | GARGPPGPAGANGAPGLRGGAG |
| 222 | GARGPPGPAGANGAPGLRGGAGE |
| 223 | GARGPPGPAGANGAPGLRGGAGEPG |
| 224 | GARGPPGPAGANGAPGLRGGAGEPGKN |
| 225 | GARGPPGPAGANGAPGLRGGAGEPGKNG |
| 226 | GARGPPGPAGANGAPGLRGGAGEPGKNGA |
| 227 | GARGPPGPAGANGAPGLRGGAGEPGKNGAK |
| 228 | GARGPPGPAGANGAPGLRGGAGEPGKNGAKGE |
| 229 | GARGPPGPAGANGAPGLRGGAGEPGKNGAKGEPG |
| 230 | GARGPPGPAGANGAPGLRGGAGEPGKNGAKGEPGPR |
| 231 | GARGPPGPAGANGAPGLRGGAGEPGKNGAKGEPGPRGER |
| 232 | GARGSDGQPGPPGPPGTA |
| 233 | GARGSDGQPGPPGPPGTAGFPGSPGAK |
| 234 | GARGSDGQPGPPGPPGTAGFPGSPGAKGEVGPA |
| 235 | GDKGDTGPPGPQGLQGLPGTGGPPGEN |
| 236 | GEKGETGAPGLK |
| 237 | GEKGETGAPGLKGENGLPGEN |
| 238 | GEKGETGAPGLKGENGLPGENGAPGPM |
| 239 | GEKGETGAPGLKGENGLPGENGAPGPMGPR |
| 240 | GEKGETGAPGLKGENGLPGENGAPGPMGPRGAPGERGRPGLPGAA |
| 241 | GEKGETGAPGLKGENGLPGENGAPGPMGPRGAPGERGRPGLPGAAGAR |
| 242 | GEMGPAGIPGAPGLM |
| 243 | GEMGPAGIPGAPGLMGAR |
| 244 | GEMGPAGIPGAPGLMGARGPPGPA |
| 245 | GEMGPAGIPGAPGLMGARGPPGPAGAN |
| 246 | GEMGPAGIPGAPGLMGARGPPGPAGANGAPGLR |
| 247 | GEMGPAGIPGAPGLMGARGPPGPAGANGAPGLRGGAGEPGKN |
| 248 | GENGLPGENGAPGPM |
| 249 | GENGLPGENGAPGPMGPR |
| 250 | GENGLPGENGAPGPMGPRG |
| 251 | GENGLPGENGAPGPMGPRGAPG |
| 252 | GENGLPGENGAPGPMGPRGAPGER |
| 253 | GENGLPGENGAPGPMGPRGAPGERG |
| 254 | GENGLPGENGAPGPMGPRGAPGERGR |
| 255 | GENGLPGENGAPGPMGPRGAPGERGRPGLPGAA |
| 256 | GENGLPGENGAPGPMGPRGAPGERGRPGLPGAAGA |
| 257 | GENGLPGENGAPGPMGPRGAPGERGRPGLPGAAGAR |
| 258 | GENGLPGENGAPGPMGPRGAPGERGRPGLPGAAGARGN |
| 259 | GEPGANGLPGAAGERGAPGFRGPAGPN |
| 260 | GEPGPKGDAGAPGAPGGKGDAGAPG |
| 261 | GEPGPKGDAGAPGAPGGKGDAGAPGE |
| 262 | GEPGPQGHAGAQGPPGPPGIN |
| 263 | GEPGPQGHAGAQGPPGPPGINGSPGGKGEMGPA |
| 264 | GEPGPQGHAGAQGPPGPPGINGSPGGKGEMGPAGIPGAPGLM |
| 265 | GEPGPRGERGEAGIPGVPGAKGEDGKDG |
| 266 | GEPGQAGPSGPPGPPGAIGPS |
| 267 | GEPGQAGPSGPPGPPGAIGPSGPAGKD |
| 268 | GEPGRDGVPGGPGMRGMPGSPGGPG |
| 269 | GERGEAGIPGVPGAKG |
| 270 | GERGEAGIPGVPGAKGEDGKDG |
| 271 | GERGEAGIPGVPGAKGEDGKDGSPGEPGAN |
| 272 | GERGGPGGPGPQGPPGK |
| 273 | GERGGPGGPGPQGPPGKN |
| 274 | GERGGPGGPGPQGPPGKNG |
| 275 | GERGGPGGPGPQGPPGKNGETGP |
| 276 | GERGGPGGPGPQGPPGKNGETGPQ |
| 277 | GERGGPGGPGPQGPPGKNGETGPQG |
| 278 | GERGGPGGPGPQGPPGKNGETGPQGPP |
| 279 | GERGGPGGPGPQGPPGKNGETGPQGPPG |
| 280 | GERGGPGGPGPQGPPGKNGETGPQGPPGP |
| 281 | GERGGPGGPGPQGPPGKNGETGPQGPPGPT |
| 282 | GERGGPGGPGPQGPPGKNGETGPQGPPGPTG |
| 283 | GERGGPGGPGPQGPPGKNGETGPQGPPGPTGPG |
| 284 | GERGGPGGPGPQGPPGKNGETGPQGPPGPTGPGG |
| 285 | GERGGPGGPGPQGPPGKNGETGPQGPPGPTGPGGD |
| 286 | GERGGPGGPGPQGPPGKNGETGPQGPPGPTGPGGDK |
| 287 | GERGGPGGPGPQGPPGKNGETGPQGPPGPTGPGGDKG |
| 288 | GERGGPGGPGPQGPPGKNGETGPQGPPGPTGPGGDKGD |
| 289 | GERGGPGGPGPQGPPGKNGETGPQGPPGPTGPGGDKGDTG |
| 290 | GERGGPGGPGPQGPPGKNGETGPQGPPGPTGPGGDKGDTGPPG |
| 291 | GERGGPGGPGPQGPPGKNGETGPQGPPGPTGPGGDKGDTGPPGPQ |
| 292 | GERGGPGGPGPQGPPGKNGETGPQGPPGPTGPGGDKGDTGPPGPQG |
| 293 | GERGGPGGPGPQGPPGKNGETGPQGPPGPTGPGGDKGDTGPPGPQGLQ |
| 294 | GERGGPGGPGPQGPPGKNGETGPQGPPGPTGPGGDKGDTGPPGPQGLQG |
| 295 | GERGLPGPPGIK |
| 296 | GERGLPGPPGIKGPA |
| 297 | GERGLPGPPGIKGPAGIPGFPGMK |
| 298 | GESGRPGPPGPSGPRGQPGVM |
| 299 | GESGRPGPPGPSGPRGQPGVMGFPGPKGN |
| 300 | GESGRPGRPGERGLPGPPGIK |
| 301 | GESGRPGRPGERGLPGPPGIKG |
| 302 | GESGRPGRPGERGLPGPPGIKGPA |
| 303 | GESGRPGRPGERGLPGPPGIKGPAGIPGFPGMK |
| 304 | GETGAPGLKGENGLPGEN |
| 305 | GETGAPGLKGENGLPGENGAPGPMGPR |
| 306 | GETGAPGLKGENGLPGENGAPGPMGPRGAPGERGRPGLPGAAGAR |
| 307 | GETGAPGLKGENGLPGENGAPGPMGPRGAPGERGRPGLPGAAGARGN |
| 308 | GETGPQGPPGPTGPG |
| 309 | GETGPQGPPGPTGPGGD |
| 310 | GETGPQGPPGPTGPGGDKGDTG |
| 311 | GETGPQGPPGPTGPGGDKGDTGPPG |
| 312 | GETGPQGPPGPTGPGGDKGDTGPPGPQ |
| 313 | GETGPQGPPGPTGPGGDKGDTGPPGPQG |
| 314 | GEVGPAGSPGSNGAPG |
| 315 | GEVGPAGSPGSNGAPGQ |
| 316 | GEVGPAGSPGSNGAPGQR |
| 317 | GEVGPAGSPGSNGAPGQRGE |
| 318 | GEVGPAGSPGSNGAPGQRGEP |
| 319 | GEVGPAGSPGSNGAPGQRGEPG |
| 320 | GEVGPAGSPGSNGAPGQRGEPGP |
| 321 | GEVGPAGSPGSNGAPGQRGEPGPQ |
| 322 | GEVGPAGSPGSNGAPGQRGEPGPQG |
| 323 | GEVGPAGSPGSNGAPGQRGEPGPQGH |
| 324 | GEVGPAGSPGSNGAPGQRGEPGPQGHA |
| 325 | GEVGPAGSPGSNGAPGQRGEPGPQGHAG |
| 326 | GEVGPAGSPGSNGAPGQRGEPGPQGHAGA |
| 327 | GEVGPAGSPGSNGAPGQRGEPGPQGHAGAQ |
| 328 | GEVGPAGSPGSNGAPGQRGEPGPQGHAGAQG |
| 329 | GEVGPAGSPGSNGAPGQRGEPGPQGHAGAQGP |
| 330 | GEVGPAGSPGSNGAPGQRGEPGPQGHAGAQGPP |
| 331 | GEVGPAGSPGSNGAPGQRGEPGPQGHAGAQGPPG |
| 332 | GEVGPAGSPGSNGAPGQRGEPGPQGHAGAQGPPGP |
| 333 | GEVGPAGSPGSNGAPGQRGEPGPQGHAGAQGPPGPP |
| 334 | GEVGPAGSPGSNGAPGQRGEPGPQGHAGAQGPPGPPG |
| 335 | GEVGPAGSPGSNGAPGQRGEPGPQGHAGAQGPPGPPGIN |
| 336 | GEVGPAGSPGSNGAPGQRGEPGPQGHAGAQGPPGPPGING |
| 337 | GEVGPAGSPGSNGAPGQRGEPGPQGHAGAQGPPGPPGINGS |
| 338 | GEVGPAGSPGSNGAPGQRGEPGPQGHAGAQGPPGPPGINGSPG |
| 339 | GEVGPAGSPGSNGAPGQRGEPGPQGHAGAQGPPGPPGINGSPGG |
| 340 | GEVGPAGSPGSNGAPGQRGEPGPQGHAGAQGPPGPPGINGSPGGK |
| 341 | GEVGPAGSPGSNGAPGQRGEPGPQGHAGAQGPPGPPGINGSPGGKG |
| 342 | GEVGPAGSPGSNGAPGQRGEPGPQGHAGAQGPPGPPGINGSPGGKGEM |
| 343 | GEVGPAGSPGSNGAPGQRGEPGPQGHAGAQGPPGPPGINGSPGGKGEMG |
| 344 | GEVGPAGSPGSNGAPGQRGEPGPQGHAGAQGPPGPPGINGSPGGKGEMGP |
| | A |
| 345 | GEVGPAGSPGSNGAPGQRGEPGPQGHAGAQGPPGPPGINGSPGGKGEMGP |
| | AG |
| 346 | GFDGRNGEKGETGAPGLK |
| 347 | GFDGRNGEKGETGAPGLKG |
| 348 | GFDGRNGEKGETGAPGLKGEN |
| 349 | GFDGRNGEKGETGAPGLKGENG |
| 350 | GFDGRNGEKGETGAPGLKGENGLPGEN |
| 351 | GFDGRNGEKGETGAPGLKGENGLPGENGAPGPMGPR |
| 352 | GFDGRNGEKGETGAPGLKGENGLPGENGAPGPMGPRGAPGERGRPGLPG |
| | AA |
| 353 | GFPGMKGHRGFDGRNGEKGETGAPGLK |
| 354 | GFPGMKGHRGFDGRNGEKGETGAPGLKGEN |
| 355 | GFPGPKGNDGAPGKN |
| 356 | GFPGPKGNDGAPGKNGER |
| 357 | GFPGPKGNDGAPGKNGERG |
| 358 | GFPGPKGNDGAPGKNGERGG |
| 359 | GFPGPKGNDGAPGKNGERGGP |
| 360 | GFPGPKGNDGAPGKNGERGGPG |
| 361 | GFPGPKGNDGAPGKNGERGGPGG |
| 362 | GFPGPKGNDGAPGKNGERGGPGGP |
| 363 | GFPGPKGNDGAPGKNGERGGPGGPG |
| 364 | GFPGPKGNDGAPGKNGERGGPGGPGP |
| 365 | GFPGPKGNDGAPGKNGERGGPGGPGPQ |
| 366 | GFPGPKGNDGAPGKNGERGGPGGPGPQG |
| 367 | GFPGPKGNDGAPGKNGERGGPGGPGPQGP |
| 368 | GFPGPKGNDGAPGKNGERGGPGGPGPQGPP |
| 369 | GFPGPKGNDGAPGKNGERGGPGGPGPQGPPG |
| 370 | GFPGPKGNDGAPGKNGERGGPGGPGPQGPPGK |
| 371 | GFPGPKGNDGAPGKNGERGGPGGPGPQGPPGKN |
| 372 | GFPGPKGNDGAPGKNGERGGPGGPGPQGPPGKNG |
| 373 | GFPGPKGNDGAPGKNGERGGPGGPGPQGPPGKNGE |
| 374 | GFPGPKGNDGAPGKNGERGGPGGPGPQGPPGKNGET |
| 375 | GFPGPKGNDGAPGKNGERGGPGGPGPQGPPGKNGETG |
| 376 | GFPGPKGNDGAPGKNGERGGPGGPGPQGPPGKNGETGP |
| 377 | GFPGPKGNDGAPGKNGERGGPGGPGPQGPPGKNGETGPQ |
| 378 | GFPGPKGNDGAPGKNGERGGPGGPGPQGPPGKNGETGPQG |
| 379 | GFPGPKGNDGAPGKNGERGGPGGPGPQGPPGKNGETGPQGPPG |
| 380 | GFPGPKGNDGAPGKNGERGGPGGPGPQGPPGKNGETGPQGPPGPTG |
| 381 | GFPGPKGNDGAPGKNGERGGPGGPGPQGPPGKNGETGPQGPPGPTGPGG |
| | D |
| 382 | GFPGPKGNDGAPGKNGERGGPGGPGPQGPPGKNGETGPQGPPGPTGPGG |
| | DK |
| 383 | GFPGSPGAKGEVG |
| 384 | GFPGSPGAKGEVGP |
| 385 | GFPGSPGAKGEVGPA |
| 386 | GFPGSPGAKGEVGPAG |
| 387 | GFPGSPGAKGEVGPAGS |
| 388 | GFPGSPGAKGEVGPAGSPG |
| 389 | GFPGSPGAKGEVGPAGSPGSN |
| 390 | GFPGSPGAKGEVGPAGSPGSNG |
| 391 | GFPGSPGAKGEVGPAGSPGSNGA |
| 392 | GFPGSPGAKGEVGPAGSPGSNGAPG |
| 393 | GFPGSPGAKGEVGPAGSPGSNGAPGQ |
| 394 | GFPGSPGAKGEVGPAGSPGSNGAPGQR |
| 395 | GFPGSPGAKGEVGPAGSPGSNGAPGQRG |
| 396 | GFPGSPGAKGEVGPAGSPGSNGAPGQRGE |
| 397 | GFPGSPGAKGEVGPAGSPGSNGAPGQRGEP |
| 398 | GFPGSPGAKGEVGPAGSPGSNGAPGQRGEPG |
| 399 | GFPGSPGAKGEVGPAGSPGSNGAPGQRGEPGPQ |
| 400 | GFPGSPGAKGEVGPAGSPGSNGAPGQRGEPGPQG |
| 401 | GFPGSPGAKGEVGPAGSPGSNGAPGQRGEPGPQGH |
| 402 | GFPGSPGAKGEVGPAGSPGSNGAPGQRGEPGPQGHA |
| 403 | GFPGSPGAKGEVGPAGSPGSNGAPGQRGEPGPQGHAG |
| 404 | GFPGSPGAKGEVGPAGSPGSNGAPGQRGEPGPQGHAGA |
| 405 | GFPGSPGAKGEVGPAGSPGSNGAPGQRGEPGPQGHAGAQ |
| 406 | GFPGSPGAKGEVGPAGSPGSNGAPGQRGEPGPQGHAGAQG |
| 407 | GFPGSPGAKGEVGPAGSPGSNGAPGQRGEPGPQGHAGAQGPP |
| 408 | GFPGSPGAKGEVGPAGSPGSNGAPGQRGEPGPQGHAGAQGPPG |
| 409 | GFPGSPGAKGEVGPAGSPGSNGAPGQRGEPGPQGHAGAQGPPGPPG |
| 410 | GFPGSPGAKGEVGPAGSPGSNGAPGQRGEPGPQGHAGAQGPPGPPGIN |
| 411 | GFPGSPGAKGEVGPAGSPGSNGAPGQRGEPGPQGHAGAQGPPGPPGING |
| 412 | GFPGSPGAKGEVGPAGSPGSNGAPGQRGEPGPQGHAGAQGPPGPPGINGS |
| | PG |
| 413 | GFPGSPGAKGEVGPAGSPGSNGAPGQRGEPGPQGHAGAQGPPGPPGINGS |
| | PGGK |
| 414 | GFRGPAGPNGIPGEKGPAGERGAPGPA |
| 415 | GGAGEPGKNGAKGEPGPR |
| 416 | GGAGEPGKNGAKGEPGPRGERGEAGIP |
| 417 | GGAGEPGKNGAKGEPGPRGERGEAGIPG |
| 418 | GGAGEPGKNGAKGEPGPRGERGEAGIPGVPGAKGEDGKDG |
| 419 | GGAGEPGKNGAKGEPGPRGERGEAGIPGVPGAKGEDGKDGSPGEPGAN |
| 420 | GGKGEMGPAGIPGAPGLM |
| 421 | GGKGEMGPAGIPGAPGLMGA |
| 422 | GGKGEMGPAGIPGAPGLMGAR |
| 423 | GGPGGPGPQGPPGKNGETGPQGPPGPTGPGGD |
| 424 | GGPGMRGMPGSPGGPGSDGKPGPPGSQGESGRPGPPGPSGPRGQPGVM |
| 425 | GGPGSDGKPGPPGSQGESGRPGPPG |
| 426 | GGPGSDGKPGPPGSQGESGRPGPPGPS |
| 427 | GGPGSDGKPGPPGSQGESGRPGPPGPSGPR |
| 428 | GGPGSDGKPGPPGSQGESGRPGPPGPSGPRGQ |
| 429 | GGPGSDGKPGPPGSQGESGRPGPPGPSGPRGQPGVM |
| 430 | GHAGAQGPPGPPGIN |
| 431 | GHAGAQGPPGPPGING |
| 432 | GHAGAQGPPGPPGINGSPG |
| 433 | GHAGAQGPPGPPGINGSPGGK |
| 434 | GHAGAQGPPGPPGINGSPGGKGEMG |
| 435 | GHAGAQGPPGPPGINGSPGGKGEMGPA |
| 436 | GHAGAQGPPGPPGINGSPGGKGEMGPAGIPGAPGLM |
| 437 | GHAGAQGPPGPPGINGSPGGKGEMGPAGIPGAPGLMG |
| 438 | GHAGAQGPPGPPGINGSPGGKGEMGPAGIPGAPGLMGA |
| 439 | GHAGAQGPPGPPGINGSPGGKGEMGPAGIPGAPGLMGAR |
| 440 | GHAGAQGPPGPPGINGSPGGKGEMGPAGIPGAPGLMGARGPPGPA |
| 441 | GHAGAQGPPGPPGINGSPGGKGEMGPAGIPGAPGLMGARGPPGPAGAN |
| 442 | GHAGAQGPPGPPGINGSPGGKGEMGPAGIPGAPGLMGARGPPGPAGANG |
| | APGLR |
| 443 | GHPGSPGSPGYQGPPGEPG |
| 444 | GHPGSPGSPGYQGPPGEPGQA |
| 445 | GHPGSPGSPGYQGPPGEPGQAG |
| 446 | GHPGSPGSPGYQGPPGEPGQAGPS |
| 447 | GHPGSPGSPGYQGPPGEPGQAGPSGPPGPPGAIGPS |
| 448 | GHPGSPGSPGYQGPPGEPGQAGPSGPPGPPGAIGPSGPA |
| 449 | GHPGSPGSPGYQGPPGEPGQAGPSGPPGPPGAIGPSGPAGKD |
| 450 | GHPGSPGSPGYQGPPGEPGQAGPSGPPGPPGAIGPSGPAGKDGES |
| 451 | GHPGSPGSPGYQGPPGEPGQAGPSGPPGPPGAIGPSGPAGKDGESGRPGRP |
| | GER |
| 452 | GHRGFDGRNGEKGETGAPGLK |
| 453 | GHRGFDGRNGEKGETGAPGLKGEN |
| 454 | GHRGFDGRNGEKGETGAPGLKGENG |
| 455 | GHRGFDGRNGEKGETGAPGLKGENGLPGEN |
| 456 | GHRGFDGRNGEKGETGAPGLKGENGLPGENGAPGPM |
| 457 | GHRGFDGRNGEKGETGAPGLKGENGLPGENGAPGPMGPR |
| 458 | GHRGFDGRNGEKGETGAPGLKGENGLPGENGAPGPMGPRGAPGER |
| 459 | GIKGPAGIP |
| 460 | GIKGPAGIPGFPG |
| 461 | GIKGPAGIPGFPGMKG |
| 462 | GIPGAPGLM |
| 463 | GIPGAPGLMG |
| 464 | GIPGAPGLMGA |
| 465 | GIPGAPGLMGAR |
| 466 | GIPGAPGLMGARGPPG |
| 467 | GIPGAPGLMGARGPPGPA |
| 468 | GIPGAPGLMGARGPPGPAG |
| 469 | GIPGAPGLMGARGPPGPAGA |
| 470 | GIPGAPGLMGARGPPGPAGAN |
| 471 | GIPGAPGLMGARGPPGPAGANGAPGLR |
| 472 | GIPGAPGLMGARGPPGPAGANGAPGLRG |
| 473 | GIPGAPGLMGARGPPGPAGANGAPGLRGGAGEPGKN |
| 474 | GIPGEKGPAGERGAPGPA |
| 475 | GIPGEKGPAGERGAPGPAG |
| 476 | GIPGEKGPAGERGAPGPAGPR |
| 477 | GIPGEKGPAGERGAPGPAGPRG |
| 478 | GIPGEKGPAGERGAPGPAGPRGA |
| 479 | GIPGEKGPAGERGAPGPAGPRGAA |
| 480 | GIPGEKGPAGERGAPGPAGPRGAAG |
| 481 | GIPGEKGPAGERGAPGPAGPRGAAGEPG |
| 482 | GIPGEKGPAGERGAPGPAGPRGAAGEPGR |
| 483 | GIPGEKGPAGERGAPGPAGPRGAAGEPGRD |
| 484 | GIPGEKGPAGERGAPGPAGPRGAAGEPGRDGVPG |
| 485 | GIPGEKGPAGERGAPGPAGPRGAAGEPGRDGVPGG |
| 486 | GIPGEKGPAGERGAPGPAGPRGAAGEPGRDGVPGGPG |
| 487 | GIPGEKGPAGERGAPGPAGPRGAAGEPGRDGVPGGPGMR |
| 488 | GIPGFPGMKGH |
| 489 | GIPGFPGMKGHR |
| 490 | GIPGFPGMKGHRGF |
| 491 | GIPGFPGMKGHRGFD |
| 492 | GIPGFPGMKGHRGFDGR |
| 493 | GIPGFPGMKGHRGFDGRN |
| 494 | GIPGFPGMKGHRGFDGRNGEK |
| 495 | GIPGFPGMKGHRGFDGRNGEKG |
| 496 | GIPGFPGMKGHRGFDGRNGEKGETGAPGLK |
| 497 | GIPGFPGMKGHRGFDGRNGEKGETGAPGLKGEN |
| 498 | GIPGFPGMKGHRGFDGRNGEKGETGAPGLKGENGLPGEN |
| 499 | GIPGFPGMKGHRGFDGRNGEKGETGAPGLKGENGLPGENGAPGPMGPR |
| 500 | GKDGESGRPGRPGERGLPGPPGIK |
| 501 | GKDGESGRPGRPGERGLPGPPGIKGPA |
| 502 | GKDGESGRPGRPGERGLPGPPGIKGPAGIPGFPGMK |
| 503 | GKDGSPGEPGANGLPGA |
| 504 | GKDGSPGEPGANGLPGAAGERGAPG |
| 505 | GKDGSPGEPGANGLPGAAGERGAPGFRGPAGPN |
| 506 | GKGEMGPAGIPGAPGLM |
| 507 | GKGEMGPAGIPGAPGLMG |
| 508 | GKGEMGPAGIPGAPGLMGA |
| 509 | GKGEMGPAGIPGAPGLMGAR |
| 510 | GKGEMGPAGIPGAPGLMGARGPPGPA |
| 511 | GKGEMGPAGIPGAPGLMGARGPPGPAGAN |
| 512 | GKGEMGPAGIPGAPGLMGARGPPGPAGANGAPGLR |
| 513 | GKPGEPGPKGDAGAPGAPGGKGDAGAPG |
| 514 | GKPGEPGPKGDAGAPGAPGGKGDAGAPGE |
| 515 | GKPGEPGPKGDAGAPGAPGGKGDAGAPGER |
| 516 | GKPGEPGPKGDAGAPGAPGGKGDAGAPGERG |
| 517 | GKPGEPGPKGDAGAPGAPGGKGDAGAPGERGPP |
| 518 | GKPGPPGSQGESGRPGPPGPSGPR |
| 519 | GKPGPPGSQGESGRPGPPGPSGPRGQ |
| 520 | GKPGPPGSQGESGRPGPPGPSGPRGQPGVM |
| 521 | GLAGYPGPAGPPGPPGPPGTS |
| 522 | GLAGYPGPAGPPGPPGPPGTSGHPGSPGSPGYQGPPGEPGQAGPSGPPGPP |
| | GAIGPS |
| 523 | GLKGENGLPGENGAPGPMGPR |
| 524 | GLMGARGPPGPAGANGAPGLR |
| 525 | GLPGAAGARGNDGARGSDGQPGPPGPPGTA |
| 526 | GLPGAAGARGNDGARGSDGQPGPPGPPGTAGFPGSPGAKGEVGPA |
| 527 | GLPGAAGERGAPGF |
| 528 | GLPGAAGERGAPGFR |
| 529 | GLPGAAGERGAPGFRG |
| 530 | GLPGAAGERGAPGFRGPAG |
| 531 | GLPGAAGERGAPGFRGPAGPN |
| 532 | GLPGAAGERGAPGFRGPAGPNGIPGEKG |
| 533 | GLPGAAGERGAPGFRGPAGPNGIPGEKGPAGER |
| 534 | GLPGAAGERGAPGFRGPAGPNGIPGEKGPAGERGAPG |
| 535 | GLPGAAGERGAPGFRGPAGPNGIPGEKGPAGERGAPGPAGPR |
| 536 | GLPGENGAPGPM |
| 537 | GLPGENGAPGPMG |
| 538 | GLPGENGAPGPMGPR |
| 539 | GLPGENGAPGPMGPRG |
| 540 | GLPGENGAPGPMGPRGAPG |
| 541 | GLPGENGAPGPMGPRGAPGERGR |
| 542 | GLPGENGAPGPMGPRGAPGERGRPG |
| 543 | GLPGENGAPGPMGPRGAPGERGRPGLPGAAGAR |
| 544 | GLPGENGAPGPMGPRGAPGERGRPGLPGAAGARGN |
| 545 | GLPGENGAPGPMGPRGAPGERGRPGLPGAAGARGNDGARG |
| 546 | GLPGENGAPGPMGPRGAPGERGRPGLPGAAGARGNDGARGSDGQ |
| 547 | GLPGENGAPGPMGPRGAPGERGRPGLPGAAGARGNDGARGSDGQPGPPG |
| | PPGTA |
| 548 | GLPGPPGIK |
| 549 | GLPGPPGIKG |
| 550 | GLPGPPGIKGP |
| 551 | GLPGPPGIKGPA |
| 552 | GLPGPPGIKGPAG |
| 553 | GLPGPPGIKGPAGIP |
| 554 | GLPGPPGIKGPAGIPG |
| 555 | GLPGPPGIKGPAGIPGF |
| 556 | GLPGPPGIKGPAGIPGFPGM |
| 557 | GLPGPPGIKGPAGIPGFPGMK |
| 558 | GLPGPPGIKGPAGIPGFPGMKG |
| 559 | GLPGPPGIKGPAGIPGFPGMKGH |
| 560 | GLPGPPGIKGPAGIPGFPGMKGHR |
| 561 | GLPGPPGIKGPAGIPGFPGMKGHRGF |
| 562 | GLPGTGGPPGEN |
| 563 | GLRGGAGEPGKNGAKGEPGPRGERGEAGIP |
| 564 | GMPGSPGGPGSDGKPGPPG |
| 565 | GMPGSPGGPGSDGKPGPPGSQ |
| 566 | GMPGSPGGPGSDGKPGPPGSQG |
| 567 | GMPGSPGGPGSDGKPGPPGSQGESGRPGPPGPSGPR |
| 568 | GMPGSPGGPGSDGKPGPPGSQGESGRPGPPGPSGPRGQPGVM |
| 569 | GMRGMPGSPGGPGSDGKPGPPGSQGESGRPGPPGPSGPRGQPGVM |
| 570 | GNDGAPGKNGERGGPGGPGPQGPPGKN |
| 571 | GNDGAPGKNGERGGPGGPGPQGPPGKNGETGPQG |
| 572 | GNDGAPGKNGERGGPGGPGPQGPPGKNGETGPQGPPGP |
| 573 | GNDGARGSDGQPGPPGPPG |
| 574 | GNDGARGSDGQPGPPGPPGTA |
| 575 | GNDGARGSDGQPGPPGPPGTAG |
| 576 | GNDGARGSDGQPGPPGPPGTAGF |
| 577 | GNDGARGSDGQPGPPGPPGTAGFPG |
| 578 | GNDGARGSDGQPGPPGPPGTAGFPGS |
| 579 | GNDGARGSDGQPGPPGPPGTAGFPGSPG |
| 580 | GNDGARGSDGQPGPPGPPGTAGFPGSPGAK |
| 581 | GNDGARGSDGQPGPPGPPGTAGFPGSPGAKG |
| 582 | GNDGARGSDGQPGPPGPPGTAGFPGSPGAKGE |
| 583 | GNDGARGSDGQPGPPGPPGTAGFPGSPGAKGEVG |
| 584 | GNDGARGSDGQPGPPGPPGTAGFPGSPGAKGEVGP |
| 585 | GNDGARGSDGQPGPPGPPGTAGFPGSPGAKGEVGPA |
| 586 | GNDGARGSDGQPGPPGPPGTAGFPGSPGAKGEVGPAG |
| 587 | GNDGARGSDGQPGPPGPPGTAGFPGSPGAKGEVGPAGS |
| 588 | GNDGARGSDGQPGPPGPPGTAGFPGSPGAKGEVGPAGSPG |
| 589 | GNDGARGSDGQPGPPGPPGTAGFPGSPGAKGEVGPAGSPGSN |
| 590 | GNDGARGSDGQPGPPGPPGTAGFPGSPGAKGEVGPAGSPGSNG |
| 591 | GNDGARGSDGQPGPPGPPGTAGFPGSPGAKGEVGPAGSPGSNGAPGQ |
| 592 | GNDGARGSDGQPGPPGPPGTAGFPGSPGAKGEVGPAGSPGSNGAPGQR |
| 593 | GNDGARGSDGQPGPPGPPGTAGFPGSPGAKGEVGPAGSPGSNGAPGQRGE |
| 594 | GNDGARGSDGQPGPPGPPGTAGFPGSPGAKGEVGPAGSPGSNGAPGQRGE |
| | PGPQ |
| 595 | GNDGARGSDGQPGPPGPPGTAGFPGSPGAKGEVGPAGSPGSNGAPGQRGE |
| | PGPQG |
| 596 | GPAGANGAPGLRGGAGEPG |
| 597 | GPAGIPGAPGLM |
| 598 | GPAGIPGAPGLMG |
| 599 | GPAGIPGAPGLMGA |
| 600 | GPAGIPGAPGLMGAR |
| 601 | GPAGIPGAPGLMGARGPPGPAGANGAPGLR |
| 602 | GPAGIPGFP |
| 603 | GPAGIPGFPG |
| 604 | GPAGIPGFPGM |
| 605 | GPAGIPGFPGMK |
| 606 | GPAGIPGFPGMKG |
| 607 | GPAGIPGFPGMKGH |
| 608 | GPAGIPGFPGMKGHR |
| 609 | GPAGIPGFPGMKGHRG |
| 610 | GPAGIPGFPGMKGHRGF |
| 611 | GPAGIPGFPGMKGHRGFD |
| 612 | GPAGIPGFPGMKGHRGFDG |
| 613 | GPAGIPGFPGMKGHRGFDGR |
| 614 | GPAGIPGFPGMKGHRGFDGRN |
| 615 | GPAGIPGFPGMKGHRGFDGRNG |
| 616 | GPAGIPGFPGMKGHRGFDGRNGE |
| 617 | GPAGIPGFPGMKGHRGFDGRNGEK |
| 618 | GPAGIPGFPGMKGHRGFDGRNGEKG |
| 619 | GPAGIPGFPGMKGHRGFDGRNGEKGE |
| 620 | GPAGIPGFPGMKGHRGFDGRNGEKGET |
| 621 | GPAGIPGFPGMKGHRGFDGRNGEKGETG |
| 622 | GPAGIPGFPGMKGHRGFDGRNGEKGETGA |
| 623 | GPAGIPGFPGMKGHRGFDGRNGEKGETGAP |
| 624 | GPAGIPGFPGMKGHRGFDGRNGEKGETGAPG |
| 625 | GPAGIPGFPGMKGHRGFDGRNGEKGETGAPGL |
| 626 | GPAGIPGFPGMKGHRGFDGRNGEKGETGAPGLK |
| 627 | GPAGIPGFPGMKGHRGFDGRNGEKGETGAPGLKG |
| 628 | GPAGIPGFPGMKGHRGFDGRNGEKGETGAPGLKGEN |
| 629 | GPAGIPGFPGMKGHRGFDGRNGEKGETGAPGLKGENGLPG |
| 630 | GPAGIPGFPGMKGHRGFDGRNGEKGETGAPGLKGENGLPGEN |
| 631 | GPAGIPGFPGMKGHRGFDGRNGEKGETGAPGLKGENGLPGENGAPGPM |
| 632 | GPAGIPGFPGMKGHRGFDGRNGEKGETGAPGLKGENGLPGENGAPGPMG |
| | PR |
| 633 | GPAGKDGESGRPGRPGERGLPGPP |
| 634 | GPAGKDGESGRPGRPGERGLPGPPG |
| 635 | GPAGKDGESGRPGRPGERGLPGPPGIK |
| 636 | GPAGKDGESGRPGRPGERGLPGPPGIKGPA |
| 637 | GPAGKDGESGRPGRPGERGLPGPPGIKGPAGIPGFPGMK |
| 638 | GPAGPNGIPGEKGPAGERGAPGPAGPR |
| 639 | GPAGPPGPPGPP |
| 640 | GPGMRGMPGSPGGPGSDGKPGPPG |
| 641 | GPGMRGMPGSPGGPGSDGKPGPPGSQGESGRPGPPGPSGPRGQPGVM |
| 642 | GPGSDGKPGPPGSQGESGRPGPPG |
| 643 | GPGSDGKPGPPGSQGESGRPGPPGPSG |
| 644 | GPGSDGKPGPPGSQGESGRPGPPGPSGPR |
| 645 | GPGSDGKPGPPGSQGESGRPGPPGPSGPRGQPGVM |
| 646 | GPGSDGKPGPPGSQGESGRPGPPGPSGPRGQPGVMGFPGPKGN |
| 647 | GPMGPRGAPGERGRPGLPGAA |
| 648 | GPMGPRGAPGERGRPGLPGAAGAR |
| 649 | GPNGIPGEKGPAGERGAPGPA |
| 650 | GPPGAIGPSGPAGKDGESGRPGRPGER |
| 651 | GPPGAIGPSGPAGKDGESGRPGRPGERGLPGPPGIK |
| 652 | GPPGEPGQAGPSGPPGPPG |
| 653 | GPPGEPGQAGPSGPPGPPGAIGPS |
| 654 | GPPGEPGQAGPSGPPGPPGAIGPSGPAGKD |
| 655 | GPPGEPGQAGPSGPPGPPGAIGPSGPAGKDGESGRPGRPGERGLPGPPGIK |
| 656 | GPPGIKGPAGIPGFPGMK |
| 657 | GPPGINGSPGGKGEMGPAGIPGAPGLM |
| 658 | GPPGPAGANGAPGLR |
| 659 | GPPGPAGANGAPGLRG |
| 660 | GPPGPAGANGAPGLRGG |
| 661 | GPPGPAGANGAPGLRGGA |
| 662 | GPPGPAGANGAPGLRGGAG |
| 663 | GPPGPAGANGAPGLRGGAGE |
| 664 | GPPGPAGANGAPGLRGGAGEP |
| 665 | GPPGPAGANGAPGLRGGAGEPG |
| 666 | GPPGPAGANGAPGLRGGAGEPGK |
| 667 | GPPGPAGANGAPGLRGGAGEPGKN |
| 668 | GPPGPAGANGAPGLRGGAGEPGKNG |
| 669 | GPPGPAGANGAPGLRGGAGEPGKNGA |
| 670 | GPPGPAGANGAPGLRGGAGEPGKNGAK |
| 671 | GPPGPAGANGAPGLRGGAGEPGKNGAKG |
| 672 | GPPGPAGANGAPGLRGGAGEPGKNGAKGE |
| 673 | GPPGPAGANGAPGLRGGAGEPGKNGAKGEPG |
| 674 | GPPGPAGANGAPGLRGGAGEPGKNGAKGEPGPR |
| 675 | GPPGPAGANGAPGLRGGAGEPGKNGAKGEPGPRG |
| 676 | GPPGPAGANGAPGLRGGAGEPGKNGAKGEPGPRGE |
| 677 | GPPGPAGANGAPGLRGGAGEPGKNGAKGEPGPRGER |
| 678 | GPPGPAGANGAPGLRGGAGEPGKNGAKGEPGPRGERG |
| 679 | GPPGPAGANGAPGLRGGAGEPGKNGAKGEPGPRGERGE |
| 680 | GPPGPAGANGAPGLRGGAGEPGKNGAKGEPGPRGERGEA |
| 681 | GPPGPPGAIGPS |
| 682 | GPPGPPGAIGPSGPA |
| 683 | GPPGPPGAIGPSGPAGKDGESGRPGRPGER |
| 684 | GPPGPPGAIGPSGPAGKDGESGRPGRPGERGLPGPPGIK |
| 685 | GPPGPPGINGSPGGKGEMGPA |
| 686 | GPPGPPGINGSPGGKGEMGPAGIPGAPGLM |
| 687 | GPPGPPGINGSPGGKGEMGPAGIPGAPGLMGA |
| 688 | GPPGPPGINGSPGGKGEMGPAGIPGAPGLMGAR |
| 689 | GPPGPPGINGSPGGKGEMGPAGIPGAPGLMGARGPPGPAGANGAPGLR |
| 690 | GPPGPPGPPGTS |
| 691 | GPPGPPGPPGTSGHPG |
| 692 | GPPGPPGPPGTSGHPGSPGSPGYQGPPGEPGQAGPSGPPGPPGAIGPS |
| 693 | GPPGPPGTAGFPGSPGAK |
| 694 | GPPGPPGTAGFPGSPGAKGEVGPA |
| 695 | GPPGPSGPRGQPGVM |
| 696 | GPPGPSGPRGQPGVMGFPGPKGNDGAPGKN |
| 697 | GPPGSQGESGRPGPPGPSGPRGQPGVM |
| 698 | GPPGTAGFPGSPGAKGEVGPA |
| 699 | GPQGHAGAQGPPGPPGIN |
| 700 | GPRGAAGEPGRDGVPGGPGMRGMPG |
| 701 | GPRGAPGERGRPGLPGAA |
| 702 | GPRGAPGERGRPGLPGAAGA |
| 703 | GPRGAPGERGRPGLPGAAGAR |
| 704 | GPRGAPGERGRPGLPGAAGARGN |
| 705 | GPRGAPGERGRPGLPGAAGARGNDGARG |
| 706 | GPRGAPGERGRPGLPGAAGARGNDGARGSDGQPGPPGPPGTA |
| 707 | GPRGQPGVMGFPGPKGN |
| 708 | GPRGQPGVMGFPGPKGNDGAPGKN |
| 709 | GPSGPAGKDGESGRPGRPGERGLPGPPGIK |
| 710 | GPSGPAGKDGESGRPGRPGERGLPGPPGIKGPA |
| 711 | GPSGPPGPPGAIGPS |
| 712 | GPSGPRGQPGVMGFPGPKGNDGAPGKN |
| 713 | GQAGPSGPPGPPGAIGPS |
| 714 | GQPGPPGPPGTA |
| 715 | GQPGPPGPPGTAGFPGSPGAK |
| 716 | GQPGPPGPPGTAGFPGSPGAKGEVGPA |
| 717 | GQPGVMGFPGPK |
| 718 | GQPGVMGFPGPKGN |
| 719 | GQPGVMGFPGPKGNDGAPGKN |
| 720 | GQRGEPGPQGHAGAQGPPGPPGIN |
| 721 | GRDGVPGGPGMRGMPGSPGGPG |
| 722 | GRDGVPGGPGMRGMPGSPGGPGSDGKPGPPGSQGESGRPGPPGPSGPRGQ |
| | PGVM |
| 723 | GRNGEKGETGAPGLKGENGLPGEN |
| 724 | GRNGEKGETGAPGLKGENGLPGENGAPGPMGPR |
| 725 | GRPGERGLPGPPGIK |
| 726 | GRPGERGLPGPPGIKGPA |
| 727 | GRPGLPGAAGAR |
| 728 | GRPGLPGAAGARGNDGARGSDGQPGPPGPPGTA |
| 729 | GRPGLPGAAGARGNDGARGSDGQPGPPGPPGTAGFPGSPGAK |
| 730 | GRPGLPGAAGARGNDGARGSDGQPGPPGPPGTAGFPGSPGAKGEVGPA |
| 731 | GRPGPPGPSGPRGQPGVM |
| 732 | GRPGRPGERGLPGPPGIK |
| 733 | GRPGRPGERGLPGPPGIKGPA |
| 734 | GSDGKPGPPGSQGESGRPGPPGPSGPRGQPGVM |
| 735 | GSDGQPGPPGPPGTA |
| 736 | GSDGQPGPPGPPGTAGFPGSPGAK |
| 737 | GSDGQPGPPGPPGTAGFPGSPGAKGEVGPA |
| 738 | GSNGAPGQRGEPGPQGHAGAQGPPGPPGIN |
| 739 | GSPGEPGANGLPGAAGERGAPGFRGPAGPN |
| 740 | GSPGGKGEMGPAGIPGAPG |
| 741 | GSPGGKGEMGPAGIPGAPGLM |
| 742 | GSPGGKGEMGPAGIPGAPGLMG |
| 743 | GSPGGKGEMGPAGIPGAPGLMGA |
| 744 | GSPGGKGEMGPAGIPGAPGLMGAR |
| 745 | GSPGGKGEMGPAGIPGAPGLMGARG |
| 746 | GSPGGKGEMGPAGIPGAPGLMGARGPPG |
| 747 | GSPGGKGEMGPAGIPGAPGLMGARGPPGPA |
| 748 | GSPGGKGEMGPAGIPGAPGLMGARGPPGPAG |
| 749 | GSPGGKGEMGPAGIPGAPGLMGARGPPGPAGAN |
| 750 | GSPGGKGEMGPAGIPGAPGLMGARGPPGPAGANGAPGLR |
| 751 | GSPGGKGEMGPAGIPGAPGLMGARGPPGPAGANGAPGLRGGAGEPGKN |
| 752 | GSPGGPGSDGKPGPPGSQGESGRPGPPGPSGPRGQPGVM |
| 753 | GSPGSNGAPGQRGEPGPQ |
| 754 | GSPGSNGAPGQRGEPGPQGHAGAQ |
| 755 | GSPGSNGAPGQRGEPGPQGHAGAQGP |
| 756 | GSPGSNGAPGQRGEPGPQGHAGAQGPP |
| 757 | GSPGSNGAPGQRGEPGPQGHAGAQGPPG |
| 758 | GSPGSNGAPGQRGEPGPQGHAGAQGPPGP |
| 759 | GSPGSNGAPGQRGEPGPQGHAGAQGPPGPP |
| 760 | GSPGSNGAPGQRGEPGPQGHAGAQGPPGPPG |
| 761 | GSPGSNGAPGQRGEPGPQGHAGAQGPPGPPGIN |
| 762 | GSPGSNGAPGQRGEPGPQGHAGAQGPPGPPGING |
| 763 | GSPGSNGAPGQRGEPGPQGHAGAQGPPGPPGINGS |
| 764 | GSPGSNGAPGQRGEPGPQGHAGAQGPPGPPGINGSPG |
| 765 | GSPGSNGAPGQRGEPGPQGHAGAQGPPGPPGINGSPGG |
| 766 | GSPGSNGAPGQRGEPGPQGHAGAQGPPGPPGINGSPGGK |
| 767 | GSPGSNGAPGQRGEPGPQGHAGAQGPPGPPGINGSPGGKG |
| 768 | GSPGSNGAPGQRGEPGPQGHAGAQGPPGPPGINGSPGGKGEM |
| 769 | GSPGSNGAPGQRGEPGPQGHAGAQGPPGPPGINGSPGGKGEMG |
| 770 | GSPGSNGAPGQRGEPGPQGHAGAQGPPGPPGINGSPGGKGEMGPA |
| 771 | GSPGSNGAPGQRGEPGPQGHAGAQGPPGPPGINGSPGGKGEMGPAG |
| 772 | GSPGSNGAPGQRGEPGPQGHAGAQGPPGPPGINGSPGGKGEMGPAGIPGA |
| | PGLM |
| 773 | GSPGSNGAPGQRGEPGPQGHAGAQGPPGPPGINGSPGGKGEMGPAGIPGA |
| | PGLMG |
| 774 | GSPGSNGAPGQRGEPGPQGHAGAQGPPGPPGINGSPGGKGEMGPAGIPGA |
| | PGLMGA |
| 775 | GSQGESGRPGPPGPSGPRGQPGVM |
| 776 | GTAGFPGSPGAKGEVGPA |
| 777 | GTSGHPGSPGSPGYQGPPGEPGQAGPSGPPGPPGAIGPS |
| 778 | GVPGGPGMRGMPGSPGGPGSDGKPGPPGSQ |
| 779 | GVPGGPGMRGMPGSPGGPGSDGKPGPPGSQGESGRPGPPGPSGPRGQPGV |
| | M |
| 780 | GYPGPAGPPGPPGPPGT |
| 781 | GYPGPAGPPGPPGPPGTS |
| 782 | GYPGPAGPPGPPGPPGTSGH |
| 783 | GYPGPAGPPGPPGPPGTSGHPG |
| 784 | GYPGPAGPPGPPGPPGTSGHPGSPGSPGYQGPPGEPGQAGPSGPPGPPGAIG |
| | PS |
| 785 | GYPGPAGPPGPPGPPGTSGHPGSPGSPGYQGPPGEPGQAGPSGPPGPPGAIG |
| | PSGPA |
| 786 | HAGAQGPPGPPGIN |
| 787 | HAGAQGPPGPPGINGSPG |
| 788 | HAGAQGPPGPPGINGSPGGKGEMG |
| 789 | HAGAQGPPGPPGINGSPGGKGEMGPA |
| 790 | HAGAQGPPGPPGINGSPGGKGEMGPAGIPGAPGLMGAR |
| 791 | HPGSPGSPGYQGPPGEPGQAGPSGPPGPPGAIGPS |
| 792 | HRGFDGRNGEKGETGAPGLKGEN |
| 793 | HRGFDGRNGEKGETGAPGLKGENGLPGENGAPGPMGPR |
| 794 | IGPSGPAGKDGESGRPGRPGERGLPGPPGIK |
| 795 | IKGPAGIPGFPG |
| 796 | IKGPAGIPGFPGMK |
| 797 | IPGAPGLMGARGPPGPAGANGAPGLR |
| 798 | IPGFPGMKGHRGFDGRN |
| 799 | KDGESGRPGRPGERGLPGPPGIK |
| 800 | KDGESGRPGRPGERGLPGPPGIKGPA |
| 801 | KDGSPGEPGANGLPGAAGERGAPGFRGPAGPN |
| 802 | KGENGLPGENGAPGPMGPR |
| 803 | KGPAGIPGFPGMK |
| 804 | KPGPPGSQGESGRPGPPGPSGPR |
| 805 | KPGPPGSQGESGRPGPPGPSGPRGQPGVM |
| 806 | LKGENGLPGENGAPGPMGPR |
| 807 | LMGARGPPGPAGANGAPGLR |
| 808 | LPGAAGARGNDGARGSDGQPGPPGPPGTA |
| 809 | LPGAAGERGAPGFRGPAGPN |
| 810 | LPGPPGIK |
| 811 | LPGPPGIKGPA |
| 812 | LPGPPGIKGPAGIPGFPGMK |
| 813 | MGARGPPGPAGANGAPGLR |
| 814 | MGARGPPGPAGANGAPGLRGGAGEPGKN |
| 815 | MGPAGIPGAPGLMGARGPPGPAGANGAPGLR |
| 816 | MGPRGAPGERGRPGLPGAAGAR |
| 817 | MPGSPGGPGSDGKPGPPGSQGESGRPGPPGPSGPRGQPGVM |
| 818 | MRGMPGSPGGPGSDGKPGPPGSQGESGRPGPPGPSGPRGQPGVM |
| 819 | NDGARGSDGQPGPPGPPGTA |
| 820 | NDGARGSDGQPGPPGPPGTAGFPGSPGAKGEVGPA |
| 821 | NGAPGPMGPRGAPGE |
| 822 | NGAPGPMGPRGAPGERGRPGLPGAAGAR |
| 823 | NGEKGETGAPGLKGENGLPGENGAPGPMGPR |
| 824 | NGETGPQGPPGPTGPGGD |
| 825 | NGLPGENGAPGPMGPR |
| 826 | NGSPGGKGEMGPAGIPGAPGLM |
| 827 | NGSPGGKGEMGPAGIPGAPGLMGARGPPGPAGANGAPGLR |
| 828 | PAGIPGAPGLM |
| 829 | PAGIPGAPGLMG |
| 830 | PAGIPGFPGMK |
| 831 | PAGIPGFPGMKG |
| 832 | PAGIPGFPGMKGHR |
| 833 | PAGIPGFPGMKGHRG |
| 834 | PAGIPGFPGMKGHRGF |
| 835 | PAGIPGFPGMKGHRGFDG |
| 836 | PAGIPGFPGMKGHRGFDGRN |
| 837 | PAGKDGESGRPGRPGERGLPGPPGIK |
| 838 | PGAAGERGAPGFRGPAGPN |
| 839 | PGANGLPGAAGERGAP |
| 840 | PGAPGLMGARGPPGPAGANGAPGLR |
| 841 | PGEPGPKGDAGAPGAPGGKGDAGAPGE |
| 842 | PGERGLPGPPGIK |
| 843 | PGERGRPGLPGAAGAR |
| 844 | PGFPGMKGH |
| 845 | PGFPGMKGHR |
| 846 | PGFPGMKGHRGF |
| 847 | PGFPGMKGHRGFDGR |
| 848 | PGLMGARGPPGPAGANGAPGL |
| 849 | PGLPGAAGARGNDGARGSDGQPGPPGPPGTAGFPGSPGAKG |
| 850 | PGPAGANGAPGLRGGAGEPGKN |
| 851 | PGPAGPPGPPGPPG |
| 852 | PGPAGPPGPPGPPGTS |
| 853 | PGPAGPPGPPGPPGTSG |
| 854 | PGPAGPPGPPGPPGTSGH |
| 855 | PGPAGPPGPPGPPGTSGHPGSPGSPGYQGPPGEPGQAGPSGP |
| 856 | PGPAGPPGPPGPPGTSGHPGSPGSPGYQGPPGEPGQAGPSGPPGPPGAIGPS |
| | GPAGK |
| 857 | PGPKGNDGAPGKN |
| 858 | PGPKGNDGAPGKNGERGGPGGPGPQGPPGKN |
| 859 | PGPMGPRGAPGERGRPGLPGAAGAR |
| 860 | PGPPGAIGPS |
| 861 | PGPPGIKGPAGIPGFPGMK |
| 862 | PGPPGPPGTAGFPGSPGAK |
| 863 | PGPPGPSGPRGQPGVM |
| 864 | PGRPGERGLPGPPGIK |
| 865 | PGSDGKPGPPGSQGESGRPGPPGPSGPRGQPGVM |
| 866 | PGSNGAPGQRGEPGPQGHAGAQGPPGPPGINGS |
| 867 | PGSPGAKGEVGPA |
| 868 | PPGAIGPSGPAGKDGESGRPGRPGER |
| 869 | PPGAIGPSGPAGKDGESGRPGRPGERGLPGPPGIK |
| 870 | PPGEPGQAGPSGPPGPPGAIGPS |
| 871 | PPGEPGQAGPSGPPGPPGAIGPSGPAGKD |
| 872 | PPGIKGPAGIPGFPGMK |
| 873 | PPGINGSPGGKGEMGPAGIPGAPGLM |
| 874 | PPGPAGANGAPGLR |
| 875 | PPGPPGAIGPS |
| 876 | PPGPPGAIGPSGPAGKDGESGRPGRPGERGLPGPPGIK |
| 877 | PPGPPGINGSPGGKGEMGPAGIPGAPGLM |
| 878 | PPGPPGTAGFPGSPGAKGEVGPA |
| 879 | PPGSQGESGRPGPPGPSGPRGQPGVM |
| 880 | PPGTAGFPGSPGAKGEVGPA |
| 881 | PQGHAGAQGPPGPPGIN |
| 882 | PSGPAGKDGESGRPGRPGERGLPGPPGIK |
| 883 | PSGPPGPPGAIGPS |
| 884 | QAGPSGPPGPPGAIGPS |
| 885 | QGPPGEPGQAGPSGPPGPPGAIGPSGPAGKD |
| 886 | QRGEPGPQGHAGAQGPPGPPGIN |
| 887 | RGFDGRNGEKGETGAPGLKGEN |
| 888 | RGLPGPPGIK |
| 889 | RGLPGPPGIKGPA |
| 890 | RGLPGPPGIKGPAGIPGFPGMK |
| 891 | RGMPGSPGGPGSDGKPGPPGSQGESGRPGPPGPSGPRGQPGVM |
| 892 | RGNDGARGSDGQPGPPGPPGTA |
| 893 | RGNDGARGSDGQPGPPGPPGTAGFPGSPGAK |
| 894 | RGNDGARGSDGQPGPPGPPGTAGFPGSPGAKGEVGPA |
| 895 | RGPPGPAGANGAPGL |
| 896 | RGPPGPAGANGAPGLR |
| 897 | RGPPGPAGANGAPGLRG |
| 898 | RGPPGPAGANGAPGLRGGAGEPGKN |
| 899 | RGPPGPAGANGAPGLRGGAGEPGKNGAK |
| 900 | RGSDGQPGPPGPPGTAGFPGSPGAK |
| 901 | RGSDGQPGPPGPPGTAGFPGSPGAKGEVGPA |
| 902 | RPGERGLPGPPGIK |
| 903 | RPGERGLPGPPGIKGPA |
| 904 | RPGLPGAAGARGNDGARGSDGQPGPPGP |
| 905 | RPGLPGAAGARGNDGARGSDGQPGPPGPPGTA |
| 906 | RPGPPGPSGPRGQPGVM |
| 907 | RPGRPGERGLPGPPGIK |
| 908 | RPGRPGERGLPGPPGIKGPA |
| 909 | SDGKPGPPGSQGESGRPGPPGPS |
| 910 | SDGKPGPPGSQGESGRPGPPGPSG |
| 911 | SDGKPGPPGSQGESGRPGPPGPSGPR |
| 912 | SDGKPGPPGSQGESGRPGPPGPSGPRG |
| 913 | SDGKPGPPGSQGESGRPGPPGPSGPRGQPG |
| 914 | SDGKPGPPGSQGESGRPGPPGPSGPRGQPGVM |
| 915 | SDGKPGPPGSQGESGRPGPPGPSGPRGQPGVMGFPGPKGN |
| 916 | SDGQPGPPGPPGTA |
| 917 | SDGQPGPPGPPGTAGFPGSPGAK |
| 918 | SDGQPGPPGPPGTAGFPGSPGAKGEVGPA |
| 919 | SGPAGKDGESGRPGRPGERGLPGPPGIK |
| 920 | SGPPGPPGAIGPS |
| 921 | SGPPGPPGAIGPSGPAGKD |
| 922 | SGPPGPPGAIGPSGPAGKDGESGRPGRPGER |
| 923 | SGPPGPPGAIGPSGPAGKDGESGRPGRPGERGLPGPPGIK |
| 924 | SGPPGPPGAIGPSGPAGKDGESGRPGRPGERGLPGPPGIKGPA |
| 925 | SGPRGQPGVMGFPGPKGN |
| 926 | SGRPGPPGPSGPRGQPGVM |
| 927 | SGRPGPPGPSGPRGQPGVMGFPGPKGN |
| 928 | SGRPGRPGERGLPGPP |
| 929 | SGRPGRPGERGLPGPPG |
| 930 | SGRPGRPGERGLPGPPGIK |
| 931 | SGRPGRPGERGLPGPPGIKGPA |
| 932 | SNGAPGQRGEPGPQGHAGAQGPPGPPGIN |
| 933 | SNGAPGQRGEPGPQGHAGAQGPPGPPGINGSPGGK |
| 934 | SNGAPGQRGEPGPQGHAGAQGPPGPPGINGSPGGKGEMGPA |
| 935 | SPGAKGEVGPAGSPGSNGAPG |
| 936 | SPGAKGEVGPAGSPGSNGAPGQ |
| 937 | SPGAKGEVGPAGSPGSNGAPGQRGE |
| 938 | SPGAKGEVGPAGSPGSNGAPGQRGEPG |
| 939 | SPGAKGEVGPAGSPGSNGAPGQRGEPGPQ |
| 940 | SPGAKGEVGPAGSPGSNGAPGQRGEPGPQG |
| 941 | SPGAKGEVGPAGSPGSNGAPGQRGEPGPQGH |
| 942 | SPGAKGEVGPAGSPGSNGAPGQRGEPGPQGHAG |
| 943 | SPGAKGEVGPAGSPGSNGAPGQRGEPGPQGHAGAQGPPGPPGIN |
| 944 | SPGEPGANGLPGAAGERGAPGFRGPAGPN |
| 945 | SPGGKGEMGPAGIPGAPGLM |
| 946 | SPGGKGEMGPAGIPGAPGLMGA |
| 947 | SPGGKGEMGPAGIPGAPGLMGAR |
| 948 | SPGGKGEMGPAGIPGAPGLMGARGPPGPAGAN |
| 949 | SPGGPGSDGKPGPPGSQGESGRPGPPG |
| 950 | SPGGPGSDGKPGPPGSQGESGRPGPPGP |
| 951 | SPGGPGSDGKPGPPGSQGESGRPGPPGPS |
| 952 | SPGGPGSDGKPGPPGSQGESGRPGPPGPSGPR |
| 953 | SPGGPGSDGKPGPPGSQGESGRPGPPGPSGPRG |
| 954 | SPGGPGSDGKPGPPGSQGESGRPGPPGPSGPRGQPGVM |
| 955 | SPGGPGSDGKPGPPGSQGESGRPGPPGPSGPRGQPGVMGFPGPKGN |
| 956 | SPGSNGAPGQRGEPGPQGHAGAQGPPG |
| 957 | SPGSNGAPGQRGEPGPQGHAGAQGPPGPPGIN |
| 958 | SPGSNGAPGQRGEPGPQGHAGAQGPPGPPGINGSPG |
| 959 | SPGSNGAPGQRGEPGPQGHAGAQGPPGPPGINGSPGGK |
| 960 | SPGSNGAPGQRGEPGPQGHAGAQGPPGPPGINGSPGGKGEMG |
| 961 | SPGSNGAPGQRGEPGPQGHAGAQGPPGPPGINGSPGGKGEMGPA |
| 962 | SPGSPGYQGPPGEPGQAGPSGPPGPPGAIGPS |
| 963 | SPGYQGPPGEPGQAGPSGPPGPPGAIGPS |
| 964 | SQGESGRPGPPGPSGPRGQPGVM |
| 965 | SQGESGRPGPPGPSGPRGQPGVMGFPGPKGN |
| 966 | TAGFPGSPGAKGEVGPA |
| 967 | TAGFPGSPGAKGEVGPAG |
| 968 | TAGFPGSPGAKGEVGPAGSPG |
| 969 | TAGFPGSPGAKGEVGPAGSPGSN |
| 970 | TGAPGLKGENGLPGENGAPGPMGPR |
| 971 | TGPQGPPGPTGPGGD |
| 972 | VGPAGSPGSNGAPGQRGEPGPQG |
| 973 | DVKSGVAVGGLAGYPGPAGPPGPPGPPGTSGHPGSPGSPGYQGPPGEPGQ |
| | AGPSGPPGPPGAIGPSGPAGKDGESGRPGRPGERGLPGPPGIKGPAGIPGFP |
| | GMKGHRGFDGRNGEKGETGAPGLKGENGLPGENGAPGPMGPRGAPGER |
| | GRPGLPGAAGARGNDGARGSDGQPGPPGPPGTAGFPGSPGAKGEVGPAG |
| | SPGSNGAPGQRGEPGPQGHAGAQGPPGPPGINGSPGGKGEMGPAGIPGAP |
| | GLMGARGPPGPAGANGAPGLRGGAGEPGKNGAKGEPGPRGERGEAGIPG |
| | VPGAKGEDGKDGSPGEPGANGLPGAAGERGAPGFRGPAGPNGIPGEKGP |
| | AGERGAPGPAGPRGAAGEPGRDGVPGGPGMRGMPGSPGGPGSDGK |
| | PGPPGSQGESGRPGPPGPSGPRGQPGVMGFPGPKGNDGAPGKNGERGGPG |
| | GPGPQGPPGKNGETGPQGPPGPTGPGGDKGDTGPPGPQGLQGLPGTGGPP |
| | GENGKPGEPGPKGDAGAPGAPGGKGDAGAPGERGPPGLAGAPGLRGGA |
| | GPPGPEGGKGAAGPPGPPGAAGTPGLQGMPGERGGLGSPGPKGDKGEPG |
| | GPGADGVPGKDGPRGPTGPIGPPGPAGQPGDKGEGGAPGLPGIAGPRGSP |
| | GERGETGPPGPAGFPGAPGQNGEPGGKGERGAPGEKGEGGPPGVAGPPG |
| | GSGPAGPPGPQGVKGERGSPGGPGAAGFPGARGLPGPPGSNGNPGPPGPS |
| | GSPGKDGPPGPAGNTGAPGSPGVSGPKGDAGQPGEKGSPGAQGPPGAPGP |
| | LGIAGITGARGLAGPPGMPGPRGSPGPQGVKGESGKPGANGLSGERGPPG |
| | PQGLPGLAGTAGEPGRDGNPGSDGLPGRDGSPGGKGDRGENGSPGAPGA |
| | PGHPGPPGPVGPAGKSGDRGESGPAGPAGAPGPAGSRGAPGPQGPRGDKG |
| | ETGERGAAGIKGHRGFPGNPGAPGSPGPAGQQGAIGSPGPAGPRGPVGPS |
| | GPPGKDGTSGHPGPIGPPGPRGNRERGSEGSPGHPGQPGPPGPPGAPGPCC |
| | GGVGAAAIAGIGGEKAGGFAPYYG |
| 974 | DVKSGVAVGGLAGYPGPAGPPGPPGPPGTSGHPGSPGSPGYQGPPGEPGQ |
| | AGPSGPPGPPGAIGPSGPAGKDGESGRPGRPGERGLPGPPGIKGPAGIPGFP |
| | GMKGHRGFDGRNGEKGETGAPGLKGENGLPGENGAPGPMGPRGAPGER |
| | GRPGLPGAAGARGNDGARGSDGQPGPPGPPGTAGFPGSPGAKGEVGPAG |
| | SPGSNGAPGQRGEPGPQGHAGAQGPPGPPGINGSPGGKGEMGPAGIPGAP |
| | GLMGARGPPGPAGANGAPGLRGGAGEPGKNGAKGEPGPRGERGEAGIPG |
| | VPGAKGEDGKDGSPGEPGANGLPGAAGERGAPGFRGPAGPNGIPGEKGP |
| | AGERGAPGPAGPRGAAGEPGRDGVPGGPGMRGMPGSPGGPGSDGKPGPP |
| | GSQGESGRPGPPGPSGPRGQPGVMGFPGPKGNDGAPGKNGERGGPGGPG |
| | PQGPPGKNGETGPQGPPGPTGPGGDKGDTGPPGPQGLQGLPGTGGPPGEN |
| | GKPGEPGPKGDAGAPGAPGGKGDAGAPGERGPPAIAGIGGEKAGGFAPY |
| | YG |
| 975 | DVKSGVAVGGLAGYPGPAGAPGPPGPPGASGHPGSPGAPGYQGPPGAPG |
| | QAGPSGAPGPPGAIGASGPAGKDGASGRPGRPGERGLPGPPGAKGPAGIPG |
| | APGMKGHRGFDGRNGEKGATGAPGLKGANGLPGENGAPGPMGPRGAPG |
| | ERGRPGAPGAAGARGADGARGSDGAPGPPGPPGAAGFPGSPGAKGEVGP |
| | AGAPGSNGAPGARGEPGPQGAAGAQGPPGAPGINGSPGAKGEMGPAGAP |
| | GAPGLMGARGPPGPAGANGAPGLRGAAGEPGKNGAKGEPGPRGARGEA |
| | GIPGAPGAKGEDGADGSPGEPGANGLPGAAGARGAPGFRGAAGPNGIPG |
| | AKGPAGERGAPGPAGPRGAAGEPGRDGAPGGPGMRGAPGSPGGPGADG |
| | KPGPPGAQGESGRPGAPGPSGPRGAPGVMGFPGAKGNDGAPGANGERGG |
| | PGAPGPQGPPGANGETGPQGAPGPTGPGGAKGDTGPPGAQGLQGLPGAG |
| | GPPGENGAPGEPGPKGAAGAPGAPGAKGDAGAPGERGPPAIAGIGGEKAG |
| | GFAPYYG |
| 976 | DVKSGVAVGGLAGYPGPAGPPGPPGPPGTSGHPGSPGSPGYQGPPGEPGQ |
| | AGPSGPPGPPGAIGPSGPAGKDGESGRPGRPGERGLPGPPGIKGPAGIPGFP |
| | GMKGHRGFDGRNGEKGETGAPGLKGENGLPGENGAPGPMGPRGAPGER |
| | GRPGLPGAAGARGNDGARGSDGQPGPPGPPGTAGFPGSPGAKGEVGPAG |
| | SPGSNGAPGQRGEPGPQGHAGAQGPPGPPGINGSPGGKGEMGPAGIPGAP |
| | GLMGARGPPGPAGANGAPGLRGGAGEPGKNGAKGEPGPRGERGEAGIPG |
| | VPGAKGEDGKDGSPGEPGANGLPGAAGERGAPGFRGPAGPNGIPGEKGP |
| | AGERGAPGPAGPRGAAGEPGRDGEPGLMGPRGLPGSPGGPGPAGKEGPV |
| | GLPGIDGRPGPIGPAGARGEPGVMGFPGPKGNDGDPGKNGDKGHAGLAG |
| | ARGAPGPDGETGAQGPPGPQGVQGGKGEQGPAGPPGFQGLPGTGGPAGE |
| | VGKPGEQGLHGEFGLPGPAGPRGERGAPGERGPPAIAGIGGEKAGGFAPY |
| | YG |
| 977 | DVKSGVAVGGLAGYPGPAGPPGPPGPPGTSGHPGSPGSPGYQGPPGEPGQ |
| | AGPSGPPGPPGAIGPSGPAGKRGESGRPGRPGERGLPGPPGIKGPAGIPGFP |
| | GMKGHRGFRGRNGEKGETGAPGLKGENGLPGENGAPGPMGPRGAPGER |
| | GRPGLPGAAGARGNRGARGSDGQPGPPGPPGTAGFPGSPGAKGRVGPAG |
| | SPGSNGAPGQRGEPGPQGHAGAQGPPGPPGINGSPGGKGEMGPAGIPGAP |
| | GLMGARGPPGPAGANGAPGLRGGAGRPGKNGAKGEPGPRGERGRAGIPG |
| | VPGAKGERGKDGSPGEPGANGLPGAAGERGAPGFRGPAGPNGIPGEKGPA |
| | GERGAPGPAGPRGAAGEPGRRGVPGGPGMRGMPGSPGGPGSRGKPGPPG |
| | SQGESGRPGPPGPSGPRGQPGVMGFPGPKGNDGAPGKNGERGGPGGPGP |
| | QGPPGKNGETGPQGPPGPTGPGGRKGDTGPPGPQGLQGLPGTGGPPGENG |
| | KPGRPGPKGDAGAPGAPGGKGRAGAPGERGPPAIAGIGGEKAGGFAPYY |
| | G |
| 978 | DVKSGVAVGGLAGYPGPAGPPGPPGPPGASGHPGSPGSPGYQGPPGEPGQ |
| | AGPAGPPGPPGAIGPSGPAGKDGESGRPGRPGERGLPGPPGIKGPAGIPGFP |
| | GMKGHRGFDGRNGEKGEAGAPGLKGENGLPGENGAPGPMGPRGAPGER |
| | GRPGLPGAAGARGNDGARGSDGQPGPPGPPGAAGFPGAPGAKGEVGPAG |
| | SPGSNGAPGQRGEPGPQGHAGAQGPPGPPGINGSPGGKGEMGPAGIPGAP |
| | GLMGARGPPGPAGANGAPGLRGGAGEPGKNGAKGEPGPRGERGEAGIPG |
| | VPGAKGEDGKDGSPGEPGANGLPGAAGERGAPGFRGPAGPNGIPGEKGP |
| | AGERGAPGPAGPRGAAGEPGRDGVPGGPGMRGMPGSPGGPGSDGKPGPP |
| | GAQGESGRPGPPGPSGPRGQPGVMGFPGPKGNDGAPGKNGERGGPGGPG |
| | PQGPPGKNGETGPQGPPGPTGPGGDKGDTGPPGPQGLQGLPGTGGPPGEN |
| | GKPGEPGPKGDAGAPGAPGGKGDAGAPGERGPPAIAGIGGEKAGGFAPY |
| | YG |
| 979 | DVKSGVAVGGLAGYPGPAGPPGPPGPPGNSGHPGSPGSPGYQGPPGEPGQ |
| | AGPNGPPGPPGAIGPSGPAGKDGESGRPGRPGERGLPGPPGIKGPAGIPGFP |
| | GMKGHRGFDGRNGEKGENGAPGLKGENGLPGENGAPGPMGPRGAPGER |
| | GRPGLPGAAGARGNDGARGSDGQPGPPGPPGNAGFPGNPGAKGEVGPAG |
| | SPGSNGAPGQRGEPGPQGHAGAQGPPGPPGINGSPGGKGEMGPAGIPGAP |
| | GLMGARGPPGPAGANGAPGLRGGAGEPGKNGAKGEPGPRGERGEAGIPG |
| | VPGAKGEDGKDGSPGEPGANGLPGAAGERGAPGFRGPAGPNGIPGEKGP |
| | AGERGAPGPAGPRGAAGEPGRDGVPGGPGMRGMPGSPGGPGSDGKPGPP |
| | GNQGESGRPGPPGPSGPRGQPGVMGFPGPKGNDGAPGKNGERGGPGGPG |
| | PQGPPGKNGETGPQGPPGPTGPGGDKGDTGPPGPQGLQGLPGTGGPPGEN |
| | GKPGEPGPKGDAGAPGAPGGKGDAGAPGERGPPAIAGIGGEKAGGFAPY |
| | YG |
| 980 | DVKSGVAVGGLAGYPGPAGAPGPPGPPGASGHPGSPGAPGYQGPPGAPG |
| | QAGPSGAPGPPGAIGASGPAGKDGASGRPGRPGERGLPGPPGAKGPAGIPG |
| | APGMKGHRGFDGRNGEKGATGAPGLKGANGLPGENGAPGPMGPRGAPG |
| | ERGRPGAPGAAGARGADGARGSDGAPGPPGPPGAAGFPGSPGAKGEVGP |
| | AGAPGSNGAPGARGEPGPQGAAGAQGPPGAPGINGSPGAKGEMGPAGAP |
| | GAPGLMGARGPPGPAGANGAPGLRGAAGEPGKNGAKGEPGPRGARGEA |
| | GIPGAPGAKGEDGADGSPGEPGANGLPGAAGARGAPGFRGAAGPNGIPG |
| | AKGPAGERGAPGPAGPRGAAGEPGRDGAPGGPGMRGAPGSPGGPGADG |
| | KPGPPGAQGESGRPGAPGPSGPRGAPGVMGFPGAKGNDGAPGANGERGG |
| | PGAPGPQGPPGANGETGPQGAPGPTGPGGAKGDTGPPGAQGLQGLPGAG |
| | GPPGENGAPGEPGPKGAAGAPGAPGAKGDAGAPGERGPPAIAGIGGEKAG |
| | GFAPYYG |
| 981 | DSYDVKSGVAVGGLAGYPGPAGPPGPPGPPGTSGHPGSPGSPGYQGPPGE |
| | PGQAGPSGPPGPPGAIGPSGPAGKDGESGRPGRPGERGLPGPPGIKGPAGIP |
| | GFPGMKGHRGFDGRNGEKGETGAPGLKGENGLPGENGAPGPMGPRGAP |
| | GERGRPGLPGAAGARGNDGARGSDGQPGPPGPPGTAGFPGSPGAKGEVG |
| | PAGSPGSNGAPGQRGEPGPQGHAGAQGPPGPPGINGSPGGKGEMGPAGIP |
| | GAPGLMGARGPPGPAGANGAPGLRGGAGEPGKNGAKGEPGPRGERGEA |
| | GIPGVPGAKGEDGKDGSPGEPGANGLPGAAGERGAPGFRGPAGPNGIPGE |
| | KGPAGERGAPGPAGPRGAAGEPGRDGVPGGPGMRGMPGSPGGPGSDGKP |
| | GPPGSQGESGRPGPPGPSGPRGQPGVMGFPGPKGNDGAPGKNGERGGPG |
| | GPGPQGPPGKNGETGPQGPPGPTGPGGDKGDTGPPGPQGLQGLPGTGGPP |
| | GENGKPGEPGPKGDAGAPGAPGGKGDAGAPGERGPPAIAGIGGEKAGGF |
| | APYYG |
| 982 | DVKSGVAVGGLAGYPGPAGPPGPPGPPGTSGHPGSPGSPGYQGPPGEPGQ |
| | AGPSGPPGPPGAIGPSGPAGKDGESGRPGRPGERGLPGPPGIKGPAGIPGFP |
| | GMKGHRGFDGRNGEKGETGAPGLKGENGLPGENGAPGPMGPRGAPGER |
| | GRPGLPGAAGARGNDGARGSDGQPGPPGPPGAIGPSGPAGKDGESGRPGR |
| | PGERGLPGPPGIKGPAGIPGFPGMKGHRGFDGRNGEKGETGAPGLKGENG |
| | LPGENGAPGPMGPRGAPGERGRPGLPGAAGARGNDGARGSDGQPGPPGP |
| | PAIAGIGGEKAGGFAPYYG |
| 983 | DVKSGVAVGGLAGYPGPAGPPGPPGPPGTSGHPGSPGSPGYQGPPGEPGQ |
| | AGPSGPPGPPGAIGPSGPAGKDGESGRPGRPGERGLPGPPGIKGPAGIPGFP |
| | GMKGHRGFDGRNGEKGETGAPGLKGENGLPGENGAPGPMGPRGAPGER |
| | GRPGLPGAAGARGNDGARGSDGQPGPPGPPGAIGPSGPAGKDGESGRPGR |
| | PGERGLPGPPGIKGPAGIPGFPGMKGHRGFDGRNGEKGETGAPGLKGENG |
| | LPGENGAPGPMGPRGAPGERGRPGLPGAAGARGNDGARGSDGQPGPPGP |
| | PAIAGIGGEKAGGFAPYYG |
| 984 | DVKSGVAVGGLAGYPGPAGPPGPPGPPGTSGHPGSPGSPGYQGPPGEPGQ |
| | AGPSGPPGPPGAIGPSGPAGKDGESGRPGRPGERGLPGPPGIKGPAGIPGFP |
| | GMKGHRGFDGRNGEKGETGAPGLKGENGLPGENGAPGPMGPRGAPGER |
| | GRPGLPGAAGARGNDGARGSDGQPGPPGPPGAIGPSGPAGKDGESGRPGR |
| | PGERGLPGPPGIKGPAGIPGFPGMKGHRGFDGRNGEKGETGAPGLKGENG |
| | LPGENGAPGPMGPRGAPGERGRPGLPGAAGARGNDGARGSDGQPGPPGP |
| | PGAIGPSGPAGKDGESGRPGRPGERGLPGPPGIKGPAGIPGFPGMKGHRGF |
| | DGRNGEKGETGAPGLKGENGLPGENGAPGPMGPRGAPGERGRPGLPGAA |
| | GARGNDGARGSDGQPGPPGPPAIAGIGGEKAGGFAPYYG |
| 985 | DVKSGVAVGGLAGYPGPAGPPGPPGPPGTSGHPGSPGSPGYQGPPGEPGQ |
| | AGPSGPPGPPGAIGPSGPAGKDGESGRPGRPGERGLPGPPGIKGPAGIPGFP |
| | GMKGHRGFDGRNGEKGETGAPGLKGENGLPGENGAPGPMGPRGAPGER |
| | GRPGLPGAAGARGNDGARGSDGQPGPPGPPGTAGFPGSPGAKGEVGPAG |
| | SPGSNGAPGQRGEPGPQGHAGAQGPPGPPGINGSPGGKGEMGPAGIPGAP |
| | GLMGARGPPGPAGANGAPGLRGGAGEPGKNGAKGEPGPRGERGEAGIPG |
| | VPGAKGEDGKDGSPGEPGANGLPGAAGERGAPGFRGPAGPNGIPGEKGP |
| | AGERGAPGPAGPRGAAGEPGRDGVPGGPGMRGMPGSPGGPGSDGKPGPP |
| | GSQGESGRPGPPGPSGPRGQPGVMGFPGPKGHRGFPGKNGERGGPGGPGP |
| | QGPPGKNGETGPQGPPGPTGPGGDKGDTGPPGPQGLQGLPGTGGPPGENG |
| | KPGEPGPKGDAGAPGAPGGKGDAGAPGERGPPAIAGIGGEKAGGFAPYY |
| | G |
| 986 | DVKSGVAVGGLAGYPGPAGPPGPPGPPGTSGHPGSPGSPGYQGPPGEPGQ |
| | AGPSGPPGPPGAIGPSGPAGKDGESGRPGRPGAAGLPGPPGIKGPAGIPGFP |
| | GMKGHRGFDGRNGEKGETGAPGLKGENGLPGAAGAPGPMGPRGAPGAA |
| | GRPGLPGAAGARGNDGARGSDGQPGPPGPPGTAGFPGSPGAKGEVGPAG |
| | SPGSNGAPGQRGEPGPQGHAGAQGPPGPPGINGSPGGKGEMGPAGIPGAP |
| | GLMGARGPPGPAGANGAPGLRGGAGEPGKNGAKGEPGPRGERGEAGIPG |
| | VPGAKGEDGKDGSPGEPGANGLPGAAGERGAPGFRGPAGPNGIPGEKGP |
| | AGERGAPGPAGPRGAAGEPGRDGVPGGPGMRGMPGSPGGPGSDGKPGPP |
| | GSQGESGRPGPPGPSGPRGQPGVMGFPGPKGNDGAPGKNGERGGPGGPG |
| | PQGPPGKNGETGPQGPPGPTGPGGDKGDTGPPGPQGLQGLPGTGGPPGEN |
| | GKPGEPGPKGDAGAPGAPGGKGDAGAPGPLVLRQLLVLVEKRPAVSLHI |
| | MVNQEDVRMPFA |
| 987 | DVKSGVAVGGLAGYPGPAGPPGPPGPPGTSGHPGSPGSPGYQGPPGEPGQ |
| | AGPSGPPGPPGAIGPSGPAGKDGESGKPGRPGERGLPGPPGIKGPAGIPGFP |
| | GMKGHRGFDGKNGEKGETGAPGLKGENGLPGENGAPGPMGPKGAPGER |
| | GKPGLPGAAGAKGNDGAKGSDGQPGPPGPPGTAGFPGSPGAKGEVGPAG |
| | SPGSNGAPGQKGEPGPQGHAGAQGPPGPPGINGSPGGKGEMGPAGIPGAP |
| | GLMGAKGPPGPAGANGAPGLKGGAGEPGKNGAKGEPGPKGEKGEAGIPG |
| | VPGAKGEDGKDGSPGEPGANGLPGAAGEKGAPGFKGPAGPNGIPGEKGP |
| | AGEKGAPGPAGPKGAAGEPGKDGVPGGPGMKGMPGSPGGPGSDGKPGPP |
| | GSQGESGKPGPPGPSGPKGQPGVMGFPGPKGNDGAPGKNGEKGGPGGPG |
| | PQGPPGKNGETGPQGPPGPTGPGGDKGDTGPPGPQGLQGLPGTGGPPGEN |
| | GKPGEPGPKGDAGAPGAPGGKGDAGAPGERGPPAIAGIGGEKAGGFAPY |
| | YG |
| 988 | DVKSGVAVGGLAGYPGPAGPPGPPGPPGTSGHPGSPGSPGYQGPPGEPGQ |
| | AGPSGPPGPPGAIGPSGPAGKDGESGRPGRPGERGLPGPPGIKGPAGIPGFP |
| | GMKGHRGFDGRNGEKGETGAPGLKGENGLPGENGAPGPMGPRGAPGER |
| | GRPGLPGAAGARGNDGARGSDGQPGPPGPPGTAGFPGAVGAKGEAGPQG |
| | PRGSEGPQGVRGEPGPPGPAGAAGPAGPPGADGQPGAKGAMGAPGIAGA |
| | PGFPGARGPPGPQGAGGPPGPKGGAGEPGAPGAKGDTGAKGERGPVGVQ |
| | GPPGPAGEEGKRGARGEPGPTGLPGPPGERGGPGFRGFPGADGIPGEKGPA |
| | GERGAPGPAGPRGAAGEPGRDGVPGGPGMRGMPGSPGGPGSDGKPGPPG |
| | SQGESGRPGPPGPSGPRGQPGVMGFPGPKGNDGAPGKNGERGGPGGPGP |
| | QGPPGKNGETGPQGPPGPTGPGGDKGDTGPPGPQGLQGLPGTGGPPGENG |
| | KPGEPGPKGDAGAPGAPGGKGDAGAPGERGPPAIAGIGGEKAGGFAPYY |
| | G |
| 989 | DVKSGVAVGGIAGYPGPAGPPGPPGPPGTSGHPGSPGSPGYQGPPGEPGQ |
| | AGPSGPPGPPGALGPSGPAGKDGESGRPGRPGERGVPGPPGVKGPAGLPG |
| | FPGMKGHRGFDGRNGEKGETGAPGLKGENGLPGENGAPGPMGPRGAPGE |
| | RGRPGLPGAAGARGNDGARGSDGQPGPPGPPGTAGFPGSPGAKGELGPA |
| | GSPGSNGAPGQRGEPGPQGHAGAQGPPGPPGLNGSPGGKGEMGPAGVPG |
| | APGIMGARGPPGPAGANGAPGIRGGAGEPGKNGAKGEPGPRGERGEAGL |
| | PGLPGAKGEDGKDGSPGEPGANGVPGAAGERGAPGFRGPAGPNGLPGEK |
| | GPAGERGAPGPAGPRGAAGEPGRDGIPGGPGMRGMPGSPGGPGSDGKPG |
| | PPGSQGESGRPGPPGPSGPRGQPGLMGFPGPKGNDGAPGKNGERGGPGGP |
| | GPQGPPGKNGETGPQGPPGPTGPGGDKGDTGPPGPQGVQGLPGTGGPPGE |
| | NGKPGEPGPKGDAGAPGAPGGKGDAGAPGERGPPAIAGIGGEKAGGFAP |
| | YYG |
| 990 | DVKSGVAVGGLAGYPGPAGPPGPPGPPGAAGHPGAPGAPGYQGPPGEPG |
| | QAGPAGPPGPPGAIGPAGPAGKDGEAGRPGRPGERGLPGPPGIKGPAGIPG |
| | FPGMKGHRGFDGRDGEKGEAGAPGLKGEDGLPGENGAPGPMGPRGAPG |
| | ERGRPGLPGAAGARGDDGARGADGQPGPPGPPGAAGFPGAPGAKGEVGP |
| | AGAPGADGAPGQRGEPGPQGHAGAQGPPGPPGIDGAPGGKGEMGPAGIP |
| | GAPGLMGARGPPGPAGADGAPGLRGGAGEPGKDGAKGEPGPRGERGEA |
| | GIPGVPGAKGEDGKDGAPGEPGADGLPGAAGERGAPGFRGPAGPDGIPGE |
| | KGPAGERGAPGPAGPRGAAGEPGRDGVPGGPGMRGMPGAPGGPGADGK |
| | PGPPGAQGEAGRPGPPGPAGPRGQPGVMGFPGPKGDDGAPGKDGERGGP |
| | GGPGPQGPPGKDGEAGPQGPPGPAGPGGDKGDAGPPGPQGLQGLPGAGG |
| | PPGEDGKPGEPGPKGDAGAPGAPGGKGDAGAPGERGPPAIAGIGGEKAGG |
| | FAPYYG |
| 991 | DVKSGVAVGGLAGYPGPAGPPGPPGPPGTSGHPGSPGSPGYQGPPGEPGQ |
| | AGPSGPPGPPGAIGPSGPAGKDGESGRPGRPGERGLPGPPGIKGPAGIPGFP |
| | GMKGHRGFDGRNGEKGETGAPGLKGENGLPGENGAPGPMGPRGAPGER |
| | GRPGLPGAAGARGNDGARGSDGQPGPPGPPGTAGFPGSPGAKGEVGPAG |
| | SPGSNGAPGQRGEPGPQGHAGAQGPPGPPGINGSPGGKGEMGPAGIEPGR |
| | PEGEKGGGRGGAPGAPGRGAGNGEGARPPPLMGGAGGNKAGPLAAPIPG |
| | VPGAKGEDGKDGSPGEPGANGLPGAAGERGAPGFRGPAGPNGIPGEKGP |
| | AGERGAPGPAGPRGAAGEPGRDGVPGGPGMRGMPGSPGGPGSDGKPGPP |
| | GSQGESGRPGPPGPSGPRGQPGVMGFPGPKGNDGAPGKNGERGGPGGPG |
| | PQGPPGKNGETGPQGPPGPTGPGGDKGDTGPPGPQGLQGLPGTGGPPGEN |
| | GKPGEPGPKGDAGAPGAPGGKGDAGAPGERGPPAIAGIGGEKAGGFAPY |
| | YG |
| 992 | DVKSGVAVGGLAGYPGPAGPPGPPGPPGTSGHPGSPGSPGYQGPPGEPGQ |
| | AGPSGPPGPPGAIGPSGPAGKDGESGRPGRPGERGLPGPPGIKGPAGIPGFP |
| | GMKGHRGFDGRNGEKGETGAPGLKGENGLPGENGAPGPMGPRGAPGER |
| | GRPGLPGAAGARGNDGARGSDGQPGPPGPPGTAGFPGSPGAKGEVGPAG |
| | SPGSNGAPGQRGEPGPQGHAGAQGPPGPPGINGSPGGKGEMGPAGIEPGR |
| | PEGEKGGGRGGAPGAPGRGAGNGEGARPPPLMGGAGGNKAGPLAAPIPG |
| | VPGAKGEDGKDGSPGEPGANGLPGAAGERGAPGFRGPAGPNGIPGEKGP |
| | AGERGAPGPAGPRGAAGEPGRDGVPGGPGMRGMPGSPGGPGSDGKPGPP |
| | GSQGESGRPGPPGPSGPRGQPGVMGFPGPKGNDGAPGKNGERGGPGGPG |
| | PQGPPGKNGETGPQGPPGPTGPGGDKGDTGPPGPQGLQGLPGTGGPPGEN |
| | GKPGEPGPKGDAGAPGAPGGKGDAGAPGERGPPAIAGIGGEKAGGFAPY |
| | YG |
| 993 | DVKSGVAVGGLAGYPGPAGPPGPPGPPGTSGHPGSPGSPGYQGPPGEPGQ |
| | AGPSGPPGPPGAIGPSGPAGKRGESGRPGRPGERGLPGPPGIKGPAGIPGFP |
| | GMKGHRGFRGRNGEKGRTGAPGLKGENGLPGENGAPGPMGPRGAPGER |
| | GRPGLPGAAGARGNRGARGSRGQPGPPGPPGTAGFPGSPGAKGRVGPAG |
| | SPGSNGAPGQRGRPGPQGHAGAQGPPGPPGINGSPGGKGRMGPAGIPGAP |
| | GLMGARGPPGPAGANGAPGLRGGAGRPGKNGAKGRPGPRGERGRAGIPG |
| | VPGAKGRRGKRGSPGEPGANGLPGAAGERGAPGFRGPAGPNGIPGEKGPA |
| | GERGAPGPAGPRGAAGRPGRRGVPGGPGMRGMPGSPGGPGSRGKPGPPG |
| | SQGESGRPGPPGPSGPRGQPGVMGFPGPKGNRGAPGKNGRRGGPGGPGP |
| | QGPPGKNGETGPQGPPGPTGPGGRKGRTGPPGPQGLQGLPGTGGPPGENG |
| | KPGRPGPKGDAGAPGAPGGKGRAGAPGERGPPAIAGIGGEKAGGFAPYY |
| | G |
| 994 | DVKSGVAVGGMGGYPGPAGPPGPPGPPGVSGHPGAPGAPGYQGPPGEPG |
| | QAGPAGPPGPPGAMGPAGPAGKDGESGRPGRPGERGFPGPPGIKGPAGMP |
| | GFPGMKGHRGFDGRNGEKGDTGAPGLKGENGLPGENGAPGPMGPRGAP |
| | GERGRPGLPGAAGARGNDGARGSDGQPGPPGPPGTAGFPGSPGAKGEVG |
| | PAGAPGPNGAPGQRGEPGLQGQAGAPGPPGPPGINGSPGGKGEMGPAGIP |
| | GAPGLIGARGPPGPPGANGVPGQRGAAGEPGKNGAKGDPGARGERGEAG |
| | IPGIAGPKGEDGKDGSPGEPGANGLPGAPGERGPPGFRGAPGANGIPGEKG |
| | PPGERGGPGPAGPRGVAGEPGRDGLPGGPGLRGIPGSPGGPGSDGKPGPPG |
| | SQGESGRPGPPAHLVQGVSLVSWVSPVQRVMTEHRVKMVNVVAQVVQD |
| | CPVQQEKTVRQVLKDLQDLPVLQEIREMQDRQDLLDCKVCLVQEALPEK |
| | MVSLESQDQKAMSEPQVFPEVRETPVLRVSVVLRQLLVLVEKRPAVSLHI |
| | MVNQEDVRMPFA |
| 995 | DVKSGVAVGGLAGYPGPAGPPGPPGPPGTSGHPGSPGSPGYQGPPGEPGQ |
| | AGPSGPPGPPGAIGPSGPAGKDGESGRPGRPGERGLPGPPGIKGPAGIPGFP |
| | GMKGHRGFDGRNGEKGETGAPGLKGENGLPGENGAPGPMGPRGAPGER |
| | GRPGLPGAAGARGNDGARGSDGQPGPPGPPGTAGFPGSPGAKGEVGPAG |
| | SPGSNGAPGQRGEPGPQGHAGAQGPPGPPGINGSPGGKGEMGPAGIPGAP |
| | GLMGARGPPGPAGANGAPGLRGGAGEPGKNGAKGEPGPRGERGEAGIPG |
| | VPGAKGEDGKDGSPGEPGANGLPGAAGERGAPGFRGPAGPNGVAGPKGP |
| | AGERGAPGPAGPKGAAGEAGRPGEAGLPGAKGMPGSPGGPGPDGKPGPP |
| | GPAGQDGRPGPPGPPGARGQAGVMGFPGPKGAAGEPGKAGERGVPGPPG |
| | AVGPAGKDGEAGAQGPPGPAGPAGERGEQGPAGPQGFQGLPGPAGPPGE |
| | AGKPGEQGVPGDLGAPGAPGARGERGAPGERGPPAIAGIGGEKAGGFAPY |
| | YG |
| 996 | DSYDVKSGVAVGGLAGYPGPAGPPGPPGPPGTSGHPGSPGSPGYQGPPGE |
| | PGQAGPSGPPGPPGAIGPSGPAGKDGESGRPGRPGERGLPGPPGIKGPAGIP |
| | GFPGMKGHRGFDGRNGEKGETGAPGLKGENGLPGENGAPGPMGPRGAP |
| | GERGRPGLPGAAGARGNDGARGSDGQPGPPGPPGTAGFPGSPGAKGEVG |
| | PAGSPGSNGAPGQRGEPGPQGHAGAQGPPGPPGINGSPGGKGEMGPAGIP |
| | GAPGLMGARGPPGPAGANGAPGLRGGAGEPGKNGAKGEPGPRGERGEA |
| | GIPGVPGAKGEDGKDGSPGEPGANGLPGAAGERGAPGFRGPAGPNGIPGE |
| | KGPAGERGAPGPAGPRGAAGEPGRDGVPGGPGMRGMPGSPGGPGSDGKP |
| | GPPGSQGESGRPGPPGPSGPRGQPGVMGFPGPKGNDGAPGKNGERGGPG |
| | GPGPQGPPGKNGETGPQGPPGPTGPGGDKGDTGPPGPQGLQGLPGTGGPP |
| | GENGKPGEPGPKGDAGAPGAPGGKGDAGAPGERGPP |
| 997 | DVKSGVAVGKTTKSGLAGYPGPAGPPGPPGPPGTSGHPGSPGSPGYQGPP |
| | GEPGQAGPSGPPGPPGAIGPSGPAGKDGESGRPGRPGERGLPGPPGIKGPA |
| | GIPGFPGMKGHRGFDGRNGEKGETGAPGLKGENGLPGENGAPGPMGPRG |
| | APGERGRPGLPGAAGARGNDGARGSDGQPGPPGPPGTAGFPGSPGAKGE |
| | VGPAGSPGSNGAPGQRGEPGPQGHAGAQGPPGPPGINGSPGGKGEMGPA |
| | GIPGAPGLMGARGPPGPAGANGAPGLRGGAGEPGKNGAKGEPGPRGERG |
| | EAGIPGVPGAKGEDGKDGSPGEPGANGLPGAAGERGAPGFRGPAGPNGIP |
| | GEKGPAGERGAPGPAGPRGAAGEPGRDGVPGGPGMRGMPGSPGGPGSDG |
| | KPGPPGSQGESGRPGPPGPSGPRGQPGVMGFPGPKGNDGAPGKNGERGGP |
| | GGPGPQGPPGKNGETGPQGPPGPTGPGGDKGDTGPPGPQGLQGLPGTGGP |
| | PGENGKPGEPGPKGDAGAPGAPGGKGDAGAPGERGPPKTTKSAIAGIGGE |
| | KAGGFAPYYG |
| 998 | DVKSGVAVGKTTKSGLAGYPGPAGPPGPPGPPGTSGHPGSPGSPGYQGPP |
| | GEPGQAGPSGPPGPPGAIGPSGPAGKDGESGRPGRPGERGLPGPPGIKGPA |
| | GIPGFPGMKGHRGFDGRNGEKGETGAPGLKGENGLPGENGAPGPMGPRG |
| | APGERGRPGLPGAAGARGNDGARGSDGQPGPPGPPGTAGFPGSPGAKGE |
| | VGPAGSPGSNGAPGQRGEPGPQGHAGAQGPPGPPGINGSPGGKGEMGPA |
| | GIPGAPGLMGARGPPGPAGANGAPGLRGGAGEPGKNGAKGEPGPRGERG |
| | EAGIPGVPGAKGEDGKDGSPGEPGANGLPGAAGERGAPGFRGPAGPNGIP |
| | GEKGPAGERGAPGPAGPRGAAGEPGRDGVPGGPGMRGMPGSPGGPGSDG |
| | KPGPPGSQGESGRPGPPGPSGPRGQPGVMGFPGPKGNDGAPGKNGERGGP |
| | GGPGPQGPPGKNGETGPQGPPGPTGPGGDKGDTGPPGPQGLQGLPGTGGP |
| | PGENGKPGEPGPKGDAGAPGAPGGKGDAGAPGERGPPKTTKSAIAGIGGE |
| | KAGGFAPYYG |
| 999 | DVKSGVAVGKTTKSGLAGYPGPAGPPGPPGPPGTSGHPGSPGSPGYQGPP |
| | GEPGQAGPSGPPGPPGAIGPSGPAGKDGESGRPGRPGAAGLPGPPGIKGPA |
| | GIPGFPGMKGHRGFDGRNGEKGETGAPGLKGENGLPGAAGAPGPMGPRG |
| | APGAAGRPGLPGAAGARGNDGARGSDGQPGPPGPPGTAGFPGSPGAKGE |
| | VGPAGSPGSNGAPGQRGEPGPQGHAGAQGPPGPPGINGSPGGKGEMGPA |
| | GIPGAPGLMGARGPPGPAGANGAPGLRGGAGEPGKNGAKGEPGPRGERG |
| | EAGIPGVPGAKGEDGKDGSPGEPGANGLPGAAGERGAPGFRGPAGPNGIP |
| | GEKGPAGERGAPGPAGPRGAAGEPGRDGVPGGPGMRGMPGSPGGPGSDG |
| | KPGPPGSQGESGRPGPPGPSGPRGQPGVMGFPGPKGNDGAPGKNGERGGP |
| | GGPGPQGPPGKNGETGPQGPPGPTGPGGDKGDTGPPGPQGLQGLPGTGGP |
| | PGENGKPGEPGPKGDAGAPGAPGGKGDAGAPGAAGPPKTTKSAIAGIGGE |
| | KAGGFAPYYG |
| 1000 | DVKSGVAVGGLAGYPGPAGPPGPPGPPGTSGHPGSPGSPGYQGPPGEPGQ |
| | AGPSGPPGPPGAIGPSGPAGKDGESGRPGRPGERGLPGPPGIKGPAGIPGFP |
| | GMKGHRGFDGRNGEKGETGAPGLKGENGLPGENGAPGPMGPRGAPGER |
| | GRPGLPGAAGARGNDGARGSDGQPGPPGPPGTAGFPGSPGAKGEVGPAG |
| | SPGSNGAPGQRGEPGPQGHAGAQGPPGPPGINGSPGGKGEMGPAGIPGAP |
| | GLMGARGPPGPAGANGAPGLRGGAGEPGKNGAKGEPGPRGERGEAGIPG |
| | VPGAKGEEGKRGARGEAGSAGPPGPPGLRGAPGFRGLPGADGRAGVMGP |
| | PGERGAPGPAGVRGAAGDAGRPGEPGLMGPRGLPGSPGGPGPAGKEGPV |
| | GLPGIDGRPGPIGPAGARGEPGVMGFPGPKGNDGDPGKNGDKGHAGLAG |
| | ARGAPGPDGETGPQGPPGPTGPGGDKGDTGPPGPQGLQGLPGTGGPPGEN |
| | GKPGEPGPKGDAGAPGAPGGKGDAGAPGERGPPAIAGIGGEKAGGFAPY |
| | YG |
| 1001 | DVKSGVAVGGMGGYPGPAGPPGPPGPPGVSGHPGAPGAPGYQGPPGEPG |
| | QAGPAGPPGPPGAMGPAGPAGKDGESGRPGRPGERGFPGPPGIKGPAGMP |
| | GFPGMKGHRGFDGRNGEKGDTGAPGLKGENGLPGENGAPGPMGPRGAP |
| | GERGRPGLPGAAGARGNDGARGSDGQPGPPGPPGTAGFPGSPGAKGEVG |
| | PAGAPGPNGAPGQRGEPGLQGQAGAPGPPGPPGINGSPGGKGEMGPAGIP |
| | GAPGLIGARGPPGPPGANGVPGQRGAAGEPGKNGAKGDPGARGERGEAG |
| | IPGIAGPKGEDGKDGSPGEPGANGLPGAPGERGPPGFRGAPGANGIPGEKG |
| | PPGERGGPGPAGPRGVAGEPGRDGLPGGPGLRGIPGSPGGPGSDGKPGPPG |
| | SQGESGRPGPPGSPGPRGQPGVMGFPGPKGNDGAPGKNGERGGPGGPGLP |
| | GPAGKNGETGPQGPPGPTGPAGDKGDAGPPGPPGLQGLPGTGGPPGENGK |
| | PGEPGPKGDVGAPGIPGGKGDAGAPGERGPPAIAGIGGEKAGGFAPYYG |
| 1002 | DVKSGVAVGGLAGYPGPAGPPGPPGPPGTSGHPGSPGSPGYQGPPGEPGQ |
| | AGPAGPPGPIGNVGPSGPAGKDGESGRPGRPGERGLPGPPGIKGPAGIPGFP |
| | GMKGHRGFDGRNGEKGETGAPGLKGENGLPGENGAPGPMGPRGAPGER |
| | GRPGLPGAAGARGNDGARGSDGQPGPPGPPGTAGFPGSPGAKGEVGPAG |
| | SPGSNGAPGQRGEPGPQGHAGAQGPPGPPGINGSPGGKGEMGPAGIPGAP |
| | GLMGARGPPGPAGANGAPGLRGGAGEPGKNGAKGEPGPRGERGEAGIPG |
| | VPGAKGEDGKDGSPGEPGANGLPGAAGERGAPGFRGPAGPNGIPGEKGP |
| | AGERGAPGPAGPRGAAGEPGRDGVPGGPGMRGMPGSPGGPGSDGKPGPP |
| | GSQGESGRPGPPGPSGPRGQPGVMGFPGPKGNDGAPGKNGERGGPGGPG |
| | PQGPPGKNGETGPQGPPGPTGPGGDKGDTGPPGPQGLQGLPGTGGPPGEN |
| | GKPGEPGPKGDAGAPGAPGGKGDAGAPGERGPPAIAGIGGEKAGGFAPY |
| | YG |
| 1007 | DVKSGVAVGGLAGYPGPAGPPGPPGPPGTSGHKGSPGSPGYQGPPGEPGQ |
| | AGPAGPPGPIGNVGPSGPAGKDGESGRPGRPGERGLPGPPGIKGPAGIPGFP |
| | GMKGHRGFDGRNGEKGETGAPGLKGENGLPGENGAPGPMGPRGAPGER |
| | GRPGLPGAAGARGNDGARGSDGQPRPPGPPGTAGFPGSPGAKGEVGPAG |
| | SPGSNGAPGQRGEPGPQGHKGAQGPPGPPGINGSPGGKGEMGPAGIPGAP |
| | GLMGARGPPGPAGANGAPGLRGGAGEPGKNGAKGEPGPRGERGEAGIPG |
| | VPGAKGEDGKDGSPGEPGANGLPGAAGERGAPGFRGPAGPNGIPGEKGP |
| | AGERGAPGPAGPRGAAGEPGRDGVPGGPGMRGMPGSPGGPGSDGKPGPP |
| | GSQGESGRPGPPGPSGPRGQPGVMGFPGPKGNDGAPGKNGERGGPGGPG |
| | PQGPPGKNGETGPQGPPGPTGPGGDKGDTGPPGPQGLQGLPGTGGPPGEN |
| | GKPGEPGPKGDAGAPGAPGGKGDAGAPGERGPPAIAGIGGEKAGGFAPY |
| | YG |
| 1008 | DSYDVKSGVAVGGLAGAPGLRGGAGPPGPEGGKGAAGPPGPPGAAGTPG |
| | LQGMPGERGGLGSPGPKGDKGEPGGPGADGVPGKDGPRGPTGPIGPPGPA |
| | GQPGDKGEGGAPGLPGIAGPRGSPGERGETGPPGPAGFPGAPGQNGEPGG |
| | KGERGAPGEKGEGGPPGVAGPPGGSGPAGPPGPQGVKGERGSPGGPGAA |
| | GFPGARGLPGPPGSNGNPGPPGPSGSPGKDGPPGPAGNTGAPGSPGVSGPK |
| | GDAGQPGEKGSPGAQGPPGAPGPLGIAGITGARGLAGPPGMPGPRGSPGP |
| | QGVKGESGKPGANGLSGERGPPGPQGLPGLAGTAGEPGRDGNPGSDGLP |
| | GRDGSPGGKGDRGENGSPGAPGAPGHPGPPGPVGPAGKSGDRGESGPAG |
| | PAGAPGPAGSRGAPGPQGPRGDKGETGERGAAGIKGHRGFPGNPGAPGSP |
| | GPAGQQGAIGSPGPAGPRGPVGPSGPPGKDGTSGHPGPIGPPGPRGNRGER |
| | GSEGSPGHPGQPGPPGPPGAPGPCCGGVGAAAIAGIGGEKAGGFAPYYG |
| 1009 | MMSFVQKGTWLLFALLHPTVILAQQEAVDGGCSHLGQSYADRDVWKPE |
| | PCQICVCDSGSVLCDDIICDDQELDCPNPEIPFGECCAVCPQPPTAPTRPPN |
| | GQGPQGPKGDPGPPGIPGRNGDPGPPGSPGSPGSPGPPGICESCPTGGQNYS |
| | PQYEAYDVKSGVAGGGIAGYPGPAGPPGPPGPPGTSGHPGAPGAPGYQGP |
| | PGEPGQAGPAGPPGPPGAIGPSGPAGKDGESGRPGRPGERGFPGPPGMKGP |
| | AGMPGFPGMKGHRGFDGRNGEKGETGAPGLKGENGVPGENGAPGPMGP |
| | RGAPGERGRPGLPGAAGARGNDGARGSDGQPGPPGPPGTAGFPGSPGAK |
| | GEVGPAGSPGSSGAPGQRGEPGPQGHAGAPGPPGPPGSNGSPGGKGEMGP |
| | AGIPGAPGLIGARGPPGPPGTNGVPGQRGAAGEPGKNGAKGDPGPRGERG |
| | EAGSPGIAGPKGEDGKDGSPGEPGANGLPGAAGERGVPGFRGPAGANGLP |
| | GEKGPPGDRGGPGPAGPRGVAGEPGRDGLPGGPGLRGIPGSPGGPGSDGK |
| | PGPPGSQGETGRPGPPGSPGPRGQPGVMGFPGPKGNDGAPGKNGERGGPG |
| | GPGPQGPAGKNGETGPQGPPGPTGPSGDKGDTGPPGPQGLQGLPGTSGPP |
| | GENGKPGEPGPKGEAGAPGIPGGKGDSGAPGERGPPGAGGPPGPRGGAGP |
| | PGPEGGKGAAGPPGPPGSAGTPGLQGMPGERGGPGGPGPKGDKGEPGSSG |
| | VDGAPGKDGPRGPTGPIGPPGPAGQPGDKGESGAPGVPGIAGPRGGPGER |
| | GEQGPPGPAGFPGAPGQNGEPGAKGERGAPGEKGEGGPPGAAGPAGGSG |
| | PAGPPGPQGVKGERGSPGGPGAAGFPGGRGPPGPPGSNGNPGPPGSSGAP |
| | GKDGPPGPPGSNGAPGSPGISGPKGDSGPPGERGAPGPQGPPGAPGPLGIA |
| | GLTGARGLAGPPGMPGARGSPGPQGIKGENGKPGPSGQNGERGPPGPQGL |
| | PGLAGTAGEPGRDGNPGSDGLPGRDGAPGAKGDRGENGSPGAPGAPGHP |
| | GPPGPVGPAGKSGDRGETGPAGPSGAPGPAGSRGPPGPQGPRGDKGETGE |
| | RGAMGIKGHRGFPGNPGAPGSPGPAGHQGAVGSPGPAGPRGPVGPSGPPG |
| | KDGASGHPGPIGPPGPRGNRGERGSEGSPGHPGQPGPPGPPGAPGPCCGAG |
| | GVAAIAGVGAEKAGGFAPYYGDEPIDFKINTDEIMTSLKSVNGQIESLISPD |
| | GSRKNPARNCRDLKFCHPELQSGEYWVDPNQGCKLDAIKVYCNMETGET |
| | CISASPLTIPQKNWWTDSGAEKKHVWFGESMEGGFQFSYGNPELPEDVLD |
| | VQLAFLRLLSSRASQNITYHCKNSIAYMDHASGNVKKALKLMGSNEGEFK |
| | AEGNSKFTYTVLEDGCTKHTGEWGKTVFQYQTRKAVRLPIVDIAPYDIGG |
| | PDQEFGADIGPVCFL |

As noted above herein, it is well known in the art that cancer cells disseminate and seed in distant organs, and they can remain dormant for many years before forming clinically detectable metastases. Additionally, disseminated tumor cells can sense and remodel the extracellular matrix (ECM) to sustain dormancy. Dormant cancer cells assemble a type III collagen-enriched ECM niche. Without wishing to be bound by any particular theory, it is thought that enriching type III collagen in the ECM can limit or prevent metastasis through disseminated tumor cell dormancy induction.

In some embodiments of the methods of treating cancer disclosed herein, contacting a cellular surface in a subject with cancer with the recombinant collagen fragment, induces cellular dormancy. In some embodiments, the cancer is characterized by the presence of a solid tumor. In some embodiments, the cancer can be a cancer of the skin, colon, oral mucosa, rectum, esophagus, thyroid, liver, pancreas, kidney, bladder, lung, brain, breast, ovary, testicle, prostate, bones, or a combination of any of the foregoing.

In some embodiments, the subject has not undergone resection of a tumor. In some embodiments, the subject has undergone resection of a primary tumor. In some embodiments, the cellular surface of the subject comprises residual cancer cells following the resection of the primary tumor.

In some embodiments of the methods of treating cancer disclosed herein, contacting a cellular surface in a subject with cancer with the fragment maintains cellular dormancy.

In some embodiments, this disclosure provides a method of inhibiting proliferation of a cancer cell, the method comprising contacting the cancer cell with a recombinant collagen fragment having a sequence identity of at least about 85% to the amino acid sequence set forth in SEQ ID NOs: 1 and/or 973. Where this specification use the phrase “1 and/or 973,” it is to be understood that any percent identity that precedes this phrase means that the collagen sequence contemplated can have the noted percent identity to either sequence individually or both sequences (i.e. as in a mixture). In some embodiments, the collagen fragment can have at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity, or similarity to SEQ ID NOs: 1 and/or 973. In some embodiments, this disclosure provides a method of inhibiting proliferation of a cancer cell, the method comprising contacting the cancer cell with a recombinant collagen fragment having a sequence identity of at least about 85% to the amino acid sequence set forth in SEQ ID NOs: 1 and/or 973. In some embodiments, the collagen fragment can have at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% sequence identity, or similarity to SEQ ID NO: 1, wherein the recombinant collagen fragment has the amino acid sequence set forth in any one of SEQ ID NOs: 974-1002.

In some embodiments, the recombinant collagen fragment can be unhydroxylated. In some embodiments, the recombinant collagen fragment can be hydroxylated.

In some embodiments, this disclosure provides a method of inhibiting proliferation of a cancer cell, the method comprising contacting the cancer cell with a recombinant collagen fragment having the amino acid sequence set forth in any one of SEQ ID NOs: 2-972.

In some embodiments, the methods of inhibiting proliferation of a cancer cell provided herein, do not comprise contacting the cancer cell with a recombinant collagen having the amino acid sequence set forth in SEQ ID NO: 973 in the absence of a pharmaceutically acceptable scaffold.

In some embodiments, the fragment is formulated in a pharmaceutically acceptable composition. In some embodiments, the pharmaceutically acceptable composition comprises least one pharmaceutically acceptable excipient. In particular embodiments, the composition can comprise a recombinant collagen fragment according to SEQ ID NOs: 1 and/or 973 and at least one pharmaceutically acceptable excipient. In still further embodiments, the composition can comprise a recombinant collagen fragment having about 85%, about 86%, about 87%, about 88%, about 89%, about 90%, about 91%, about 92%, about 93%, about 94%, about 95%, about 96%, about 97%, about 98%, about 99%, or 100% identity, or similarity, to SEQ ID NO: 1 or to SEQ ID NO: 973, wherein the recombinant collagen fragment has the amino acid sequence set forth in any one of SEQ ID NOs: 974-1002 and at least one pharmaceutically acceptable excipient. In some embodiments, the composition can comprise a recombinant collagen fragment having the amino acid sequence of any one of SEQ ID NOs: 2-972, and at least one pharmaceutically acceptable excipient.

In another embodiment, the composition can comprise a recombinant collagen fragment according to SEQ ID NOs: 1 and/or 973 and a plurality of hydrolysis products having such sequences that can be the same or different, according to any one of SEQ ID NOs: 2-972, and at least one pharmaceutically acceptable excipient. In certain embodiments, the number of hydrolysis products present in the plurality of hydrolysis products in the composition can increase with time, with temperature, pH, or as a result of other conditions that would typically cause a recombinant collagen fragment, such as a recombinant collagen fragment according to SEQ ID NO: 1 or 973 to hydrolyze or otherwise break down. In other embodiments, the composition can be stabilized with one or more stabilizers so that the concentrations of the recombinant collagen fragment according to SEQ ID NO: 1 and/or 973 and the concentrations each of the fragments in the plurality of fragments, remain substantially constant (i.e. vary by no more than +5% by HPLC over a given time period) or remain constant. In certain embodiments, the recombinant collagen fragment, such as a recombinant collagen fragment according to SEQ ID NO: 1 can be hydrolyzed, such that less than about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90%, or from about 90% to about 100% of non-hydrolyzed recombinant fragment remains in the composition as measured by HPLC. In other embodiments, the composition can comprise a mixture of a recombinant collagen fragment (e.g., a recombinant collagen fragment according to SEQ ID NO: 1) and a plurality of hydrolyzed products of that recombinant collagen fragment (e.g., a plurality of collagen fragments according to any of SEQ ID NOs: 2-972), such that the weight of the hydrolyzed products in the composition is less than about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90%, or from about 90% to about 100% of the weight of the collagen-related protein in the composition.

Pharmaceutically acceptable compositions for use in the methods disclosed herein have the desired degree of purity in a physiologically acceptable carrier, excipient or stabilizer (Remington's Pharmaceutical Sciences (1990) Mack Publishing Co., Easton, PA). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

In some embodiments, topical mixtures comprising a recombinant collagen fragment can be prepared for local administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, bandages, dermal patches or any other formulations suitable for topical administration. The topical mixtures described herein can be formulated for topical application to the skin and mucous membranes in the form of gels, creams, and lotions for transdermal delivery.

Transdermal patches, including iontophoretic and electrophoretic devices, are well known to those of skill in the art, and can be used to administer a recombinant collagen fragment. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957, each of which is herein incorporated by reference in its entirety.

### B. Therapeutic Biomaterial

In some embodiments of the methods disclosed herein, the present disclosure provides a therapeutic biomaterial comprising a recombinant collagen fragment having a sequence identity of at least about 85% to the amino acid sequence set forth in SEQ ID NO: 1 and a pharmaceutically acceptable scaffold. In some embodiments of the methods disclosed herein, the present disclosure provides a therapeutic biomaterial comprising a recombinant collagen fragment having a sequence comprising the amino acid sequence set forth in any one of SEQ ID NOs: 2-972, and a pharmaceutically acceptable scaffold. In some embodiments of the methods disclosed herein, the present disclosure provides a therapeutic biomaterial comprising a recombinant collagen fragment having a sequence identity of at least about 85% to the amino acid sequence set forth in SEQ ID NO: 1, wherein the recombinant collagen fragment has the amino acid sequence set forth in any one of SEQ ID NOs: 974-1002, and a pharmaceutically acceptable scaffold.

As used herein, a pharmaceutically acceptable scaffold can comprise a synthetic, polymeric, non-immunogenic, three-dimensional, scaffold. Suitable scaffolds can be biocompatible and biodegradable support structures, and can be a solid, a liquid, or a gel scaffold, such as an injectable gel scaffold. Suitable scaffolds can also include those made from nanomaterials, such as nanopolymers and nanofibers. Such a scaffold can be placed upon a cellular surface in a subject, for example, a human subject, after injury or surgery to the cellular surface. Suitable scaffolds include any conventionally used and/or available scaffold for surgery, wound healing and/or tissue repair and/or engineering. For example, some smart thermo-responsive polymers like chitosan, polyvinylpyrrolidone, alginate, and poly(ε-caprolactone) can be used to create biocompatible and biodegradable scaffolds. These processed thermo-responsive biomaterials possess 3D architectures similar to human structures, and can be used as active agent delivery systems.

Additional polymers that are suitable for use in the pharmaceutically acceptable scaffold described herein include but are not limited to: biphasic crosslinked hyaluronic acid comprising crosslinked HA and uncrosslinked HA, polyvinylpyrrolidone, polyacrylamide, poly(ethylene oxide), poly(2-oxazoline)s and protein, polyethylenimine and protein, carboxymethylcellulose, chondroitin sulfate, acetylated hyaluronate, and zinc hyaluronate.

Accordingly, in some embodiments of the methods disclosed herein, the present disclosure provides a therapeutic biomaterial comprising a recombinant collagen fragment having a sequence identity of at least about 85% to the amino acid sequence set forth in SEQ ID NO: 1 and a biphasic crosslinked hyaluronic acid comprising crosslinked HA and uncrosslinked HA. In some embodiments of the methods disclosed herein, the present disclosure provides a therapeutic biomaterial comprising a recombinant collagen fragment having a sequence comprising the amino acid sequence set forth in any one of SEQ ID NOs: 2-972, and a biphasic crosslinked hyaluronic acid comprising crosslinked HA and uncrosslinked HA. In some embodiments of the methods disclosed herein, the present disclosure provides a therapeutic biomaterial comprising a recombinant collagen fragment having a sequence identity of at least about 85% to the amino acid sequence set forth in SEQ ID NO: 1, wherein the recombinant collagen fragment has the amino acid sequence set forth in any one of SEQ ID NOs: 974-1002 and a biphasic crosslinked hyaluronic acid comprising crosslinked HA and uncrosslinked HA.

Accordingly, in some embodiments of the methods disclosed herein, the present disclosure provides a therapeutic biomaterial comprising a recombinant collagen fragment having a sequence identity of at least about 85% to the amino acid sequence set forth in SEQ ID NO: 1 and polyvinylpyrrolidone. In some embodiments of the methods disclosed herein, the present disclosure provides a therapeutic biomaterial comprising a recombinant collagen fragment having a sequence comprising the amino acid sequence set forth in any one of SEQ ID NOs: 2-972, and polyvinylpyrrolidone. In some embodiments of the methods disclosed herein, the present disclosure provides a therapeutic biomaterial comprising a recombinant collagen fragment having a sequence identity of at least about 85% to the amino acid sequence set forth in SEQ ID NO: 1, wherein the recombinant collagen fragment has the amino acid sequence set forth in any one of SEQ ID NOs: 974-1002 and a polyvinylpyrrolidone.

In some embodiments of the methods disclosed herein, the present disclosure provides a therapeutic biomaterial comprising a recombinant collagen fragment having a sequence identity of at least about 85% to the amino acid sequence set forth in SEQ ID NO: 1 and polyacrylamide. In some embodiments of the methods disclosed herein, the present disclosure provides a therapeutic biomaterial comprising a recombinant collagen fragment having a sequence comprising the amino acid sequence set forth in any one of SEQ ID NOs: 2-972, and polyacrylamide. In some embodiments of the methods disclosed herein, the present disclosure provides a therapeutic biomaterial comprising a recombinant collagen fragment having a sequence identity of at least about 85% to the amino acid sequence set forth in SEQ ID NO: 1, wherein the recombinant collagen fragment has the amino acid sequence set forth in any one of SEQ ID NOs: 974-1002 and polyacrylamide.

In some embodiments of the methods disclosed herein, the present disclosure provides a therapeutic biomaterial comprising a recombinant collagen fragment having a sequence identity of at least about 85% to the amino acid sequence set forth in SEQ ID NO: 1 and poly(ethylene oxide). In some embodiments of the methods disclosed herein, the present disclosure provides a therapeutic biomaterial comprising a recombinant collagen fragment having a sequence comprising the amino acid sequence set forth in any one of SEQ ID NOs: 2-972, and poly(ethylene oxide). In some embodiments of the methods disclosed herein, the present disclosure provides a therapeutic biomaterial comprising a recombinant collagen fragment having a sequence identity of at least about 85% to the amino acid sequence set forth in SEQ ID NO: 1, wherein the recombinant collagen fragment has the amino acid sequence set forth in any one of SEQ ID NOs: 974-1002 and poly(ethylene oxide).

In some embodiments of the methods disclosed herein, the present disclosure provides a therapeutic biomaterial comprising a recombinant collagen fragment having a sequence identity of at least about 85% to the amino acid sequence set forth in SEQ ID NO: 1 and poly(2-oxazoline)s. In some embodiments of the methods disclosed herein, the present disclosure provides a therapeutic biomaterial comprising a recombinant collagen fragment having a sequence comprising the amino acid sequence set forth in any one of SEQ ID NOs: 2-972, and poly(2-oxazoline)s. In some embodiments of the methods disclosed herein, the present disclosure provides a therapeutic biomaterial comprising a recombinant collagen fragment having a sequence identity of at least about 85% to the amino acid sequence set forth in SEQ ID NO: 1, wherein the recombinant collagen fragment has the amino acid sequence set forth in any one of SEQ ID NOs: 974-1002 and poly(2-oxazoline)s.

In some embodiments of the methods disclosed herein, the present disclosure provides a therapeutic biomaterial comprising a recombinant collagen fragment having a sequence identity of at least about 85% to the amino acid sequence set forth in SEQ ID NO: 1 and polyethylenimine. In some embodiments of the methods disclosed herein, the present disclosure provides a therapeutic biomaterial comprising a recombinant collagen fragment having a sequence comprising the amino acid sequence set forth in any one of SEQ ID NOs: 2-972, and polyethylenimine. In some embodiments of the methods disclosed herein, the present disclosure provides a therapeutic biomaterial comprising a recombinant collagen fragment having a sequence identity of at least about 85% to the amino acid sequence set forth in SEQ ID NO: 1, wherein the recombinant collagen fragment has the amino acid sequence set forth in any one of SEQ ID NOs: 974-1002 and polyethylenimine.

In some embodiments of the methods disclosed herein, the present disclosure provides a therapeutic biomaterial comprising a recombinant collagen fragment having a sequence identity of at least about 85% to the amino acid sequence set forth in SEQ ID NO: 1 and carboxymethylcellulose. In some embodiments of the methods disclosed herein, the present disclosure provides a therapeutic biomaterial comprising a recombinant collagen fragment having a sequence comprising the amino acid sequence set forth in any one of SEQ ID NOs: 2-972, and carboxymethylcellulose In some embodiments of the methods disclosed herein, the present disclosure provides a therapeutic biomaterial comprising a recombinant collagen fragment having a sequence identity of at least about 85% to the amino acid sequence set forth in SEQ ID NO: 1, wherein the recombinant collagen fragment has the amino acid sequence set forth in any one of SEQ ID NOs: 974-1002 and carboxymethylcellulose.

In some embodiments of the methods disclosed herein, the present disclosure provides a therapeutic biomaterial comprising a recombinant collagen fragment having a sequence identity of at least about 85% to the amino acid sequence set forth in SEQ ID NO: 1 and chondroitin sulfate. In some embodiments of the methods disclosed herein, the present disclosure provides a therapeutic biomaterial comprising a recombinant collagen fragment having a sequence comprising the amino acid sequence set forth in any one of SEQ ID NOs: 2-972, and chondroitin sulfate. In some embodiments of the methods disclosed herein, the present disclosure provides a therapeutic biomaterial comprising a recombinant collagen fragment having a sequence identity of at least about 85% to the amino acid sequence set forth in SEQ ID NO: 1, wherein the recombinant collagen fragment has the amino acid sequence set forth in any one of SEQ ID NOs: 974-1002 and chondroitin sulfate.

In some embodiments of the methods disclosed herein, the present disclosure provides a therapeutic biomaterial comprising a recombinant collagen fragment having a sequence identity of at least about 85% to the amino acid sequence set forth in SEQ ID NO: 1 and acetylated hyaluronate. In some embodiments of the methods disclosed herein, the present disclosure provides a therapeutic biomaterial comprising a recombinant collagen fragment having a sequence comprising the amino acid sequence set forth in any one of SEQ ID NOs: 2-972, and acetylated hyaluronate. In some embodiments of the methods disclosed herein, the present disclosure provides a therapeutic biomaterial comprising a recombinant collagen fragment having a sequence identity of at least about 85% to the amino acid sequence set forth in SEQ ID NO: 1, wherein the recombinant collagen fragment has the amino acid sequence set forth in any one of SEQ ID NOs: 974-1002 and acetylated hyaluronate.

In some embodiments of the methods disclosed herein, the present disclosure provides a therapeutic biomaterial comprising a recombinant collagen fragment having a sequence identity of at least about 85% to the amino acid sequence set forth in SEQ ID NO: 1 and zinc hyaluronate. In some embodiments of the methods disclosed herein, the present disclosure provides a therapeutic biomaterial comprising a recombinant collagen fragment having a sequence comprising the amino acid sequence set forth in any one of SEQ ID NOs: 2-972, and zinc hyaluronate. In some embodiments of the methods disclosed herein, the present disclosure provides a therapeutic biomaterial comprising a recombinant collagen fragment having a sequence identity of at least about 85% to the amino acid sequence set forth in SEQ ID NO: 1, wherein the recombinant collagen fragment has the amino acid sequence set forth in any one of SEQ ID NOs: 974-1002 and zinc hyaluronate.

In some embodiments, the polydispersity of the collagen fragments disclosed herein can be about 1. In some embodiments the polydispersity of the collagen fragments disclosed herein can range from about 1 to about 2. In some embodiments the polydispersity of the collagen fragments disclosed herein can range from about 1 to about 3 or from about 1 to about 5.

In some embodiments the pharmaceutically acceptable scaffold can be for use in the treatment of a cancer in a subject in need thereof. In some embodiments, the cancer is characterized by the presence of a solid tumor. In some embodiments, the cancer can be a cancer of the skin, colon, oral mucosa, rectum, esophagus, thyroid, liver, pancreas, kidney, bladder, lung, brain, breast, ovary, testicle, prostate, bones, or a combination of any of the foregoing. In some embodiments, the biomaterial can be adhered directly onto the cancer cells.

In some embodiments of the methods described herein, the biomaterial can comprise a recombinant collagen fragment that can be unhydroxylated. In some embodiments of the methods described herein, the biomaterial can comprise a recombinant collagen fragment that can be hydroxylated.

In some embodiments, the biomaterial can be for use in treating a wound resulting from resection of a primary tumor. In some embodiments, the biomaterial can be adhered directly onto the wound. In some embodiments, the biomaterial can be for use in maintaining cellular dormancy after resection of a primary tumor. In some embodiments, the biomaterial can be for use in maintaining cellular dormancy in a subject that has not undergone resection of a primary tumor.

In some embodiments, the biomaterial can be adhered directly onto the cells. Without wishing to be bound by a particular theory, the placement of the therapeutic biomaterial comprising a recombinant collagen fragment directly to the wound or cellular surface, is thought to enrich type III collagen in the ECM, and to limit or prevent metastasis through disseminated tumor cell dormancy induction.

In some embodiments, the therapeutic biomaterial can be a protein polyurethane alloy. In some embodiments, the protein can be any of the collagen fragments described herein (or a combination thereof) and the fragment(s) migrate out of the alloy to contact a cellular surface when the alloy is exposed to water, a buffer solution, and/or to a cellular surface.

In some embodiments, the alloy can comprise about 10 wt % to about 50 wt % of the collagen fragment(s) and about 50 wt % to about 90 wt % of the polyurethane. In some embodiments, the alloy can comprise about 20 wt % to about 35 wt % of the protein and about 65 wt % to about 80 wt % of the polyurethane. In some embodiments, the alloy can be free of, or can be substantially free of, particles of the recombinant collagen fragment having an average diameter of greater than 1 micron. In some embodiments, the protein polyurethane alloy can be adhered mechanically, such as with sutures or glue, or can be otherwise attached directly onto a cellular surface in a subject with cancer. In some embodiments, the protein polyurethane alloy can be applied as a dry film or powder which can be naturally hydrated by the subject or hydrated in place after application with a suitable water or buffer. In embodiments where the protein polyurethane alloy, or hyaluronic acid protein alloy is applied as a dry film or powder to a cellular surface, and without wishing to be bound by a particular theory, it is believed that, after application, the film or powder will be hydrated by the cellular surface. Alternatively, the film or powder can be hydrated by application of a suitable buffer or water. In some embodiments, films can be prepared by drying any of the alloys described herein using any standard drying method known in the art, such as in a continuous oven. Powder forms can be prepared by spray drying or lyophilizing a solution, grinding a previously dried alloy, or by any other methods of preparation known in the art. Again, and without wishing to be bound by a particular theory, the placement of the any of the alloys described herein comprising a recombinant collagen fragment directly onto the cellular surface is thought to enrich type III collagen in the ECM, thereby limiting or preventing metastasis through disseminated tumor cell dormancy induction.

In some embodiments, the pharmaceutically acceptable scaffold can comprise a protein hyaluronic acid alloy. In some embodiments, the alloy can comprise about 10 wt % to about 50 wt % of the fragment and about 50 wt % to about 90 wt % of the hyaluronic acid. In some embodiments, the alloy can comprise about 20 wt % to about 35 wt % of the protein and about 65 wt % to about 80 wt % of the hyaluronic acid. In some embodiments the hyaluronic acid or the protein, or both, and the protein are crosslinked to themselves, to each other, or both. In some embodiments, the protein can be dissolved, suspended, or otherwise present in a crosslinked hyaluronic matrix. In some embodiments, the protein migrates out of the alloy to contact the cellular surface when the alloy is exposed to water or a buffer solution.

In some embodiments, the pharmaceutically acceptable scaffold can comprise a protein dissolved in a gelatin matrix. In some embodiments, the alloy can comprise about 10 wt % to about 50 wt % of the fragment and about 50 wt % to about 90 wt % of the gelatin. In some embodiments, the alloy can comprise about 20 wt % to about 35 wt % of the protein and about 65 wt % to about 80 wt % of the gelatin. In some embodiments, the gelatin can be crosslinked. In some embodiments, the protein migrates out of the alloy to contact the cellular surface when the alloy is exposed to water or a buffer solution.

In some embodiments, the present disclosure provides methods of treating a cancer in a subject in need thereof, the methods comprising contacting a cellular surface on the subject with a therapeutic biomaterial comprising a pharmaceutically acceptable scaffold and an effective amount of a recombinant collagen having the amino acid sequence set forth in SEQ ID NO: 973.

The amino acid sequence of SEQ ID NO: 973 is:

DVKSGVAVGGLAGYPGPAGPPGPPGPPGTSGHPGSPGSPGYQGPPGEPGQAGPSG
PPGPPGAIGPSGPAGKDGESGRPGRPGERGLPGPPGIKGPAGIPGFPGMKGHRGFD
GRNGEKGETGAPGLKGENGLPGENGAPGPMGPRGAPGERGRPGLPGAAGARGN
DGARGSDGQPGPPGPPGTAGFPGSPGAKGEVGPAGSPGSNGAPGQRGEPGPQGH
AGAQGPPGPPGINGSPGGKGEMGPAGIPGAPGLMGARGPPGPAGANGAPGLRGG
AGEPGKNGAKGEPGPRGERGEAGIPGVPGAKGEDGKDGSPGEPGANGLPGAAGE
RGAPGFRGPAGPNGIPGEKGPAGERGAPGPAGPRGAAGEPGRDGVPGGPGMRG
MPGSPGGPGSDGKPGPPGSQGESGRPGPPGPSGPRGQPGVMGFPGPKGNDGAPG
KNGERGGPGGPGPQGPPGKNGETGPQGPPGPTGPGGDKGDTGPPGPQGLQGLPG
TGGPPGENGKPGEPGPKGDAGAPGAPGGKGDAGAPGERGPPGLAGAPGLRGGA
GPPGPEGGKGAAGPPGPPGAAGTPGLQGMPGERGGLGSPGPKGDKGEPGGPGAD
GVPGKDGPRGPTGPIGPPGPAGQPGDKGEGGAPGLPGIAGPRGSPGERGETGPPG
PAGFPGAPGQNGEPGGKGERGAPGEKGEGGPPGVAGPPGGSGPAGPPGPQGVKG
ERGSPGGPGAAGFPGARGLPGPPGSNGNPGPPGPSGSPGKDGPPGPAGNTGAPGS
PGVSGPKGDAGQPGEKGSPGAQGPPGAPGPLGIAGITGARGLAGPPGMPGPRGSP
GPQGVKGESGKPGANGLSGERGPPGPQGLPGLAGTAGEPGRDGNPGSDGLPGRD
GSPGGKGDRGENGSPGAPGAPGHPGPPGPVGPAGKSGDRGESGPAGPAGAPGPA
GSRGAPGPQGPRGDKGETGERGAAGIKGHRGFPGNPGAPGSPGPAGQQGAIGSP
GPAGPRGPVGPSGPPGKDGTSGHPGPIGPPGPRGNRERGSEGSPGHPGQPGPPGPP
GAPGPCCGGVGAAAIAGIGGEKAGGFAPYYG

In some embodiments, the present disclosure provides methods of inhibiting proliferation of a cancer cell, the method comprising contacting the cancer cell with a therapeutic biomaterial comprising a pharmaceutically acceptable scaffold and a recombinant collagen fragment having the amino acid sequence set forth in SEQ ID NO: 973.

In some embodiments, the present disclosure provides methods of inducing cellular dormancy in a subject in need thereof, the method comprising contacting a cellular surface in the subject with a therapeutic biomaterial comprising a pharmaceutically acceptable scaffold and a recombinant collagen having the amino acid sequence set forth in SEQ ID NO: 973.

In some embodiments, the cancer can be characterized by the presence of a solid tumor. In some embodiments, the cancer can be a cancer of the skin, colon, oral mucosa, rectum, esophagus, thyroid, liver, pancreas, kidney, bladder, lung, brain, breast, ovary, testicle, prostate, bones, or a combination of any of the foregoing. In some embodiments, the recombinant collagen can be unhydroxylated. In some embodiments, the recombinant collagen can be hydroxylated.

In some embodiments of the methods of using a recombinant collagen having the amino acid sequence set forth in SEQ ID NO: 973 provided herein, the biomaterial can be a protein polyurethane alloy. In some embodiments of the methods of using a recombinant collagen having the amino acid sequence set forth in SEQ ID NO: 973 provided herein, the biomaterial can comprise a pharmaceutically acceptable scaffold. In some embodiments, the biomaterial can be adhered directly onto the cancer cells.

Protein polyurethane alloys described herein comprise a protein, such as any of the fragments disclosed herein, including for example SEQ ID NOs. 1-1002, dissolved within a polyurethane, or a plurality of polyurethanes. In some embodiments, the protein polyurethane alloys described herein comprise the collagen fragment having a sequence identity of at least about 85% to the amino acid sequence set forth in SEQ ID NOs: 1 and/or 973. In some embodiments, the protein polyurethane alloys described herein comprise the recombinant collagen fragment having at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity, or similarity to SEQ ID NOs: 1 and/or 973. In some embodiments, the protein polyurethane alloys described herein comprise the collagen fragment having the amino acid sequence set forth in SEQ ID NO: 1. In some embodiments, the protein polyurethane alloys described herein comprise the collagen fragment having the amino acid sequence set forth in any one of SEQ ID NOs: 2-972. In some embodiments, the protein polyurethane alloys described herein comprises the recombinant collagen having the amino acid sequence set forth in SEQ ID NO: 973 dissolved within a polyurethane, or a plurality of polyurethanes. In some embodiments, the protein polyurethane alloys described herein can comprise a recombinant collagen having an amino acid sequence set forth in any one of SEQ ID NOs: 974-1002 dissolved within a polyurethane, or a plurality of polyurethanes.

In particular embodiments, the protein polyurethane alloys described herein can comprise a protein that is miscible with only one of a plurality of phases of the polyurethane, or the plurality of polyurethanes, with which it is blended. For example, in some embodiments, the protein polyurethane alloy can include a protein that is miscible with only the hard phase of the polyurethane, or the plurality of polyurethanes, having both a hard phase and a soft phase. Protein polyurethane alloys described herein can be free of or substantially free of protein in the form of particles dispersed in a polyurethane. For example, in some embodiments, the protein polyurethane alloys can be free of or substantially free of protein particles having an average diameter of greater than 1 micron (μm). In some embodiments, the protein polyurethane alloys can be free of, or substantially free of, particles of recombinant collagen fragment having an average diameter of greater than 1 micron (μm). In some embodiments, the polyurethane can also comprise urea linkages. In such embodiments, the polyurethane is a polyurethane urea.

In some embodiments, the proteins for use in therapeutic biomaterials comprising a protein polyurethane alloy can be succinylated proteins. A succinylated protein is a protein modified with the addition of a succinyl group to a side chain of an amino acid in the protein. Most commonly, the succinyl group is added to lysine side chains. The method of adding a succinyl group to a side chain of an amino acid in a protein is commonly referred to as protein succinylation. Addition of succinyl groups on a protein can alter the protein's functional and structural properties. In some cases, the addition of a relatively large modification like a succinyl moiety can be expected to alter the tertiary structure of the protein. Further, with the addition of a succinyl moiety, a lysine side chain can be altered from a primary amine to an acid, making it more hydrophilic, and can change its charge from positive to negative in physiological pH. Succinylation can be accomplished using techniques and chemistry well known the art.

Suitable polyurethanes for use in the therapeutic biomaterials comprising the protein polyurethane alloy described herein include those that comprise at least two phases including a “soft phase” and a “hard phase.” The soft phase is formed from polyol segments within the polyurethane that separate from the urethane-containing phase due to differences in polarity. The urethane-containing phase is referred to as the hard phase. This phase separation is well known in the art and is the basis of the many of the properties of polyurethanes.

The soft phase is typically elastomeric at room temperature, and typically has a softening point or glass transition temperature (Tg) below room temperature. The Tg can be measured by Dynamic Mechanical Analysis (DMA) and quantified by either the peak of tan(δ) or the onset of the drop in storage modulus. Alternately, Tg can be measured by Differential Scanning calorimetry (DSC). In some cases, there can be crystallinity in the soft phase, which can be seen as a melting point, typically between 0° C. and about 60° C.

The hard phase typically has a Tg or melting point above room temperature, more typically above about 80° C. The softening of the hard phase can be measured by measuring the onset of the drop in storage modulus (sometimes referred to as stiffness) as measured by DMA.

The “soft phase” for the polyurethane or the protein polyurethane alloy including the polyurethane comprises the polyol component of the polyurethane. Its function is to be soft and flexible at temperatures above its Tg to lend toughness, elongation, and flexibility to the polyurethane. Typical soft segments can comprise polyether polyols, polyester polyols, polycarbonate polyols, and mixtures thereof. The soft segments typically range in molecular weight from about 250 D to greater than about 5 kD. The “hard phase” for the polyurethane or the protein polyurethane alloy including the polyurethane comprises the urethane segments of the polymer that are imparted by the isocyanate(s) used to connect the polyols along with short chain diols such as butane diol, propane diol, and the like. In some embodiments, the chain extender can include amines such as ethylene diamine, hexamethylene diamine and the like to introduce urea bonds. Urea bonds can also be introduced by adding water which reacts with the isocyanate to produce an amine and carbon dioxide which can act as a foaming agent. Thus the term polyurethane is understood in some embodiments to include urea linkages in the hard phase. Typical isocyanates useful for the present polyurethanes include, but are not limited to, hexamethylene diisocyanate, isophorone diisocyanate, methylene diisocyanate, toluene diisocyanate, phenyl diisocyanate, and the like. These molecules are more polar and stiffer than the polyols used to make the soft segment. Therefore, the hard segment is stiffer and has a higher softening point compared to the soft segment. The function of the hard phase is to provide, among other properties, strength, temperature resistance, and abrasion resistance to the polyurethane.

In some embodiments described herein, the protein can be miscible with only the hard phase, leaving soft phase transitions substantially unaltered. Without wishing to be bound by particular theory, it is believed that when the protein is dissolved in the hard phase, it significantly increases the temperature at which the hard phase begins to soften, thus increasing the temperature resistance of the alloy.

In a protein polyurethane alloy including one or more miscible proteins and polyurethanes, the one or more proteins can be dissolved within the hard phase of the one or more polyurethanes. The protein polyurethane alloy can include at least one protein miscible with the hard phase of one or more polyurethanes in the alloy. In some embodiments, the protein polyurethane alloy can include a plurality of proteins and/or a plurality of polyurethane hard phases that are miscible with each other. In all of these embodiments, and without wishing to be bound by a particular theory, the protein, or plurality of proteins, is believed to be dissolved in the hard phase of the polyurethane, or plurality of polyurethanes.

One or more proteins dissolved within the hard phase of one or more polyurethanes can form a homogenous mixture when blended. In some embodiments, the protein polyurethane alloy can include a plurality of proteins dissolved within one or more polyurethanes such that the proteins and the polyurethane(s) form a homogenous mixture when blended and dried. Typically, the protein polyurethane alloy including a homogenous mixture of protein and polyurethane does not include a substantial amount of protein not dissolved in the polyurethane. That said, and in some embodiments, the protein polyurethane alloy can include a fraction of protein dispersed within the polyurethane.

Suitable polyurethanes according to embodiments described herein include, but are not limited to, aliphatic polyurethanes, aromatic polyurethanes, bio-based polyurethanes, or acrylic acid modified polyurethanes. Suitable polyurethanes are commercially available from manufacturers including Covestro, Lubrizol, Hauthaway, Stahl, and the like. In some embodiments, a polyurethane for a protein polyurethane alloy can be bio-polyurethane. In some embodiments, the polyurethane is a water-dispersible polyurethane. In some embodiments, the polyurethane can be a polyester polyurethane. In some embodiments, the polyurethane can be a polyether polyurethane. In some embodiments, the polyurethane can be a polycarbonate-based polyurethane. In some embodiments, the polyurethane can be an aliphatic polyester polyurethane. In some embodiments, the polyurethane can be an aliphatic polyether polyurethane. In some embodiments, the polyurethane can be an aliphatic polycarbonate polyurethane. In some embodiments, the polyurethane can be an aromatic polyester polyurethane. In some embodiments, the polyurethane can be an aromatic polyether polyurethane. In some embodiments, the polyurethane can be an aromatic polycarbonate polyurethane.

In some embodiments, the polyurethane can have a soft segment selected from the group consisting of: polyether polyols, polyester polyols, polycarbonate polyols, and mixtures thereof. In some embodiments, the polyurethane can have a hard segment comprising diisocyanates and optionally short chain diols or diamines. Suitable diisocyanates can be selected from the group consisting of: aliphatic diisocyanates such as hexamethylene diisocyanate, isophorone diisocyanate; aromatic diisocyanates such as 4,4′ diphenyl methylene diisocyanate, toluene diisocyanate, phenyl diisocyanate, and mixtures thereof. Suitable short chain diols include ethylene glycol, propane diol, butane diol, 2,2 methyl 1,3 propane diol, pentane diol, hexane diol and mixtures thereof. Suitable short chain diamines include ethylene diamine, hexamethylene diamine, and mixtures thereof. In some embodiments, crosslinkers such as multifunctional alcohols, for example, trimethylol propane triol, or diamines such as ethylene diamine or 4,4′diamino, diphenyl diamine.

Exemplary commercial polyurethanes, include but are not limited to L3360 and Hauthane HD-2001 available from C.L. Hauthaway & Sons Corporation, SANCURE™ polyurethanes available from Lubrizol Corporation, BONDTHANE™ polyurethanes, for example UD-108, UD-250, and UD-303 available from Bond Polymers International, EPOTAL® ECO 3702 and EPOTAL® P100 ECO from BASF, and Permutex Evo EX-RC-2214 (RC-2214) from Stahl. L3360 is a aliphatic polyester polyurethane polymer aqueous dispersion having a 35% solids content, a viscosity of 50 to 500 cps (centipoise), and a density of about 8.5 lb/gal (pounds per gallon). HD-2001 is an aliphatic polyester polyurethane polymer aqueous dispersion having a 40% solids content, a viscosity of 50 to 500 cps, and a density of about 8.9 lb/gal. BONDTHANE™ UD-108 is an aliphatic polyether polyurethane polymer aqueous dispersion having a 33% solids content, a viscosity of 300 cps, and a density of 8.7 lb/gal. BONDTHANE™ UD-250 is an aliphatic polyester polyurethane polymer aqueous dispersion having a 35% solids content, a viscosity of 200 cps, and a density of 8.8 lb/gal. BONDTHANE™ UD-303 is an aliphatic polyether polyurethane polymer aqueous dispersion having a 35% solids content, a viscosity of less than 500 cps, and a density of 8.7 lb/gal. EPTOAL® P100 ECO is a polyester polyurethane elastomer aqueous dispersion having approximately 40% solids and a viscosity of about 40 mPas. RC-2214 is an aliphatic polyether polyurethane polymer aqueous dispersion having a 58-60% solids content, a viscosity of 4,000 to 15,000 cps, and a density of about 8.9 lb/gal.

Exemplary bio-based polyurethanes include, but are not limited to, L3360 available from C.L. Hauthaway & Sons Corporation, IMPRANIL® Eco DLS, IMPRANIL® Eco DL 519, IMPRANIL® Eco DLP-R, and IMPRAPERM® DL 5249 available from Covestro. IMPRANIL® Eco DLS is an anionic, aliphatic polyester polyurethane polymer aqueous dispersion having approximately 50% solids content, a viscosity of less than 1,200 MPas, and a density of about 1.1 g/cc. IMPRANIL® Eco DL 519 is an anionic, aliphatic polyester polyurethane polymer aqueous dispersion. IMPRANIL® Eco DLP-R is an anionic, aliphatic polyester polyurethane polymer aqueous dispersion. IMPRAPERM® DL 5249 is an anionic aliphatic polyester-polyurethane polymer aqueous dispersion.

In some embodiments, the polyurethane can include reactive groups that can be cross-linked with a protein. Exemplary reactive groups include, but are not limited to, a sulfonate, an aldehyde, a carboxylic acid or ester, a blocked isocyanate, or the like, and combinations thereof. In such embodiments, the polyurethane can be crosslinked to the protein in the protein polyurethane alloy through the reaction of a reactive group on the protein with the reactive group present in the polyurethane.

In some embodiments, the amount of protein (the recombinant collagen fragment having at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity, or similarity to SEQ ID NOs: 1 and/or 973, or the collagen fragment having the amino acid sequence set forth in any one of SEQ ID NOs: 2-972, or the recombinant collagen having the amino acid sequence set forth in SEQ ID NO: 973, or the recombinant collagen having the amino acid sequence set forth in any one of SEQ ID NOs: 974-1002, in the protein polyurethane alloy can range from about 5 wt % to about 50 wt % of protein, including subranges. For example, in some embodiments, the amount of protein in the polyurethane alloy can range from about 5 wt % to about 50 wt %, about 10 wt % to about 50 wt %, about 15 wt % to about 50 wt %, about 20 wt % to about 50 wt %, about 25 wt % to about 50 wt %, about 30 wt % to about 50 wt %, about 35 wt % to about 50 wt %, about 40 wt % to about 50 wt %, about 45 wt % to about 50 wt %, about 5 wt % to about 45 wt %, about 5 wt % to about 40 wt %, about 5 wt % to about 35 wt %, about 5 wt % to about 30 wt %, about 5 wt % to about 25 wt %, about 5 wt % to about 20, about 5 wt % to about 15 wt %, or about 5 wt % to about 10 wt % or within a range having any two of these values as endpoints, inclusive of the endpoints. In some embodiments, the amount of protein in the protein polyurethane alloy can range from about 20 wt % to about 35 wt %.

In some embodiments, the amount of polyurethane in the protein polyurethane alloy can range from about 50 wt % to about 95 wt %, including subranges. For example, in some embodiments, the amount of polyurethane in the protein polyurethane alloy can range from about 50 wt % to about 95 wt %, about 55 wt % to about 95 wt %, about 60 wt % to about 95 wt %, about 65 wt % to about 95 wt %, about 70 wt % to about 95 wt %, about 75 wt % to about 95 wt %, about 80 wt % to about 95 wt %, about 85 wt % to about 95 wt %, about 90 wt % to about 95 wt %, about 50 wt % to about 90 wt %, about 50 wt % to about 85 wt %, about 50 wt % to about 80 wt %, about 50 wt % to about 75 wt %, about 50 wt % to about 70 wt %, about 50 wt % to about 65 wt %, about 50 wt % to about 60 wt %, or about 50 wt % to about 55 wt %, or within a range having any two of these values as endpoints, inclusive of the endpoints. In some embodiments, the amount of polyurethane in the protein polyurethane alloy can range from about 65 wt % to about 80 wt %.

Any of the above-listed ranges for the weight percentages of protein and polyurethane in the protein polyurethane alloy can be combined. For example, in some embodiments, the weight percentages of protein and polyurethane in the protein polyurethane alloy can be any of the following. The amount of protein in the polyurethane alloy can range from about 5 wt % to about 50 wt % and the amount of polyurethane in the protein polyurethane alloy can range from about 50 wt % to about 95 wt %. The amount of protein in the polyurethane alloy can range from about 15 wt % to about 50 wt % and the amount of polyurethane in the protein polyurethane alloy can range from about 50 wt % to about 85 wt %. The amount of protein in the polyurethane alloy can range from about 20 wt % to about 50 wt % and the amount of polyurethane in the protein polyurethane alloy can range from about 50 wt % to about 80 wt %. The amount of protein in the polyurethane alloy can range from about 25 wt % to about 50 wt % and the amount of polyurethane in the protein polyurethane alloy can range from about 50 wt % to about 75 wt %. The amount of protein in the polyurethane alloy can range from about 30 wt % to about 50 wt % and the amount of polyurethane in the protein polyurethane alloy can range from about 50 wt % to about 70 wt %. The amount of protein in the polyurethane alloy can range from about 10 wt % to about 40 wt % and the amount of polyurethane in the protein polyurethane alloy can range from about 60 wt % to about 90 wt %. The amount of protein in the polyurethane alloy can range from about 15 wt % to about 40 wt % and the amount of polyurethane in the protein polyurethane alloy can range from about 60 wt % to about 85 wt %. The amount of protein in the polyurethane alloy can range from about 20 wt % to about 40 wt % and the amount of polyurethane in the protein polyurethane alloy can range from about 60 wt % to about 80 wt %. The amount of protein in the polyurethane alloy can range from about 20 wt % to about 35 wt % and the amount of polyurethane in the protein polyurethane alloy can range from about 65 wt % to about 80 wt %. In some embodiments, the above-listed weight percent values and ranges can be based on the total weight of the protein polyurethane alloy. In some embodiments, the above-listed weight percent values and ranges can be based on the total weight of only protein and polyurethane in a protein polyurethane alloy. Unless otherwise specified, a weight percent value or range for the polyurethane and the protein is based on the total weight of only protein and polyurethane in a protein polyurethane alloy.

In some embodiments, the sum of the amount of protein (the recombinant collagen fragment having at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity, or similarity to SEQ ID NOs: 1 and/or 973, or the collagen fragment having the amino acid sequence set forth in any one of SEQ ID NOs: 2-972, or the recombinant collagen having the amino acid sequence set forth in SEQ ID NO: 973) plus the amount of polyurethane in the protein polyurethane alloy can be about 80 wt % or more. For example, in some embodiments, the sum of the amount of protein plus the amount of polyurethane in the protein polyurethane alloy can range from about 80 wt % to 100 wt %, about 82 wt % to 100 wt %, about 84 wt % to 100 wt %, about 86 wt % to 100 wt %, about 88 wt % to 100 wt %, about 90 wt % to 100 wt %, about 92 wt % to 100 wt %, about 94 wt % to 100 wt %, about 96 wt % to 100 wt %, or about 98 wt % to 100 wt %.

In some embodiments, the protein polyurethane alloy can include water making up a portion of the total weight percent of the material. In some embodiments, the amount of water in the protein polyurethane alloy can range from about 1 wt % to about 10 wt %, including subranges. For example, in some embodiments, the amount of water in the protein polyurethane alloy can range from about 1 wt % to about 10 wt %, about 2 wt % to about 10 wt %, about 3 wt % to about 10 wt %, about 4 wt % to about 10 wt %, about 5 wt % to about 10 wt %, about 6 wt % to about 10 wt %, about 7 wt % to about 10 wt %, about 8 wt % to about 10 wt %, about 1 wt % to about 9 wt %, about 1 wt % to about 8 wt %, about 1 wt % to about 7 wt %, about 1 wt % to about 6 wt %, about 1 wt % to about 5 wt %, about 1 wt % to about 4 wt %, or about 1 wt % to about 3 wt %, or within a range having any two of these values as endpoints, inclusive of the endpoints.

### EXAMPLES

It is understood that the examples and embodiments described herein are for illustrative purposes only and that various modifications or changes in light thereof will be suggested to persons skilled in the art and are to be included within the spirit and purview of this application.

### Example 1: Effective Coagulation of Recombinant Collagen Protein in Polyurethane

To determine whether recombinant collagen could be effectively coagulated with commercially available polyurethane dispersions designed for medical applications, the 50 KDa recombinant collagen protein (“50 KDa rCol”) comprising the amino acid sequence of SEQ ID NO: 1 was formulated in various polyurethane compositions, as described below.

50 KDa rCol: Baymedix® AD111

A sample was prepared by dissolving 0.86 g of 50 KDa rCol into 4.29 mL of de-ionized water and stirring with a magnetic stir bar at 600 rpm for 30 min at 20° C. After stirring for 30 min, 6.86 g of Baymedix® AD111 (Covestro AG, Leverkusen, Germany) polyurethane dispersion was added to the solution and stirred at 600 rpm for 30 minutes. The polyurethane and 50 KDa rCol solution was then pipetted into a Teflon® evaporating dish with a diameter of 10 cm. The dish was dried in an oven at 45° C. overnight. After drying, the dried solution was conditioned at standard reference atmosphere (23° C., 50% humidity) for 24 hours to create a 50 KDa rCol-AD111 coagulated film.

The film was tested using a DMA-850 from TA Instruments. A 1 cm×2.5 cm strip was cut from each film using a metal die. The cut film samples were loaded into the film and fiber tension clamp for testing. During testing, a pre-load of 0.01 N was applied to the cut film samples. The instrument was cooled to −80° C., held for 1 minute, then the temperature was ramped at 4° C./minute to 200° C., or until the sample was too weak to be held in tension. During the temperature ramp, the sample was oscillated 0.1% strain at a frequency of 1 Hz. The storage modulus, loss modulus, and tan (8) were plotted with temperature for both films. The resulting second storage modulus transition (taken as the onset point of the last decrease in the storage modulus measured, i.e. second DMA modulus transition onset temperature) was 125.0° C. for the 50 KDa rCol-AD111 film. The control sample of AD111 was too soft for DMA testing.

Additionally, 4 tensile specimens (according to the Standard Test Method for Tensile Properties of Plastics “ASTM D638”) were each cut from the dried and conditioned sample film using a metal die. The cut film samples were loaded into an INSTRON® 5960 (Instron, Norwood, MA) series machine and pulled in tension at 100 millimeters/minute until break. The average Young's modulus, average tensile strength (maximum tensile stress), and average elongation at break were recorded. The Young's modulus was 131 MPa, the maximum tensile stress was 4.8 MPa, and the elongation at break was 209%, for the 50 KDa rCol-AD111 film.

50 KDa rCol: Baymedix CD102

A sample was prepared by dissolving 0.75 g of 50 KDa rCol into 3.75 mL of de-ionized water and stirring with a magnetic stir bar at 600 rpm for 30 min at 20° C. After stirring for 30 min, 7.5 g of Baymedix® CD102 (Covestro AG, Leverkusen, Germany) polyurethane dispersion was added to the solution and stirred at 600 rpm for 30 minutes. The polyurethane and 50 KDa rCol solution was then pipetted into a Teflon® evaporating dish with a diameter of 10 cm. The dish was dried in an oven at 45° C. overnight. After drying, the dried solution was conditioned at standard reference atmosphere (23° C., 50% humidity) for 24 hours to create a 50 KDa rCol polyurethane coagulated film.

Dynamic mechanical analysis (DMA) testing was performed as outlined above. The resulting second storage modulus transition (taken as the onset point of the last decrease in the storage modulus measured, i.e. the second DMA modulus transition onset temperature) for the 50 KDa rCol-CD102 film was 155.9° C., a 27.3° C. increase over the CD102 control sample (128.6° C.).

Tensile testing was performed as outlined above. The average Young's modulus was 48 MPa, the average tensile stress measured was 13 MPa, and the average elongation at break was 754% for the 50 KDa rCol-CD102 film.

50 KDa rCol: Baymedix® CD104

A sample was prepared by dissolving 1.25 g of 50 KDa rCol into 6.25 mL of de-ionized water and stirring with a magnetic stir bar at 600 rpm for 30 min at 20° C. After stirring for 30 min, 12.5 g of Baymedix® CD104 (Covestro AG, Leverkusen, Germany) polyurethane dispersion was added to the solution and stirred at 600 rpm for 30 minutes. The polyurethane and 50 KDa rCol solution was then pipetted into a Teflon® evaporating dish with a diameter of 10 cm. The dish was dried in an oven at 45° C. overnight. After drying, the dried solution was conditioned at standard reference atmosphere (23° C., 50% humidity) for 24 hours to create a 50 KDa rCol polyurethane coagulated film.

DMA testing was performed as outlined above. The resulting second storage modulus transition (taken as the onset point of the last decrease in the storage modulus measured, i.e. the second DMA modulus transition onset temperature) for the 50 KDa rCol-CD104 film was 109.7° C., a 37.8° C. increase over the CD104 control sample (71.9° C.).

Tensile testing was performed as outlined above. The average Young's modulus was 150 MPa, the average tensile stress measured was 19 MPa, and the average elongation at break was 598% for the 50 KDa rCol-CD104 film.

50 KDa rCol: Baymedix® FD103

A sample was prepared by dissolving 0.94 g of 50 KDa rCol into 4.72 mL of de-ionized water and stirring with a magnetic stir bar at 600 rpm for 30 min at 20° C. After stirring for 30 min, 6.34 g of Baymedix® FD103 (Covestro AG, Leverkusen, Germany) polyurethane dispersion was added to the solution and stirred at 600 rpm for 30 minutes. The polyurethane and 50 KDa rCol solution was then pipetted into a Teflon® evaporating dish with a diameter of 10 cm. The dish was dried in an oven at 45° C. overnight. After drying, the dried solution was conditioned at standard reference atmosphere (23° C., 50% humidity) for 24 hours to create a 50 KDa rCol polyurethane coagulated film.

DMA testing was performed as outlined above. The resulting second storage modulus transition (taken as the onset point of the last decrease in the storage modulus measured, i.e. the second DMA modulus transition onset temperature) for the 50 KDa rCol-FD103 film was 178.1° C., a 14.2° C. increase over the FD103 control sample (163.9° C.).

Tensile testing was performed as outlined above. The average Young's modulus was 45 MPa, the average tensile stress measured was 19 MPa, and the average elongation at break was 414% for the 50 KDa rCol-FD103 film.

### Baymedix® FD103 Foam

A sample is prepared by mechanical foaming of Baymedix® FD103. After frothing, the foam is dried at elevated temperatures (e.g., 120° C.) for a sufficiently long time (e.g., 10-30 min) depending on the foam thickness. After that, the dried foam is soaked with 50 KDa rCol de-ionized water solution at certain concentrations (e.g., 200 mg/ml).

### Baymedix® FD103: AD111 Foam

A sample is prepared by mechanical foaming of Baymedix® FD103: AD111 mixture. After frothing, the foam is dried at elevated temperatures (e.g., 120° C.) for a sufficiently long time (e.g., 10-30 min) depending on the foam thickness. After that, the dried foam is soaked with 50 KDa rCol de-ionized water solution at certain concentrations (e.g., 200 mg/ml).

50 KDa rCol: Baymedix® FD103 Foam

A sample is prepared by mechanical foaming of the mixture of 50 KDa rCol de-ionized water solution and Baymedix® FD103. After frothing, the foam is dried at elevated temperatures (e.g., 120° C.) for a sufficiently long time (e.g., 10-30 min) depending on the foam thickness. After that, the dried foam is soaked with 50 KDa rCol de-ionized water solution at certain concentrations (e.g., 200 mg/ml).

50 KDa rCol: Baymedix® FD103: AD111 Foam

A 16.7% (w/w) stock solution of 50 KDa rCol in water was prepared. Baymedix FD103 and Baymedix® AD111 are each aqueous polyurethane dispersions. A stock solution of Baymedix FD103: AD111 (Baymedix® FD103 and Baymedix® AD111) was prepared at a ratio of 70:30 based on the weight of polyurethane dispersion. A 10% (w/w) stock solution of Rheolate 208 (Elementis, London, UK) was prepared in water. 9.16 g of the recombinant collagen stock solution and 11.50 g of the Baymedix FD103: AD111 stock solution were mixed in a 50 ml beaker with an overhead mixer. After that, 1.55 g of Rheolate 208 stock solution was added and the solution was mixed further. Then 0.72 g of ChemTex 2216 (Cumberland, RI), 0.65 g of ChemTex 2317, and 0.03 g of ChemTex 2243 were added and the resulting mixture was mixed further. The resulting mixture was mechanically frothed by vigorously mixing with an overhead mixer. After frothing to introduce air bubbles, the foamed mixture was coated on a glass plate on a TQC film (TQC Automatic Film Applicator, Gardco) at a gap setting of 600 μm. The coating was dried at 75° C. for 30 minutes, and a porous foam sheet was achieved after drying.

This example demonstrates that the 50 KDa recombinant collagen protein comprising the amino acid sequence of SEQ ID NO: 1 can be effectively coagulated with polyurethane dispersions designed for medical applications.

50 KDa rCol: Hauthaway L3360 Polyurethane Dispersion

A sample was prepared by dissolving 1 g of 50 KDa rCol into 5 mL of de-ionized water and stirring with a magnetic stir bar at 1000 rpm for 1 hour at 20° C. After stirring for 1 hour, 6.7 g of L3360 (Hauthaway L3360 Polyurethane Dispersion) was added to the solution and stirred at 1000 rpm for 30 minutes. The polyurethane and 50 KDa rCol solution was then pipetted into a Teflon evaporating dish with a diameter of 10 cm. The dish was dried in an oven at 45° C. overnight. After drying, the dried sample was conditioned at standard reference atmosphere (23° C., 50% humidity) for 24 hours to create a 50 KDa rCol polyurethane alloy film.

The film was tested using a DMA-850 from TA Instruments. The resulting second storage modulus transition (taken as the onset point of the last decrease in the storage modulus measured, i.e. second DMA modulus transition onset temperature) for the 50 KDa rCol polyurethane alloy was 177.8° C., a 62.9° C. increase over the control sample. The storage modulus measured for the control sample was 114.9° C.

Tensile testing was performed by loading into an Instron® 5960 series machine and pulled in tension at 100 millimeters/minute until break. The average Young's modulus was 161 MPa, the average tensile stress measured was 17 MPa, and the average elongation at break was 173% for the 50 KDa rCol polyurethane alloy.

### Example 2: In Vivo Evaluation of Collagen-Supplemented Extra Cellular Matrix

The anti-cancer activity of the extracellular matrix (ECM) was evaluated by treating with commercial bovine collagen III (“bovine collagen III) (Uniprot sequence Q08E14; SEQ ID NO: 1009) and a 50 KDa recombinant collagen protein (SEQ ID NO: 1). Treatment solutions of bovine collagen III or SEQ ID NO: 1 were mixed with 4T1-Luc-1A4 murine breast carcinoma cells prior to subcutaneous implantation in female Balb/c mice. Tumors were collected at end of life, photographed, then bisected. The experimental details are results are below.

### 4T1-Luc2-1A4 Cells

Murine breast carcinoma cells (4T1-Luc2-1A4) were grown in Park Memorial Institute (RPMI) 1640 Medium (ThermoFisher Scientific), 1 mM Sodium Pyruvate, 10 mM HEPES buffer, 2.8 mL 45% Glucose (1.25 g), 10% NHI Fetal Bovine Serum (FBS), 1% PSG. The cells were dissociated with 0.25% trypsin/2.21 mM EDTA in Hanks' Balanced Salt Solution (HBSS) and 5.00E+05 trypan-excluding cells were resuspended in 200 μL serum-free RPMI 1640 medium. The collagen was added to the cells during the preparation of the inoculum. Each inoculum was supplemented with 1.2 mL of collagen. Test samples were prepared at different concentrations as shown below. Bovine Collagen III was obtained from Nippi-inc (Japan; catalog number PSC-3-100-20).

Eight groups of animals (1 control and 7 experimental groups; N=8) were included in the study, and treatments were administered as shown in Table 2. The inoculating dose for each animal was 5.00E+05 cells.

TABLE 2

| | | Concentration |
|---|---|---|
| Group | Treatment | (mg/mL) |
| 1 | Control | 1.5 |
| 2 | Hydroxylated Bovine Collagen III | 1.5 |
| 3 | Hydroxylated Bovine Collagen III + 0.1% | 1.5 |
| | EtOH | |
| 4 | Hydroxylated Bovine Collagen III + 0.01% | 1.5 |
| | EtOH | |
| 5 | 200 mg/ml SEQ ID NO: 1 | 200 |
| 6 | 10 mg/ml SEQ ID NO: 1 | 10 |
| 7 | 1.5 mg/ml SEQ ID NO: 1 | 1.5 |
| 8 | 10 mg/ml hydroxylated SEQ ID NO: 1 | 10 |

Each mouse was injected subcutaneously with 200 μL of inoculum (right axilla (high)). All procedures carried out in this experiment were conducted in compliance with the applicable laws, regulations and guidelines of the National Institutes of Health (NIH). Cell viabilities were measured before and after injection as shown in Table 3.

TABLE 3

| Viability (pre-injection) | |
|---|---|
| Viability (post-injection) | All groups 84% |
| Group 1 | 83% |
| Group 2 | 80% |
| Group 3 | 74% |
| Group 4 | 79% |
| Group 5 | 90% |
| Group 6 | 81% |
| Group 7 | 83% |
| Group 8 | 84% |

At day 23, tissue was excised, photographs were taken, and the tumor was bisected. Half of the tumor was placed in 10% Neutral Buffered Formalin (NBF) (Sigma) for 24 hours, and transferred to 70% EtOH for processing to FFPE blocks. Day 23 was chosen for evaluation because this was the last day all animals remained on study. The blocks were stored at ambient temperature. The other tumor half was snap frozen in liquid nitrogen. Tissues were stored at −80° C. A summary of the group tolerance to treatment and response to treatment is provided in Table 4.

TABLE 4

| | | Weight Change | Deaths in | Median Tumor | Median | Increase | Tumor- |
|---|---|---|---|---|---|---|---|
| | | in Treatment | Treatment | Doubling | ΔT/ΔC | In Time to | Free |
| Group | | Window | Window | Time | Day 23 | Progression | Survivors |
| # | Route | (%) | (%) | (Days) | (%) | (%) | (%) |
| 1 | SC | 15.3 | 0.0 | 5.0 | NA | NA | 0.0 |
| 2 | SC | 11.8 | 0.0 | 5.8 | 60 | 16.0 | 0.0 |
| 3 | SC | 15.2 | 12.5 | 6.2 | 70 | 8.0 | 0.0 |
| 4 | SC | 14.9 | 0.0 | 6.6 | 58 | ≥16.0 | 0.0 |
| 5 | SC | 6.6 | 0.0 | 6.2 | 51 | ≥16.0 | 0.0 |
| 6 | SC | 13.5 | 12.5 | 5.5 | 64 | 16.0 | 0.0 |
| 7 | SC | 8.4 | 0.0 | 5.6 | 63 | 12.0 | 0.0 |
| 8 | SC | 20.3 | 0.0 | 5.1 | 75 | 4.0 | 0.0 |

*Median ΔT/ΔC (%) − ΔC and ΔT − Individual animal endpoints that are calculated for each animal as follows: ΔT = Tt − T0 and ΔC = Ct − C0, where Tt and T0 are the tumor burdens of a treated animal at time t or at the initiation of dosing, respectively. ΔC reflects similar calculations for the control animals.

### Results

All treatments were well tolerated, resulting in no body weight loss over the duration of the study (Table 3). One mouse in Group 3 was found dead on Day 28, and one mouse in Group 6 was euthanized on Day 28 for severely impaired movement. No other treated animal was euthanized for any reason other than reaching institutional animal care and use committee (IACUC) mandated maximum tumor volume or study end. Although the deaths seen in Groups 3 and 6 are outside the traditional treatment window of 14 days post treatment, both animals are considered to be deaths in the treatment window due to the nature of the administration of the treatment and its uncertain impact on disease progression.

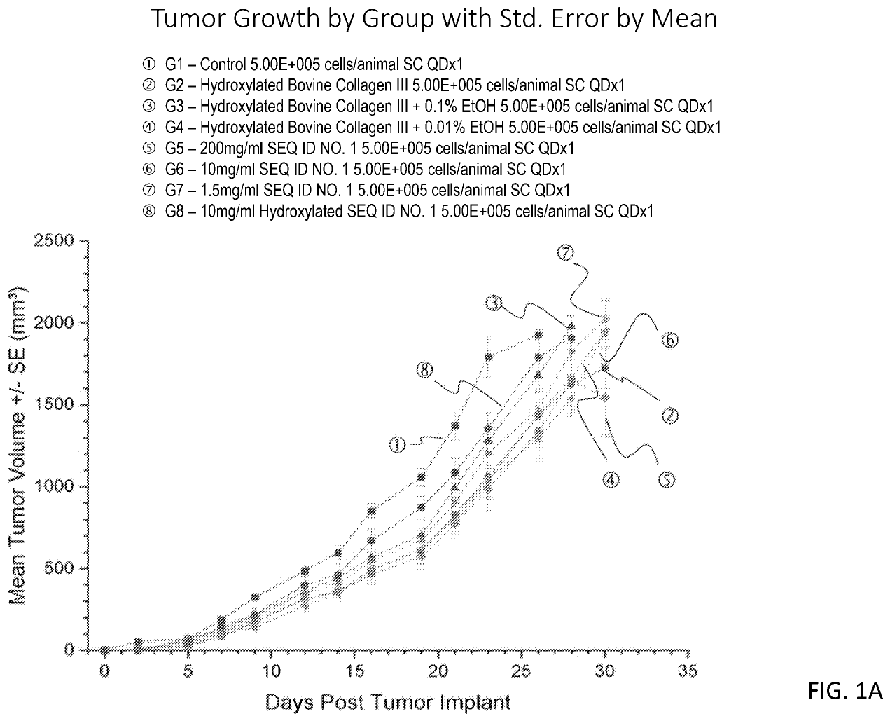

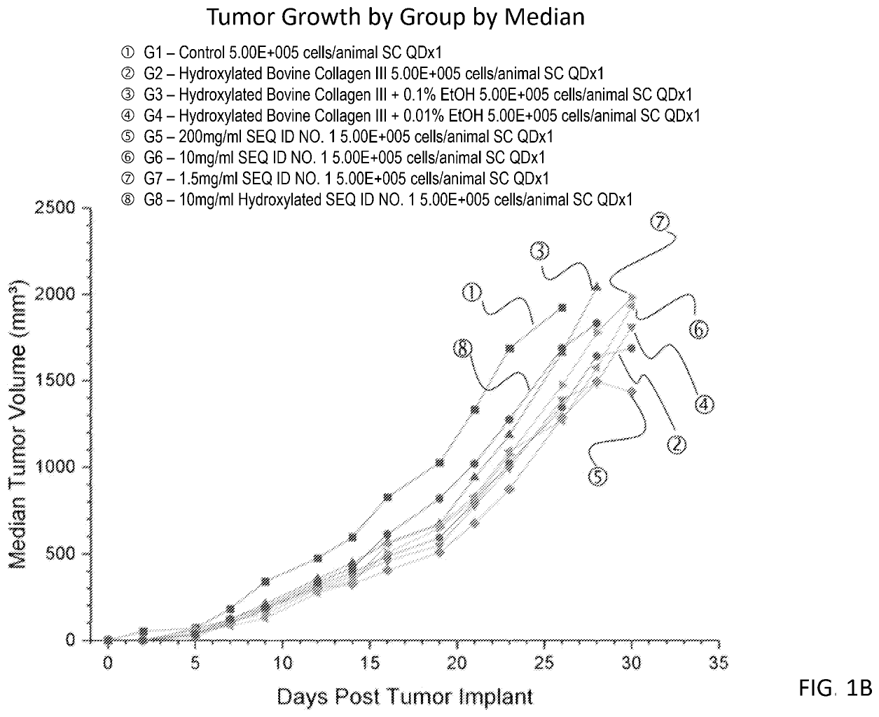

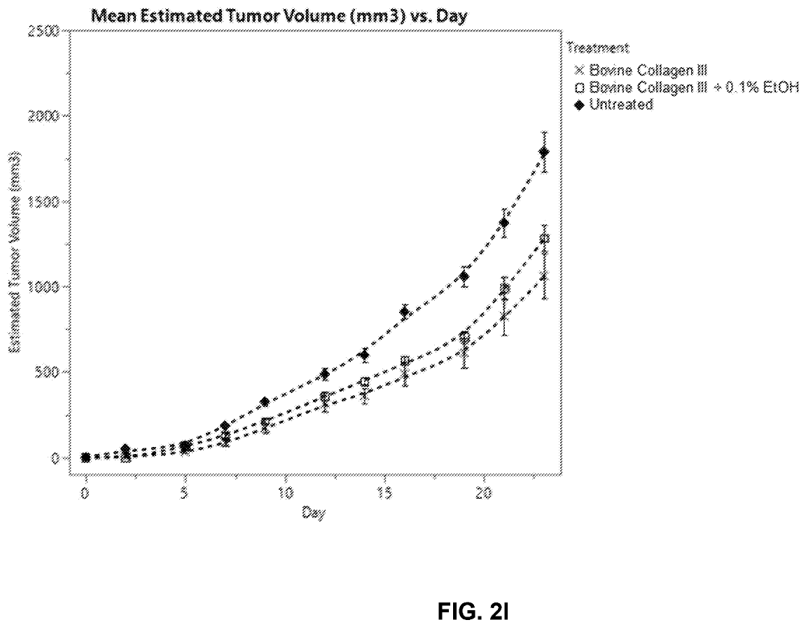

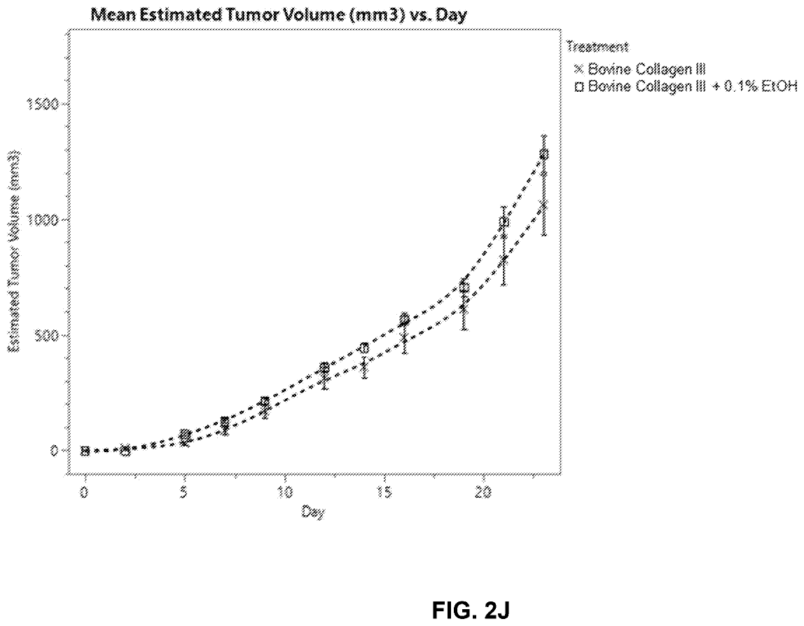

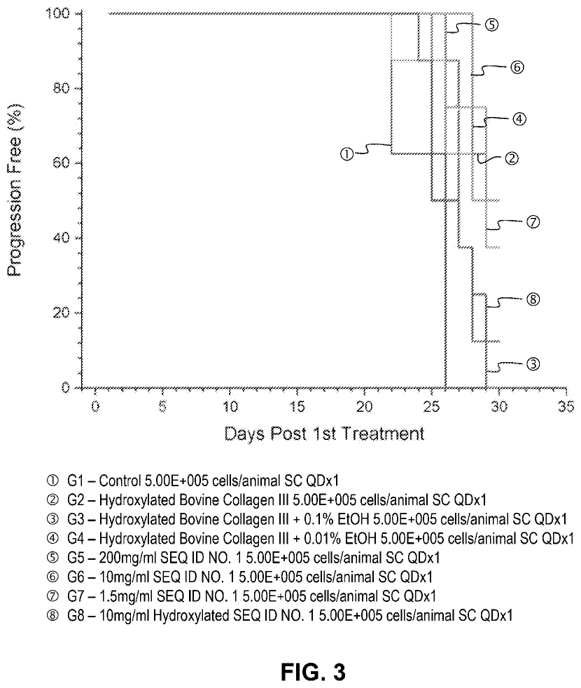

Inoculation with 5.00E+05 cells per animal in the Control Group (Group 1) resulted in a median tumor doubling time of 5.0 days and a median time to progression of 25 days. All tumor growth assessments were compared to Group 1 (untreated control) (FIGS. 1, 2A-21, 3, and 4).

The addition of bovine collagen (Group 2) resulted in a median tumor doubling time of 5.8 days, a 60% Day 23 median ΔT/ΔC value, and an increase in time to progression (ITP) of 16.0%. There were no tumor-free survivors in this group.

The addition of bovine collagen and 0.1% EtOH (Group 3) or bovine collagen and 0.01% EtOH (Group 4) resulted in median tumor doubling times of 6.2 and 6.6 days, Day 23 median ΔT/ΔC values of 70% and 58% and ITP values of 8.0% and ≥16.0%, respectively. There were no tumor-free survivors in these groups.

Surprisingly, tumor treatment with hydroxylated collagens did not show statistically significant tumor growth suppression, as compared to untreated control (data not shown). Without wishing to be bound by a particular theory, it is believed that residual ethanol in the hydroxylated collagen samples interfered with the experimental results. See e.g., Alcohol Res. 2015; 37(2): 311-322 (2015), which is incorporated by reference herein, explaining that ethanol can induce tumor growth. To investigate the effect of ethanol, 0.1% and 0.01% ethanol was added to Groups 3 and 4 (bovine collagen controls) to determine ethanol's effect on tumor size, in comparison to Groups 5-8. In all cases the addition of ethanol induced tumor growth and suppressed the effect of the bovine collagen as seen in Group 2 (bovine collagen alone).

The addition of SEQ ID NO: 1 at 200 mg/mL, 10 mg/mL or 1.5 mg/mL (Groups 5, 6, and 7, respectively) resulted in median tumor doubling times ranging from 5.5 to 6.2 days, with Day 23 median ΔT/ΔC values ranging from 51% to 64% and ITP values ranging from 12.0% to ≥16.0%. There were no tumor-free survivors in these groups. Collagen #1 was most efficacious at the 200 mg/ml dose (Group 5).

The addition of SEQ ID NO: 1 at 10 mg/mL (Group 8) resulted in a median tumor doubling time of 5.1 days, a 75% Day 23 median ΔT/ΔC value and an ITP value of 4.0%. There were no tumor-free survivors in this group.

This example demonstrates that treatment with SEQ ID NO: 1 results in anti-cancer activity that is as effective as treatment with bovine collagen.

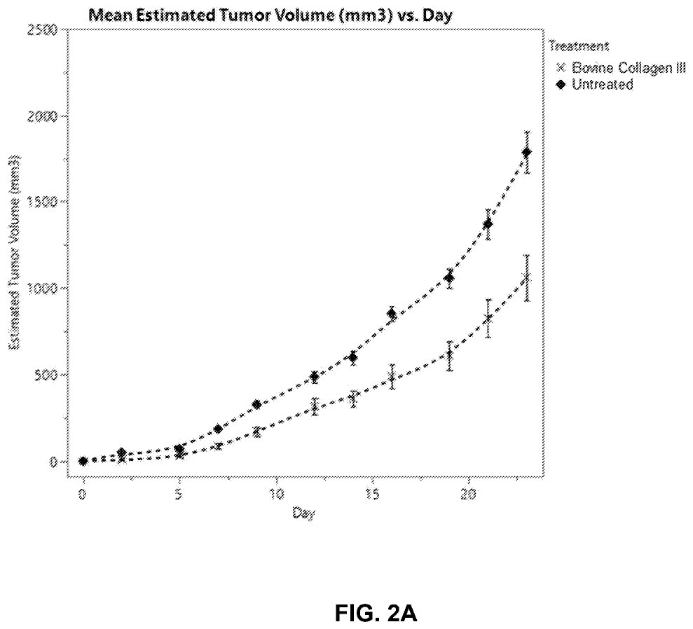

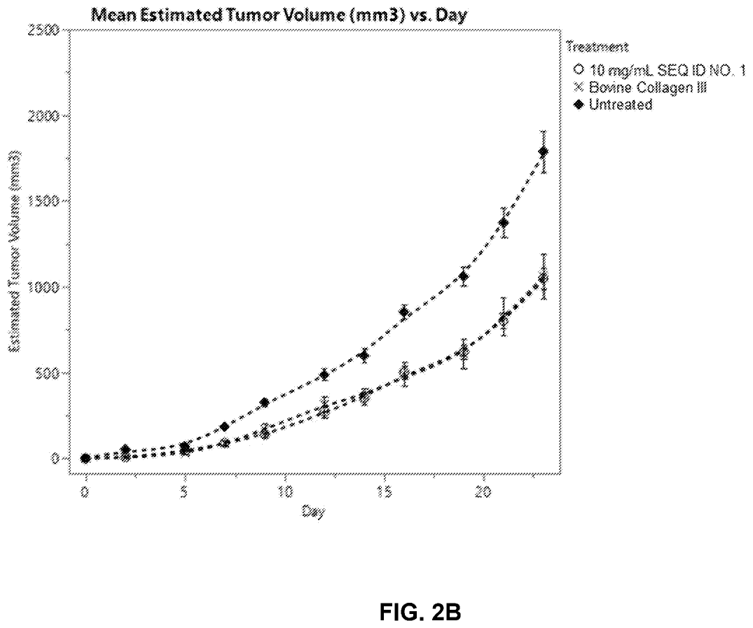

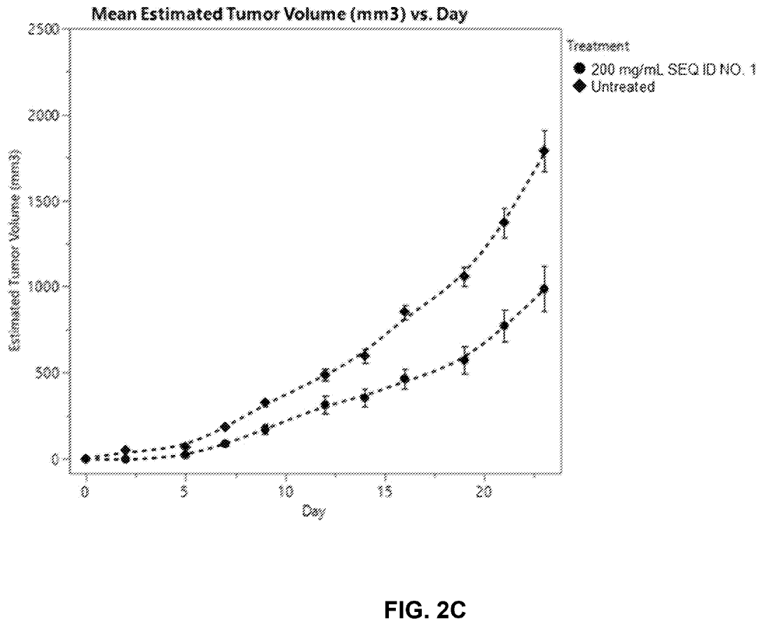

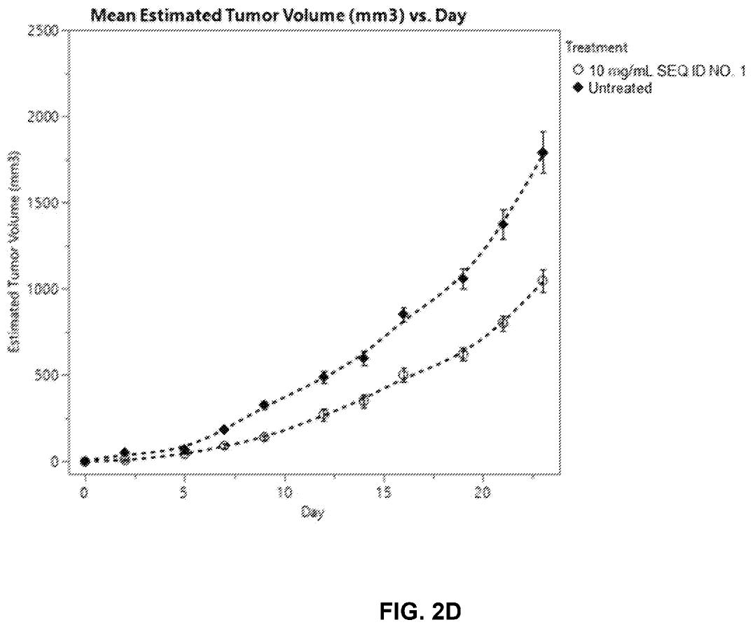

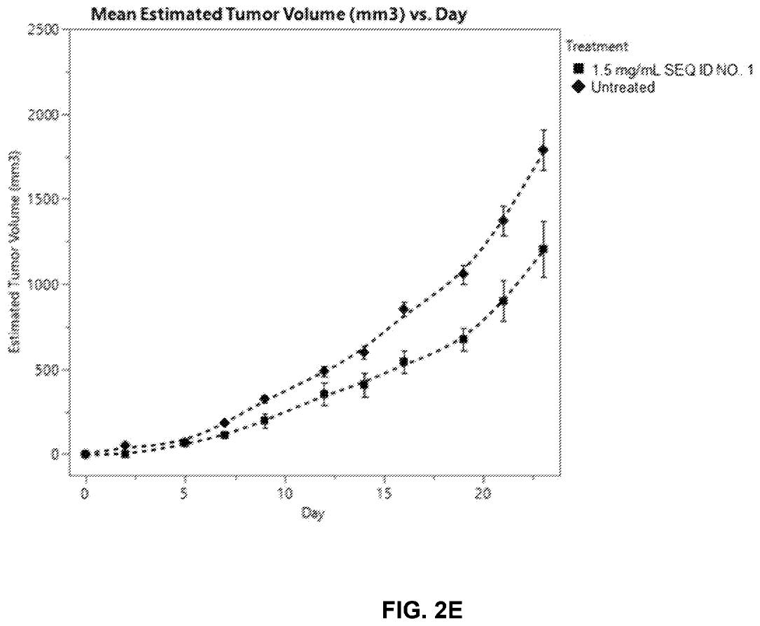

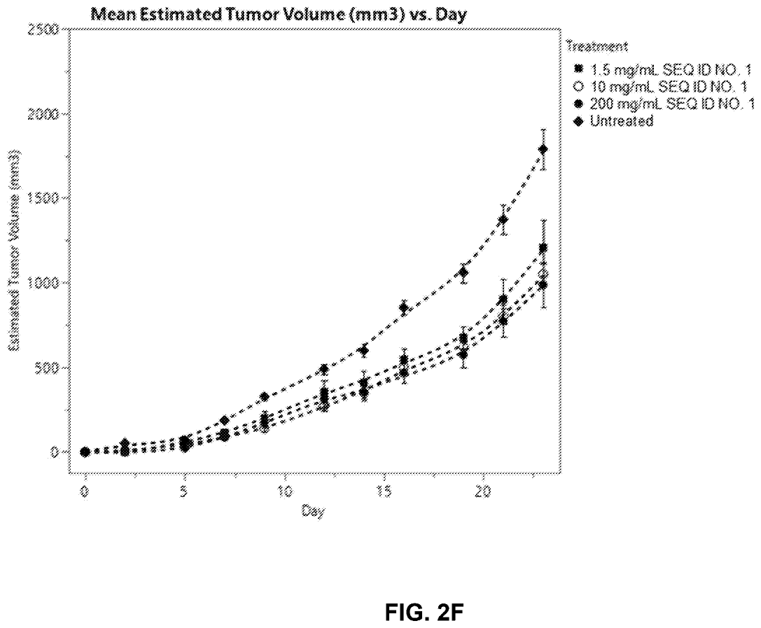

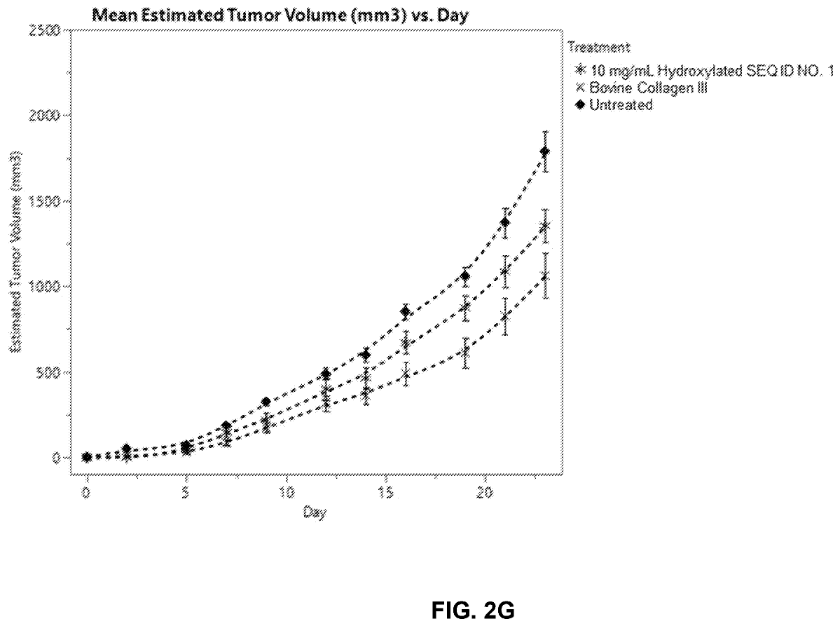

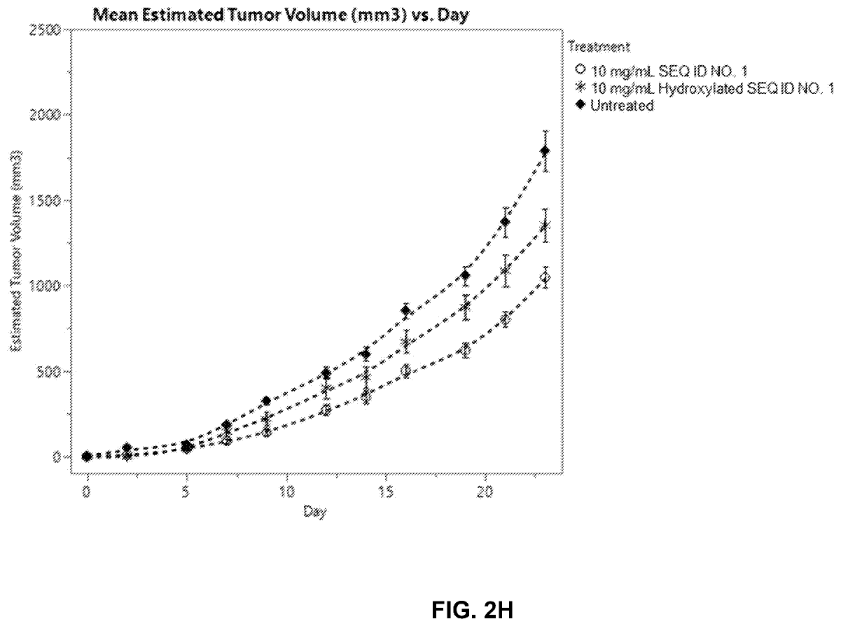

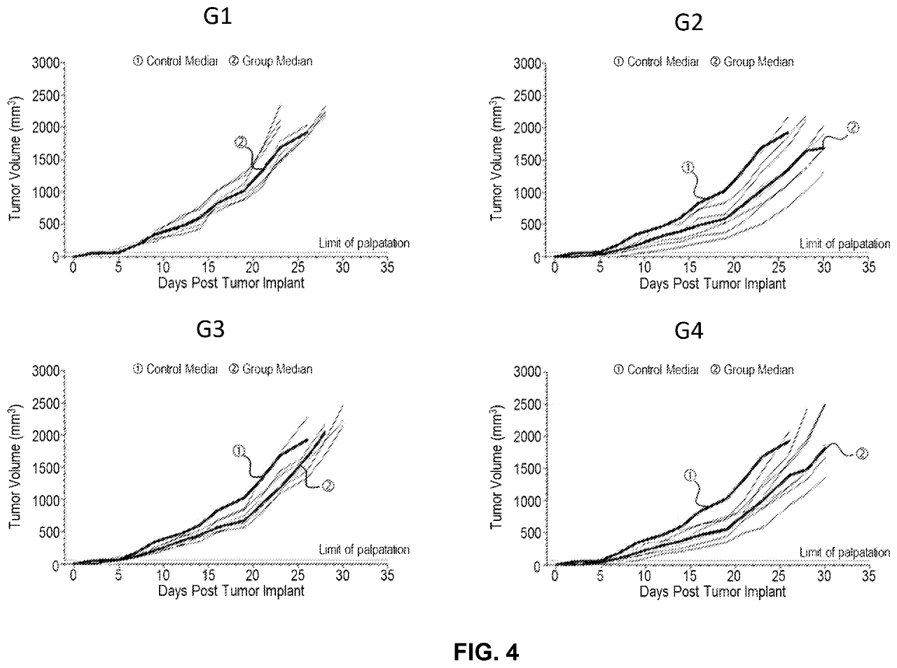

The effect of treatment on estimated tumor volumes is shown in FIGS. 2A-2J. The tumor volume is noted as “estimated” because it is an indirect measurement of the tumor volume. True tumor volumes were calculated after each animal was euthanized and the tumors were removed (FIGS. 1A, 1B, and 4).

### Anatomic Pathology Analysis

To understand the effect of SEQ ID NO: 1 on cancer dormancy, H&E and Masson's Trichrome stained 4T1-Luc-1A4 murine breast carcinoma tumor blocks from the female BALB/c mice in the example above were analyzed for relative amounts of viable tumor cells, necrosis, and collagen in viable tumor areas, compared to untreated samples. Tumor blocks were obtained from mice administered 5E+05 4T1-Luc1A4 cells/animal combined control (Group 1), with 1.5 mg/ml bovine Collagen III (Group 2), or with 200, 10, or 1.5 mg/ml Collagen #1 (Groups 5, 6, and 7, respectively) via subcutaneous injection (200 μL) (experiments described above). H&E and Masson's Trichrome stained slides were digitally scanned from three separate regions of each tumor block (beginning, middle, and end) and scanned images were examined and recorded (Table 4). Fourteen tumor blocks from the following animals were stained with H&E, Masson's Trichrome, and immunohistochemistry (IHC).

Digital scans at 40× were taken from all slides (H&E, Masson's Trichrome, and immunohistochemistry (IHC)) and evaluated. The percent approximations for relative area of viable tumor cells, necrosis, and collagen within viable tumor cell area were generated solely via evaluation of scanned images, and are set forth in Table 5.

TABLE 5

| | | % Area | | % Area | | % Collagen in Non- | |
|---|---|---|---|---|---|---|---|
| | | Necrosis | | Tumor Cells | | Necrotic Tumor Area | |
| Group | N | Mean | SD | Mean | SD | Mean | SD |
| 1 - Control | 2 | 50.83 | 10.21 | 49.17 | 10.21 | 7.50 | 2.74 |
| 2 - 1.5 mg ml hydroxylated Bovine | 3 | 58.33 | 7.50 | 41.67 | 7.50 | 8.33 | 2.50 |
| Collagen III | | | | | | | |
| 5 - 200 mg/ml SEQ ID NO: 1 | 3 | 40 56 | 10.44 | 59.44 | 10.44 | 10.56 | 1.67 |
| 6 - 10 mg/ml SEQ ID NO: 1 | 3 | 52.78 | 25.14 | 47.22 | 25.14 | 10 00 | 250 |
| 7- 1.5 mg/ml SEQ ID NO: 1 | 3 | 48.89 | 14.74 | 51.11 | 14.74 | 9.44 | 3.91 |

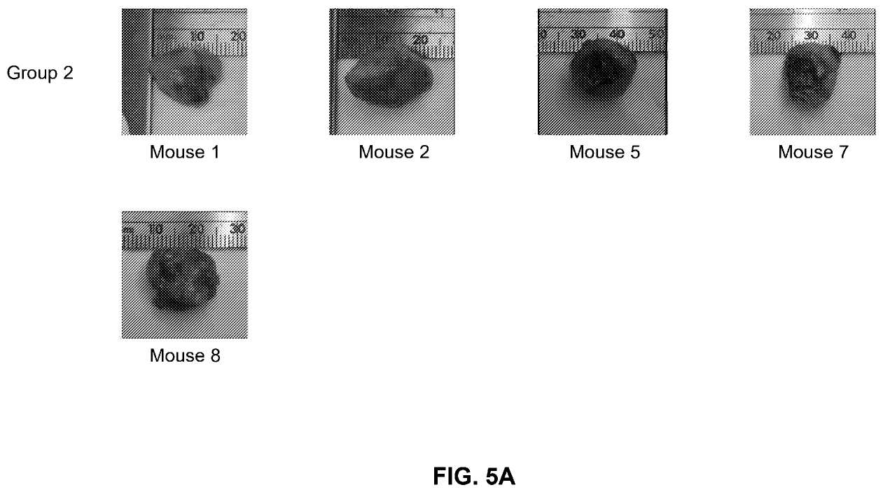

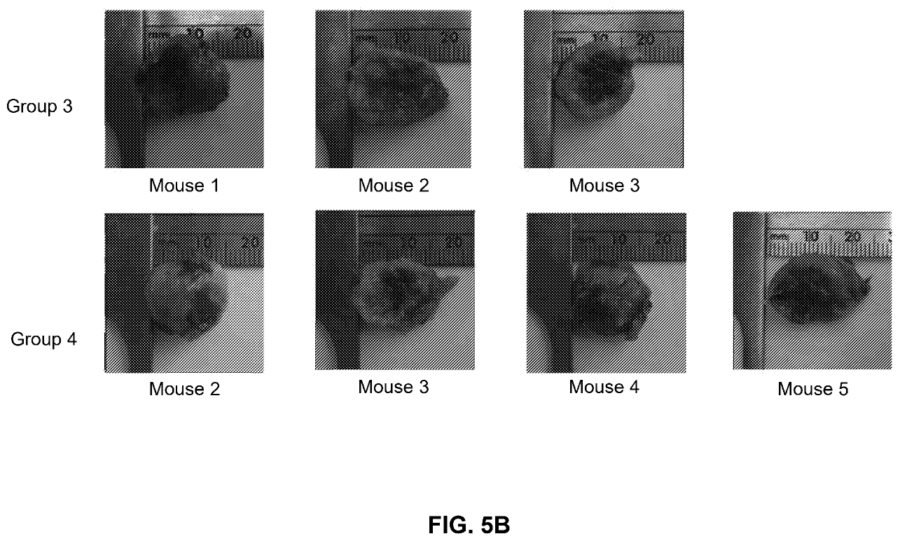

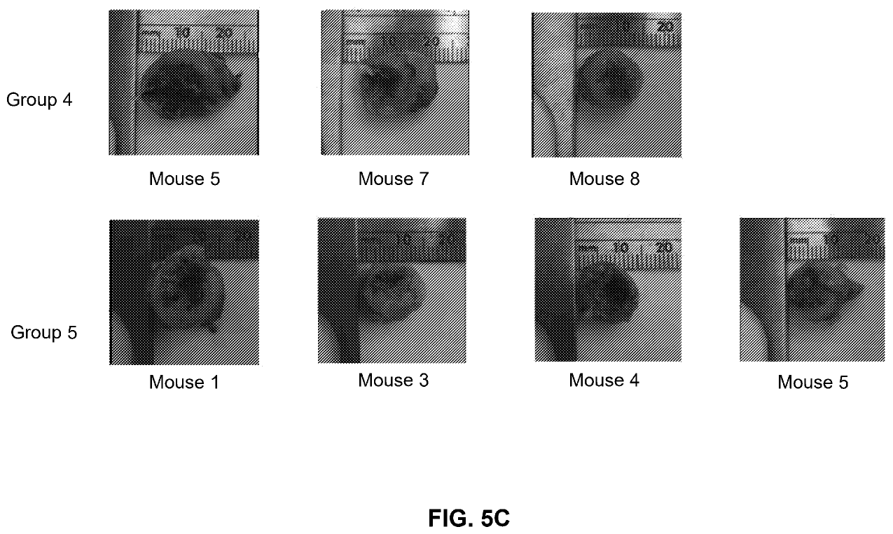

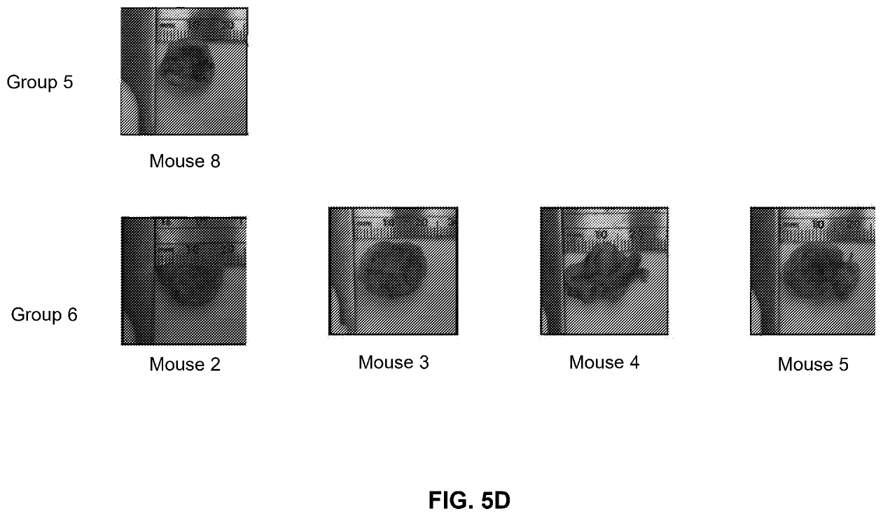

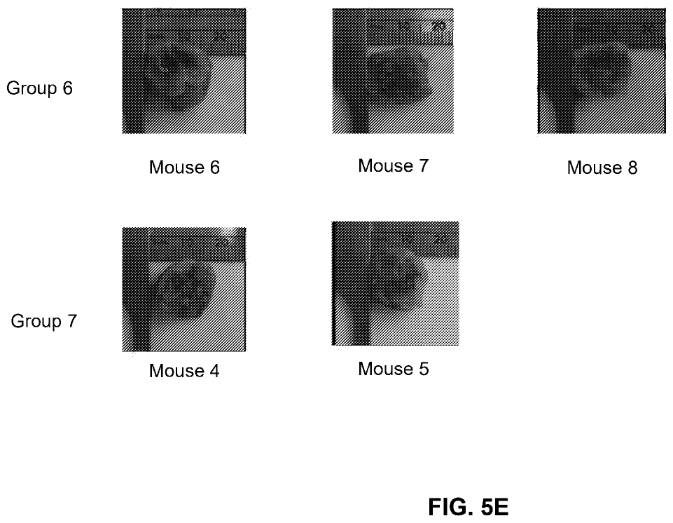

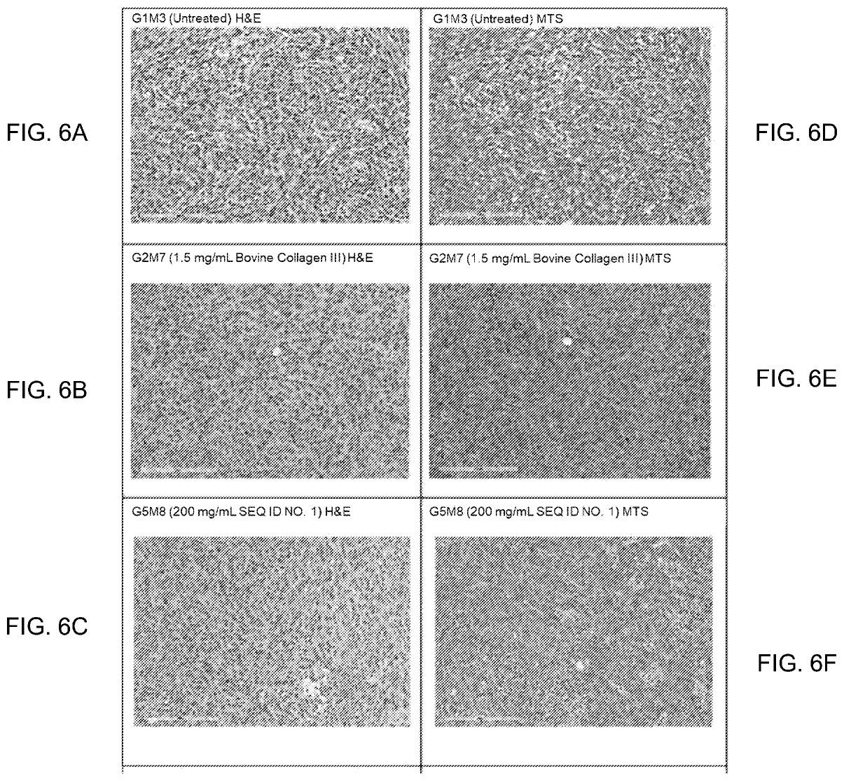

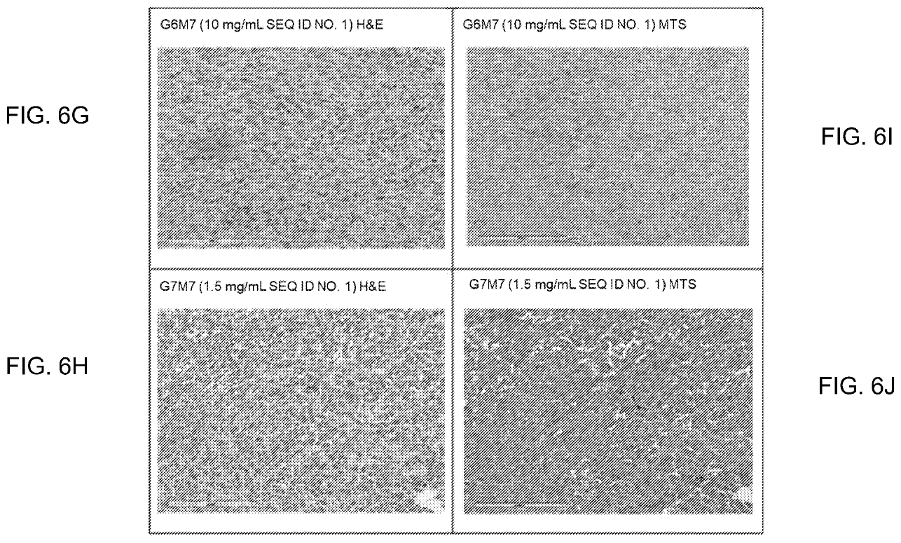

In all groups, regardless of the region of the tumor block, the area of necrosis was localized to the center of the tumor and comprised approximately 40 to 60% of the total tumor area on average for each group (FIGS. 5A-5E). The area of viable tumor cells was mostly located along the periphery of tumors and was comprised of approximately 40 to 60% of the total tumor area overall, regardless of group or region of tumor in the block. Groups administered 200 mg/ml SEQ ID NO: 1, 10 mg/ml SEQ ID NO: 1, and 1.5 mg/ml SEQ ID NO: 1, or bovine collagen had a greater overall mean percentage of collagen in viable tumor area compared to control. Additionally, all three groups administered SEQ ID NO: 1 had a greater mean percentage of collagen in viable tumor area compared to Group 2 animals administered 1.5 mg/mL bovine collagen. Representative photomicrographs of subcutaneous tumors from untreated and treated BALB/c Mice are shown in FIGS. 6A-6J.

Group 2 animals administered 1.5 mg/mL bovine collagen had the lowest average percentage of collagen in viable tumor area among test article-treated groups (mean 8.33 [±2.50], range 5 to 10). However, the mean percentage of collagen in viable tumor area was still greater than that of untreated control animals.

Group 5 animals (200 mg/mL SEQ ID NO: 1) had the greatest relative mean percentage of collagen in viable tumor areas (mean 10.56 [±1.67%], range 10 to 15%) compared with control and all other groups.

Group 6 animals (10 mg/mL SEQ ID NO: 1) had a greater mean relative percentage of collagen in viable tumor percent area (mean 10.00 [±2.50%], range 5 to 15) compared to control animals, Group 2 animals administered 1.5 mg/mL bovine collagen, and Group 7 animals administered 1.5 mg/mL SEQ ID NO: 1.

Group 7 animals administered 1.5 mg/mL SEQ ID NO: 1 had a greater mean percentage of collagen in viable tumor (mean 9.44 [±3.91%], range 5 to 15) than control animals and Group 4 animals administered 1.5 mg/mL Commercial Collagen III.

### Mass Spectrometry Analysis of Tumor Tissue

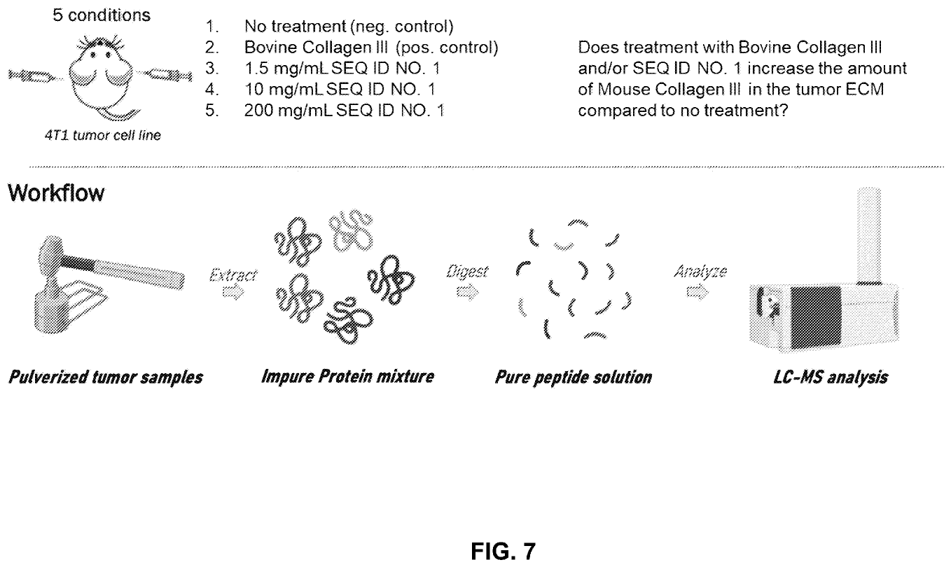

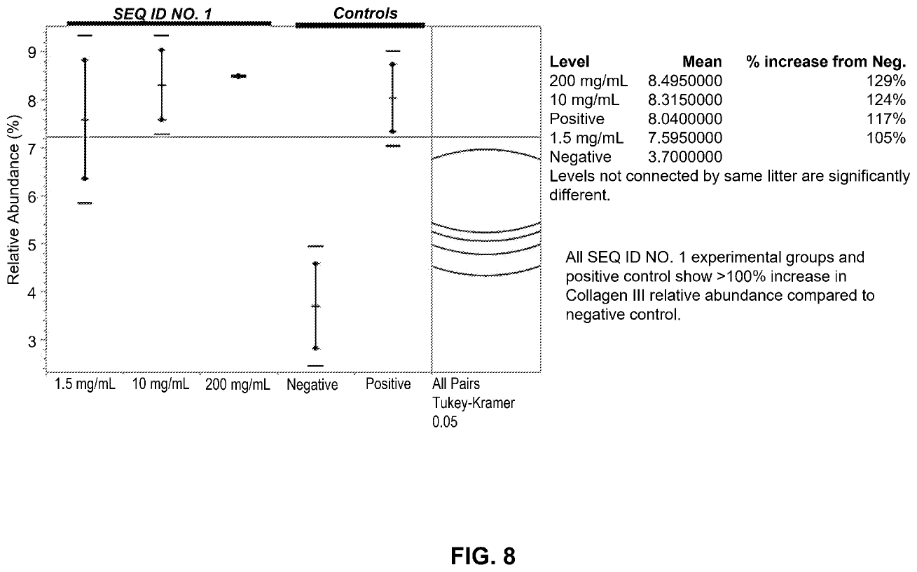

Extracted tumor tissue was evaluated by mass spectrometry and the results are shown in FIGS. 7 and 8. The process is provided below.

Samples were loaded onto the LC-MS (Agilent 6545XT AdvanceBio LC/Q-TOF) for analysis. LC separation was achieved using an Agilent peptide mapping column (Agilent AdvanceBio Peptide Mapping 120 Å, 2.1×150 mm, 2.7 μm, Cat. #: 653750-902) running at 0.4 mL/min flow rate using a 140-minute gradient LC method. The gradient parameters were the following (Solvent A: 0.1% formic acid, Solvent B: 80% acetonitrile, 0.1% formic acid): 0-3 min: 2-10% B, 3-107 min: 10-30% B, 107-121 min: 30-40% B, 121-126 min: 40-60% B, 126-127:60-100% B, 127-137 min: 100% B, 137-138 min: 100-0% B, 138-140 min: 0% B.

Each sample was analyzed on the LC-MS using a conventional auto MS/MS method (data-dependent mode). The MS was run at a resolution of ˜30,000 across 100-3000 m/z with an acquisition rate of 6 spectra/s. MS/MS analysis was performed on the top 8 precursor ions across 50-3000 m/z with 3 spectra/s acquisition rate in each cycle with a normalized (based on size and charge) collision energy and dynamic exclusion of 2 spectra, released after 0.1 min.

The identification and quantification of peptides was achieved using Byologic software (Protein Metrics Inc., San Carlos, CA). All raw Agilent *.d data files were searched against a Mus musculus (mouse) proteome protein database (Uniprot, Proteome ID: UP000000589) concatenated with SEQ ID NO: 1. Peptide search parameters were set to fully-specific trypsin digestion, allowing up to two missed cleavages. Precursor mass tolerance was set to 15 ppm with fragment mass tolerance set to 20 ppm with QTOF/HCD fragmentation type.

Carbamidomethylation (Cys; +57.021464 Da) was set as a fixed modification. The following were set as rare (1 occurrence per peptide) variable modifications: Gln->pyro-Glu (N-terminal Gln; −17.026549 Da), Glu->pyro-Glu (N-terminal Glu; −18.010565), carbamylation (peptide N-terminal; +43.005814), phosphorylation (Ser, Thr, Tyr; +79.966331), and oxidation (Met; +15.994915). Deamidation (Asn; +0.984016) was set as a common variable modification. Hydroxyproline (up to three per peptide) and hydroxylysine were assigned as variable modifications only on “collagen-family” proteins to improve the peptide coverage.

After peptide identification and sequence matching, all scans with a score <100 were filtered out of the data set. The relative protein abundance for each sample was calculated by summing the XIC area of all peptide matches for a specific protein and dividing by the total peptide XIC area for that sample. Relative protein abundances were then compared for the proteins of interest between experimental conditions and negative (Group 1)/positive (Group 2) control groups.

As shown in FIG. 8, and Table 5, collagen was enriched over 100% in each experimental group compared to the negative control.

The collagen compositions of the tumor samples, as determined by mass spectrometry, are set forth in Table 6.

TABLE 6

| | | Group 2 | | | |
|---|---|---|---|---|---|
| | | 50% + | | | |
| | | 1.5 mg/ | Group 2 | | Group 5 |
| | | mL | 1.5 | Group 6 | 200 |
| | | bovine | mg/mL | 10 mg/mL | mg/mL |
| | Group 1 | collagen | SEQ ID | SEQ ID | SEQ ID |
| Protein | (Negative) | (Positive) | NO: 1 | NO: 1 | NO: 1 |
| Collagen alpha-1(I) chain | 11.77 | 19.75 | 17.26 | 16.05 | 18.40 |
| Collagen alpha-1(III) chain | 3.70 | 8.04 | 7.60 | 8.32 | 8.50 |
| Collagen alpha-1(V) chain | 0.10 | 0.18 | 0.11 | 0.14 | 0.13 |
| Collagen alpha-1(VI) chain | 0.00 | 0.04 | 0.04 | 0.03 | 0.02 |
| Collagen alpha-1(VII) | 0.10 | 0.06 | 0.13 | 0.14 | 0.11 |
| chain | | | | | |
| Collagen alpha-2(I) chain | 9.63 | 12.22 | 12.24 | 10.24 | 12.65 |
| Collagen alpha-2(IV) chain | 0.03 | 0.05 | 0.10 | 0.11 | 0.12 |
| Collagen alpha-2(V) chain | 0.16 | 0.16 | 0.19 | 0.19 | 0.14 |
| Collagen alpha-2(VI) chain | 0.06 | 0.05 | 0.05 | 0.05 | 0.04 |

This example demonstrates that treatment with bovine collagen and treatment with SEQ ID NO: 1 increases the amount of collagen in the tumor ECM. Treatment with SEQ ID NO: 1 is as effective as treatment with commercially available bovine Collagen III.

### In Vitro Cancer Cell Line Viability Assays

An in vitro cell viability assay was performed to evaluate whether SEQ ID NO: 1 (50 KDa rCol protein) induced dormancy in various cancers.

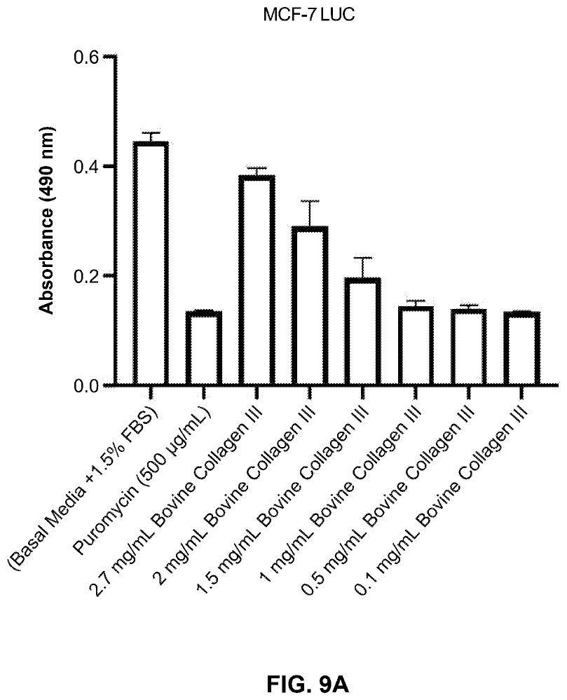

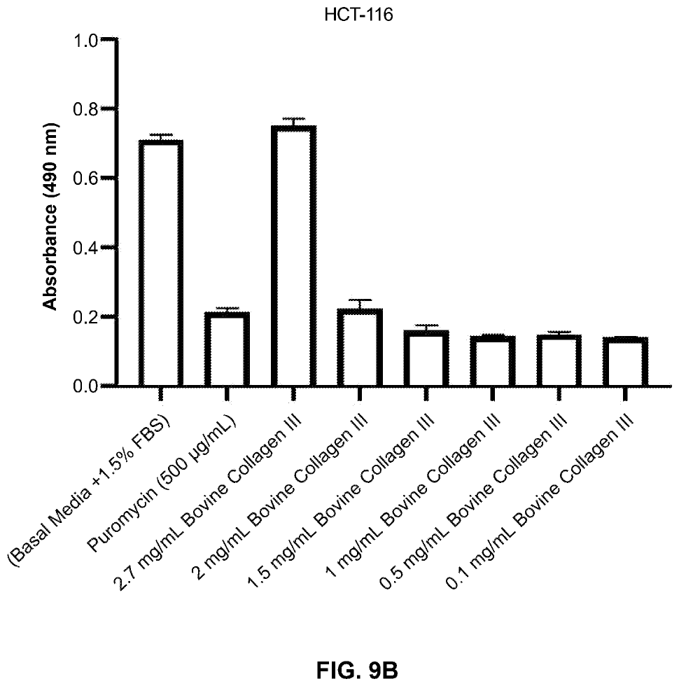

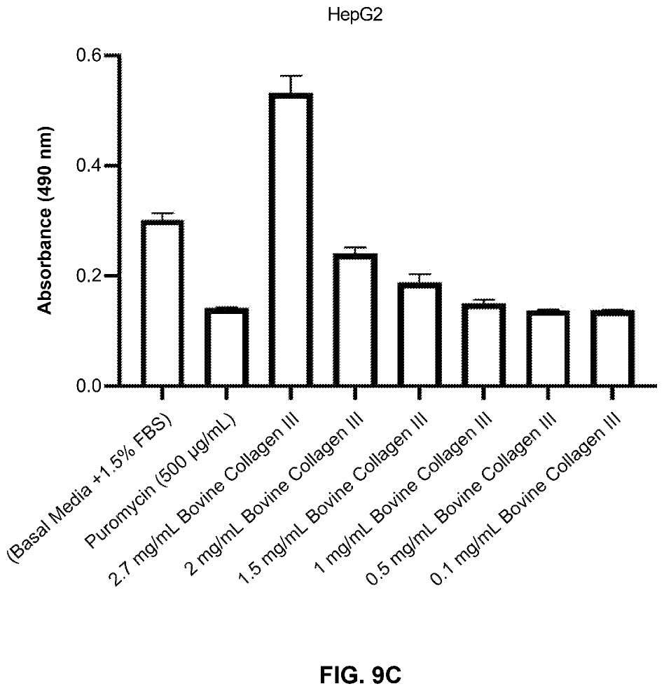

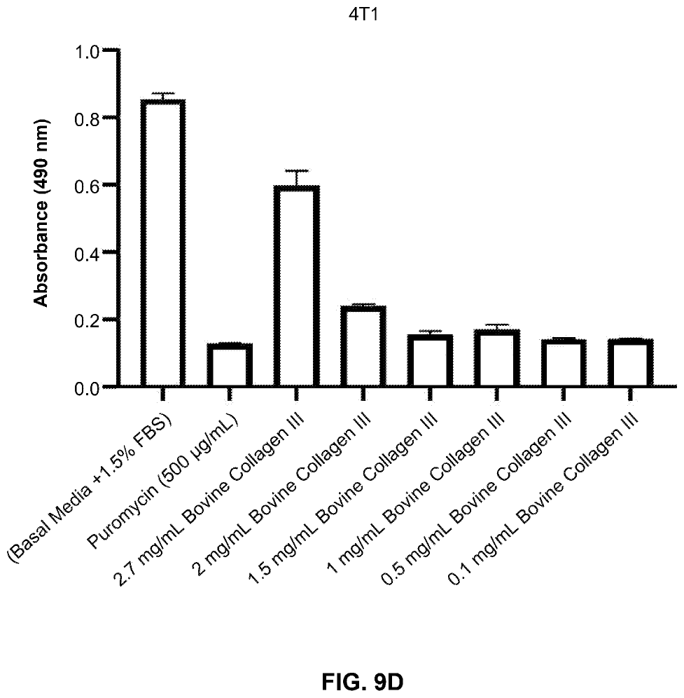

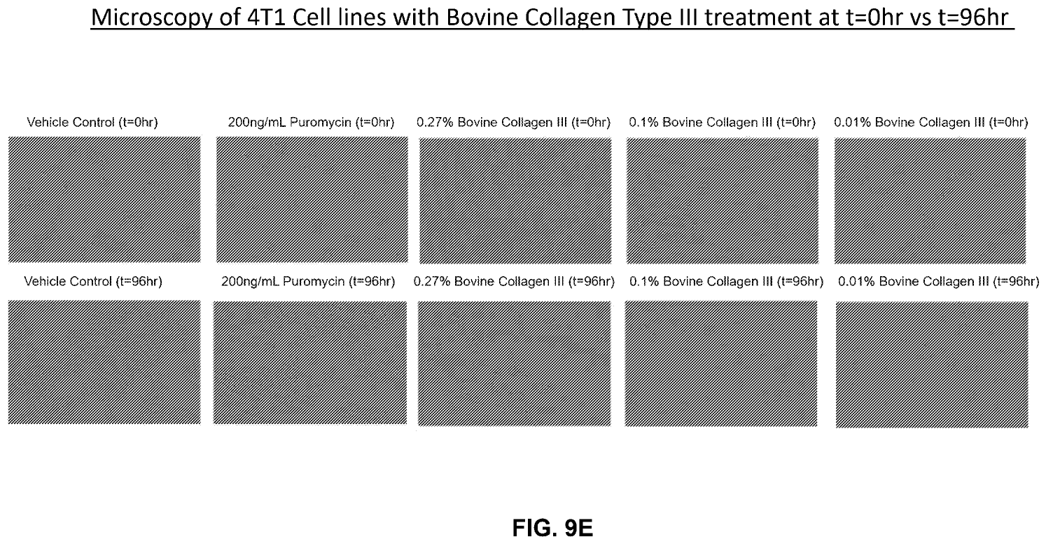

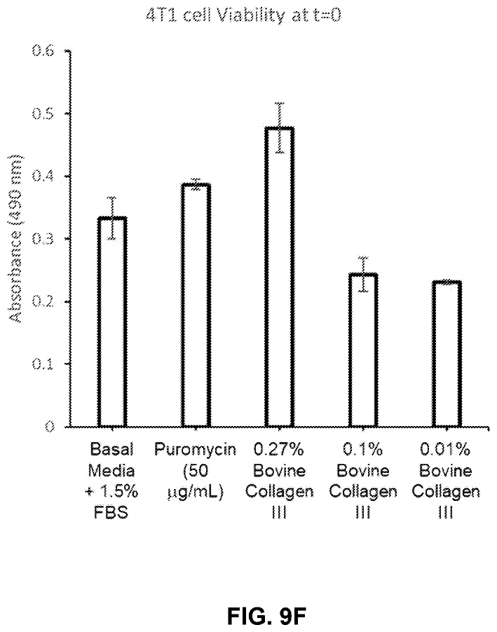

Fourteen different conditions (negative control (basal media+1.5% FBS), positive control (puromycin (500 μg/mL), 6 different SEQ ID NO: 1 concentrations, and 6 different bovine collagen Type III concentrations were compared. The cell lines 4T1, HCT 116, Hep G2, and MCF7 were treated with SEQ ID NO: 1 and bovine collagen. Criteria for the cell lines chosen included (1) cell doubling time ≤30 hr (2) cells from human origin, and (3) cells that were able to grow on plastic without a Matrigel ECM matrix. The levels of proliferation between the different treatments were assayed, as described, and the results are shown in FIGS. 9A-9F (bovine) and 10A-10C (hCOL3). Fourteen conditions were selected to evaluate each cell line.
1. Negative control—Untreated (n=6 minimal)
2. Positive control—Puromycin (A potent translational inhibitor that causes rapid cell death) (n=6 minimal)
3. 2% w/w SEQ ID NO: 1 (n=6 minimal) [Final solution concentration]
4. 1% w/w SEQ ID NO: 1 (n=6 minimal) [Final solution concentration]
5. 0.5% w/w SEQ ID NO: 1 (n=6 minimal) [Final solution concentration]
6. 0.25% w/w SEQ ID NO: 1 (n=6 minimal) [Final solution concentration]
7. 0.125% w/w SEQ ID NO: 1 (n=6 minimal) [Final solution concentration]
8. 0.0625% w/w SEQ ID NO: 1 (n=6 minimal) [Final solution concentration]
9. 2.7 mg/mL Bovine Collagen Type III (n=6 minimal) [Final solution concentration]
10. 2 mg/mL Bovine Collagen Type III (n=6 minimal) [Final solution concentration]
11. 1.5 mg/mL Bovine Collagen Type III (n=6 minimal) [Final solution concentration]
12. 1 mg/mL Bovine Collagen Type III (n=6 minimal) [Final solution concentration]
13. 0.5 mg/mL Bovine Collagen Type III (n=6 minimal) [Final solution concentration]
14. 0.1 mg/mL Bovine Collagen Type III (n=6 minimal) [Final solution concentration]

The results for bovine collagen are shown in FIGS. 9A-9F. Surprisingly, higher bovine collagen concentrations correlated with higher absorbance implying greater cellular proliferation. This data notwithstanding, lower bovine collagen concentrations (e.g. 1 mg/mL, 0.5 mg/mL, and 0.1 mg/mL) had reduced absorbance relative to the higher concentrations, implying reduced cellular proliferation. The apparent disparity resulting from bovine collagen concentration suggests that bovine collagen interferes with the assay and, more likely than not, that at 2.7 mg/mL, 2 mg/mL, and 1.5 mg/mL, bovine collagen inhibits cellular growth despite the data presented in FIGS. 9A-9E for the higher concentrations. This is supported by the data in FIG. 9F, which indicates the effect of bovine collagen at different concentrations on 4T1 cells, measured by the proliferation assay reading at time zero. Furthermore, microscope images in FIG. 9E show that bovine collagen inhibits cellular growth at all concentrations, including high concentrations of 2.7 mg/mL.

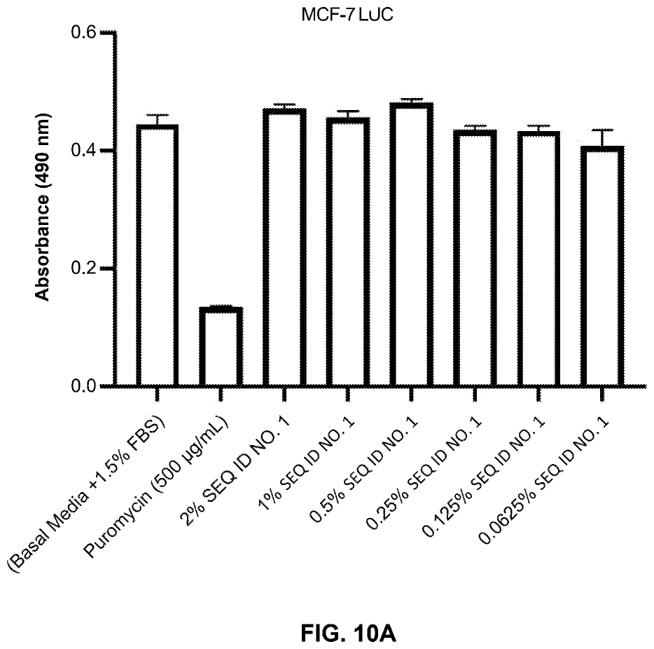

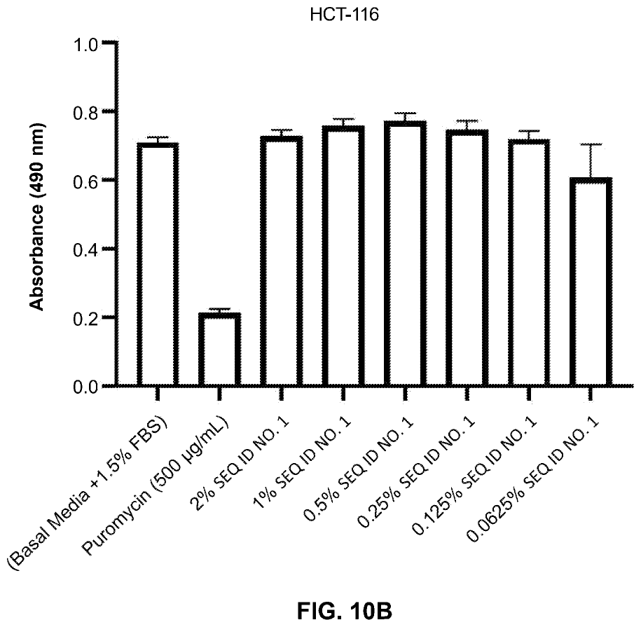

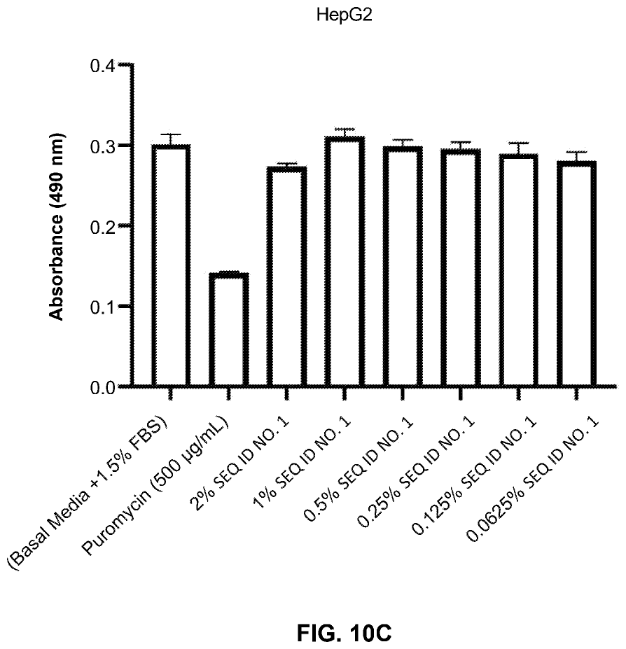

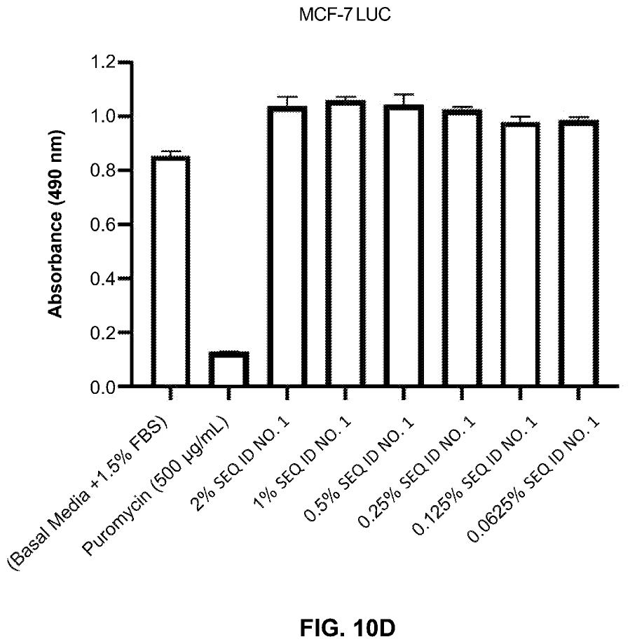

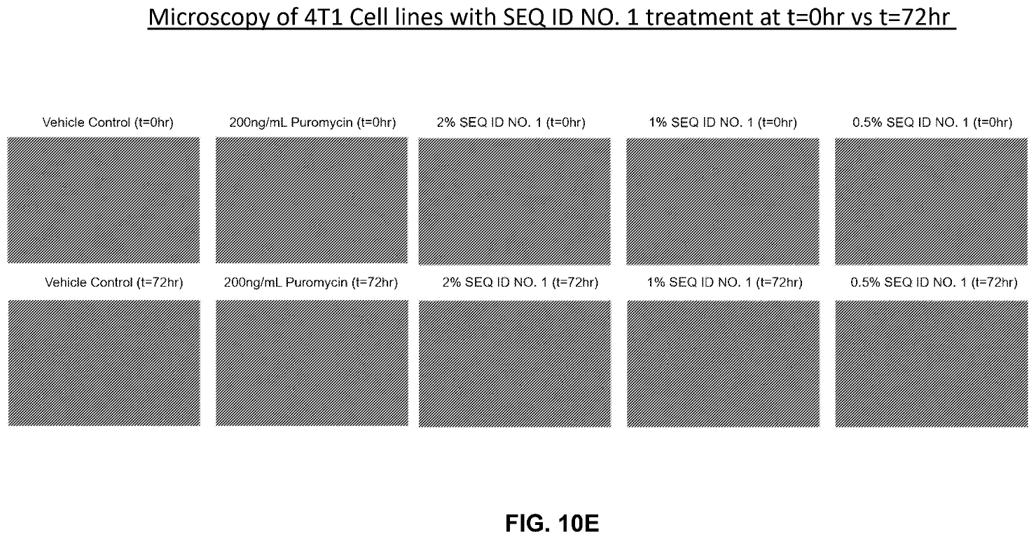

The results for SEQ ID NO: 1 are shown in FIGS. 10A-10E. Based on the in vivo data presented above, it was expected that SEQ ID NO:1 would inhibit cellular proliferation in vitro. Despite these expectations, FIGS. 10A-10E suggest that SEQ ID NO: 1 did not inhibit cellular growth in vitro. Without wishing to be bound by a particular theory, it is believed that the conditions used to plate the varying cell types in this example—which differed from the conditions used for the bovine collagen data presented in FIGS. 9A-9F—affected the ability of SEQ ID NO: 1 to inhibit cellular proliferation.

### Example 3: Application of Therapeutic Biomaterial with Recombinant Collagen Fragment

T-HEp3 cells expressing a CDK2 biosensor are injected orthotopically in mice. At the time of tumor resection, a recombinant collagen fragment-loaded therapeutic biomaterial (or DPBS-loaded control) is applied to the wound area. Mice are monitored for recurrence of tumor cells after resection of the primary tumor, and compared to the control group. Intravital imaging is used to view cancer cells. Cells that are growth arrested in GO are determined by nuclear localization of the CDK2 sensor.

### Example 4: Application of Therapeutic Biomaterial with Recombinant Collagen Fragment

T-HEp3 cells expressing a CDK2 biosensor are injected orthotopically in mice. The tumor is resected and sutured. After the skin has healed, the sutures are removed and a scar is visible. A recombinant collagen fragment-loaded therapeutic biomaterial (or DPBS-loaded control) is applied to the scar. Mice are monitored for recurrence of tumor cells at the scar site, compared to the control group. Intravital imaging is used to view cancer cells. Cells that are growth arrested in GO are determined by nuclear localization of the CDK2 sensor.

### Example 5: Application of Therapeutic Biomaterial with Recombinant Collagen Fragment

T-HEp3 cells expressing a CDK2 biosensor are injected orthotopically in mice. The tumor is resected and sutured. A recombinant collagen fragment-loaded therapeutic biomaterial (or DPBS-loaded control) is applied as a wound dressing immediately after surgery. Mice are monitored for recurrence of tumor cells following removal of the wound dressing, compared to the control group. Intravital imaging is used to view cancer cells. Cells that are growth arrested in GO are determined by nuclear localization of the CDK2 sensor.

### Example 6: Application of Therapeutic Biomaterial with Recombinant Collagen Fragment

T-HEp3 cells expressing a CDK2 biosensor are injected orthotopically in mice. The tumor is resected and the recombinant collagen fragment-loaded therapeutic biomaterial (or DPBS-loaded control) is applied internally to the resection site and remains there while sutures are applied. Mice are monitored for recurrence of tumor cells following removal of the sutures, compared to the control group. Intravital imaging is used to view cancer cells. Cells that are growth arrested in GO are determined by nuclear localization of the CDK2 sensor.

### Examples 7-68—Investigation of Therapeutic Biomaterials

Mixtures of HA and collagens, and mixtures of various other additional polymers and collagens, were investigated for use as therapeutic biomaterials, as described herein.

### Example 7

Polyethylene oxide (PEO) with a molecular weight of 400 kDa was purchased from Sigma (Sigma-Aldrich, St. Louis, MO) (“Polymer 1”). A recombinant collagen fragment with a molecular weight of 50 kDa having SEQ ID NO: 1 was prepared in yeast, as described in PCT/US2022/027016, which is incorporated herein by reference in its entirety (“Polymer 2”). A 5 wt % polyethylene oxide (PEO) solution was prepared by dissolving 2.5 g PEO in 47.5 ml deionized (DI) water and mixing on a stir plate at 400 RPM for 16 hours. A 5% (w/v) solution including the recombinant collagen fragment with the amino acid sequences of SEQ ID NO: 1 was produced by dissolving 5 grams of the 50 kDa protein into 100 ml of water. The two solutions were mixed at a 1:1 (w/w) ratio in a 50 ml conical tube and mixed at 10 RPM for 16 hours. The conical tube was placed vertically on a rack for overnight before assessing the solution stability. The final solution was turbid and slowly phase separated, with white or yellow-ish aggregates settled at bottom of the tube.

### Example 8

A mixture including polyethylene oxide (PEO) and a recombinant collagen fragment with a molecular weight of 50 kDa having SEQ ID NO: 1 was prepared and dried, and visually examined. PEO with a molecular weight of 100 kDa was purchased from Sigma (“Polymer 1”). The remainder of the method was the same as set forth in Example 7. The final solution was transparent. The final solution was phase separated with a hazy solution on top, and hazy and slightly yellow solution on bottom.

### Example 9

A first polyvinylpyrrolidone (PVP) and collagen (recombinant collagen fragment with a molecular weight of 50 kDa having SEQ ID NO: 1) mixture was prepared and dried, and visually examined. Example 9 was prepared using the same method described in Example 7, except that Polymer 1 was replaced with polyvinylpyrrolidone (Sigma; molecular weight of 10 kDa). The final solutions were transparent. About 13 grams (g) of solution was cast into a 100 ml Polytetrafluoroethylene (PTFE) evaporating dish and dried at 45° C. overnight. The dried films were visually transparent.

This example demonstrates that solutions including 10 kDa PVP and the recombinant collagen fragment with a molecular weight of 50 kDa having SEQ ID NO: 1 are fully miscible and can be prepared as a film or article.

### Example 10

A second polyvinylpyrrolidone (PVP) and collagen (recombinant collagen fragment with a molecular weight of 50 kDa having SEQ ID NO: 1) mixture was prepared, dried, and visually examined. Example 10 was prepared using the method described in Example 7, except that Polymer 1 was replaced with polyvinylpyrrolidone (Sigma; molecular weight of 40 kDa). The final solutions were transparent. Films were prepared using the method described in Example 9. The dried films were visually transparent.

This example demonstrates that solutions including 40 kDa PVP and the recombinant collagen fragment with a molecular weight of 50 kDa having SEQ ID NO: 1 are fully miscible and can be prepared as a film or article.

### Example 11

A third polyvinylpyrrolidone (PVP) and collagen (recombinant collagen fragment with a molecular weight of 50 kDa having SEQ ID NO: 1) mixture was prepared and dried, and visually examined. Example 11 was prepared using the method described in Example 7, except that Polymer 1 was replaced with polyvinylpyrrolidone (Sigma; molecular weight of 360 kDa). The final solutions were transparent. Films were prepared using the same method in Example 9. The dried films were visually transparent.

This example demonstrates that solutions including 360 kDa PVP and the recombinant collagen fragment with a molecular weight of 50 kDa having SEQ ID NO: 1 are fully miscible and can be prepared as a film.

### Example 12

A first chitosan and collagen (recombinant collagen fragment with a molecular weight of 50 kDa having SEQ ID NO: 1) mixture was prepared and visually examined. Example 12 was prepared using the method described in Example 7, except that Polymer 1 was replaced with chitosan (molecular weight range of 50 kDa to 190 kD; Sigma). The chitosan solution was pH adjusted to pH 4 using 0.287 ml of acetic acid from Sigma to fully solubilize the solution during mixing. The final solution was phase separated into two layers. The viscosity was determined visually by inspecting the difference between the two layers. The top layer had lower viscosity and was more transparent, whereas the bottom layer had higher viscosity and was more turbid. The viscosity was determined visually by inspecting the difference between the two layers.

### Example 13

A polyethylene glycol and collagen (recombinant collagen fragment with a molecular weight of 50 kDa having SEQ ID NO: 1) mixture was prepared and dried, and visually examined. Example 14 was prepared using the method described in Example 7, except that Polymer 1 was replaced with polyethylene glycol (molecular weight of 35 kDa Sigma). The final solution was phase separated with a transparent and clear solution on the top and a transparent and slightly yellow solution on the bottom

### Example 14

A first alginic acid and collagen (recombinant collagen fragment with a molecular weight of 50 kDa having SEQ ID NO: 1) mixture was prepared and dried, and visually examined. Examples 14 was prepared using the method described in Example 7, except that Polymer 1 was replaced with medium viscosity alginic acid sodium salt from brown algae (Sigma). The viscosity of 2% alginic acid sodium salt in water at 25° C. was equal to or greater than 2,000 cps. A 2.5 wt % alginic acid solution was prepared by dissolving 2.5 g alginic acid sodium salt in 97.5 ml deionized (“DI”) water and mixing on a stir plate at 400 RPM for 24 hours due to the high viscosity of the solution. The viscosity was determined visually by inspecting the difference between the two layers. The two solutions were mixed at a 1:1 (v/v) ratio in a 50 ml conical tube and mixed at 10 RPM for 16 hours. The conical tube was placed vertically on a rack for overnight before assessing the solution stability. The final solution was transparent. A film was prepared using the method set forth in Example 9. The dried film was visually hazy with uniformly distributed haziness throughout the film.

### Example 15

A second alginic acid and collagen (recombinant collagen fragment with a molecular weight of 50 kDa having SEQ ID NO: 1) mixture was prepared and dried, and visually examined. Example 15 was prepared using the method described in Example 7, except that Polymer 1 was replaced with low viscosity alginic acid sodium salt from brown algae (Sigma). The viscosity of 1% alginic acid sodium salt in water at 25° C. was about 4-12 cps. Both alginic acid sodium salt and collagen stock solutions were 5 wt %. The two solutions were mixed at a 1:1 (v/v) ratio in a 50 ml conical tube and mixed at 10 RPM for 16 hours. The final solution was transparent. A film was prepared using the method set forth in Example 9. The dried film was visually hazy with uniformly distributed haziness throughout the film.

### Example 16

A polyacrylamide and collagen (recombinant collagen fragment with a molecular weight of 50 kDa having SEQ ID NO: 1) mixture was prepared and dried, and visually examined. Example 16 was prepared using the method described in Example 7, except that Polymer 1 was replaced with polyacrylamide (molecular weight range of 5,000 kDa to 6,000 kDa; Sigma). The remainder of the method was the same as set forth in Example 7. The final solution was transparent. A film was prepared using the same method described in Example 9. The dried film was visually transparent.

This example demonstrates that solutions including 5,000 kDa to 6,000 kDa polyacrylamide and the recombinant collagen fragment with a molecular weight of 50 kDa having SEQ ID NO: 1 are miscible and can be prepared as a film or article.

### Example 17

A polyacrylamide and collagen (recombinant collagen fragment with a molecular weight of 50 kDa having SEQ ID NO: 1) mixture was prepared and dried, and visually examined. Example 17 was prepared using the method described in Example 7, except that Polymer 1 was replaced with polyacrylamide (molecular weight of 40 kDa; Sigma). The remainder of the method was the same as set forth in Example 7. The final solution was transparent. A film was prepared using the method described in Example 9, except using an aluminum weighing pan. The dried film was visually transparent.

This example demonstrates that solutions including 40 kDa polyacrylamide and the recombinant collagen fragment with a molecular weight of 50 kDa having SEQ ID NO: 1 are miscible and can be prepared as a film or article.

### Example 18

A first polyvinyl alcohol (PVA) and collagen (recombinant collagen fragment with a molecular weight of 50 kDa having SEQ ID NO: 1) mixture was prepared and visually examined. Example 18 was prepared using the method described in Example 7, except that Polymer 1 was replaced with PVA (molecular weight range of 89 kDa to 98 kDa; Sigma). A 4 wt % PVA solution was prepared by dissolving 2 g PVA in 48 ml DI water and mixing on a stir plate at 400 RPM for 16 hours. The two solutions were mixed at a 1:1 (w/w) ratio in a 50 ml conical tube and mixed at 10 RPM for 16 hours. The final solution was phase separated into two layers. The top layer was turbid and there was a large amount of white powder-like aggregates settled at the bottom.

### Example 19

A second PVA and collagen (recombinant collagen fragment with a molecular weight of 50 kDa having SEQ ID NO: 1) mixture was prepared and visually examined. Example 19 was prepared using the method described in Example 18, except that Polymer 1 was replaced with a 4% PVA (w/v) solution (unknown molecular weight; Fisher Scientific). The remainder of the method was the same as set forth in Example 18. The final solution was phase separated into two layers. The viscosity was determined visually by inspecting the difference between the two layers. The top layer had lower viscosity and was more turbid with small, suspended particles. The bottom layer had higher viscosity and was transparent.

### Example 20

A first hyaluronic acid (HA) and collagen (recombinant collagen fragment with a molecular weight of 50 kDa having SEQ ID NO: 1) mixture was prepared and dried, and visually examined. Example 20 was prepared using the method described in Example 7, except that Polymer 1 was replaced with hyaluronic acid (HA) (molecular weight of 1,000 kDa purchased from Pure Health Botanicals (Saint Charles, IL)). A 2.5 wt % HA solution was prepared by dissolving 5 g HA in 195 ml DI water and mixing with a high shear impeller at 400 RPM for 40 hours due to the high viscosity of the solution. A 5% (w/v) collagen solution including the recombinant collagen fragment of SEQ ID NO: 1 was produced as described in Example 7. The HA solution and collagen solution were mixed at a 2:1 (w/w) ratio in a 50 ml conical tube and mixed at 10 RPM for 16 hours to maintain the dry mass ratio of 1:1. The final solution was transparent. A film was prepared using the method described in Example 9. The dried film was visually hazy with uniformly distributed haziness throughout the film.

### Example 21

A second hyaluronic acid (HA) and collagen (recombinant collagen fragment with a molecular weight of 50 kDa having SEQ ID NO: 1) mixture was prepared and dried, and visually examined. Example 21 was prepared using the method in Example 7, except that Polymer 1 was replaced with non-animal-based HA (molecular weight of 50 kDa; Pure Health Botanicals (Saint Charles, IL) and DSM (Kaiseraugst, Switzerland)). A 5 wt % HA solution was prepared by dissolving 5 g HA in 95 ml DI water and mixing on stir plate at 400 RPM for 16 hours. A 5% (w/v) collagen solution including the recombinant collagen fragment of SEQ ID NO: 1 was produced as described in Example 7. The two solutions were mixed at a 1:1 (w/w) ratio in a 50 ml conical tube and mixed at 10 RPM for 16 hours. The final solution was transparent. A film was prepared using the method described in Example 9. The dried film was visually hazy with uniformly distributed haziness throughout the film. In addition, an aliquot of HA and collagen solution mixture was placed in a fridge at 4° C. overnight and the solution remained transparent.

### Example 22

A first gelatin and HA mixture was prepared and visually examined. Example 22 was prepared using the method in Example 21, except that Polymer 2 was replaced with low molecular weight beef gelatin dietary supplement (Great Lakes Wellness, Grayslake, IL). A 2.2 wt % gelatin solution was prepared by dissolving 1 g gelatin in 45 ml 0.01N HCl and mixing with a high shear impeller at 400 RPM for 16 hours due to the high viscosity of the solution. A 5 wt % 50 kDa HA solution was prepared using the method described in Example 21. The HA solution and gelatin solution were mixed at a 1:2.4 (w/w) ratio in a 50 ml conical tube and mixed at 10 RPM for 16 hours to maintain the dry mass ratio of 1:1. The final solution was phase separated into two layers. The viscosity was determined visually by inspecting the difference between the two layers. The top layer had lower viscosity and was more transparent. The bottom layer had higher viscosity and was more turbid.

### Example 23

A second gelatin and HA mixture was prepared and visually examined. Example 23 was prepared using the method described in Example 22, except that Polymer 2 was replaced with gelatin from porcine skin (molecular weight of 300 kDa; Sigma). A 1.1 wt % gelatin solution was prepared by dissolving 0.5 g gelatin in 45 ml 0.01N HCl and mixing with a high shear impeller at 400 RPM for 16 hours due to the high viscosity of the solution. A 5 wt % 50 kDa HA solution was prepared using the method described in Example 21. The HA solution and gelatin solution were mixed at a 1:4.5 (w/w) ratio in a 50 ml conical tube and mixed at 10 RPM for 16 hours to maintain the dry mass ratio of 1:1. The final solution was phase separated with a large number of white aggregates settled at the bottom and scattered white aggregates suspended or floating at the top.

### Example 24

A HA and collagen type I mixture was prepared and visually examined. Example 24 was prepared using the method described in Example 21 except that Polymer 2 was replaced with collagen type I (molecular weight of 414 kDa; Sigma). A 0.7 wt % collagen type I solution was prepared by dissolving 0.3 g collagen type I in 45 ml 0.01N HCl and mixing with a high shear impeller at 400 RPM for 16 hours due to the high viscosity of the solution. A 5 wt % 50 kDa HA solution was prepared using the method described in Example 21. The HA solution and collagen type I solution were mixed together in a 1:7 (w/w) ratio in a 50 ml Falcon conical tube and mixed with Hula mixer at 10 RPM for 16 hours to maintain the dry mass ratio of 1:1. The final solution was phase separated with a large number of white fibrils settled at the bottom and scattered white fibrils suspended or floating at the top.

### Example 25

A cellulase and HA mixture was prepared and visually examined. Example 25 was prepared using the method described in Example 21, except that Polymer 2 was replaced with cellulase (Sunson Industry Group Co., Ltd.). A 5% (w/v) cellulase solution was prepared by dissolving 2.5 g cellulase in 47.5 ml DI water and mixing on stir plate at 400 RPM for 16 hours. A 5 wt % 50 kDa HA solution was prepared using the same method described in Example 21. The two solutions were mixed together in a 1:1 (w/w) ratio in a 50 ml conical tube and mixed at 10 RPM for 16 hours. The final solution was turbid with yellow powders settled at the bottom.

### Example 26

A HA, chitosan, and collagen (recombinant collagen fragment with a molecular weight of 50 kDa having SEQ ID NO: 1) mixture was prepared and visually examined. Example 26 was prepared using the method described in Example 21 except the chitosan that was used in Example 12 was added as Polymer 3. A 5 wt % 50 kDa HA solution was prepared by dissolving 5 g HA in 95 ml DI water and mixing on stir plate at 400 RPM for 16 hours A 5% (w/v) collagen solution including the recombinant collagen fragment of SEQ ID NO: 1 was produced by dissolving 5 grams of the 50 kDa protein into 100 ml of water, as described in Example 7. A 5 wt % chitosan solution was prepared by dissolving 2.5 g chitosan in 47.5 ml DI water and mixing on stir plate at 400 RPM for 16 hours. The chitosan solution was pH adjusted to pH 4 using 0.287 ml acetic acid from Sigma to fully solubilize the solution during mixing. The three solutions were mixed at a 1:1:1 (w/v) ratio in a 50 ml conical tube and mixed at 10 RPM for 16 hours. The final solution was phase separated with large chunks of yellow aggregates suspended or floating in the low viscosity medium.

### Example 27

A mixture of hyaluronic acid and a recombinant collagen fragment with a molecular weight of 50 kDa having SEQ ID NO: 1 (hydroxylated to 22%) was prepared and visually examined. The recombinant collagen fragment was hydroxylated by the process described in International Application No. WO2021/163485, and the percent hydroxylation was measured by the process described in International Application No. WO2021/163485, the disclosure of which is incorporated herein in its entirety. Example 27 was prepared using the method in Example 21 except that Polymer 2 was replaced with a 22% hydroxylated collagen with a molecular weight of 50 kDa. The 22% hydroxylated collagen solution had a solid content of 1.3% and pH was about 6. A 5 wt % 50 kDa HA solution (Polymer 1) was prepared by dissolving 5 g HA in 95 ml DI water and mixing on stir plate at 400 RPM for 16 hours. Two solutions were mixed at a 1:1 (w/w) ratio in a 15 ml Falcon tube and mixed at 10 RPM for 16 hours. The final solution was turbid indicating that at this hydroxylation level the two polymers are not miscible in solution.

### Example 28

A mixture of hyaluronic acid and a recombinant collagen fragment with a molecular weight of 50 kDa having SEQ ID NO: 1 (hydroxylated to 44%) was prepared and visually examined. The recombinant collagen fragment was hydroxylated by the process described in International Application No. WO2021/163485, and the percent hydroxylation was measured by the process described in International Application No. WO2021/163485, the disclosure of which is incorporated herein in its entirety. Example 28 was prepared using the same method described in Example 27 except using 44% hydroxylated collagen with a molecular weight of 50 kDa as Polymer 2. The 44% hydroxylated collagen solution had a solid content of 1.3% and pH was about 6. A 5 wt % 50 kDa HA solution (Polymer 1) was prepared by dissolving 5 g HA in 95 ml DI water and mixing on stir plate at 400 RPM for 16 hours. The two solutions were mixed at a 1:1 (w/w) ratio in a 15 ml conical tube and mixed at 10 RPM for 16 hours. The final solution was turbid and slowly phase separated, with white aggregates settled at bottom of the tube.

### Example 29

A biphasic crosslinked hyaluronic acid (comprising crosslinked HA and uncrosslinked HA) and collagen (recombinant collagen fragment with a molecular weight of 50 kDa having SEQ ID NO: 1) mixture was prepared and visually examined. Example 29 was prepared using the method described in Example 21, except that Polymer 1 was replaced with biphasic crosslinked HA (particle size of 1.25-2 mm; Beijing Mengbring Bio-Sci-Tec. Co., Ltd.). A 2.4% (w/v) HA solution was used. A solution of 20 wt % of the recombinant collagen fragment of SEQ ID NO: 1 was prepared by dissolving 2 g of the 50 kDa recombinant collagen in 18 ml DI water and mixing at 10 RPM for 4 hours (Polymer 2). The two solutions were mixed together in a 1:1 (w/w) ratio in a 50 ml conical tube and mixed at 10 RPM for 16 hours. The mixed solution was visually transparent.

This example demonstrates that solutions including a biphasic crosslinked HA and the recombinant collagen fragment with a molecular weight of 50 kDa having SEQ ID NO: 1 are fully miscible.

### Example 30

A monophasic crosslinked hyaluronic acid and collagen (recombinant collagen fragment with a molecular weight of 50 kDa having SEQ ID NO: 1) mixture was prepared and visually examined. Example 30 was prepared using the same method in Example 21, except that Polymer 1 was replaced with monophasic crosslinked HA (Bloomage Biotechnology Corp. (Jinan, Shandong, China)). A 2% (w/v) monophasic crosslinked HA solution was used. A 20 wt % collagen solution comprising the recombinant collagen fragment of SEQ ID NO: 1 was prepared by dissolving 2 g of the 50 kDa recombinant collagen in 18 ml DI water and mixing at 10 RPM for 4 hours. The two solutions were mixed at a 1:1 (w/w) ratio in a 50 ml conical tube and mixed at 10 RPM for 16 hours. The solution was visually transparent.

This example demonstrates that solutions including a monophasic crosslinked HA and the recombinant collagen fragment with a molecular weight of 50 kDa having SEQ ID NO: 1 are fully miscible.

### Example 31

A biphasic crosslinked hyaluronic acid (comprising crosslinked HA and uncrosslinked HA) and collagen (recombinant collagen fragment with a molecular weight of 50 kDa having SEQ ID NO: 1) mixture was prepared and visually examined. Example 31 was prepared using the method described in Example 30, except that Polymer 1 was replaced with biphasic crosslinked HA (Restylane). A 2.0% (w/v) biphasic crosslinked HA solution was used. A 40 wt % solution of the recombinant collagen fragment of SEQ ID NO: 1 was prepared by dissolving 4 g of the 50 kDa recombinant collagen in 16 ml DI water and mixing at 10 RPM for 4 hours. The two solutions were mixed at a 1:3, 1:1, 10:3 and 100:3 (w/w) ratio of HA:collagen in a 1 ml Eppendorf tube with a pipette and mixed at 10 RPM for 16 hours. The solutions were transparent visually.

This example demonstrates that solutions including a biphasic crosslinked hyaluronic acid and the recombinant collagen fragment with a molecular weight of 50 kDa having SEQ ID NO: 1 are fully miscible.

### Example 32

A 5% hyaluronic acid and collagen (recombinant collagen fragment with a molecular weight of 50 kDa having SEQ ID NO: 1) mixture was prepared and visually examined. Example 32 was prepared using the method described in Example 21, except that the pH of the 5 wt % 50 kDa HA solution was adjusted to pH 5.2-7.2 with sodium hydroxide prior to the addition of a collagen solution comprising the recombinant collagen fragment of SEQ ID NO: 1. The two solutions were mixed together in a 1:1 (w/w) ratio in a 50 ml tube and mixed at 10 RPM for 16 hours. The solutions were visually transparent. Films were prepared using the method of Example 9. The dried films were visually hazy with uniformly distributed haziness throughout the film.

This example demonstrates that solutions including 5% hyaluronic acid at pH 5.2-7.2 and the recombinant collagen fragment with a molecular weight of 50 kDa having SEQ ID NO: 1 are fully miscible.

### Example 33

A 5% hyaluronic acid and collagen (recombinant collagen fragment with a molecular weight of 50 kDa having SEQ ID NO: 1) mixture was prepared at different pHs and visually examined. Example 33 was prepared using the method described in Example 21, except the pH of the 5 wt % 50 kDa HA solution was adjusted to pH of 9, 10, 11, and 12 with sodium hydroxide prior to the addition of the recombinant collagen fragment of SEQ ID NO: 1. The two solutions were mixed at a 1:1 (w/w) ratio in a 20 ml glass vial and mixed with a stir bar at 400 RPM for 30 minutes due to lower viscosity of the basic solutions. The solutions were then readjusted to pH 7±1 with concentrated hydrochloric acid while mixing with a stir bar at 400 RPM. The solutions were visually transparent prior and after pH adjusted to 7±1.

This example demonstrates that solutions including 5% hyaluronic acid at pH 9, 10, 11, 12, and the recombinant collagen fragment with a molecular weight of 50 kDa having SEQ ID NO: 1 are fully miscible.

### Example 34

A hyaluronic acid and collagen mixture was prepared and visually examined. Example 34 was prepared using the method in Example 21 except that Polymer 1 was replaced with 2% HumaColl21® solution (Geltor). A 2 wt % 50 kDa HA solution was prepared using the method described in Example 21. The two solutions were mixed at a 1:1 (w/w) ratio in a 2 ml glass vial and mixed with on a stir plate at 400 RPM for 1 hour. The final solution was visually transparent.

This example demonstrates that solutions including 2% HumaColl21® solution and the recombinant collagen fragment with a molecular weight of 50 kDa having SEQ ID NO: 1 are fully miscible.

### Example 35

Hyaluronic acid and full-length recombinant collagen and a 50 kDa recombinant collagen fragment are blended, mixed with HA, and visually examined. A full-length recombinant collagen and a 50 kDa recombinant collagen fragment are blended and mixed with HA. The solutions are visually inspected. A blend of 50 kDa recombinant collagen fragment with full length recombinant collagen and/or hydrolyzed recombinant collagen (or combinations of any of these three) is mixed with HA, and visually examined. Then, the blend of the full-length recombinant collagen and the 50 kDa recombinant collagen fragment and a blend of the blend of the full length recombinant collagen and a hydrolyzed full length recombinant collagen are mixed with HA. The solutions are visually inspected.

### Example 36

A poly(2-ethyl-2-oxazoline) (PEOx) solution was mixed with a solution comprising the recombinant collagen fragment with a molecular weight of 50 kDa having SEQ ID NO: 1. Example 36 was prepared using the same method described in Example 7, except that Polymer 1 was replaced with PEOx (molecular weight of 50 kDa; Sigma) 1. The remainder of the method was the same as set forth in Example 7. The final solution was visually transparent showing that the polymers were miscible in solution. A film was prepared using the same method as described in Example 9 except in an aluminum weighing pan. The dried film visually appeared to be phase separated during drying.

### Example 37

A polyethylenimine (PEI) solution was mixed with a solution including the recombinant collagen fragment with a molecular weight of 50 kDa having SEQ ID NO: 1. Example 37 was prepared using the method described in Example 7, except that Polymer 1 was replaced with PEI (molecular weight of 100 kDa; Polysciences (Warrington, Pennsylvania)). A 5 wt % polyethylenimine solution was prepared by dissolving 0.5 g of PEI in 9.5 g of Milli-Q water at 80° C. using a stirrer bar at 450 RPM for 3 hours, or until the solution was completely transparent. The solution was then cooled to room temperature and the pH was adjusted to 7 using 12N HCl. The remainder of the method was the same as set forth in Example 7. The solution was transparent showing that the polymers were miscible in solution. A film was prepared using the method set forth in Example 3. The dried film was visually opaque.

### Example 38

A first sodium carboxymethylcellulose (NaCMC) and collagen (recombinant collagen fragment with a molecular weight of 50 kDa having SEQ ID NO: 1) mixture was prepared and dried, and visually examined. Example 38 was prepared using the method as described in Example 7, except that Polymer 1 was replaced with sodium carboxymethylcellulose (molecular weight of ˜250 kDa and a degree of substitution of 1.22; Sigma). A 5 wt % sodium carboxymethylcellulose solution was prepared by dissolving 0.5 g of sodium carboxymethylcellulose in 9.5 g DI water and mixing in a conical tube for 16 hours at 5 RPM. The two solutions were mixed at a 1:1 (v/v) ratio in a 50 ml conical tube and mixed at 10 RPM for 16 hours. The final solution was transparent and slightly yellow showing that the solutions were miscible. A film was prepared using the method set forth in Example 9, except using an aluminum weighing pan. The dried film was visually hazy with uniformly distributed haziness throughout the film.

### Example 39

A second sodium carboxymethylcellulose and collagen (recombinant collagen fragment with a molecular weight of 50 kDa having SEQ ID NO: 1) mixture was prepared and dried, and visually examined. Example 39 was prepared using the same method described in Example 7, except that Polymer 1 was replaced with sodium carboxymethylcellulose (molecular weight of ˜250 kDa and a degree of substitution of 0.79; Sigma). A 2.5 wt % sodium carboxymethylcellulose solution was prepared by dissolving 0.25 g of sodium carboxymethylcellulose in 9.75 g DI water and mixing in a conical tube for 16 hours at 5 RPM. A 2.5 wt % collagen solution comprising the recombinant collagen fragment with amino acid sequence of SEQ ID NO: 1 was prepared by dissolving 0.25 g of the 50 kDa recombinant collagen in 9.75 g DI water. The two solutions were mixed at a 1:1 (v/v) ratio in a 50 ml conical tube and mixed at 10 RPM for 16 hours. The final solution was transparent showing that the polymers were miscible in solution. A film was prepared using the method set forth in Example 9, except using an aluminum weighing pan. The dried film was visually hazy with uniformly distributed haziness throughout the film.

### Example 40

A third sodium carboxymethylcellulose and collagen (recombinant collagen fragment with a molecular weight of 50 kDa having SEQ ID NO: 1) mixture was prepared and dried, and visually examined. Example 41 was prepared using the method described in Example 7, except that Polymer 1 was replaced with using sodium carboxymethylcellulose (high viscosity of 2,700 cps for 1% aqueous solution and a degree of substitution of 0.87; Sigma). A 2.5 wt % sodium carboxymethylcellulose solution was prepared by dissolving 0.25 g of sodium carboxymethylcellulose in 9.75 g DI water and mixing in a conical tube for 16 hours at 5 RPM. A 2.5 wt % solution comprising the recombinant collagen fragment with the amino acid sequence of SEQ ID NO: 1 was produced by dissolving 0.25 g of the recombinant collagen fragment into 9.75 g of water. The two solutions were mixed at a 1:1 (v/v) ratio in a 50 ml conical tube and mixed at 10 RPM for 16 hours. The final solution was transparent showing that the polymers were miscible in solution. A film was prepared using the method set forth in Example 9, except using an aluminum weighing pan. The dried film was visually hazy with uniformly distributed haziness throughout the film.

### Example 41 (HA/SEQ ID NO: 1007)

A recombinant collagen fragment having the amino acid sequence of SEQ ID NO: 1007 and HA mixture was prepared, dried, and visually examined. SEQ ID NO: 1007 is 98% identical to SEQ ID NO: 1. Example 41 was prepared using the method as described in Example 21, except that Polymer 2 was replaced with a collagen polymer having amino acid sequence of SEQ ID NO: 1007. A 5 wt % solution comprising the recombinant collagen fragment having amino acid sequence of SEQ ID NO: 1007 was made by dissolving 0.25 g of the recombinant collagen fragment in 4.75 g of water. A 5 wt % 50 kDa HA solution was prepared by dissolving 0.25 g of 50 kDa HA (Polymer 1) in 4.75 g of water. The two solutions were mixed at a 1:1 (w/w) ratio in a 50 ml conical tube and mixed at 10 RPM for 16 hours. The conical tube was placed vertically on a rack overnight to assess the solution stability. The final solution was transparent. A film was prepared using the method set forth in Example 9, except using an aluminum pan. The dried film was visually hazy with uniformly distributed haziness throughout the film.

### Example 42 (HA/SEQ ID NO: 1008)

A recombinant collagen fragment having the amino acid sequence of SEQ ID NO: 1008 and HA mixture was prepared, dried, and visually examined. SEQ ID NO: 1008 is 50% identical to SEQ ID NO: 1. Example 42 was prepared using the method described in Example 21, except that Polymer 2 was replaced with a collagen polymer having amino acid sequence of SEQ ID NO: 1008. A 5 wt % collagen solution comprising the collagen polymer having amino acid sequence of SEQ ID NO: 1008 was prepared by dissolving 0.25 g of the recombinant collagen fragment in 4.75 g of Milli-Q Water (water purified using a Millipore Milli-Q lab water system; “MQ water”) and purified through centrifugation at 3214 relative centrifugal force (RCF) for 1 hour. A 5 wt % 50 kDa HA (Polymer 1) solution was prepared using the same method described in Example 41. The supernatant of the collagen solution and the HA solution were mixed at a 1:1 (w/w) ratio in a 50 ml Falcon tube and mixed at 10 RPM for 16 hours. The solution was transparent Showing that the two polymers were miscible in solution. The dried film was visually hazy.

### Example 43 (HA/SEQ ID NO: 973)

A full-length collagen and HA mixture was prepared, dried, and visually examined. Example 43 was prepared using the method as described in Example 41, except that Polymer 2 was replaced with a recombinant collagen fragment with a molecular weight of 50 kDa and having SEQ ID NO: 973. A 5 wt % collagen solution comprising the recombinant collagen fragment of SEQ ID NO: 973 was prepared by dissolving 0.25 g of the recombinant collagen in 4.75 g of MQ water. A 5 wt % 50 kDa HA solution was prepared using the method described in Example 42. The remainder of the method was the same as set forth in Example 42. The solution was transparent showing that the polymers were miscible in solution. The dried film was visually hazy with uniformly distributed haziness throughout the film.

### Example 44 (Chondroitin Sulfate/Collagen)

A chondroitin sulfate and collagen (recombinant collagen fragment with a molecular weight of 50 kDa having SEQ ID NO: 1) mixture was prepared, dried, and visually examined. Example 44 was prepared using the method described in Example 7, except that Polymer 1 was replaced with chondroitin sulfate from bovine trachea (molecular weight of 50 kDa; EMD Millipore (Burlington, MA)). A 5 wt % chondroitin sulfate solution was prepared by dissolving 0.5 g of chondroitin sulfate in 9.5 g MQ Water. The remainder of the method was the same as set forth in Example 7. The final solution was transparent showing that the polymers were miscible in solution. A film was prepared using the same method as described in Example 9, except using an aluminum weighing pan. The dried film was visually hazy with uniformly distributed haziness throughout the film.

### Example 45 (AcHA/Collagen)

An acetylated hyaluronate (AcHA) and collagen (recombinant collagen fragment with a molecular weight of 50 kDa having SEQ ID NO: 1) mixture was prepared, dried, and visually examined. Example 45 was prepared using the method described in Example 21, except that Polymer 1 was replaced with sodium acetylated hyaluronate (Bloomage Biotechnology Corp., Ltd.). A 5 wt % Acetylated Hyaluronic Acid (AcHA) solution was prepared by dissolving 0.25 g of AcHA in 4.75 g of MQ Water. A 5 wt % solution comprising the recombinant collagen fragment of SEQ ID NO: 1 was prepared by dissolving 0.25 g of collagen in 4.75 g of water. The remainder of the method was the same as set forth in Example 21. The solution was transparent showing that the polymers were miscible in solution. A film was prepared using the same method described in Example 9, except using an aluminum pan. The dried film was visually hazy with uniformly distributed haziness throughout the film.

### Example 46 (ZnHA/Collagen)

A Zinc hyaluronate (ZnHA) and collagen (recombinant collagen fragment with a molecular weight of 50 kDa having SEQ ID NO: 1) mixture was prepared and dried, and visually examined. Example 46 was prepared using the method described in Example 21, except that Polymer 1 was replaced with (Zinc Hyaluronate (ZnHA) (molecular weight of 170 kDa; Bloomage Biotechnology Corp., Ltd.). A 5 wt % Zinc Hyaluronate (ZnHA) solution was prepared by dissolving 0.25 g of ZnHA in 4.75 g of MQ Water. A 5 wt % solution comprising the recombinant collagen fragment of SEQ ID NO: 1 was prepared by dissolving 0.25 g of collagen in 4.75 g of water. The remainder of the method was the same as set forth in Example 21. The solution was turbid indicating aggregation of particles. A film was prepared using the same method as described in Example 9, except using an aluminum pan. The dried film was transparent indicating that the polymers were miscible.

### Example 47 (HA/Collagen Salt Doping Study)

A hyaluronic acid and collagen (recombinant collagen fragment with a molecular weight of 50 kDa having SEQ ID NO: 1) solution was prepared, dried, and visually examined. Example 47 was prepared using the method as described in Example 21, except that the solution comprising the recombinant collagen fragment with a molecular weight of 50 kDa having SEQ ID NO: 1 was prepared in a NaCl solution. A 5 wt % collagen solution was prepared by dissolving 0.25 g in 4.75 g in a 0.05 mM solution of NaCl. A 5 wt % 50 kDa HA solution was prepared using the same method as described in Example 41. The two solutions were mixed at a 1:1 (w/w) ratio in a 50 ml conical tube and mixed at 10 RPM for 4 hours. The solution was transparent showing that the polymers were miscible in higher salt content solutions. A film was prepared using the same method as described in Example 9, except using an aluminum pan. The dried film was hazy.

### Example 48 (HA/Collagen Dialysis)

A hyaluronic acid and collagen (recombinant collagen fragment with a molecular weight of 50 kDa having SEQ ID NO: 1) solution was prepared, dried, and visually examined. Example 48 was prepared using the method described in Example 21, except the hyaluronic and collagen solution was dialyzed. A 5 wt % collagen solution comprising the recombinant collagen fragment of SEQ ID NO: 1 was prepared by dissolving 0.25 g of the recombinant collagen in 4.75 g of MQ water. A 5 wt % 50 kDa HA solution was prepared by dissolving 0.25 g of hyaluronic acid powder in 4.95 g of MQ water. The two solutions were mixed at a 1:1 (w/w) ratio in a 50 ml conical tube and mixed at 10 RPM for 4 hours. Then, 9 g of the combined solution was loaded into a 10 ml volume 3.5-5 kDa molecular weight cutoff (MWCO) membrane. A dialysate reservoir was prepared with a Nalgene bottle containing 500 ml of MQ water and a stir bar to keep the surrounding solution gently rotating. The membrane containing the solution was submerged in the dialysate reservoir and dialyzed at room temperature for 16 hours. The solution was pipetted out of the membrane and transferred into a 50 ml conical tube. The solution was transparent showing that the polymers were miscible in lower salt content solutions. A film was prepared using the same method as described in Example 9, except using an aluminum pan. The dried film was visually hazy with uniformly distributed haziness throughout the film.

### Example 49 (HA and Hydrolyzed Recombinant Collagen Fragment-5 mg/mL Papain)

A hyaluronic acid and hydrolyzed collagen solution (hydrolyzed fragments of recombinant collagen fragment with a molecular weight of 50 kDa having SEQ ID NO: 1) was prepared, dried, and visually examined. A 5 wt % solution comprising the recombinant collagen fragment with the amino acid sequences of SEQ ID NO: 1 was made by dissolving 0.25 g of the collagen in 4.75 g of water. A 10 wt % Papain enzyme solution was made by dissolving 100 mg Papain (200 TU/mg, sourced from BIO-CAT) in 900 μl of water. A hydrolyzed recombinant fragment solution was made by adding 250 μL of 10 wt % Papain enzyme solution into 4.75 ml of 5 wt % recombinant collagen solution and incubating at 60° C. for 2 hours for enzymatic hydrolysis, and then at 90° C. for 10 minutes to deactivate the enzyme activity. After that, the hydrolyzed solution was centrifuged at 3214 relative centrifugal force (RCF) for 20 minutes. The supernatant solution was collected to mix with the following HA solution. A 5 wt % recombinant collagen fragment and HA solution was prepared by dissolving 0.25 g of 50 kDa HA in 4.75 g of water. The two solutions were mixed at a 1:1 (w/w) ratio in a 50 ml conical tube and mixed at 5 RPM for 2 hours. The conical tube was placed vertically on a rack overnight to assess the solution stability. The final solution was transparent. A film was prepared using the method set forth in Example 9 except using a silicone mold. The dried film was visually transparent showing that the polymers were fully miscible.

### Example 50 (PVP and Hydrolyzed Recombinant Collagen Fragment-5 mg/mL Papain)

A polyvinylpyrrolidone and hydrolyzed recombinant collagen solution (hydrolyzed fragments of recombinant collagen fragment with a molecular weight of 50 kDa having SEQ ID NO: 1) was prepared, dried, and visually examined. Example 50 was prepared using the method described in Example 49, except that Polymer 1 was replaced with polyvinylpyrrolidone (molecular weight of 40 kDa; Sigma). A 5 wt % 40 kDa PVP solution was prepared by dissolving 0.25 g of 40 kDa PVP in 4.75 g of water. The hydrolyzed recombinant collagen solution was prepared using the method described in Example 43. The two solutions were mixed at a 1:1 (w/w) ratio in a 50 ml conical tube and mixed at 5 RPM for 2 hours. The conical tube was placed vertically on a rack overnight to assess the solution stability. The final solution was transparent. A film was prepared using the method set forth in Example 9, except using a silicone mold. The dried film was visually transparent showing that the polymers were miscible in solution and solid.

### Example 51 (HA and Blend of Recombinant Collagen Fragment and Hydrolyzed Recombinant Collagen Fragment—0.1 mg/mL Papain)

A solution of hyaluronic acid, a recombinant collagen fragment with a molecular weight of 50 kDa having SEQ ID NO: 1, and partially hydrolyzed recombinant collagen fragments was prepared, dried, and visually examined. A 5 wt % solution comprising the recombinant collagen fragment having the amino acid sequence of SEQ ID NO: 1 was made by dissolving 0.25 g of the recombinant collagen fragment in 4.75 g of water. A blend of recombinant collagen fragment and hydrolyzed recombinant collagen fragment solution was prepared by partially hydrolyzing the 5 wt % collagen solution. A 1 wt % Papain enzyme solution was made by dissolving 10 mg Papain (200 TU/mg, sourced from BIO-CAT) in 990 μl of water. A solution of partially hydrolyzed recombinant fragments was made by adding 50 μl of 1 wt % Papain enzyme solution into 4.95 ml of 5 wt % recombinant collagen solution and incubating at 60° C. for 2 hours for enzymatic hydrolysis, and then at 90° C. for 10 minutes to deactivate the enzyme activity (Polymer 2). A 5 wt % 50 kDa HA (Polymer 1) solution was prepared by dissolving 0.25 g of 50 kDa HA in 4.75 g of water. The two solutions were mixed at a 1:1 (w/w) ratio in a 50 ml conical tube and mixed at 5 RPM for 2 hours. The conical tube was placed vertically on a rack overnight to assess the solution stability. The final solution was transparent showing that the polymers were miscible in solution. A film was prepared using the method set forth in Example 9, except using a silicone mold. The dried film was visually hazy with uniformly distributed haziness throughout the film.

### Example 52 (PVP and Blend of Recombinant Collagen Fragment and Hydrolyzed Recombinant Collagen Fragment—0.1 mg/mL Papain)

A solution of polyvinylpyrrolidone, a recombinant collagen fragment with a molecular weight of 50 kDa having SEQ ID NO: 1, and partially hydrolyzed recombinant collagen fragments was prepared, dried, and visually examined. Example 52 was prepared using the method described in Example 51, except that Polymer 1 was replaced with polyvinylpyrrolidone (molecular weight of 40 kDa; Sigma). A 5 wt % 40 kDa PVP solution was prepared by dissolving 0.25 g of 40 kDa PVP in 4.75 g of water. A blend of recombinant collagen fragment and hydrolyzed recombinant collagen fragment solution was prepared using the method described in Example 51. The two solutions were mixed at a 1:1 (w/w) ratio in a 50 ml conical tube and mixed at 5 RPM for 2 hours. The conical tube was placed vertically on a rack overnight to assess the solution stability. The final solution was transparent. A film was prepared using the method set forth in Example 9, except using an aluminum weighing pan. The dried film was visually transparent showing that the polymers were miscible in solution and solid.

### Example 53 (Biphasic HA and Blend of Recombinant Collagen Fragment and Hydrolyzed Recombinant Collagen Fragment—0.1 mg/mL Papain)

A solution of biphasic crosslinked hyaluronic acid (comprising crosslinked HA and uncrosslinked HA) and a blend of a recombinant collagen fragment with a molecular weight of 50 kDa having SEQ ID NO: 1, and partially hydrolyzed recombinant collagen fragments was prepared and visually examined. Example 53 was prepared using the method described in Example 51, except Polymer 1 was replaced with a 2.4% (w/v) biphasic crosslinked HA with a particle size of 0.10-0.25 mm (Shandong Runxin Biotechnology Co., Ltd. (Qufu City, Shandong Province, China). A solution containing a blend of recombinant collagen fragment with a molecular weight of 50 kDa having SEQ ID NO: 1 and hydrolyzed recombinant collagen fragments was prepared using the method as described in Example 51. The two solutions were mixed at a 1:1 (w/w) ratio in a 50 ml conical tube and vortexed for 3 minutes and mixed at 5 RPM for 3 hours. The conical tube was placed vertically on a rack overnight to assess the solution stability. The solution was visually slightly hazy due to visible crosslinked HA particle texture.

### Example 54 (Sodium CMC and Blend of Recombinant Collagen Fragment and Hydrolyzed Recombinant Collagen Fragment—0.1 mg/mL Papain)

A solution of sodium carboxymethylcellulose and blend of a recombinant collagen fragment with a molecular weight of 50 kDa having SEQ ID NO: 1, and partially hydrolyzed recombinant collagen fragments was prepared, dried, and visually examined. Example 54 was prepared using the method described in Example 51, except that Polymer 1 was replaced with sodium carboxymethylcellulose (molecular weight of ˜250 kDa and a degree of substitution of 1.22; Sigma). A 5 wt % sodium carboxymethylcellulose solution was prepared by dissolving 0.5 g of sodium carboxymethylcellulose in 9.5 g DI water and mixing in a conical tube for 16 hours at 5 RPM. A blend of recombinant collagen fragment and hydrolyzed recombinant collagen fragment solution was prepared using the method described in Example 51. The two solutions were mixed at a 1:1 (w/w) ratio in a 50 ml conical tube and mixed at 5 RPM for 2 hours. The conical tube was placed vertically on a rack overnight to assess the solution stability. The final solution was transparent showing that the polymers were miscible in solution. A film was prepared using the method set forth in Example 9 except using an aluminum weighing pan. The dried film was visually hazy with uniformly distributed haziness throughout the film.

### Example 55 (Sodium Carboxymethylcellulose and SEQ ID NO: 1007)

A sodium carboxymethylcellulose and collagen polymer having the amino acid sequence set forth in SEQ ID NO: 1007 solution was prepared, dried, and visually examined. SEQ ID NO: 1007 is 98% identical to SEQ ID NO: 1. A 5 wt % solution comprising the recombinant collagen polymer having the amino acid sequence set forth in SEQ ID NO: 1007 was made by dissolving 0.5 g of the recombinant collagen fragment in 9.5 g of water. A 5 wt % sodium carboxymethylcellulose (molecular weight of ˜250 kDa and a degree of substitution of 1.22; Sigma) solution was prepared by dissolving 0.5 g of sodium carboxymethylcellulose in 9.5 g DI water and mixing in a conical tube for 16 hours at 5 RPM. The two solutions were mixed at a 1:1 (w/w) ratio in a 50 ml conical tube and mixed at 5 RPM for 2 hours. The conical tube was placed vertically on a rack overnight to assess the solution stability. The final solution was transparent showing that the polymers were miscible in solution. A film was prepared using the method set forth in Example 9, except using a silicone mold. The dried film was visually hazy with uniformly distributed haziness throughout the film.

### Example 56 (PVP and SEQ ID NO: 1007)

A polyvinylpyrrolidone and collagen polymer having the amino acid sequence set forth in SEQ ID NO: 1007 solution was prepared, dried, and visually examined. SEQ ID NO: 1007 is 98% identical to SEQ ID NO: 1. A 5 wt % solution comprising the recombinant collagen fragment having amino acid sequence set forth in SEQ ID NO: 1007 was made by dissolving 0.5 g of the recombinant collagen fragment in 9.5 g of water. A 5 wt % 40 kDa PVP solution was prepared by dissolving 0.25 g of 40 kDa PVP in 4.75 g of water. The two solutions were mixed at a 1:1 (w/w) ratio in a 50 ml conical tube and mixed at 5 RPM for 2 hours. The conical tube was placed vertically on a rack overnight to assess the solution stability. The final solution was transparent. A film was prepared using the method set forth in Example 9, except using a silicone mold. The dried film was visually transparent indicating that the polymers were miscible in solution and in the solid state.

### Example 57 (Biphasic HA and SEQ ID NO: 1007)

A biphasic crosslinked hyaluronic acid (comprising crosslinked HA and uncrosslinked HA) and blend of recombinant collagen fragment having the amino acid sequence set forth in SEQ ID NO: 1007 and hydrolyzed recombinant collagen fragment solution was prepared and visually examined. SEQ ID NO: 1007 is 98% identical to SEQ ID NO: 1. A 2.4% (w/v) biphasic crosslinked HA with a particle size of 0.10-0.25 mm purchased from Shandong Runxin Biotechnology Co., Ltd. (Qufu City, Shandong Province, China) was used as Polymer 1. A 5 wt % solution comprising the recombinant collagen fragment having the amino acid sequence set forth in SEQ ID NO: 1007 was made by dissolving 0.5 g of the recombinant collagen fragment in 9.5 g of water. The two solutions were mixed at a 1:1 (w/w) ratio in a 50 ml conical tube and vortexed for 3 minutes and mixed at 5 RPM for 3 hours. The conical tube was placed vertically on a rack overnight to assess the solution stability. The solution was visually transparent. In addition, the same conical tube was placed vertically on a rack in a fridge at 4° C. overnight and the solution remained transparent showing that the polymers were miscible in solution.

### Example 58 (HA: SEQ ID NO: 1 at Different Ratios)

A hyaluronic acid (HA) and collagen (recombinant collagen fragment with a molecular weight of 50 kDa having SEQ ID NO: 1) mixture was prepared and dried, and visually examined. A 5 wt % 50 kDa HA solution was prepared by dissolving 2.5 g HA in 47.5 g of DI water. A 5% (w/v) collagen solution including the recombinant collagen fragment of SEQ ID NO: 1 was made by dissolving 2.5 g of the recombinant collagen fragment in 47.5 g of water. The two solutions were mixed at a 1:9, 1:3, 2:3, 1:1, 3:2, 3:1, and 9:1 (w/w) ratio of HA:collagen with 10 g total solution weight in 50 ml conical tubes and mixed at 5 RPM for 3 hours. The conical tubes were placed vertically on a rack overnight to assess the solution stability. The final solutions were transparent showing that all ratios are miscible in solution. Films were prepared using the same method as described in Example 9 except using a silicone mold. The dried film with HA:collagen at a ratio of 1:9 was visually transparent. The dried film with HA:collagen at a ratio of 1:3 was visually transparent to slightly hazy. The dried films of the remaining ratios were visually hazy with uniformly distributed haziness throughout the film showing that miscibility in the solid state is composition dependent. In addition, aliquots of HA:collagen solution mixtures for those seven ratios were placed in a fridge at 4° C. overnight and the solutions remained transparent.

### Example 59 (HA: 5% Hydroxylation)

A mixture of hyaluronic acid and a 5% hydroxylated recombinant collagen fragment with a molecular weight of 50 kDa having SEQ ID NO: 1 was prepared and visually examined. The recombinant collagen fragment was hydroxylated by the process described in International Application No. WO2021/163485, and the percent hydroxylation was measured by the process described in International Application No. WO2021/163485, the disclosure of which is incorporated herein in its entirety. Example 59 was prepared using the method described in Example 27, except that Polymer 2 was replaced with a 5.41% hydroxylated collagen (molecular weight of 50 kDa). A 2 wt % 50 kDa HA solution (polymer 1) was prepared by dissolving 0.2 g of HA in 9.8 g of MQ water. A 5% hydroxylated collagen had a solid content of 12% and pH was about 6. The hydroxylated collagen solution was then diluted to 2 wt % with water and purified by centrifugation at 15,000 RPM for 10 min. The supernatants of the hydroxylated collagen solution and the HA solution were mixed at a 1:1 (w/w) ratio in a 15 ml Falcon tube and mixed at 10 RPM for 16 hours. The solution was transparent showing that the polymers were miscible in solution. A film was prepared using the method set forth in Example 9, except in a silicon mold. The dried film was visually hazy.

### Example 60 (HA: 12% Hydroxylation)

A mixture of hyaluronic acid and a 12% hydroxylated recombinant collagen fragment with a molecular weight of 50 kDa having SEQ ID NO: 1 was prepared and visually examined. The recombinant collagen fragment was hydroxylated by the process described in International Application No. WO2021/163485, and the percent hydroxylation was measured by the process described in International Application No. WO2021/163485, the disclosure of which is incorporated herein in its entirety. Example 60 was prepared using the method described in Example 27, except using a 12.47% hydroxylated collagen with a molecular weight of 50 kDa as Polymer 2. A 2 wt % 50 kDa HA solution (Polymer 1) was prepared by dissolving 0.2 g of HA in 9.8 g of MQ water. A 12% hydroxylated collagen had a solid content of 3% and pH was about 6. The hydroxylated collagen solution was then diluted to 2 wt % with water and purified by centrifugation at 15,000 RPM for 10 min. The supernatants of the hydroxylated collagen solution and the HA solution were at a 1:1 (w/w) ratio in a 15 ml Falcon tube and mixed at 10 RPM for 16 hours. The solution was transparent showing that the polymers were miscible in solution. A film was prepared using the method set forth in Example 9, except in a silicone mold. The dried film was visually hazy.

### Example 61 (HA: 5% Hydroxylation in HCl)

A mixture of hyaluronic acid and a 5% hydroxylated recombinant collagen fragment with a molecular weight of 50 kDa having SEQ ID NO: 1 was prepared and visually examined. The recombinant collagen fragment was hydroxylated by the process described in International Application No. WO2021/163485, and the percent hydroxylation was measured by the process described in International Application No. WO2021/163485, the disclosure of which is incorporated herein in its entirety. Example 61 was prepared using the method described in Example 27, except using a 5.41% hydroxylated collagen with a molecular weight of 50 kDa as Polymer 2. A 2 wt % 50 kDa HA solution (Polymer 1) was prepared by dissolving 0.2 g of HA in 9.8 g of MQ water. A 5% hydroxylated collagen had a solid content of 12% and pH was about 6. The hydroxylated collagen solution was then diluted to 2 wt % with water and pH adjusted to ˜2.2 using 12N HCl. The collagen solution was purified by centrifugation at 15,000 RPM for 10 minutes. The supernatants of the hydroxylated collagen solution and the HA solution were mixed at a 1:1 (w/w) ratio in a 15 ml Falcon tube and mixed at 10 RPM for 16 hours. The solution was turbid. The collagen and HA solution was neutralized using 10N NaOH. The solution was transparent. A film was prepared using the method set forth in Example 9, except in a silicon mold. The dried film was visually hazy.

### Example 62 (HA: 12% Hydroxylation in HCl)

A mixture of hyaluronic acid and a 12% hydroxylated recombinant collagen fragment with a molecular weight of 50 kDa having SEQ ID NO: 1 was prepared and visually examined. The recombinant collagen fragment was hydroxylated by the process described in International Application No. WO2021/163485, and the percent hydroxylation was measured by the process described in International Application No. WO2021/163485, the disclosure of which is incorporated herein in its entirety. Example 62 was prepared using the method described in Example 27, except using a 12.47% hydroxylated collagen with a molecular weight of 50 kDa as Polymer 2. A 2 wt % 50 kDa HA solution (Polymer 1) was prepared by dissolving 0.2 g of HA in 9.8 g of MQ water. A 12% hydroxylated collagen had a solid content of 3% and pH was about 6. The hydroxylated collagen solution was then diluted to 2 wt % with water and pH adjusted to ˜2.2 using 12N HCl. The collagen solution was purified by centrifugation at 15,000 RPM for 10 minutes. The supernatants of the hydroxylated collagen solution and the HA solution were mixed at a 1:1 (w/w) ratio in a 15 ml Falcon tube and mixed at 10 RPM for 16 hours. The solution was turbid. The collagen and HA solution was neutralized using 10N NaOH. The solution was transparent. A film was prepared using the method set forth in Example 9, except in a silicon mold. The dried film was visually hazy.

### Example 63 (SEQ ID NO: 1 Foam Preparation)

A stock solution of collagen (SEQ ID NO: 1) in water was prepared at a weight concentration of 16.7% (w/w). Baymedix FD103 and Baymedix® AD111 are each aqueous polyurethane dispersions. A stock solution of Baymedix (Bayer MaterialScience, Pittsburgh, PA) FD103: AD111 was prepared at a ratio of 70:30 based on the weight of polyurethane dispersion. A stock solution of Rheolate 208 (Elementis, London, UK) in water was prepared at a weight concentration of 10% (w/w). 9.16 g of recombinant collagen stock solution and 11.50 g of Baymedix FD103: AD111 stock solution were mixed in a 50 ml beaker with an overhead mixer. After that, 1.55 g of Rheolate 208 stock solution was added and mixed. Then 0.72 g of ChemTex (Cumberland, RI) 2216, 0.65 g of ChemTex 2317, and 0.03 g of ChemTex 2243 were added and the resulting mixture was mixed further. The resulting mixture was mechanically frothed by vigorously mixing with the overhead mixer. After frothing, the frothed mixture was coated on a glass plate at a gap setting of 600 μm. The coating was dried at 75° C. for 30 minutes, and a porous foam sheet was achieved after drying.

### Example 64 (Collagen Protein Release Kinetics)

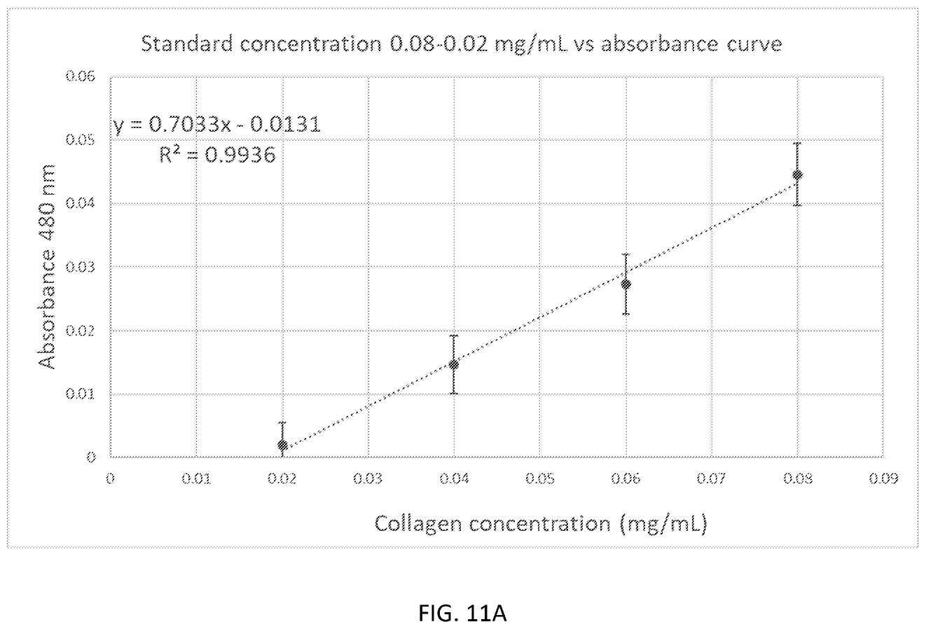

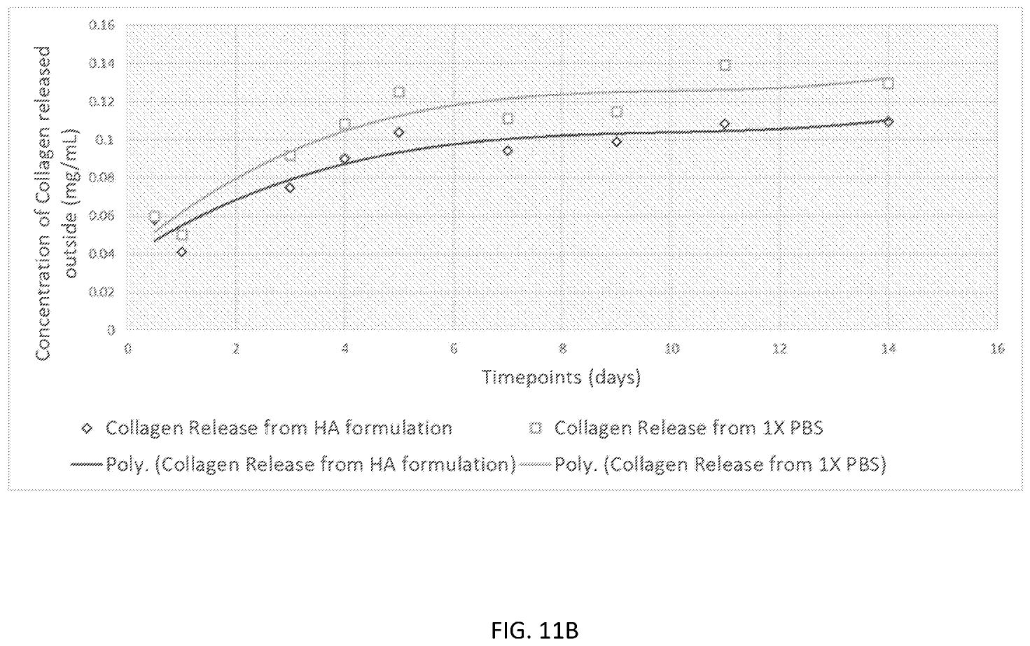

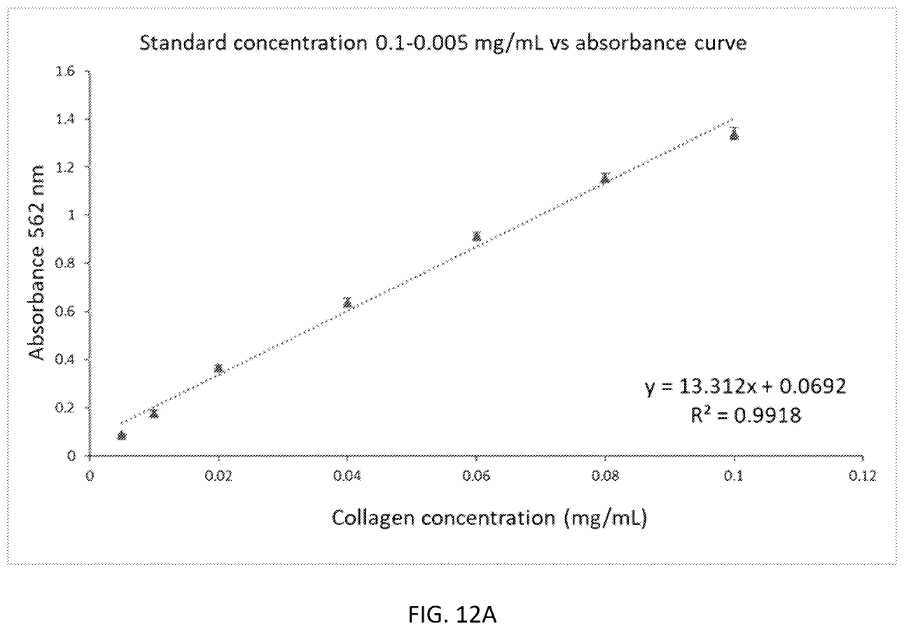

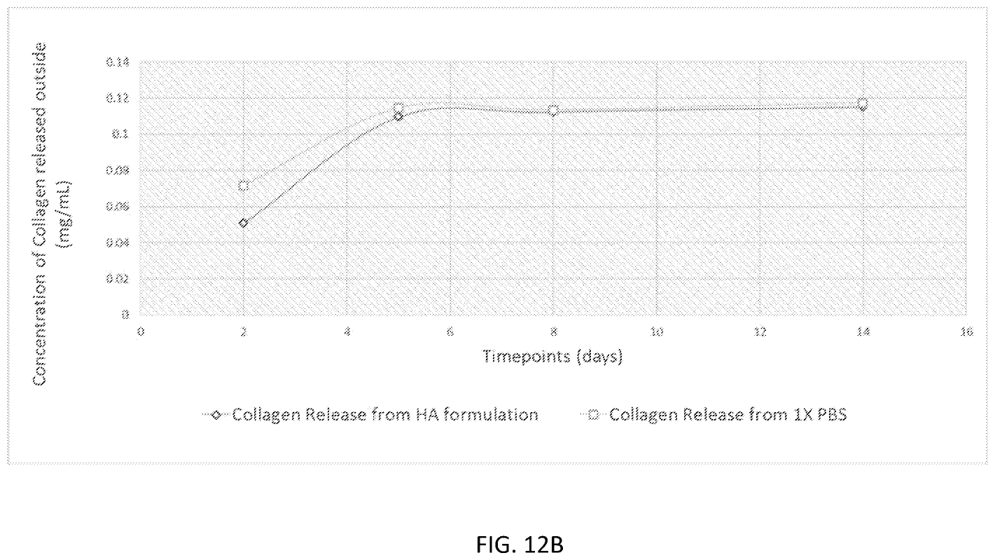

The release kinetics of a solution of (A) a recombinant collagen having amino acid sequence of SEQ ID NO: 1, and (B) a formulation of a recombinant collagen having amino acid sequence of SEQ ID NO: 1 and hyaluronic acid were investigated. The results are shown in FIGS. 11A, 11B, 12A, and 12B.

### Analysis

Standard curve: Triplicate absorbance values obtained of each sample and dilution were averaged and subtracted from the blank (1×PBS+0.5% sodium benzoate) value to obtain mean absorbance values (MAV) only from collagen in each sample/dilution. MAV of serial dilutions from 10 to 0.5 mg/mL were plotted against their respective concentrations. The same was done for dilutions from 0.08-0.02 mg/mL. A linear trendline was plotted for the 0.08-0.02 mg/mL range. The equation y=0.7033x−0.0131 was obtained with an R² value of 0.9936. This equation was used to calculate protein concentrations of the test samples of both control (A) and formulation (B) from their respective MAVs (FIG. 11A). Collagen mass per sample was calculated by multiplying the total solution volume present outside during the diffusion experiment (90 mL, 89.8 mL, 89.6 mL or 89.4 mL).

Concentrations were plotted against the timepoints to obtain a release trend. 2 such trends were obtained finally, one for Solution (A) and the other for Formulation (B) (FIG. 11B). As shown, in the figures, the protein is gradually released from both solution A and formulation B, which contains HA. The rate at which protein is released from formulation B appears to be slightly slower than from the solution not containing HA. This indicates a weak, but measurable, interaction between the HA and the collagen, which is likely because the solutions are miscible.

### Micro BCA Assay

The Micro BCA™ Protein Assay Kit protocol was referred to for analysis using the micro BCA Assay.

### Analysis

Triplicate absorbance values obtained from each sample and dilution were averaged and subtracted from the blank (1×PBS+0.5% sodium benzoate) value to obtain mean absorbance values (MAV) only from collagen in each sample/dilution. Standard deviation and error were calculated for each value.

MAV of serial dilutions from 10 to 0.5 mg/mL were plotted against their respective concentrations. The same was done for dilutions from 0.08-0.02 mg/mL. A linear trendline was plotted for the 0.08-0.02 mg/mL range. The equation y=13.312x+0.0692 was obtained with an R² value of 0.9918. This equation was used to calculate protein concentrations of the test samples of both control (A) and formulation (B) from their respective MAVs (FIG. 12A). Collagen mass per sample was calculated by multiplying the total solution volume present outside during the diffusion experiment (90 mL, 89.8 mL, 89.6 mL or 89.4 mL). Concentrations were plotted against the timepoints to obtain a trend of the amount of protein releasing out from a solution/formulation. Two such trends were obtained finally, one for Solution (A) and the other for Formulation (B) (FIG. 12B). As shown, protein gradually diffused from both Solution (A) and Formulation (B). Protein appears to diffuse more slowly from Formulation (B), indicating an attraction between the HA and the collagen.

### Example 65 (Fluorescent Labeling for Collagen Protein Release Kinetics)

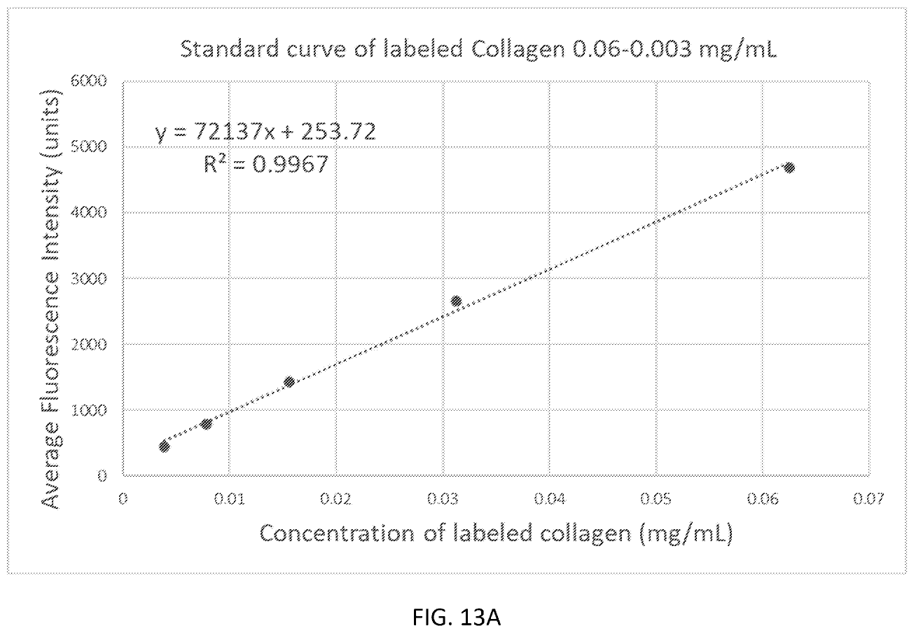

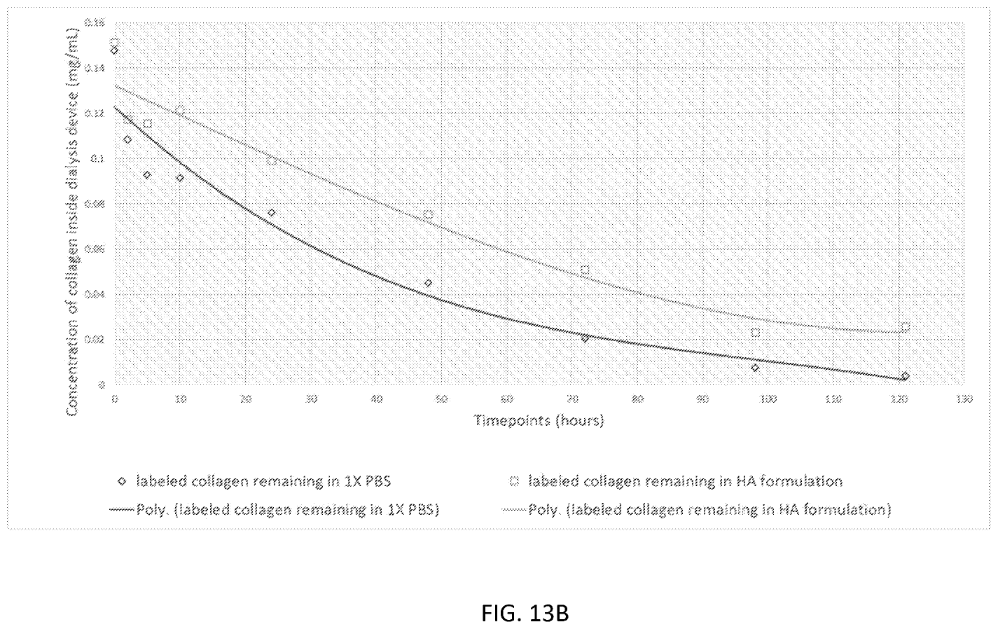

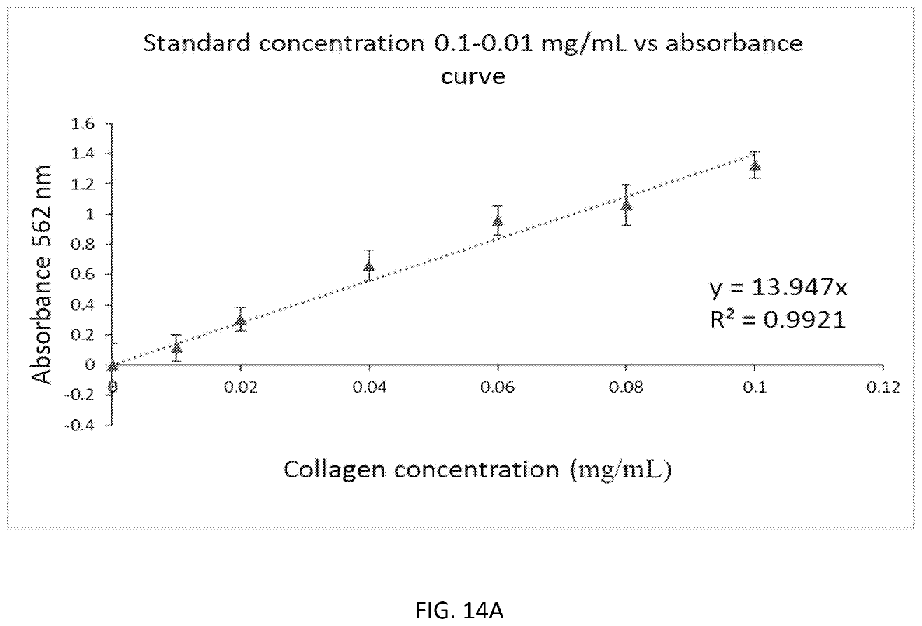

The release kinetics of Control solution (A) a recombinant collagen having amino acid sequence of SEQ ID NO: 1, and Formulation (B) a formulation of a recombinant collagen having amino acid sequence of SEQ ID NO: 1 and hyaluronic acid were fluorescently labeled for displaying release kinetics data. The results are shown in FIGS. 13A and 13B.

### Analysis

All dilutions for standard curve and timepoints from inside for (A) and (B) were pipetted on 96 well plates. 3 technical replicates of 100 μL each were pipetted. The fluorescence intensity was measured by setting excitation and emission wavelengths at 680 and 702 nm.

Standard curve: Triplicate intensity values obtained of each sample and dilution were averaged to obtain mean intensity values (MIV). MIV of serial dilutions from 0.0625, 0.03125, 0.015625, 0.0078125, 0.00390625 mg/mL were plotted against their respective concentrations. A linear trendline was plotted (FIG. 13A). The equation y=72137x+253.72 was obtained with an R2 value of 0.9967. This equation was used to calculate protein concentrations of the test samples of both solution (A) and formulation (B) from their respective MIVs. Unlabeled collagen concentration was calculated by multiplying labeled concentration values by 50. These concentrations were plotted against timepoints to give a trend of the amount of protein remaining inside the solution/formulation (FIG. 13B). As shown in FIG. 13B, the collagen diffuses from both Solution (A) and Formulation (B) at a gradual rate. The collagen appears to diffuse more slowly from the Formulation with HA (Formulation (B)), indicating an attraction between the HA and the collagen.

### Example 66 (Collagen Protein Release Kinetics/Crosslinked HA)

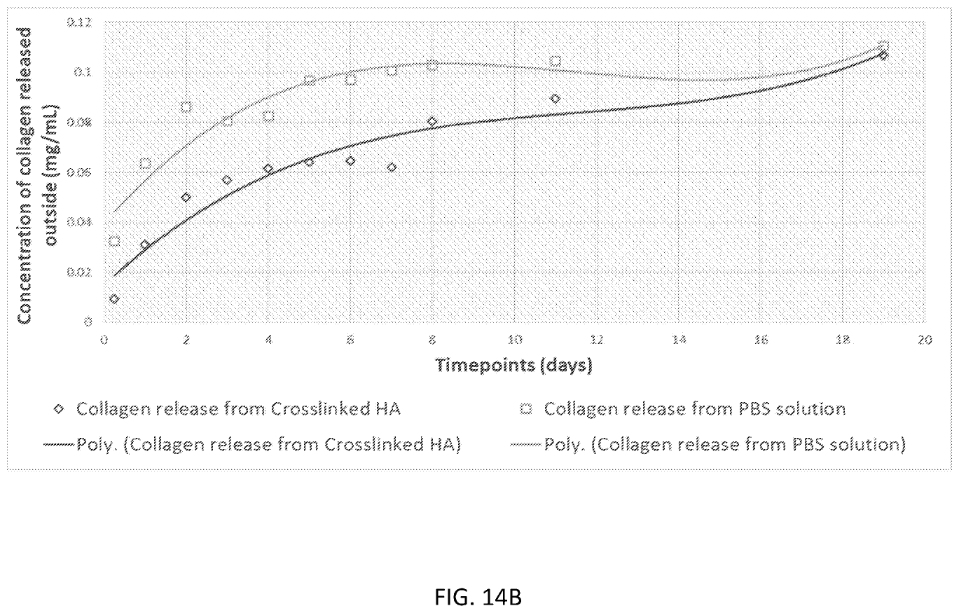

The release kinetics of a solution of (A) a 1% solution of recombinant collagen having amino acid sequence of SEQ ID NO: 1, and (B) a formulation of a recombinant collagen having amino acid sequence of SEQ ID NO: 1 and crosslinked hyaluronic acid were investigated. The results are shown in FIGS. 14A and 14B.

### Analysis

Triplicate absorbance values were obtained of each sample and dilutions were averaged and subtracted from the blank (1×PBS+ value to obtain mean absorbance values (MAV) only from collagen in each sample/dilution. Standard deviation and error were calculated for each value.

MAV of serial dilutions from 0.2 to 0.01 mg/mL were plotted against their respective concentrations. A linear trendline was plotted for the 0.2-0.01 mg/mL range (FIG. 14A). The equation y=13.947x was obtained with an R2 value of 0.9921. The trendline is in good agreement with expectation and is suitable for use converting absorbance values to concentration for the diffusion experiment.

This equation was used to calculate collagen concentrations of the test samples of both Control (A) and Formulation (B) from their respective MAVs. Collagen mass per sample was calculated by multiplying the total solution volume present outside during the diffusion experiment (95 mL, 94 mL, 93 mL or 92 mL).

Concentrations were plotted against the timepoints to obtain a trend of the amount of collagen releasing out from a solution/formulation (FIG. 14B). Two such trends were obtained finally, one for collagen solution (A) and the other for collagen/HA formulation (B). As shown in FIG. 7B, the collagen release rate from the control solution (A) was similar to previous control examples, however, the collagen released much more slowly from the crosslinked HA/collagen formulation (B). For example, the amount of protein released from Formulation (B) at 8 days required only 3 days on average to diffuse from the control solution (A). After 19 days the collagen had fully released from the formulation (B).

### Example 67

A polyethylene glycol (PEG) and collagen mixture was prepared and dried, and visually examined. Example 67 was prepared using the method described in Example 7, except Polymer 1 was replaced with polyethylene glycol (molecular weight of 8 kDa; Sigma). The final solution was visually transparent, indicating that the two polymers were miscible in solution. Films were prepared using the method described in Example 9. The dried film separated into two phases where small, cracked pieces of a brittle and opaque material was surrounded by a ring of a brown and transparent material.

### Example 68

A hyaluronic acid and collagen mixture was prepared and visually examined. Example 68 was prepared using the method described in Example 20 except Polymer 1 was replaced with HumaColl21® powder from Geltor. A 2 wt % 50 kDa HA solution was prepared using the method described in Example 20. The two solutions were mixed together in a 1:1 (w/w) ratio in a 15 ml conical tube and mixed with on a stir plate at 10 RPM for 16 hours. The final solution was visually transparent. A film was prepared using the method described in Example 9, except using an aluminum weighing pan. The dried film was transparent with a milky ring along the edge of the film, which was likely the preservative.

This example demonstrates that solutions including HumaColl21® powder and 50 kDa HA are miscible.

### Example 69

A series of in vivo experiments using the chick chorioallantoic membrane (CAM) assay are performed. T-HEp3 cells are inoculated on CAMs with type I, III or IV collagen or with Dulbecco's phosphate-buffered saline (DPBS) as vehicle control. T-HEp3 cells co-injected with the recombinant collagen fragment and the vehicle control are observed for tumor cell growth. T-HEp3 cells are injected into the right flank and T-HEp3 cells with type III collagen contralaterally in nude mice. Tumor growth is observed.

Mice are orthotopically injected with 4T1 or D2A1 cells and co-injected with the recombinant collagen fragment. Phospho-histone H3 protein levels are observed to determine cell proliferation. Nuclear dormancy markers are observed and live-cell imaging of T-HEp3 cells expressing the CDK2 cell cycle sensor is performed to determine induction of dormancy.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

1. A method of treating a cancer in a subject in need thereof, the method comprising contacting a cellular surface on the subject with an effective amount of a recombinant collagen fragment having a sequence identity of at least about 85% to the amino acid sequence set forth in SEQ ID NO: 1, or having the amino acid sequence set forth in any one of SEQ ID NOs: 2-972, SEQ ID NO: 973, or SEQ ID NO: 974-1002.

2.-3. (canceled)

4. The method of claim 1, wherein the recombinant collagen fragment has the amino acid sequence set forth in SEQ ID NO: 1.

5.-8. (canceled)

9. The method of claim 1, wherein the cancer is characterized by the presence of a solid tumor.

10.-21. (canceled)

22. The method of claim 1, wherein the recombinant collagen fragment is formulated in a pharmaceutically acceptable composition.

23. The method of claim 1, wherein the recombinant collagen fragment is formulated in a therapeutic biomaterial.

24. The method of claim 23, wherein the therapeutic biomaterial is a protein polyurethane alloy.

25. The method of claim 24, wherein the protein is dissolved in the polyurethane.

26. (canceled)

27. The method of claim 24, wherein the alloy comprises about 10 wt % to about 50 wt % of the fragment and about 50 wt % to about 90 wt % of the polyurethane.

28. The method of claim 24, wherein the alloy comprises about 20 wt % to about 35 wt % of the protein and about 65 wt % to about 80 wt % of the polyurethane.

29. The method of claim 24, wherein the alloy is free of, or substantially free of, particles of the recombinant collagen fragment having an average diameter of greater than 1 micron.

30. A therapeutic biomaterial comprising a recombinant collagen fragment having a sequence identity of at least about 85% to the amino acid sequence set forth in SEQ ID NO: 1, SEQ ID NOs: 2-972, SEQ ID NO: 973, or SEQ ID NOs: 974-1002, and a pharmaceutically acceptable scaffold.

31.-32. (canceled)

33. The therapeutic biomaterial of claim 30, wherein the collagen fragment has the amino acid sequence set forth in SEQ ID NO: 1.

34.-42. (canceled)

43. The therapeutic biomaterial of claim 30, wherein the pharmaceutically acceptable scaffold comprises a mixture of (i) a member selected from the group consisting of hyaluronic acid, polyvinylpyrrolidone, polyacrylamide, or poly(ethylene oxide), or poly(2-oxazoline) s, or polyethylenimine, or carboxymethylcellulose, or chondroitin sulfate, or an acetylated hyaluronate, or zinc hyaluronate, and (ii) the recombinant collagen fragment.

44.-57. (canceled)

58. A treatment method comprising applying the therapeutic biomaterial of any one of claims 30-57 for treating a wound resulting from resection of a primary tumor.

59.-81. (canceled)
